(19) United States
(12) Patent Application Publication
(10) Pub. No.: US 2024/0254203 A1
(43) Pub. Date: Aug. 1, 2024

(54) ANTIBODY-BASED ANTIVIRUS COMPOSITIONS AND METHODS OF USE

(71) Applicant: Achelois BioPharma, Inc., Redwood City, CA (US)

(72) Inventors: Michael CHEN, Palo Alto, CA (US); Chang-Zheng CHEN, Palo Alto, CA (US); Yiling LUO, South San Francisco, CA (US); Hua ZHOU, San Mateo, CA (US)

(21) Appl. No.: 18/560,621

(22) PCT Filed: May 19, 2022
(86) PCT No.: PCT/US2022/030114

§ 371 Date: Nov. 13, 2023

Related U.S. Application Data
(60) Provisional application No. 63/191,212, filed on May 20, 2021.
(60) Provisional application No. 63/191,216, filed on May 20, 2021.

(51) Int. Cl. C07K 16/10; A61P 31/14; C07K 14/005

(52) U.S. Cl. CPC C07K 16/1003; A61P 31/14; C07K 14/005; C07K 16/1018; C07K 2317/31; C07K 2317/35; C07K 2317/622; C07K 2319/02; C07K 2319/73; C12N 2760/16122; C12N 2760/18422; C12N 2760/20222; C12N 2770/20021; C12N 2770/24122

(57) ABSTRACT

Provided herein are antibody-based antiviruses comprising a fusion protein that comprises a transmembrane polypeptide and an antibody which binds to a surface protein of a virus, wherein the fusion protein is expressed at a valency of at least about 10 copies on a surface of the antivirus, and wherein the antibody neutralizes the virus when expressed within the fusion protein on the surface of the antivirus but does not neutralize the virus when expressed as an isolated antibody. Further provided herein are antibody-based antiviruses for treating a viral infection (e.g., SARS-COV-2).

### CROSS-REFERENCE

This application claims the benefit of U.S. Provisional Application No. 63/191,212 filed May 20, 2021, and U.S. Provisional Application No. 63/191,216 filed May 20, 2021, each of which is incorporated herein by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

In one aspect, provided herein is an antivirus comprising a fusion protein that comprises a transmembrane polypeptide and an antibody which binds to a surface protein of a virus wherein the fusion protein is expressed at a valency of at least about 10 copies on a surface of the antivirus, wherein the antibody neutralizes the virus when expressed within the fusion protein on the surface of the antivirus, but does not neutralize the virus when expressed as an isolated antibody. In some embodiments, the fusion protein further comprises an oligomerization domain. In some embodiments, the oligomerization domain is a dimerization domain, a trimerization domain, or a tetramerization domain. In some embodiments, the dimerization domain comprises a leucine zipper dimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the tetramerization domain comprises an influenza neuraminidase stem domain. In some embodiments, the oligomerization domain comprises an amino acid sequence that is at least about 80%, 85%, 90%, 95%, or 99% identical to any one of SEQ ID NOs: 30-43. In some embodiments, when the fusion protein is expressed on the surface of the antivirus, the oligomerization domain is outside of the antivirus. In some embodiments, when the fusion protein is expressed on the surface of the antivirus, the oligomerization domain is outside of the antivirus and adjacent to a signal peptide. In some embodiments, when the fusion protein is expressed on the surface of the antivirus, the oligomerization domain is inside of the antivirus. In some embodiments, when the fusion protein is expressed on the surface of the antivirus, the oligomerization domain is inside of the antivirus and adjacent to the transmembrane polypeptide. In some embodiments, the fusion protein comprises a signal peptide. In some embodiments, domains of the fusion protein are arranged from the N-terminus to the C-terminus in the following orders: (a) signal peptide, antibody which binds to a surface protein of a virus, oligomerization domain, transmembrane polypeptide, and cytosolic domain; (b) signal peptide, antibody which binds to a surface protein of a virus, transmembrane polypeptide, oligomerization domain, and cytosolic domain; or (c) signal peptide, oligomerization domain, antibody which binds to a surface protein of a virus, transmembrane polypeptide, and cytosolic domain. In some embodiments, the antibody comprises a single chain variable fragment (scFv), a single domain antibody, an Fv, a VH domain, a VL domain, a Fab fragment, a monoclonal antibody, F(ab′), F(ab′)2, single chain antibodies, diabodies, or a scFv-Fc. In some embodiments, the antibody binds specifically to the surface protein of the virus. In some embodiments, the antibody is a multispecific antibody. In some embodiments, the multispecific antibody binds specifically to more than one epitope on the surface protein of the virus. In some embodiments, the multispecific antibody comprises a tandem scFv format. In some embodiments, the virus comprises SARS COV-1, SARS COV-2, influenza, or MERS CoV virus. In some embodiments, the antibody comprises an amino acid sequence from at least one complementarity determining region of 0304-4A10, 2M-14E5, 9A1, COV2-2021, COV2-2026, COV2-2146, 2M-10B11, C021, C018 and BG4-5. In some embodiments, the antibody comprises an amino acid sequence at least about 80%, 85%, 90%, 95%, or 99% identical to any one of SEQ ID NOs: 6-14, 20-28 and 81-82. In some embodiments, the transmembrane polypeptide anchors the fusion protein to a bilayer of the antivirus. In some embodiments, the transmembrane polypeptide comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA). In some embodiments, the transmembrane polypeptide comprises a transmembrane domain comprising an amino acid sequence at least about 80%, 85%, 90%, 95%, or 99% identical to any one of SEQ ID NOs.: 44-52. In some embodiments, the transmembrane polypeptide comprises an amino acid sequence at least about 80%, 85%, 90%, 95%, or 99% identical to that set forth in SEQ ID NO: 29. In some embodiments, the fusion protein is expressed at a valency of about 10 copies on a surface of the antivirus. In some embodiments, the fusion protein is expressed at a valency of about 10 to 15 copies on a surface of the antivirus. In some embodiments, the fusion protein is expressed at a valency of at least about 25 copies on a surface of the antivirus. In some embodiments, the fusion protein is expressed at a valency of at least about 50 copies on a surface of the antivirus. In some embodiments, the fusion protein is expressed at a valency of at least about 100 copies on a surface of the antivirus. In some embodiments, the fusion protein is expressed at a valency of at least about 200 copies on a surface of the antivirus. In some embodiments, the fusion protein is expressed at a valency of at least about 400 copies on a surface of the antivirus. In some embodiments, the fusion protein is expressed at a valency of at least about 600 copies on a surface of the antivirus. In some embodiments, the fusion protein is expressed at a valency of at least about 1000 copies on a surface of the antivirus. In some embodiments, the antivirus is an enveloped particle. In some embodiments, the antivirus does not comprise viral genetic material. In some embodiments, the antivirus comprises a lipid bilayer. In some embodiments, the antivirus is a virus. In some embodiments, the antivirus is a replication incompetent virus. In some embodiments, the antivirus is a replication competent virus. In some embodiments, the antivirus is a viral-like particle. In some embodiments, the fusion protein comprises an amino acid sequence at least about 80%, 85%, 90%, 95%, or 99% identical to any one of SEQ ID NOs: 59-70, 72, 74, and 78. In some embodiments, the antivirus further comprises a second fusion protein that comprises transmembrane polypeptide and a second antibody which binds to a surface protein of the virus, wherein the second antibody comprises a CDR sequence that has less than 100% sequence identity to an equivalent CDR sequence of the antibody. In some embodiments, the fusion protein and the second fusion protein comprise the same transmembrane polypeptide. In some embodiments, the fusion protein and the second fusion protein comprise different transmembrane polypeptides. In some embodiments, the second antibody binds to the same surface protein as the antibody. In some embodiments, the second antibody binds to a different surface protein as the antibody. In some embodiments, the second antibody is a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc. In some embodiments, the second antibody binds specifically to the surface protein of the virus. In some embodiments, the second antibody is a multispecific antibody. In some embodiments, the second multispecific antibody binds specifically to more than one epitope on the surface protein of the virus. In some embodiments, the second antibody comprises an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15 and BG10-19. In some embodiments, the second antibody comprises an amino acid sequence at least about 80%, 85%, 90%, 95%, or 99% identical to any one of SEQ ID NOs: 1-28, and 81-86. In some embodiments, the second antibody neutralizes the virus when expressed within the fusion protein but does not neutralize the virus when expressed as an isolated antibody. In some embodiments, the second antibody neutralizes the virus when expressed within the fusion protein and neutralizes the virus when expressed as an isolated antibody. In some embodiments, the second fusion protein further comprises an oligomerization domain. In some embodiments, the oligomerization domain is a dimerization domain or a trimerization domain. In some embodiments, the oligomerization domain comprises the D4 trimerization domain of VSV-G protein. In some embodiments, the antibody comprises a single chain variable fragment (scFv) and an amino acid sequence from at least one complementarity determining region of 0304-4A10, 2M-14E5, 9A1, COV2-2021, COV2-2026, COV2-2146, 2M-10B11, C021, C018, and BG4-5. The antivirus of claim 46, wherein the second fusion protein comprises an amino acid sequence at least about 80%, 85%, 90%, 95%, or 99% identical to any one of SEQ ID NOs: 53-72, 74-76, or 78. In some embodiments, (a) the antibody comprises a scFv and an amino acid sequence from at least one complementarity determining region of 0304-4A10, 2M-14E5, 9A1, COV2-2021, COV2-2026, COV2-2146, 2M-10B11, C021, C018 and BG4-5; and (b) the transmembrane polypeptide comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA). In some embodiments, (a) the antibody comprises a scFv and an amino acid sequence from at least one complementarity determining region of 0304-4A10, 2M-14E5, 9A1, COV2-2021, COV2-2026, COV2-2146, 2M-10B11, C021, C018 and BG4-5; (b) the transmembrane polypeptide comprises a transmembrane domain comprising an amino acid sequence at least about 80%, 85%, 90%, 95%, or 99% identical to one of SEQ ID NOs.: 44-52. In some embodiments, (a) the antibody comprises a scFv and an amino acid sequence at least about 80%, 85%, 90%, 95%, or 99% identical to any one of SEQ ID NOs: 6-14, 20-28 and 81-82; and (b) the transmembrane polypeptide comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA). In some embodiments, (a) the antibody comprises a scFv and an amino acid sequence from at least one complementarity determining region of 0304-4A10, 2M-14E5, 9A1, COV2-2021, COV2-2026, COV2-2146, 2M-10B11, C021, C018 and BG4-5; and (b) the oligomerization domain comprises a leucine zipper dimerization domain, a D4 trimerization domain of VSV-G protein, a Dengue E protein post-fusion trimerization domain, a foldon trimerization domain, or an influenza neuraminidase stem tetramerization domain. In some embodiments, (a) the antibody comprises a single chain variable fragment (scFv) and an amino acid sequence from at least one complementarity determining region of 0304-4A10, 2M-14E5, 9A1, COV2-2021, COV2-2026, COV2-2146, 2M-10B11, C021, C018 and BG4-5; and (b) the oligomerization domain comprises an amino acid sequence at least about 80%, 85%, 90%, 95%, or 99% identical to any one of SEQ ID NOs: 30-43. In some embodiments, (a) the antibody comprises a scFv and an amino acid sequence at least about 80%, 85%, 90%, 95%, or 99% identical to any one of SEQ ID NOs: 6-14, 20-28 and 81-82; and (b) the oligomerization domain comprises a leucine zipper dimerization domain, a D4 trimerization domain of VSV-G protein, a Dengue E protein post-fusion trimerization domain, a foldon trimerization domain, or an influenza neuraminidase stem tetramerization domain. In some embodiments, (a) the antibody comprises a scFv and an amino acid sequence from at least one complementarity determining region of 0304-4A10, 2M-14E5, 9A1, COV2-2021, COV2-2026, COV2-2146, 2M-10B11, C021, C018 and BG4-5; and (b) the transmembrane polypeptide comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA); and (c) the oligomerization domain comprises a leucine zipper dimerization domain, a D4 trimerization domain of VSV-G protein, a Dengue E protein post-fusion trimerization domain, a foldon trimerization domain, or an influenza neuraminidase stem tetramerization domain. In some embodiments, (a) the antibody comprises a scFv and an amino acid sequence from at least one complementarity determining region of 0304-4A10, 2M-14E5, 9A1, COV2-2021, COV2-2026, COV2-2146, 2M-10B11, C021, C018 and BG4-5; (b) the transmembrane polypeptide comprises a transmembrane domain comprising an amino acid sequence at least about 80%, 85%, 90%, 95%, or 99% identical to one of SEQ ID NOs.: 44-52; and (c) the oligomerization domain comprises a leucine zipper dimerization domain, a D4 trimerization domain of VSV-G protein, a Dengue E protein post-fusion trimerization domain, a foldon trimerization domain, an influenza neuraminidase stem tetramerization domain. In some embodiments, (a) the antibody comprises a scFv and an amino acid sequence from at least one complementarity determining region of 0304-4A10, 2M-14E5, 9A1, COV2-2021, COV2-2026, COV2-2146, 2M-10B11, C021, C018 and BG4-5; (b) the transmembrane polypeptide comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA); and (c) the oligomerization domain comprises an amino acid sequence at least about 80%, 85%, 90%, 95%, or 99% identical to any one of SEQ ID NOs: 30-43. In some embodiments, (a) the antibody comprises a scFv and an amino acid sequence from at least one complementarity determining region of 0304-4A10, 2M-14E5, 9A1, COV2-2021, COV2-2026, COV2-2146, 2M-10B11, C021, C018 and BG4-5; (b) the transmembrane polypeptide comprises a transmembrane domain comprising an amino acid sequence at least about 80%, 85%, 90%, 95%, or 99% identical to one of SEQ ID NOs.: 44-52; and (c) the oligomerization domain comprises an amino acid sequence at least about 80%, 85%, 90%, 95%, or 99% identical to any one of SEQ ID NOs: 30-43. In some embodiments, (a) the antibody comprises a scFv and an amino acid sequence at least about 80%, 85%, 90%, 95%, or 99% identical to any one of SEQ ID NOs: 6-14, 20-28 and 81-82; (b) the transmembrane polypeptide comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA); and (c) the oligomerization domain comprises a leucine zipper dimerization domain, a D4 trimerization domain of VSV-G protein, a Dengue E protein post-fusion trimerization domain, a foldon trimerization domain, an influenza neuraminidase stem tetramerization domain. In some embodiments, (a) the antibody comprises a scFv and an amino acid sequence at least about 80%, 85%, 90%, 95%, or 99% identical to any one of SEQ ID NOs: 6-14, 20-28 and 81-82; (b) the transmembrane polypeptide comprises a transmembrane domain comprising an amino acid sequence at least about 80%, 85%, 90%, 95%, or 99% identical to one of SEQ ID NOs.: 44-52; and (c) the oligomerization domain comprises a leucine zipper dimerization domain, a D4 trimerization domain of VSV-G protein, a Dengue E protein post-fusion trimerization domain, a foldon trimerization domain, an influenza neuraminidase stem tetramerization domain. In some embodiments, (a) the antibody comprises a scFv and an amino acid sequence at least about 80%, 85%, 90%, 95%, or 99% identical to any one of SEQ ID NOs: 6-14, 20-28 and 81-82; (b) the transmembrane polypeptide comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA); and (c) the oligomerization domain comprises an amino acid sequence at least about 80%, 85%, 90%, 95%, or 99% identical to any one of SEQ ID NOs: 30-43. In some embodiments, (a) the second antibody comprises a scFv and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19; and (b) the transmembrane polypeptide of the second fusion protein comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA). In some embodiments, (a) the second antibody comprises a scFv and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19; and (b) the transmembrane polypeptide of the second fusion protein comprises a transmembrane domain comprising an amino acid sequence at least about 80%, 85%, 90%, 95%, or 99% identical to one of SEQ ID NOs.: 44-52. In some embodiments, (a) the second antibody comprises a scFv and an amino acid sequence at least about 80%, 85%, 90%, 95%, or 99% identical to any one of SEQ ID NOs: 1-28; and (b) the transmembrane polypeptide of the second fusion protein comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA). In some embodiments, (a) the second antibody comprises a scFv and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19; and (b) the oligomerization domain of the second fusion protein comprises a leucine zipper dimerization domain, a D4 trimerization domain of VSV-G protein, a Dengue E protein post-fusion trimerization domain, a foldon trimerization domain, or an influenza neuraminidase stem tetramerization domain. In some embodiments, (a) the second antibody comprises a single chain variable fragment (scFv) and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19; and (b) the oligomerization domain of the second fusion protein comprises an amino acid sequence at least about 80%, 85%, 90%, 95%, or 99% identical to any one of SEQ ID NOs: 30-43. In some embodiments, (a) the second antibody comprises a scFv and an amino acid sequence at least about 80%, 85%, 90%, 95%, or 99% identical to any one of SEQ ID NOs: 1-28; and (b) the oligomerization domain of the second fusion protein comprises a leucine zipper dimerization domain, a D4 trimerization domain of VSV-G protein, a Dengue E protein post-fusion trimerization domain, a foldon trimerization domain, or an influenza neuraminidase stem tetramerization domain. In some embodiments, (a) the second antibody comprises a scFv and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19; and (b) the transmembrane polypeptide of the second fusion protein comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA); and (c) the oligomerization domain of the second fusion protein comprises a leucine zipper dimerization domain, a D4 trimerization domain of VSV-G protein, a Dengue E protein post-fusion trimerization domain, a foldon trimerization domain, or an influenza neuraminidase stem tetramerization domain. In some embodiments, (a) the second antibody comprises a scFv and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19; (b) the transmembrane polypeptide of the second fusion protein comprises a transmembrane domain comprising an amino acid sequence at least about 80%, 85%, 90%, 95%, or 99% identical to one of SEQ ID NOs.: 44-52; and (c) the oligomerization domain of the second fusion protein comprises a leucine zipper dimerization domain, a D4 trimerization domain of VSV-G protein, a Dengue E protein post-fusion trimerization domain, a foldon trimerization domain, an influenza neuraminidase stem tetramerization domain. In some embodiments, (a) the second antibody comprises a scFv and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19; (b) the transmembrane polypeptide of the second fusion protein comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA); and (c) the oligomerization domain of the second fusion protein comprises an amino acid sequence at least about 80%, 85%, 90%, 95%, or 99% identical to any one of SEQ ID NOs: 30-43. In some embodiments, (a) the second antibody comprises a scFv and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19; (b) the transmembrane polypeptide of the second fusion protein comprises a transmembrane domain comprising an amino acid sequence at least about 80%, 85%, 90%, 95%, or 99% identical to one of SEQ ID NOs.: 44-52; and (c) the oligomerization domain of the second fusion protein comprises an amino acid sequence at least about 80%, 85%, 90%, 95%, or 99% identical to any one of SEQ ID NOs: 30-43. In some embodiments, (a) the second antibody comprises a scFv and an amino acid sequence at least about 80%, 85%, 90%, 95%, or 99% identical to any one of SEQ ID NOs: 1-28 and 81-86; (b) the transmembrane polypeptide of the second fusion protein comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA); and (c) the oligomerization domain of the second fusion protein comprises a leucine zipper dimerization domain, a D4 trimerization domain of VSV-G protein, a Dengue E protein post-fusion trimerization domain, a foldon trimerization domain, an influenza neuraminidase stem tetramerization domain. In some embodiments, (a) the second antibody comprises a scFv and an amino acid sequence at least about 80%, 85%, 90%, 95%, or 99% identical to any one of SEQ ID NOs: 1-28 and 81-86; (b) the transmembrane polypeptide of the second fusion protein comprises a transmembrane domain comprising an amino acid sequence at least about 80%, 85%, 90%, 95%, or 99% identical to one of SEQ ID NOs.: 44-52; and (c) the oligomerization domain of the second fusion protein comprises a leucine zipper dimerization domain, a D4 trimerization domain of VSV-G protein, a Dengue E protein post-fusion trimerization domain, a foldon trimerization domain, an influenza neuraminidase stem tetramerization domain. In some embodiments, (a) the second antibody comprises a scFv and an amino acid sequence at least about 80%, 85%, 90%, 95%, or 99% identical to any one of SEQ ID NOs: 1-28 and 81-86; (b) the transmembrane polypeptide of the second fusion protein comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA); and (c) the oligomerization domain of the second fusion protein comprises an amino acid sequence at least about 80%, 85%, 90%, 95%, or 99% identical to any one of SEQ ID NOs: 30-43. In some embodiments, the antibody is multispecific antibody that comprises a tandem scFv format comprising an amino acid sequence from at least one complementarity determining region of 0304-4A10, 2M-14E5, 9A1, COV2-2021, COV2-2026, COV2-2146, 2M-10B11, C021, C018 and BG4-5 and an amino acid sequence from at least one complementarity determining region of 0304-4A10, 2M-14E5, 9A1, COV2-2021, COV2-2026, COV2-2146, 2M-10B11, C021, C018 and BG4-5 and the fusion protein oligomerization domain comprises the D4 trimerization domain of VSV-G protein. In some embodiments, the second antibody is multispecific antibody that comprises a tandem scFv format comprising an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19 and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19 and the second fusion protein oligomerization domain comprises the D4 trimerization domain of VSV-G protein.

In another aspect, provided herein is a composition comprising an antibody-based antivirus wherein the antibody-based antivirus comprises an enveloped particle that displays at least about 10 copies of an antibody on a surface of the antivirus, wherein the antibody binds to at least one surface protein of a virus, wherein one of the at least one surface protein of the virus comprises an oligomerized format, wherein the antibody neutralizes the virus when expressed on the surface of the antivirus, but does not neutralize the virus when expressed as an isolated antibody.

In another aspect, provided herein is a composition comprising an antibody-based antivirus wherein the antibody-based antivirus comprises an enveloped particle that displays at least about 10 copies of an antibody on a surface of the antivirus, wherein the antibody comprises an oligomerized format and binds to at least one surface protein of a virus, wherein one of the at least one surface protein of the virus comprises an oligomerized format, wherein the antibody neutralizes the virus when expressed as an isolated antibody.

### BRIEF DESCRIPTION OF THE DRAWINGS

The novel features of the disclosure are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates that SARS COV-2 virions enter host cells through multivalent interactions with host cell receptors.

FIG. 2 illustrates that SARS COV-2 entry can be blocked by neutralizing antibodies.

FIG. 3 illustrates that antivirus-multivalent antibody particles may block SARS COV-2 infection more effectively.

FIG. 4 illustrates that antivirus avidity may be optimized by displaying oligomerized antibody to mirror viral spike oligomerization.

FIG. 5 illustrates that trimeric antiviruses may have block SARS COV-2 entry through multivalent binding.

FIG. 6A illustrates one embodiment of design and production of antiviruses displaying monomeric or trimeric scFv antibodies.

FIG. 6B illustrates another embodiment of design and production of antiviruses displaying monomeric or trimeric scFv antibodies.

FIG. 6C illustrates the qualitative analyses of monomeric or trimeric display of scFv on antiviruses by Western blot under reducing or non-reducing condition.

FIG. 6D and FIG. 6E illustrate quantitative Western blot analyses of antiviruses displaying monomeric or trimeric scFv antibodies.

FIG. 7A and FIG. 7B illustrate neutralization effects of soluble scFv antibodies and antiviruses displaying monomeric or trimeric scFv antibodies on SARS COV-2 pseudovirus.

FIG. 7C illustrates inhibition of live SARS COV-2 infection by monomeric and trimeric aRBD:C021 antivirus.

FIG. 8A illustrates the 3-D structural visualization of SARS COV-2 spike protein.

FIG. 8B illustrates production and quantitation of antiviruses displaying non-neutralizing SARS COV-2 scFv antibodies.

FIG. 8C illustrates the binding affinity of SARS COV-2 scFv non-neutralizing antibodies and the IC₅₀ values their corresponding antiviruses.

FIG. 8D illustrates the neutralizing efficacy of antiviruses displaying trimeric SARS CoV-2 scFv antibodies targeting the RBD region of the spike protein.

FIG. 8E illustrates the efficiency of SARS COV-2 infection suppression by antivirus targeting the RBD region of the SARS COV-2 spike protein.

FIG. 8F illustrates the neutralizing efficacy of antiviruses displaying trimeric SARS CoV-2 scFv antibodies targeting the NTD region of the spike protein.

FIG. 8G illustrates the efficiency of SARS COV-2 infection suppression by antivirus targeting the NTD region of the SARS COV-2 spike protein.

FIG. 8H illustrates the neutralizing efficacy of antiviruses displaying trimeric SARS CoV-2 scFv antibodies targeting the S2 region of the spike protein.

FIG. 8I illustrates the efficiency of SARS COV-2 infection suppression by antivirus targeting the S2 region of the SARS COV-2 spike protein.

FIG. 8J illustrates inhibition of SARS COV-2 pseudovirus infection by antivirus displaying neutralizing scFv antibodies.

FIG. 8K and FIG. 8L illustrate fold suppression and stoichiometric neutralization ratios of inhibition of SARS COV-2 pseudovirus by antivirus.

FIG. 9A illustrates the design and production of antiviruses for SARS COV-1, MERS CoV and H5N1 influenza.

FIG. 9B illustrates the production of antiviruses for SARS COV-1, MERS COV and H5N1 influenza by Western blot.

FIG. 9C illustrates inhibition of SARS COV-1, MERS CoV and H5N1 Influenza pseudoviruses infection by αRBD:80R/VM, αNTD:7D10/VM, and αHA:FI6/D4 antiviruses, respectively.

FIG. 10A illustrates the pre-attachment and post-attachment viral neutralization by multivalent antibody antiviruses.

FIG. 10B illustrates pre-attachment and post-attachment neutralization of SARS CoV-1 pseudovirus by αRBD:H4/VM antivirus.

FIG. 10C illustrates pre-attachment and post-attachment neutralization of SARS CoV-2 pseudovirus by αRBD:80R/VM antivirus.

FIG. 11A illustrates bi-specific antiviruses displaying tandem scFv antibodies with distinct antigen recognition.

FIG. 11B illustrates bi-specific antiviruses displaying mixed monomeric and trimeric scFv antibodies with distinct antigen recognition.

FIG. 11C illustrates monospecific antiviruses displaying monomeric or trimeric scFv antibodies and bispecific antiviruses displaying mixed or tandem bispecific scFv antibodies.

FIG. 11D illustrates validation of tandem bi-specific antiviruses by Western-blot.

FIG. 11E illustrates neutralization of SARS COV-2 pseudovirus by tandem scFv (αRBD-αNTD)/D4 bi-specific antiviruses.

FIG. 11F illustrates neutralization of H5N1 Influenza pseudovirus by tandem scFv (αHA-αNA)/D4 bi-specific antiviruses.

FIG. 11G illustrates validation of mixed bi-specific antiviruses by Western-blot.

FIG. 11H illustrates neutralization of SARS COV-2 pseudovirus by bi-specific antivirus co-displaying αRBD:C018/VM and αNTD:CV26/D4 scFv antibodies.

FIG. 11I illustrates neutralization of live SARS COV-2 in PRNT by bispecific antiviruses displaying tandem (αRBD-αNTD)/D4 scFv antibodies or mixed (αRBD:C018/VM)(αNTD:CV26/D4) scFv antibodies.

FIG. 12A illustrates neutralizing effects of αRBD:C021/D4 antivirus on SARS COV-2 variants with spike mutations.

FIG. 12B illustrates neutralizing effects of REGN 0933 antibody on SARS COV-2 variants with spike mutations.

FIG. 12C illustrates neutralizing effects of REGN 0989 antibody on SARS COV-2 variants with spike mutations.

FIG. 12D illustrates neutralizing effects of tandem (αRBD-αNTD)/D4 bi-specific antivirus on SARS COV-2 variants with spike mutations.

FIG. 12E illustrates neutralizing effects of mixed (αRBD:C018/VM)(αNTD:CV26/D4) bi-specific antivirus on SARS COV-2 variants with spike mutations.

FIG. 13A illustrates neutralizing effects of αRBD:BG4-5/VM antivirus on SARS CoV-2 variants with spike mutations.

FIG. 13B illustrates neutralizing effects of αRBD:BG7-15/D4 antivirus on SARS CoV-2 variants with spike mutations.

FIG. 13C illustrates neutralizing effects of αRBD:BG10-19/D4 antivirus on SARS CoV-2 variants with spike mutations.

FIG. 13D illustrates neutralizing effects of mixed (αRBD:BG10-19/D4)(αNTD:CV21/VM) bi-specific antivirus on SARS COV-2 variants with spike mutations.

FIG. 13E illustrates neutralizing effects of mixed (αRBD:BG10-19/D4)(αNTD:CV26/VM) bi-specific antivirus on SARS COV-2 variants with spike mutations.

FIG. 13F illustrates neutralizing effects of mixed (αRBD:BG10-19/D4)(αRBD:BG4-5/VM) bi-specific antivirus on SARS COV-2 variants with spike mutations.

FIG. 13G illustrates neutralizing effects of αRBD:BG10-19/D4 antivirus on circulating SARS COV-2 wild type, Beta variant and Delta variant.

FIG. 13H illustrates neutralizing effects of mixed (αRBD:BG10-19/D4)(αNTD:CV21/VM) bi-specific antivirus on circulating SARS COV-2 wild type, Beta variant and Delta variant.

FIG. 13I illustrates neutralizing effects of mixed (αRBD:BG10-19/D4)(αRBD:BG4-5/VM) bi-specific antivirus on circulating SARS COV-2 wild type, Beta variant and Delta variant.

FIGS. 14A-14B illustrate in vivo treatment effect of αRBD:BG10-19/D4 antivirus on SARS COV-2 infection in transgenic mice.

FIG. 15A illustrates vector design for a monomeric display vector expressing a fusion protein comprising a protein linked to the VSVG transmembrane and intracellular domains.

FIG. 15B illustrates vector design for a trimeric display vector expressing a fusion protein comprising a protein linked to the D4 post-fusion trimerization domain, transmembrane domain and intracellular domain of VSV-G.

FIGS. 16A-16C illustrate generation of monomeric enveloped particles.

FIGS. 17A-17C illustrate generation of trimeric enveloped particles.

FIGS. 18A-18C illustrate generation of generation of mixed monomeric and trimeric enveloped particles.

FIG. 19A-19C illustrate vectors with various D4 configurations.

FIGS. 20A-20C illustrate vectors with various oligomerization domain configurations.

FIGS. 21A-21D illustrate production and quantitation of antiviruses displaying various scFv antibodies targeting SARS COV-2 and H5N1 Influenza.

FIGS. 22A-22C illustrate another embodiment of design and production of antiviruses displaying monomeric scFv antibodies with and without viral genomes.

FIG. 23A illustrates western blot analysis of monomeric αCoV1:80R/VM antiviruses with viral genomes.

FIG. 23B illustrates western blot analysis of monomeric αCoV1:80R/VM antiviruses without viral genomes.

FIG. 23C illustrates comparison of pseudovirus neutralization effects of αCoV1:80R/VM antiviruses with and without viral genomes.

FIG. 24A illustrates western blot analysis of monomeric αNTD:CV21/VM antiviruses with viral genomes.

FIG. 24B illustrates western blot analysis of monomeric αNTD:CV21/VM antiviruses without viral genomes.

FIG. 24C illustrates comparison of pseudovirus neutralization effects of αNTD:CV21/VM antiviruses with and without viral genomes.

FIG. 25A illustrates western blot analysis of monomeric αRBD:C021/VM antiviruses with viral genomes

FIG. 25B illustrates western blot analysis of monomeric αRBD:C021/VM antiviruses without viral genomes.

FIG. 25C illustrates comparison of pseudovirus neutralization effects of αRBD:C021/VM antiviruses with and without viral genomes.

FIGS. 26A-26C illustrate another embodiment of design and production of antiviruses displaying trimeric scFv antibodies with and without viral genomes.

FIG. 27A illustrates western blot analysis of trimeric αNTD:CV21/D4 antiviruses with viral genomes.

FIG. 27B illustrates western blot analysis of trimeric αNTD:CV21/D4 antiviruses without viral genomes.

FIG. 27C illustrates comparison of pseudovirus neutralization effects of αNTD:CV21/D4 antiviruses with and without viral genomes.

FIG. 28A illustrates western blot analysis of trimeric αRBD:C021/D4 antiviruses with viral genomes.

FIG. 28B illustrates western blot analysis of trimeric αRBD:C021/D4 antiviruses without viral genomes.

FIG. 28C illustrates comparison of pseudovirus neutralization effects of trimeric αRBD:C021/D4 antiviruses with and without viral genomes.

FIG. 29A illustrates another embodiment of design of antiviruses displaying trimeric multispecific tandem scFv antibodies.

FIG. 29B illustrates western blot analysis of trimeric multispecific tandem (αRBD-αNTD)/D4 antiviruses with viral genomes.

FIG. 29C illustrates western blot analysis of trimeric multispecific tandem (αRBD-αNTD)/D4 antiviruses without viral genomes.

FIG. 29D illustrates comparison of pseudovirus neutralization effects of trimeric multispecific tandem (αRBD-αNTD)/D4 antiviruses with and without viral genomes.

### DETAILED DESCRIPTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

### Antivirus Compositions

The COVID-19 pandemic has caused tremendous losses in human life and economic activities. Current strategies such as antibody therapies for neutralizing viruses are not entirely effective. This is in part due to viruses being able to adapt strategies to effectively gain entry of host cells while evading the control by host immune systems. Nearly all viruses utilize a multivalent strategy for attachment and entry of host cells. Each virion display hundreds of copies of spike proteins, which can simultaneously interact with multiple copies of host cell receptors and attachment proteins. In the case of coronaviruses, host cell receptors angiotensin-converting enzyme 2 (ACE2) and dipeptidyl peptidase 4 (DPP4) are used as entry receptors for SARS COV-1/2 and MERS coronaviruses, respectively. The densely packed spike proteins on the virions enable them to interact with multiple copies of ACE2 or DPP4 on the host cell surface. The boost in functional affinity that viruses receive through multivalent interactions is exponential, and nearly all enveloped and non-enveloped viruses use this multivalent strategy for attachment and host-cell entry. This provides a tremendous advantage to viruses. Most notably, the multivalent strategy enables viruses to turn relatively weak monovalent interactions with millimolar binding affinities into super-strong multivalent interactions with functional affinities in the nanomolar to picomolar range, in turn creating a high threshold for low or monovalent binders, such as neutralizing antibodies and recombinant protein inhibitors, to overcome. Moreover, viruses harness high mutation rates and multivalent binding to host cells to facilitate immune evasion. Spike mutagenesis and novel glycosylation patterns can effectively disrupt the neutralizing function of antibodies and other low-valency viral-blocking agents with little impact on viral attachment and entry. The current development of viral neutralization molecules does not address the multivalent nature of virions and host cell interaction. It is likely that mutations that are resistant to current combinations of clinically-tested neutralization antibodies will emerge and eventually render these therapies ineffective. Described herein are antibody-based antivirus (antivirus) particles displaying multiple copies of antibodies, that effectively counteracts the multivalent interactions between viruses and host cell proteins and have improved potency against viruses such as coronavirus.

Described herein, in some embodiments, are antiviruses comprising a fusion protein that comprises a transmembrane polypeptide and an antibody which binds to a surface protein of a virus. In some embodiments, the viral surface protein is from SARS COV-1, SARS COV-2, influenza, or MERS CoV virus. In some embodiments, the viral surface protein is from SARS-CoV-1. In some embodiments, the viral surface protein is from SARS-COV-2. In some embodiments, the viral protein is from influenza. In some embodiments, the viral surface protein is from MERS-COV.

Described herein, in some embodiments, are antiviruses comprising a fusion protein that comprises a transmembrane polypeptide and an antibody which binds to a surface protein of a virus wherein the fusion protein is expressed at a valency of at least about 10 copies on a surface of the antivirus, wherein the antibody neutralizes the virus when displayed as a fusion protein on the surface of the antivirus, the virus when expressed as an isolated antibody. Further described herein, in some embodiments, are antiviruses comprising a fusion protein that comprises a transmembrane polypeptide, an oligomerization domain, and an antibody which binds to a surface protein of a virus wherein the fusion protein is expressed at a valency of at least about 10 copies on a surface of the antivirus, and the antivirus neutralizes the virus when the fusion protein is bound to the surface protein of the virus. Further described herein, in some embodiments, are antiviruses comprising (a) a first fusion protein that comprises a first transmembrane polypeptide and a first antibody which binds to a surface protein of a virus wherein the first fusion protein is expressed at a valency of at least about 10 copies on a surface of the antivirus, (b) a second fusion protein that comprises a second transmembrane polypeptide and second antibody which binds to a surface protein of the virus, wherein the second antibody comprises a CDR sequence that has less than 100% sequence identity to an equivalent CDR sequence of the first antibody, wherein the antivirus neutralizes the virus when either the first fusion protein or second fusion protein is bound to the surface protein of the virus. Further described herein, in some embodiments, are antiviruses comprising a fusion protein that comprises a transmembrane polypeptide and a multispecific antibody which binds to a surface protein of a virus wherein the fusion protein is expressed at a valency of at least about 10 copies on a surface of the antivirus, and the antivirus neutralizes the virus when the fusion protein is bound to the surface protein of the virus.

Described herein, in some embodiments, are antiviruses comprising a fusion protein that comprises a transmembrane polypeptide and an antibody which binds to a surface protein of a virus, wherein the fusion protein further comprises an oligomerization domain. In some embodiments, the oligomerization domain is a dimerization domain. In some embodiments, the dimerization domain comprises a leucine zipper dimerization domain. In some embodiments, the oligomerization domain is a trimerization domain. In some embodiments, the oligomerization domain comprises the post-fusion D4 trimerization domain of VSV-G protein. In some embodiments, the oligomerization domain comprises the post-fusion trimerization domain of Dengue E protein. In some embodiments, the oligomerization domain comprises the foldon trimerization domain. In some embodiments, the oligomerization domain is a tetramerization domain. In some embodiments, the oligomerization domain is a tetramerization domain. In some embodiments, the tetramerization domain comprises an influenza neuraminidase stem domain.

Described herein, in some embodiments, are antiviruses comprising a fusion protein that comprises a transmembrane polypeptide and an antibody which binds to a surface protein of a virus. In some embodiments, the antibody comprises a single chain variable fragment (scFv), a single domain antibody, an Fv, a VH domain, a VL domain, a Fab fragment, a monoclonal antibody, F(ab′), F(ab′)2, single chain antibodies, diabodies, or a scFv-Fc. In some embodiments, the antibody binds specifically to the surface protein of the virus. In some embodiments, the antibody is a multispecific antibody. In some embodiments, the multispecific antibody binds specifically to more than one epitope on the surface protein of the virus. In some embodiments, the multispecific antibody comprises a tandem scFv format.

Described herein, in some embodiments, are antiviruses comprising a fusion protein that comprises a transmembrane polypeptide and an antibody which binds to a surface protein of a virus, wherein the virus comprises SARS COV-1, CoV-2, influenza, or MERS CoV virus. In some embodiments, the antibody comprises an amino acid sequence from at least one complementarity determining region of 80R, H4, 7D10, 1E01, F16, 0304-4A10, 2M-14E5, 9A1, COV2-2021, COV2-2026, COV2-2146, 2M-10B11, C021, C018, BG4-5, BG7-15, or BG10-19. In some embodiments, the antibody comprises an amino acid sequence according to any one of SEQ ID NOs: 1-28 and 81-86. In some embodiments, the antibody comprises a variable domain of heavy chain amino acid sequence according to any one of SEQ ID NOs: 1-14, 81, 83 and 85. In some embodiments, the antibody comprises a variable domain of light chain amino acid sequence according to any one of SEQ ID NOs: 15-28, 82, 84 and 86. In some embodiments, the antibody neutralizes the virus when expressed within the fusion protein but does not neutralize the virus when expressed as an isolated antibody. In some embodiments, the antibody neutralizes the virus when expressed within the fusion protein and neutralizes the virus when expressed as an isolated antibody.

Various antiviruses are contemplated herein. In some embodiments, the antivirus is recombinant and genetically encoded. In some embodiments, the antivirus is an enveloped particle. In some embodiments, the antivirus is not a lentiviral particle. In some embodiments, the antivirus does not comprise viral genetic material. In some embodiments, the antivirus comprises a lipid bilayer. In some embodiments, the antivirus is a virus. In some embodiments, the antivirus is a replication incompetent virus. In some embodiments, the antivirus is a replication competent virus. In some embodiments, the antivirus is a viral-like particle. In some embodiments, the antivirus is an extracellular vesicle. In some embodiments, the antivirus is an exosome. In some embodiments, the antivirus is an ectosome.

Antiviruses as described herein, in some embodiments, comprise a fusion protein, wherein the fusion protein is expressed at multiple copies on a surface of the antivirus. In some embodiments, the fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the antivirus. In some embodiments, the fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the antivirus. In some embodiments, the fusion protein is expressed at a valency of at least or about 10 copies on a surface of the antivirus. In some embodiments, the fusion protein is expressed at a valency of at least or about 25 copies on a surface of the antivirus. In some embodiments, the fusion protein is expressed at a valency of at least or about 50 copies on a surface of the antivirus. In some embodiments, the fusion protein is expressed at a valency of at least or about 75 copies on a surface of the antivirus. In some embodiments, the fusion protein is expressed at a valency of at least or about 100 copies on a surface of the antivirus. In some embodiments, the fusion protein is expressed at a valency of at least or about 125 copies on a surface of the antivirus. In some embodiments, the fusion protein is expressed at a valency of at least or about 150 copies on a surface of the antivirus. In some embodiments, the fusion protein is expressed at a valency of at least or about 175 copies on a surface of the antivirus. In some embodiments, the fusion protein is expressed at a valency of at least or about 200 copies on a surface of the antivirus. In some embodiments, the fusion protein is expressed at a valency of at least or about 225 copies on a surface of the antivirus. In some embodiments, the fusion protein is expressed at a valency of at least or about 250 copies on a surface of the antivirus. In some embodiments, the fusion protein is expressed at a valency of at least or about 275 copies on a surface of the antivirus. In some embodiments, the fusion protein is expressed at a valency of at least or about 300 copies on a surface of the antivirus. In some embodiments, the fusion protein is expressed at a valency of at least or about 400 copies on a surface of the antivirus. In some embodiments, the fusion protein is expressed at a valency of at least or about 500 copies on a surface of the antivirus. In some embodiments, the fusion protein is expressed at a valency of at least or about 600 copies on a surface of the antivirus. In some embodiments, the fusion protein is expressed at a valency of at least or about 700 copies on a surface of the antivirus. In some embodiments, the fusion protein is expressed at a valency of at least or about 800 copies on a surface of the antivirus. In some embodiments, the fusion protein is expressed at a valency of at least or about 900 copies on a surface of the antivirus. In some embodiments, the fusion protein is expressed at a valency of at least or about 1000 copies on a surface of the antivirus.

In some embodiments, the antivirus is an enveloped particle. The enveloped particle as described herein, in some embodiments, comprise a fusion protein, wherein the fusion protein is expressed at multiple copies on a surface of the enveloped particle. In some embodiments, the fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the enveloped particle. In some embodiments, the fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the enveloped particle. In some embodiments, the fusion protein is expressed at a valency of at least or about 10 copies on a surface of the enveloped particle. In some embodiments, the fusion protein is expressed at a valency of at least or about 25 copies on a surface of the enveloped particle. In some embodiments, the fusion protein is expressed at a valency of at least or about 50 copies on a surface of the enveloped particle. In some embodiments, the fusion protein is expressed at a valency of at least or about 75 copies on a surface of the enveloped particle. In some embodiments, the fusion protein is expressed at a valency of at least or about 100 copies on a surface of the enveloped particle. In some embodiments, the fusion protein is expressed at a valency of at least or about 125 copies on a surface of the enveloped particle. In some embodiments, the fusion protein is expressed at a valency of at least or about 150 copies on a surface of the enveloped particle. In some embodiments, the fusion protein is expressed at a valency of at least or about 175 copies on a surface of the enveloped particle. In some embodiments, the fusion protein is expressed at a valency of at least or about 200 copies on a surface of the enveloped particle. In some embodiments, the fusion protein is expressed at a valency of at least or about 225 copies on a surface of the enveloped particle. In some embodiments, the fusion protein is expressed at a valency of at least or about 250 copies on a surface of the enveloped particle. In some embodiments, the fusion protein is expressed at a valency of at least or about 275 copies on a surface of the enveloped particle. In some embodiments, the fusion protein is expressed at a valency of at least or about 300 copies on a surface of the enveloped particle. In some embodiments, the fusion protein is expressed at a valency of at least or about 400 copies on a surface of the enveloped particle. In some embodiments, the fusion protein is expressed at a valency of at least or about 500 copies on a surface of the enveloped particle. In some embodiments, the fusion protein is expressed at a valency of at least or about 600 copies on a surface of the enveloped particle. In some embodiments, the fusion protein is expressed at a valency of at least or about 700 copies on a surface of the enveloped particle. In some embodiments, the fusion protein is expressed at a valency of at least or about 800 copies on a surface of the enveloped particle. In some embodiments, the fusion protein is expressed at a valency of at least or about 900 copies on a surface of the enveloped particle. In some embodiments, the fusion protein is expressed at a valency of at least or about 1000 copies on a surface of the enveloped particle.

In some embodiments, the antivirus is a viral-like particle. The viral-like particle as described herein, in some embodiments, comprise a fusion protein, wherein the fusion protein is expressed at multiple copies on a surface of the viral-like particle. In some embodiments, the fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the viral-like particle. In some embodiments, the fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the viral-like particle. In some embodiments, the fusion protein is expressed at a valency of at least or about 10 copies on a surface of the viral-like particle. In some embodiments, the fusion protein is expressed at a valency of at least or about 25 copies on a surface of the viral-like particle. In some embodiments, the fusion protein is expressed at a valency of at least or about 50 copies on a surface of the viral-like particle. In some embodiments, the fusion protein is expressed at a valency of at least or about 75 copies on a surface of the viral-like particle. In some embodiments, the fusion protein is expressed at a valency of at least or about 100 copies on a surface of the viral-like particle. In some embodiments, the fusion protein is expressed at a valency of at least or about 125 copies on a surface of the viral-like particle. In some embodiments, the fusion protein is expressed at a valency of at least or about 150 copies on a surface of the viral-like particle. In some embodiments, the fusion protein is expressed at a valency of at least or about 175 copies on a surface of the viral-like particle. In some embodiments, the fusion protein is expressed at a valency of at least or about 200 copies on a surface of the viral-like particle. In some embodiments, the fusion protein is expressed at a valency of at least or about 225 copies on a surface of the viral-like particle. In some embodiments, the fusion protein is expressed at a valency of at least or about 250 copies on a surface of the viral-like particle. In some embodiments, the fusion protein is expressed at a valency of at least or about 275 copies on a surface of the viral-like particle. In some embodiments, the fusion protein is expressed at a valency of at least or about 300 copies on a surface of the viral-like particle. In some embodiments, the fusion protein is expressed at a valency of at least or about 400 copies on a surface of the viral-like particle. In some embodiments, the fusion protein is expressed at a valency of at least or about 500 copies on a surface of the viral-like particle. In some embodiments, the fusion protein is expressed at a valency of at least or about 600 copies on a surface of the viral-like particle. In some embodiments, the fusion protein is expressed at a valency of at least or about 700 copies on a surface of the viral-like particle. In some embodiments, the fusion protein is expressed at a valency of at least or about 800 copies on a surface of the viral-like particle. In some embodiments, the fusion protein is expressed at a valency of at least or about 900 copies on a surface of the viral-like particle. In some embodiments, the fusion protein is expressed at a valency of at least or about 1000 copies on a surface of the viral-like particle.

In some embodiments, the antivirus is an extracellular vesicle. The extracellular vesicle as described herein, in some embodiments, comprise a fusion protein, wherein the fusion protein is expressed at multiple copies on a surface of the extracellular vesicle. In some embodiments, the fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the extracellular vesicle. In some embodiments, the fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the extracellular vesicle. In some embodiments, the fusion protein is expressed at a valency of at least or about 10 copies on a surface of the extracellular vesicle. In some embodiments, the fusion protein is expressed at a valency of at least or about 25 copies on a surface of the extracellular vesicle. In some embodiments, the fusion protein is expressed at a valency of at least or about 50 copies on a surface of the extracellular vesicle. In some embodiments, the fusion protein is expressed at a valency of at least or about 75 copies on a surface of the extracellular vesicle. In some embodiments, the fusion protein is expressed at a valency of at least or about 100 copies on a surface of the extracellular vesicle. In some embodiments, the fusion protein is expressed at a valency of at least or about 125 copies on a surface of the extracellular vesicle. In some embodiments, the fusion protein is expressed at a valency of at least or about 150 copies on a surface of the extracellular vesicle. In some embodiments, the fusion protein is expressed at a valency of at least or about 175 copies on a surface of the extracellular vesicle. In some embodiments, the fusion protein is expressed at a valency of at least or about 200 copies on a surface of the extracellular vesicle. In some embodiments, the fusion protein is expressed at a valency of at least or about 225 copies on a surface of the extracellular vesicle. In some embodiments, the fusion protein is expressed at a valency of at least or about 250 copies on a surface of the extracellular vesicle. In some embodiments, the fusion protein is expressed at a valency of at least or about 275 copies on a surface of the extracellular vesicle. In some embodiments, the fusion protein is expressed at a valency of at least or about 300 copies on a surface of the extracellular vesicle. In some embodiments, the fusion protein is expressed at a valency of at least or about 400 copies on a surface of the extracellular vesicle. In some embodiments, the fusion protein is expressed at a valency of at least or about 500 copies on a surface of the extracellular vesicle. In some embodiments, the fusion protein is expressed at a valency of at least or about 600 copies on a surface of the extracellular vesicle. In some embodiments, the fusion protein is expressed at a valency of at least or about 700 copies on a surface of the extracellular vesicle. In some embodiments, the fusion protein is expressed at a valency of at least or about 800 copies on a surface of the extracellular vesicle. In some embodiments, the fusion protein is expressed at a valency of at least or about 900 copies on a surface of the extracellular vesicle. In some embodiments, the fusion protein is expressed at a valency of at least or about 1000 copies on a surface of the extracellular vesicle.

In some embodiments, the antivirus is an exosome. The exosome as described herein, in some embodiments, comprise a fusion protein, wherein the fusion protein is expressed at multiple copies on a surface of the exosome. In some embodiments, the fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the exosome. In some embodiments, the fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the exosome. In some embodiments, the fusion protein is expressed at a valency of at least or about 10 copies on a surface of the exosome. In some embodiments, the fusion protein is expressed at a valency of at least or about 25 copies on a surface of the exosome. In some embodiments, the fusion protein is expressed at a valency of at least or about 50 copies on a surface of the exosome. In some embodiments, the fusion protein is expressed at a valency of at least or about 75 copies on a surface of the exosome. In some embodiments, the fusion protein is expressed at a valency of at least or about 100 copies on a surface of the exosome. In some embodiments, the fusion protein is expressed at a valency of at least or about 125 copies on a surface of the exosome. In some embodiments, the fusion protein is expressed at a valency of at least or about 150 copies on a surface of the exosome. In some embodiments, the fusion protein is expressed at a valency of at least or about 175 copies on a surface of the exosome. In some embodiments, the fusion protein is expressed at a valency of at least or about 200 copies on a surface of the exosome. In some embodiments, the fusion protein is expressed at a valency of at least or about 225 copies on a surface of the exosome. In some embodiments, the fusion protein is expressed at a valency of at least or about 250 copies on a surface of the exosome. In some embodiments, the fusion protein is expressed at a valency of at least or about 275 copies on a surface of the exosome. In some embodiments, the fusion protein is expressed at a valency of at least or about 300 copies on a surface of the exosome. In some embodiments, the fusion protein is expressed at a valency of at least or about 400 copies on a surface of the exosome. In some embodiments, the fusion protein is expressed at a valency of at least or about 500 copies on a surface of the exosome. In some embodiments, the fusion protein is expressed at a valency of at least or about 600 copies on a surface of the exosome. In some embodiments, the fusion protein is expressed at a valency of at least or about 700 copies on a surface of the exosome. In some embodiments, the fusion protein is expressed at a valency of at least or about 800 copies on a surface of the exosome. In some embodiments, the fusion protein is expressed at a valency of at least or about 900 copies on a surface of the exosome. In some embodiments, the fusion protein is expressed at a valency of at least or about 1000 copies on a surface of the exosome.

In some embodiments, the antivirus is an ectosome. The ectosome as described herein, in some embodiments, comprise a fusion protein, wherein the fusion protein is expressed at multiple copies on a surface of the ectosome. In some embodiments, the fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the ectosome. In some embodiments, the fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the ectosome. In some embodiments, the fusion protein is expressed at a valency of at least or about 10 copies on a surface of the ectosome. In some embodiments, the fusion protein is expressed at a valency of at least or about 25 copies on a surface of the ectosome. In some embodiments, the fusion protein is expressed at a valency of at least or about 50 copies on a surface of the ectosome. In some embodiments, the fusion protein is expressed at a valency of at least or about 75 copies on a surface of the ectosome. In some embodiments, the fusion protein is expressed at a valency of at least or about 100 copies on a surface of the ectosome. In some embodiments, the fusion protein is expressed at a valency of at least or about 125 copies on a surface of the ectosome. In some embodiments, the fusion protein is expressed at a valency of at least or about 150 copies on a surface of the ectosome. In some embodiments, the fusion protein is expressed at a valency of at least or about 175 copies on a surface of the ectosome. In some embodiments, the fusion protein is expressed at a valency of at least or about 200 copies on a surface of the ectosome. In some embodiments, the fusion protein is expressed at a valency of at least or about 225 copies on a surface of the ectosome. In some embodiments, the fusion protein is expressed at a valency of at least or about 250 copies on a surface of the ectosome. In some embodiments, the fusion protein is expressed at a valency of at least or about 275 copies on a surface of the ectosome. In some embodiments, the fusion protein is expressed at a valency of at least or about 300 copies on a surface of the ectosome. In some embodiments, the fusion protein is expressed at a valency of at least or about 400 copies on a surface of the ectosome. In some embodiments, the fusion protein is expressed at a valency of at least or about 500 copies on a surface of the ectosome. In some embodiments, the fusion protein is expressed at a valency of at least or about 600 copies on a surface of the ectosome. In some embodiments, the fusion protein is expressed at a valency of at least or about 700 copies on a surface of the ectosome. In some embodiments, the fusion protein is expressed at a valency of at least or about 800 copies on a surface of the ectosome. In some embodiments, the fusion protein is expressed at a valency of at least or about 900 copies on a surface of the ectosome. In some embodiments, the fusion protein is expressed at a valency of at least or about 1000 copies on a surface of the ectosome.

In some embodiments, the antivirus is a virus. The virus as described herein, in some embodiments, comprise a fusion protein, wherein the fusion protein is expressed at multiple copies on a surface of the virus. In some embodiments, the fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the virus. In some embodiments, the fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the virus. In some embodiments, the fusion protein is expressed at a valency of at least or about 10 copies on a surface of the virus. In some embodiments, the fusion protein is expressed at a valency of at least or about 25 copies on a surface of the virus. In some embodiments, the fusion protein is expressed at a valency of at least or about 50 copies on a surface of the virus. In some embodiments, the fusion protein is expressed at a valency of at least or about 75 copies on a surface of the virus. In some embodiments, the fusion protein is expressed at a valency of at least or about 100 copies on a surface of the virus. In some embodiments, the fusion protein is expressed at a valency of at least or about 125 copies on a surface of the virus. In some embodiments, the fusion protein is expressed at a valency of at least or about 150 copies on a surface of the virus. In some embodiments, the fusion protein is expressed at a valency of at least or about 175 copies on a surface of the virus. In some embodiments, the fusion protein is expressed at a valency of at least or about 200 copies on a surface of the virus. In some embodiments, the fusion protein is expressed at a valency of at least or about 225 copies on a surface of the virus. In some embodiments, the fusion protein is expressed at a valency of at least or about 250 copies on a surface of the virus. In some embodiments, the fusion protein is expressed at a valency of at least or about 275 copies on a surface of the virus. In some embodiments, the fusion protein is expressed at a valency of at least or about 300 copies on a surface of the virus. In some embodiments, the fusion protein is expressed at a valency of at least or about 400 copies on a surface of the virus. In some embodiments, the fusion protein is expressed at a valency of at least or about 500 copies on a surface of the virus. In some embodiments, the fusion protein is expressed at a valency of at least or about 600 copies on a surface of the virus. In some embodiments, the fusion protein is expressed at a valency of at least or about 700 copies on a surface of the virus. In some embodiments, the fusion protein is expressed at a valency of at least or about 800 copies on a surface of the virus. In some embodiments, the fusion protein is expressed at a valency of at least or about 900 copies on a surface of the virus. In some embodiments, the fusion protein is expressed at a valency of at least or about 1000 copies on a surface of the virus.

In some embodiments, the antivirus is a replication incompetent virus. The replication incompetent virus as described herein, in some embodiments, comprise a fusion protein, wherein the fusion protein is expressed at multiple copies on a surface of the replication incompetent virus. In some embodiments, the fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the replication incompetent virus. In some embodiments, the fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the replication incompetent virus. In some embodiments, the fusion protein is expressed at a valency of at least or about 10 copies on a surface of the replication incompetent virus. In some embodiments, the fusion protein is expressed at a valency of at least or about 25 copies on a surface of the replication incompetent virus. In some embodiments, the fusion protein is expressed at a valency of at least or about 50 copies on a surface of the replication incompetent virus. In some embodiments, the fusion protein is expressed at a valency of at least or about 75 copies on a surface of the replication incompetent virus. In some embodiments, the fusion protein is expressed at a valency of at least or about 100 copies on a surface of the replication incompetent virus. In some embodiments, the fusion protein is expressed at a valency of at least or about 125 copies on a surface of the replication incompetent virus. In some embodiments, the fusion protein is expressed at a valency of at least or about 150 copies on a surface of the replication incompetent virus. In some embodiments, the fusion protein is expressed at a valency of at least or about 175 copies on a surface of the replication incompetent virus. In some embodiments, the fusion protein is expressed at a valency of at least or about 200 copies on a surface of the replication incompetent virus. In some embodiments, the fusion protein is expressed at a valency of at least or about 225 copies on a surface of the replication incompetent virus. In some embodiments, the fusion protein is expressed at a valency of at least or about 250 copies on a surface of the replication incompetent virus. In some embodiments, the fusion protein is expressed at a valency of at least or about 275 copies on a surface of the replication incompetent virus. In some embodiments, the fusion protein is expressed at a valency of at least or about 300 copies on a surface of the replication incompetent virus. In some embodiments, the fusion protein is expressed at a valency of at least or about 400 copies on a surface of the replication incompetent virus. In some embodiments, the fusion protein is expressed at a valency of at least or about 500 copies on a surface of the replication incompetent virus. In some embodiments, the fusion protein is expressed at a valency of at least or about 600 copies on a surface of the replication incompetent virus. In some embodiments, the fusion protein is expressed at a valency of at least or about 700 copies on a surface of the replication incompetent virus. In some embodiments, the fusion protein is expressed at a valency of at least or about 800 copies on a surface of the replication incompetent virus. In some embodiments, the fusion protein is expressed at a valency of at least or about 900 copies on a surface of the replication incompetent virus. In some embodiments, the fusion protein is expressed at a valency of at least or about 1000 copies on a surface of the replication incompetent virus.

In some embodiments, the antivirus is a replication competent virus. The replication competent virus as described herein, in some embodiments, comprise a fusion protein, wherein the fusion protein is expressed at multiple copies on a surface of the replication competent virus. In some embodiments, the fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the replication competent virus. In some embodiments, the fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the replication competent virus. In some embodiments, the fusion protein is expressed at a valency of at least or about 10 copies on a surface of the replication competent virus. In some embodiments, the fusion protein is expressed at a valency of at least or about 25 copies on a surface of the replication competent virus. In some embodiments, the fusion protein is expressed at a valency of at least or about 50 copies on a surface of the replication competent virus. In some embodiments, the fusion protein is expressed at a valency of at least or about 75 copies on a surface of the replication competent virus. In some embodiments, the fusion protein is expressed at a valency of at least or about 100 copies on a surface of the replication competent virus. In some embodiments, the fusion protein is expressed at a valency of at least or about 125 copies on a surface of the replication competent virus. In some embodiments, the fusion protein is expressed at a valency of at least or about 150 copies on a surface of the replication competent virus. In some embodiments, the fusion protein is expressed at a valency of at least or about 175 copies on a surface of the replication competent virus. In some embodiments, the fusion protein is expressed at a valency of at least or about 200 copies on a surface of the replication competent virus. In some embodiments, the fusion protein is expressed at a valency of at least or about 225 copies on a surface of the replication competent virus. In some embodiments, the fusion protein is expressed at a valency of at least or about 250 copies on a surface of the replication competent virus. In some embodiments, the fusion protein is expressed at a valency of at least or about 275 copies on a surface of the replication competent virus. In some embodiments, the fusion protein is expressed at a valency of at least or about 300 copies on a surface of the replication competent virus. In some embodiments, the fusion protein is expressed at a valency of at least or about 400 copies on a surface of the replication competent virus. In some embodiments, the fusion protein is expressed at a valency of at least or about 500 copies on a surface of the replication competent virus. In some embodiments, the fusion protein is expressed at a valency of at least or about 600 copies on a surface of the replication competent virus. In some embodiments, the fusion protein is expressed at a valency of at least or about 700 copies on a surface of the replication competent virus. In some embodiments, the fusion protein is expressed at a valency of at least or about 800 copies on a surface of the replication competent virus. In some embodiments, the fusion protein is expressed at a valency of at least or about 900 copies on a surface of the replication competent virus. In some embodiments, the fusion protein is expressed at a valency of at least or about 1000 copies on a surface of the replication competent virus.

Antiviruses as described herein, in some embodiments, comprise a second fusion protein to further increase the valency of antivirus, wherein the second fusion protein is expressed at multiple copies on a surface of the antivirus. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the antivirus. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the antivirus. In some embodiments, the second fusion protein is expressed at a valency of at least or about 10 copies on a surface of the antivirus. In some embodiments, the second fusion protein is expressed at a valency of at least or about 25 copies on a surface of the antivirus. In some embodiments, the second fusion protein is expressed at a valency of at least or about 50 copies on a surface of the antivirus. In some embodiments, the second fusion protein is expressed at a valency of at least or about 75 copies on a surface of the multivalent particle. In some embodiments, the second fusion protein is expressed at a valency of at least or about 100 copies on a surface of the antivirus. In some embodiments, the second fusion protein is expressed at a valency of at least or about 125 copies on a surface of the antivirus. In some embodiments, the second fusion protein is expressed at a valency of at least or about 150 copies on a surface of the antivirus. In some embodiments, the second fusion protein is expressed at a valency of at least or about 175 copies on a surface of the antivirus. In some embodiments, the second fusion protein is expressed at a valency of at least or about 200 copies on a surface of the antivirus. In some embodiments, the second fusion protein is expressed at a valency of at least or about 225 copies on a surface of the antivirus. In some embodiments, the second fusion protein is expressed at a valency of at least or about 250 copies on a surface of the antivirus. In some embodiments, the second fusion protein is expressed at a valency of at least or about 275 copies on a surface of the antivirus. In some embodiments, the second fusion protein is expressed at a valency of at least or about 300 copies on a surface of the antivirus.

The enveloped particle, as described herein, in some embodiments, comprise a second fusion protein, wherein the second fusion protein is expressed at multiple copies on a surface of the enveloped particle. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the enveloped particle. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the enveloped particle. In some embodiments, the second fusion protein is expressed at a valency of at least or about 10 copies on a surface of the enveloped particle. In some embodiments, the second fusion protein is expressed at a valency of at least or about 25 copies on a surface of the enveloped particle. In some embodiments, the second fusion protein is expressed at a valency of at least or about 50 copies on a surface of the enveloped particle. In some embodiments, the second fusion protein is expressed at a valency of at least or about 75 copies on a surface of the enveloped particle. In some embodiments, the second fusion protein is expressed at a valency of at least or about 100 copies on a surface of the enveloped particle. In some embodiments, the second fusion protein is expressed at a valency of at least or about 125 copies on a surface of the enveloped particle. In some embodiments, the second fusion protein is expressed at a valency of at least or about 150 copies on a surface of the enveloped particle. In some embodiments, the second fusion protein is expressed at a valency of at least or about 175 copies on a surface of the enveloped particle. In some embodiments, the second fusion protein is expressed at a valency of at least or about 200 copies on a surface of the enveloped particle. In some embodiments, the second fusion protein is expressed at a valency of at least or about 225 copies on a surface of the enveloped particle. In some embodiments, the second fusion protein is expressed at a valency of at least or about 250 copies on a surface of the enveloped particle. In some embodiments, the second fusion protein is expressed at a valency of at least or about 275 copies on a surface of the enveloped particle. In some embodiments, the second fusion protein is expressed at a valency of at least or about 300 copies on a surface of the enveloped particle. In some embodiments, the second fusion protein is expressed at a valency of at least or about 400 copies on a surface of the enveloped particle. In some embodiments, the second fusion protein is expressed at a valency of at least or about 500 copies on a surface of the enveloped particle. In some embodiments, the second fusion protein is expressed at a valency of at least or about 600 copies on a surface of the enveloped particle. In some embodiments, the second fusion protein is expressed at a valency of at least or about 700 copies on a surface of the enveloped particle. In some embodiments, the second fusion protein is expressed at a valency of at least or about 800 copies on a surface of the enveloped particle. In some embodiments, the second fusion protein is expressed at a valency of at least or about 900 copies on a surface of the enveloped particle. In some embodiments, the second fusion protein is expressed at a valency of at least or about 1000 copies on a surface of the enveloped particle.

The viral-like particle as described herein, in some embodiments, comprise a second fusion protein, wherein the second fusion protein is expressed at multiple copies on a surface of the viral-like particle. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the viral-like particle. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the viral-like particle. In some embodiments, the second fusion protein is expressed at a valency of at least or about 10 copies on a surface of the viral-like particle. In some embodiments, the second fusion protein is expressed at a valency of at least or about 25 copies on a surface of the viral-like particle. In some embodiments, the second fusion protein is expressed at a valency of at least or about 50 copies on a surface of the viral-like particle. In some embodiments, the second fusion protein is expressed at a valency of at least or about 75 copies on a surface of the viral-like particle. In some embodiments, the second fusion protein is expressed at a valency of at least or about 100 copies on a surface of the viral-like particle. In some embodiments, the second fusion protein is expressed at a valency of at least or about 125 copies on a surface of the viral-like particle. In some embodiments, the second fusion protein is expressed at a valency of at least or about 150 copies on a surface of the viral-like particle. In some embodiments, the second fusion protein is expressed at a valency of at least or about 175 copies on a surface of the viral-like particle. In some embodiments, the second fusion protein is expressed at a valency of at least or about 200 copies on a surface of the viral-like particle. In some embodiments, the second fusion protein is expressed at a valency of at least or about 225 copies on a surface of the viral-like particle. In some embodiments, the second fusion protein is expressed at a valency of at least or about 250 copies on a surface of the viral-like particle. In some embodiments, the second fusion protein is expressed at a valency of at least or about 275 copies on a surface of the viral-like particle. In some embodiments, the second fusion protein is expressed at a valency of at least or about 300 copies on a surface of the viral-like particle. In some embodiments, the second fusion protein is expressed at a valency of at least or about 300 copies on a surface of the viral-like particle. In some embodiments, the second fusion protein is expressed at a valency of at least or about 400 copies on a surface of the viral-like particle. In some embodiments, the second fusion protein is expressed at a valency of at least or about 500 copies on a surface of the viral-like particle. In some embodiments, the second fusion protein is expressed at a valency of at least or about 600 copies on a surface of the viral-like particle. In some embodiments, the second fusion protein is expressed at a valency of at least or about 700 copies on a surface of the viral-like particle. In some embodiments, the second fusion protein is expressed at a valency of at least or about 800 copies on a surface of the viral-like particle. In some embodiments, the second fusion protein is expressed at a valency of at least or about 900 copies on a surface of the viral-like particle. In some embodiments, the second fusion protein is expressed at a valency of at least or about 1000 copies on a surface of the viral-like particle.

The extracellular vesicle, as described herein, in some embodiments, comprise a second fusion protein, wherein the second fusion protein is expressed at multiple copies on a surface of the extracellular vesicle. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the extracellular vesicle. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the extracellular vesicle. In some embodiments, the second fusion protein is expressed at a valency of at least or about 10 copies on a surface of the extracellular vesicle. In some embodiments, the second fusion protein is expressed at a valency of at least or about 25 copies on a surface of the extracellular vesicle. In some embodiments, the second fusion protein is expressed at a valency of at least or about 50 copies on a surface of the extracellular vesicle. In some embodiments, the second fusion protein is expressed at a valency of at least or about 75 copies on a surface of the extracellular vesicle. In some embodiments, the second fusion protein is expressed at a valency of at least or about 100 copies on a surface of the extracellular vesicle. In some embodiments, the second fusion protein is expressed at a valency of at least or about 125 copies on a surface of the extracellular vesicle. In some embodiments, the second fusion protein is expressed at a valency of at least or about 150 copies on a surface of the extracellular vesicle. In some embodiments, the second fusion protein is expressed at a valency of at least or about 175 copies on a surface of the extracellular vesicle. In some embodiments, the second fusion protein is expressed at a valency of at least or about 200 copies on a surface of the extracellular vesicle. In some embodiments, the second fusion protein is expressed at a valency of at least or about 225 copies on a surface of the extracellular vesicle. In some embodiments, the second fusion protein is expressed at a valency of at least or about 250 copies on a surface of the extracellular vesicle. In some embodiments, the second fusion protein is expressed at a valency of at least or about 275 copies on a surface of the extracellular vesicle. In some embodiments, the second fusion protein is expressed at a valency of at least or about 300 copies on a surface of the extracellular vesicle. In some embodiments, the second fusion protein is expressed at a valency of at least or about 400 copies on a surface of the extracellular vesicle. In some embodiments, the second fusion protein is expressed at a valency of at least or about 500 copies on a surface of the extracellular vesicle. In some embodiments, the second fusion protein is expressed at a valency of at least or about 600 copies on a surface of the extracellular vesicle. In some embodiments, the second fusion protein is expressed at a valency of at least or about 700 copies on a surface of the extracellular vesicle. In some embodiments, the second fusion protein is expressed at a valency of at least or about 800 copies on a surface of the extracellular vesicle. In some embodiments, the second fusion protein is expressed at a valency of at least or about 900 copies on a surface of the extracellular vesicle. In some embodiments, the second fusion protein is expressed at a valency of at least or about 1000 copies on a surface of the extracellular vesicle.

The exosome, as described herein, in some embodiments, comprise a second fusion protein, wherein the second fusion protein is expressed at multiple copies on a surface of the exosome. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the exosome. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the exosome. In some embodiments, the second fusion protein is expressed at a valency of at least or about 10 copies on a surface of the exosome. In some embodiments, the second fusion protein is expressed at a valency of at least or about 25 copies on a surface of the exosome. In some embodiments, the second fusion protein is expressed at a valency of at least or about 50 copies on a surface of the exosome. In some embodiments, the second fusion protein is expressed at a valency of at least or about 75 copies on a surface of the exosome. In some embodiments, the second fusion protein is expressed at a valency of at least or about 100 copies on a surface of the exosome. In some embodiments, the second fusion protein is expressed at a valency of at least or about 125 copies on a surface of the exosome. In some embodiments, the second fusion protein is expressed at a valency of at least or about 150 copies on a surface of the exosome. In some embodiments, the second fusion protein is expressed at a valency of at least or about 175 copies on a surface of the exosome. In some embodiments, the second fusion protein is expressed at a valency of at least or about 200 copies on a surface of the exosome. In some embodiments, the second fusion protein is expressed at a valency of at least or about 225 copies on a surface of the exosome. In some embodiments, the second fusion protein is expressed at a valency of at least or about 250 copies on a surface of the exosome. In some embodiments, the second fusion protein is expressed at a valency of at least or about 275 copies on a surface of the exosome. In some embodiments, the second fusion protein is expressed at a valency of at least or about 300 copies on a surface of the exosome. In some embodiments, the second fusion protein is expressed at a valency of at least or about 400 copies on a surface of the exosome. In some embodiments, the second fusion protein is expressed at a valency of at least or about 500 copies on a surface of the exosome. In some embodiments, the second fusion protein is expressed at a valency of at least or about 600 copies on a surface of the exosome. In some embodiments, the second fusion protein is expressed at a valency of at least or about 700 copies on a surface of the exosome. In some embodiments, the second fusion protein is expressed at a valency of at least or about 800 copies on a surface of the exosome. In some embodiments, the second fusion protein is expressed at a valency of at least or about 900 copies on a surface of the exosome. In some embodiments, the second fusion protein is expressed at a valency of at least or about 1000 copies on a surface of the exosome.

The ectosome, as described herein, in some embodiments, comprises a second fusion protein, wherein the second fusion protein is expressed at multiple copies on a surface of the ectosome. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the ectosome. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the ectosome. In some embodiments, the second fusion protein is expressed at a valency of at least or about 10 copies on a surface of the ectosome. In some embodiments, the second fusion protein is expressed at a valency of at least or about 25 copies on a surface of the ectosome. In some embodiments, the second fusion protein is expressed at a valency of at least or about 50 copies on a surface of the ectosome. In some embodiments, the second fusion protein is expressed at a valency of at least or about 75 copies on a surface of the ectosome. In some embodiments, the second fusion protein is expressed at a valency of at least or about 100 copies on a surface of the ectosome. In some embodiments, the second fusion protein is expressed at a valency of at least or about 125 copies on a surface of the ectosome. In some embodiments, the second fusion protein is expressed at a valency of at least or about 150 copies on a surface of the ectosome. In some embodiments, the second fusion protein is expressed at a valency of at least or about 175 copies on a surface of the ectosome. In some embodiments, the second fusion protein is expressed at a valency of at least or about 200 copies on a surface of the ectosome. In some embodiments, the second fusion protein is expressed at a valency of at least or about 225 copies on a surface of the ectosome. In some embodiments, the second fusion protein is expressed at a valency of at least or about 250 copies on a surface of the ectosome. In some embodiments, the second fusion protein is expressed at a valency of at least or about 275 copies on a surface of the ectosome. In some embodiments, the second fusion protein is expressed at a valency of at least or about 300 copies on a surface of the ectosome. In some embodiments, the second fusion protein is expressed at a valency of at least or about 400 copies on a surface of the ectosome. In some embodiments, the second fusion protein is expressed at a valency of at least or about 500 copies on a surface of the ectosome. In some embodiments, the second fusion protein is expressed at a valency of at least or about 600 copies on a surface of the ectosome. In some embodiments, the second fusion protein is expressed at a valency of at least or about 700 copies on a surface of the ectosome. In some embodiments, the second fusion protein is expressed at a valency of at least or about 800 copies on a surface of the ectosome. In some embodiments, the second fusion protein is expressed at a valency of at least or about 900 copies on a surface of the ectosome. In some embodiments, the second fusion protein is expressed at a valency of at least or about 1000 copies on a surface of the ectosome.

The virus as described herein, in some embodiments, comprise a second fusion protein, wherein the second fusion protein is expressed at multiple copies on a surface of the virus. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the virus. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the virus. In some embodiments, the second fusion protein is expressed at a valency of at least or about 10 copies on a surface of the virus. In some embodiments, the second fusion protein is expressed at a valency of at least or about 25 copies on a surface of the virus. In some embodiments, the second fusion protein is expressed at a valency of at least or about 50 copies on a surface of the virus. In some embodiments, the second fusion protein is expressed at a valency of at least or about 75 copies on a surface of the virus. In some embodiments, the second fusion protein is expressed at a valency of at least or about 100 copies on a surface of the virus. In some embodiments, the second fusion protein is expressed at a valency of at least or about 125 copies on a surface of the virus. In some embodiments, the second fusion protein is expressed at a valency of at least or about 150 copies on a surface of the virus. In some embodiments, the second fusion protein is expressed at a valency of at least or about 175 copies on a surface of the virus. In some embodiments, the second fusion protein is expressed at a valency of at least or about 200 copies on a surface of the virus. In some embodiments, the second fusion protein is expressed at a valency of at least or about 225 copies on a surface of the virus. In some embodiments, the second fusion protein is expressed at a valency of at least or about 250 copies on a surface of the virus. In some embodiments, the second fusion protein is expressed at a valency of at least or about 275 copies on a surface of the virus. In some embodiments, the second fusion protein is expressed at a valency of at least or about 300 copies on a surface of the virus. In some embodiments, the second fusion protein is expressed at a valency of at least or about 300 copies on a surface of the virus. In some embodiments, the second fusion protein is expressed at a valency of at least or about 400 copies on a surface of the virus. In some embodiments, the second fusion protein is expressed at a valency of at least or about 500 copies on a surface of the virus. In some embodiments, the second fusion protein is expressed at a valency of at least or about 600 copies on a surface of the virus. In some embodiments, the second fusion protein is expressed at a valency of at least or about 700 copies on a surface of the virus. In some embodiments, the second fusion protein is expressed at a valency of at least or about 800 copies on a surface of the virus. In some embodiments, the second fusion protein is expressed at a valency of at least or about 900 copies on a surface of the virus. In some embodiments, the second fusion protein is expressed at a valency of at least or about 1000 copies on a surface of the virus.

The replication incompetent virus as described herein, in some embodiments, comprise a second fusion protein, wherein the second fusion protein is expressed at multiple copies on a surface of the replication incompetent virus. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the replication incompetent virus. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the replication incompetent virus. In some embodiments, the second fusion protein is expressed at a valency of at least or about 10 copies on a surface of the replication incompetent virus. In some embodiments, the second fusion protein is expressed at a valency of at least or about 25 copies on a surface of the replication incompetent virus. In some embodiments, the second fusion protein is expressed at a valency of at least or about 50 copies on a surface of the replication incompetent virus. In some embodiments, the second fusion protein is expressed at a valency of at least or about 75 copies on a surface of the replication incompetent virus. In some embodiments, the second fusion protein is expressed at a valency of at least or about 100 copies on a surface of the replication incompetent virus. In some embodiments, the second fusion protein is expressed at a valency of at least or about 125 copies on a surface of the replication incompetent virus. In some embodiments, the second fusion protein is expressed at a valency of at least or about 150 copies on a surface of the replication incompetent virus. In some embodiments, the second fusion protein is expressed at a valency of at least or about 175 copies on a surface of the replication incompetent virus. In some embodiments, the second fusion protein is expressed at a valency of at least or about 200 copies on a surface of the replication incompetent virus. In some embodiments, the second fusion protein is expressed at a valency of at least or about 225 copies on a surface of the replication incompetent virus. In some embodiments, the second fusion protein is expressed at a valency of at least or about 250 copies on a surface of the replication incompetent virus. In some embodiments, the second fusion protein is expressed at a valency of at least or about 275 copies on a surface of the replication incompetent virus. In some embodiments, the second fusion protein is expressed at a valency of at least or about 300 copies on a surface of the replication incompetent virus. In some embodiments, the second fusion protein is expressed at a valency of at least or about 300 copies on a surface of the replication incompetent virus. In some embodiments, the second fusion protein is expressed at a valency of at least or about 400 copies on a surface of the replication incompetent virus. In some embodiments, the second fusion protein is expressed at a valency of at least or about 500 copies on a surface of the replication incompetent virus. In some embodiments, the second fusion protein is expressed at a valency of at least or about 600 copies on a surface of the replication incompetent virus. In some embodiments, the second fusion protein is expressed at a valency of at least or about 700 copies on a surface of the replication incompetent virus. In some embodiments, the second fusion protein is expressed at a valency of at least or about 800 copies on a surface of the replication incompetent virus. In some embodiments, the second fusion protein is expressed at a valency of at least or about 900 copies on a surface of the replication incompetent virus. In some embodiments, the second fusion protein is expressed at a valency of at least or about 1000 copies on a surface of the replication incompetent virus.

The replication competent virus as described herein, in some embodiments, comprise a second fusion protein, wherein the second fusion protein is expressed at multiple copies on a surface of the replication competent virus. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the replication competent virus. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the replication competent virus. In some embodiments, the second fusion protein is expressed at a valency of at least or about 10 copies on a surface of the replication competent virus. In some embodiments, the second fusion protein is expressed at a valency of at least or about 25 copies on a surface of the replication competent virus. In some embodiments, the second fusion protein is expressed at a valency of at least or about 50 copies on a surface of the replication competent virus. In some embodiments, the second fusion protein is expressed at a valency of at least or about 75 copies on a surface of the replication competent virus. In some embodiments, the second fusion protein is expressed at a valency of at least or about 100 copies on a surface of the replication competent virus. In some embodiments, the second fusion protein is expressed at a valency of at least or about 125 copies on a surface of the replication competent virus. In some embodiments, the second fusion protein is expressed at a valency of at least or about 150 copies on a surface of the replication competent virus. In some embodiments, the second fusion protein is expressed at a valency of at least or about 175 copies on a surface of the replication competent virus. In some embodiments, the second fusion protein is expressed at a valency of at least or about 200 copies on a surface of the replication competent virus. In some embodiments, the second fusion protein is expressed at a valency of at least or about 225 copies on a surface of the replication competent virus. In some embodiments, the second fusion protein is expressed at a valency of at least or about 250 copies on a surface of the replication competent virus. In some embodiments, the second fusion protein is expressed at a valency of at least or about 275 copies on a surface of the replication competent virus. In some embodiments, the second fusion protein is expressed at a valency of at least or about 300 copies on a surface of the replication competent virus. In some embodiments, the second fusion protein is expressed at a valency of at least or about 300 copies on a surface of the replication competent virus. In some embodiments, the second fusion protein is expressed at a valency of at least or about 400 copies on a surface of the replication competent virus. In some embodiments, the second fusion protein is expressed at a valency of at least or about 500 copies on a surface of the replication competent virus. In some embodiments, the second fusion protein is expressed at a valency of at least or about 600 copies on a surface of the replication competent virus. In some embodiments, the second fusion protein is expressed at a valency of at least or about 700 copies on a surface of the replication competent virus. In some embodiments, the second fusion protein is expressed at a valency of at least or about 800 copies on a surface of the replication competent virus. In some embodiments, the second fusion protein is expressed at a valency of at least or about 900 copies on a surface of the replication competent virus. In some embodiments, the second fusion protein is expressed at a valency of at least or about 1000 copies on a surface of the replication competent virus.

Described herein, in some embodiments, are antiviruses comprising improved binding properties. In some embodiments, the antiviruses comprise a binding affinity (e.g., K_{D}) to the viral protein of less than 100 pM, less than 200 pM, less than 300 pM, less than 400 pM, less than 500 pM, less than 600 pM, less than 700 pM, less than 800 pM, or less than 900 pM In some embodiments, the antivirus comprises a K_{D} of less than 1 nM, less than 1.2 nM, less than 2 nM, less than 5 nM, or less than 10 nM. In some instances, the antivirus comprises a K_{D} of less than 1 nM. In some instances, the antivirus comprises a K_{D} of less than 1.2 nM. In some instances, the antivirus comprises a K_{D} of less than 2 nM. In some instances, the antivirus comprises a K_{D} of less than 5 nM. In some instances, the antivirus comprises a K_{D} of less than 10 nM.

In some embodiments, the antivirus comprises an IC50 of less than 20 picomolar (pM) in a neutralization assay. In some embodiments, the antivirus comprises an IC50 of less than 15 picomolar (pM) in a neutralization assay. In some embodiments, the antivirus comprises an IC50 of less than 10 picomolar (pM) in a neutralization assay. In some embodiments, the antivirus comprises an IC50 of less than 5 picomolar (pM) in a neutralization assay. In some embodiments, the antivirus comprises an IC50 of less than 2.5 picomolar (pM) in a neutralization assay. In some embodiments, the antivirus comprises an IC50 of less than 1 picomolar (pM) in a neutralization assay. In some embodiments, the antivirus comprises an IC50 of less than 0.5 picomolar (pM) in a neutralization assay.

### Oligomerization Domain

In some embodiments, the antivirus comprises an oligomerization domain. In some embodiments, the oligomerization domain is a dimerization domain. In some embodiments, the dimerization domain comprises a leucine zipper dimerization domain. In some embodiments, the oligomerization domain is a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the oligomerization domain is a tetramerization domain. In some embodiments, the tetramerization domain comprises an influenza neuraminidase stem domain.

TABLE 1
Exemplary Oligomerization Domain Sequences

| | | | SEQ ID |
|---|---|---|---|
| Name | Valence | Amino Acid Sequences | No. |
| D4 Variation 1 | Trimer | IGTALQVKMPKSHKAIQADGWMCHASK | 30 |
| | | WVTTCDFRWYGPKYITHSIRSFTPSVEQ | |
| | | CKESIEQTKQGTWLNPGFPPQSCGYATV | |
| | | TD7AEAVIVQVTPHHVLVDEYTGEWVD | |
| | | SQFIN8GKCSNYICPTVHNSTTWHSDYK | |
| | | VKGLCDSNLISMDI | |
| D4 Variation 2 | Trimer | IQADGWMCHASKWVTTCDFRWYGPKY | 31 |
| | | ITHSIRSFTPSVEQCKESIEQTKQGTWLNP | |
| | | GFPPQSCGYATVIDAEAVIVQVTPHHVL | |
| | | VDEYTGEWVDSQFINGKCSNYICPTVHN | |
| | | STTWHSDYKVKGLCDSNL | |
| D4 Variation 3 | Trimer | IQADGWMCHASKWVTTCDFRWYGPKY | 32 |
| | | ITHSIRSFTPSVEQCKESIEQTKQGTWLNP | |
| | | GFPPQSCGYATVIDAEAVIVQVTPHHVL | |
| | | VDEYTGEWVDSQFINGKCSNYICPTVHN | |
| | | STT | |
| D4 Variation 4 | Trimer | IQADGWMCHASKWVTTCDFRWYGPKY | 33 |
| | | ITHSIRSFTPSVEQCKESIEQTKQGTWLNP | |
| | | GFPPQSCGYATVIDAEAVIVQVTPHHVL | |
| | | VDEYTGEWVDSQFING | |
| D4 Variation 5 | Trimer | IQADGWMCHASKWVTTCDFRWYGPKY | 34 |
| | | ITHSIRSFTPSVEQCKESIEQTKQGTWLNP | |
| | | GFPPQSCGYATVTDAEAVIVQVTPHHVL | |
| Foldon | Trimer | GYIPEAPRDGQAYVRKDGEWVLLSTFL | 35 |
| Leucine Zipper | Dimer | RMKQLEDKVEELLSKQYHLENEVARLK | 36 |
| V1 | | KLVGER | |
| Leucine Zipper | Dimer | RMKQLEDKVEELLSKNYHLENEVARLK | 37 |
| V2 | | KLVGER | |
| Neuraminidase | Tetramer | MNPNQKIITIGSICLVVGLISLILQIGNIISI | 38 |
| Stem V1 | | WISHSIQT | |
| Neuraminidase | Tetramer | MNPNQKIITIGSICMVTGIVSLMLQIGNM | 39 |
| Stem V2 | | ISIWVSHSIHTGNQHQSEPISNTNFLTEKA | |
| | | VASVKLAGNSSLCPIN | |
| Dengue E Fusion | Trimer | KLCIEAKISNTTTDSRCPTQGEATLVEEQ | 40 |
| V1 | | DTNFVCRRTFVDRGHGNGCGLFGKGSLI | |
| | | TCAKFKCVTKL | |
| Dengue E Fusion | Trimer | IELLKTEVTNPAVLRKLCIEAKISNTTTDS | 41 |
| V2 | | RCPTQGEATLVEEQDTNFVCRRTFVDRG | |
| | | HGNGCGLFGKGSLITCAKFKCVTKL | |
| Dengue E Fusion | Trimer | KLCIEAKISNTTTDSRCPTQGEATLVEEQ | 42 |
| V3 | | DTNFVCRRTFVDRGHGNGCGLFGKGSLI | |
| | | TCAKFKCVTKLEGKIVQYENLKYSVI | |
| Dengue E Fusion | Trimer | EAKISNTTTDSRCPTQGEATLVEEQDTNF | 43 |
| V4 | | VCRRTFVDRGHGNGCGLFGKGSLITCAK | |
| | | FK | |

In some embodiments, the oligomerization domain comprises an amino acid sequence disclosed in Table 1, or an amino acid sequence that is substantially identical to an amino acid sequence in Table 1 (e.g. 80%, 85%, 90%, 95%, 96%, 97%, 98%, 99% sequence identity). In some instances, the oligomerization domain comprises an amino acid sequence comprising at least a portion having at least or about 10, 20, 30, 40, 50, 60, 70, 80, 90, 100, 110, 120, 130 amino acid sequences of any sequence according to Table 1. In some embodiments, the oligomerization domain comprises an amino acid sequence that has at least 95% sequence identity to an amino acid sequence according to any one of SEQ ID NOs: 30-43.

### Antibodies

Described herein, in some embodiments, are antiviruses comprising a fusion protein that comprises a transmembrane polypeptide and an antibody which binds to a surface protein of a virus. In some embodiments, the antivirus does not comprise viral genetic material. In some embodiments, the antivirus neutralizes the virus when the fusion protein is bound to the surface protein of the virus.

In some embodiments, the antibody comprises a single chain variable fragment (scFv), a single domain antibody, an Fv, a VH domain, a VL domain, a Fab fragment, a monoclonal antibody, F(ab′), F(ab′)2, single chain antibodies, diabodies, or a scFv-Fc. In some embodiments, the antibody binds specifically to the surface protein of the virus. In some embodiments, the antibody is a multispecific antibody. In some embodiments, the multispecific antibody binds specifically to more than one epitope on the surface protein of the virus. In some embodiments, the multispecific antibody comprises a tandem scFv format.

Described herein, in some embodiments, are antiviruses comprising a fusion protein that comprises a transmembrane polypeptide and an antibody which binds to a surface protein of a virus, wherein the virus comprises SARS COV-1, CoV-2, influenza, or MERS CoV virus. In some embodiments, the antibody comprises an amino acid sequence from at least one complementarity determining region of 80R, H4, 7D10, 1E01, F16, 0304-4A10, 2M-14E5, 9A1, COV2-2021, COV2-2026, COV2-2146, 2M-10B11, C021, C018, BG4-5, BG7-15 or BG10-19.

In some embodiments, the antibody comprises the scFv and an amino acid sequence from at least one complementarity determining region of 80R. In some embodiments, the antibody comprises the scFv and an amino acid sequence from at least one complementarity determining region of H4. In some embodiments, the antibody comprises the scFv and an amino acid sequence from at least one complementarity determining region of 7D10. In some embodiments, the antibody comprises the scFv and an amino acid sequence from at least one complementarity determining region of COV2-2021. In some embodiments, the antibody comprises the scFv and an amino acid sequence from at least one complementarity determining region of C021. In some embodiments, the antibody comprises the scFv and an amino acid sequence from at least one complementarity determining region of C018.

In some embodiments, the antibody neutralizes the virus when expressed within the fusion protein but does not neutralize the virus when expressed as an isolated antibody. In some embodiments, the antibody neutralizes the virus when expressed within the fusion protein and neutralizes the virus when expressed as an isolated antibody.

In some embodiments, the antibody comprises a variable domain of heavy chain amino acid sequence of at least 75% sequence identity to an amino acid sequence according to any one of SEQ ID NOs: 1-14. In some embodiments, the antibody comprises a variable domain of heavy chain amino acid sequence of at least 80% sequence identity to an amino acid sequence according to any one of SEQ ID NOs: 1-14, 81, 83 and 85. In some embodiments, the antibody comprises a variable domain of heavy chain amino acid sequence of at least 85% sequence identity to an amino acid sequence according to any one of SEQ ID NOs: 1-14, 81, 83 and 85. In some embodiments, the antibody comprises a variable domain of heavy chain amino acid sequence of at least 90% sequence identity to an amino acid sequence according to any one of SEQ ID NOs: 1-14, 81, 83 and 85. In some embodiments, the antibody comprises a variable domain of heavy chain amino acid sequence of at least 95% sequence identity to an amino acid sequence according to any one of SEQ ID NOs: 1-14, 81, 83 and 85. In some embodiments, the antibody comprises a variable domain of heavy chain amino acid sequence of at least 98% sequence identity to an amino acid sequence according to any one of SEQ ID NOs: 1-14, 81, 83 and 85. In some embodiments, the antibody comprises a variable domain of heavy chain amino acid sequence of at least 99% sequence identity to an amino acid sequence according to any one of SEQ ID NOs: 1-14, 81, 83 and 85.

In some embodiments, the antibody comprises a variable domain of heavy chain amino acid sequence of at least 75% sequence homology to an amino acid sequence according to any one of SEQ ID NOs: 1-14, 81, 83 and 85. In some embodiments, the antibody comprises a variable domain of heavy chain amino acid sequence of at least 80% sequence homology to an amino acid sequence according to any one of SEQ ID NOs: 1-14, 81, 83 and 85. In some embodiments, the antibody comprises a variable domain of heavy chain amino acid sequence of at least 85% sequence homology to an amino acid sequence according to any one of SEQ ID NOs: 1-14, 81, 83 and 85. In some embodiments, the antibody comprises a variable domain of heavy chain amino acid sequence of at least 90% sequence homology to an amino acid sequence according to any one of SEQ ID NOs: 1-14, 81, 83 and 85. In some embodiments, the antibody comprises a variable domain of heavy chain amino acid sequence of at least 95% sequence homology to an amino acid sequence according to any one of SEQ ID NOs: 1-14, 81, 83 and 85. In some embodiments, the antibody comprises a variable domain of heavy chain amino acid sequence of at least 98% sequence homology to an amino acid sequence according to any one of SEQ ID NOs: 1-14, 81, 83 and 85. In some embodiments, the antibody comprises a variable domain of heavy chain amino acid sequence of at least 99% sequence homology to an amino acid sequence according to any one of SEQ ID NOs: 1-14, 81, 83 and 85.

In some instances, the antibody comprises a variable domain of heavy chain amino acid sequence comprising at least a portion having at least or about 10, 20, 30, 40, 50, 60, 70, 80, 90, 100, 110, or more than 110 amino acids of SEQ ID NOs: 1-14, 81, 83 and 85.

In some embodiments, the antibody comprises a variable domain of light chain amino acid sequence of at least 75% sequence identity to an amino acid sequence according to any one of SEQ ID NOs: 15-28, 82, 84 and 86. In some embodiments, the antibody comprises a variable domain of light chain amino acid sequence of at least 80% sequence identity to an amino acid sequence according to any one of SEQ ID NOs: 15-28, 82, 84 and 86. In some embodiments, the antibody comprises a variable domain of light chain amino acid sequence of at least 85% sequence identity to an amino acid sequence according to any one of SEQ ID NOs: 15-28, 82, 84 and 86. In some embodiments, the antibody comprises a variable domain of light chain amino acid sequence of at least 90% sequence identity to an amino acid sequence according to any one of SEQ ID NOs: 15-28, 82, 84 and 86. In some embodiments, the antibody comprises a variable domain of light chain amino acid sequence of at least 95% sequence identity to an amino acid sequence according to any one of SEQ ID NOs: 15-28, 82, 84 and 86. In some embodiments, the antibody comprises a variable domain of light chain amino acid sequence of at least 98% sequence identity to an amino acid sequence according to any one of SEQ ID NOs: 15-28, 82, 84 and 86. In some embodiments, the antibody comprises a variable domain of light chain amino acid sequence of at least 99% sequence identity to an amino acid sequence according to any one of SEQ ID NOs: 15-28, 82, 84 and 86.

In some embodiments, the antibody comprises a variable domain of light chain amino acid sequence of at least 75% sequence homology to an amino acid sequence according to any one of SEQ ID NOs: 15-28, 82, 84 and 86. In some embodiments, the antibody comprises a variable domain of light chain amino acid sequence of at least 80% sequence homology to an amino acid sequence according to any one of SEQ ID NOs: 15-28, 82, 84 and 86. In some embodiments, the antibody comprises a variable domain of light chain amino acid sequence of at least 85% sequence homology to an amino acid sequence according to any one of SEQ ID NOs: 15-28, 82, 84 and 86. In some embodiments, the antibody comprises a variable domain of light chain amino acid sequence of at least 90% sequence homology to an amino acid sequence according to any one of SEQ ID NOs: 15-28, 82, 84 and 86. In some embodiments, the antibody comprises a variable domain of light chain amino acid sequence of at least 95% sequence homology to an amino acid sequence according to any one of SEQ ID NOs: 15-28, 82, 84 and 86. In some embodiments, the antibody comprises a variable domain of light chain amino acid sequence of at least 98% sequence homology to an amino acid sequence according to any one of SEQ ID NOs: 15-28, 82, 84 and 86. In some embodiments, the antibody comprises a variable domain of light chain amino acid sequence of at least 99% sequence homology to an amino acid sequence according to any one of SEQ ID NOs: 15-28, 82, 84 and 86.

In some instances, the antibody comprises a variable domain of light chain amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800 amino acids of SEQ ID NOs: 15-28, 82, 84 and 86.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. Typically, techniques for determining sequence identity include comparing two nucleotide or amino acid sequences and the determining their percent identity. Sequence comparisons, such as for the purpose of assessing identities, may be performed by any suitable alignment algorithm, including but not limited to the Needleman-Wunsch algorithm (see, e.g., the EMBOSS Needle aligner available at www.ebi.ac.uk/Tools/psa/emboss_needle/, optionally with default settings), the BLAST algorithm (see, e.g., the BLAST alignment tool available at blast.ncbi.nlm.nih.gov/Blast.cgi, optionally with default settings), and the Smith-Waterman algorithm (see, e.g., the EMBOSS Water aligner available at www.ebi.ac.uk/Tools/psa/emboss_water/, optionally with default settings). Optimal alignment may be assessed using any suitable parameters of a chosen algorithm, including default parameters. The “percent identity”, also referred to as “percent homology”, between two sequences may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin and Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the sequences being compared. Default parameters are provided to optimize searches with short query sequences, for example, with the blastp program. The program also allows use of an SEG filter to mask-off segments of the query sequences as determined by the SEG program of Wootton and Federhen, Computers and Chemistry 17: 149-163 (1993). High sequence identity generally includes ranges of sequence identity of approximately 80% to 100% and integer values there between.

Described herein, in some embodiments, are antiviruses comprising a fusion protein that comprises an antibody and a transmembrane domain, wherein the antivirus further comprises a second fusion protein. In some embodiments, the antivirus further comprises a second fusion protein that comprises transmembrane polypeptide and a second antibody which binds to a surface protein of the virus, wherein the second antibody comprises a CDR sequence that has less than 100% sequence identity to an equivalent CDR sequence of the antibody. In some embodiments, the fusion protein and the second fusion protein comprise the same transmembrane polypeptide. In some embodiments, the fusion protein and the second fusion protein comprise different transmembrane polypeptides. In some embodiments, the second antibody binds to the same surface protein as the antibody. In some embodiments, the second antibody binds to a different surface protein as the antibody.

In some embodiments, the second antibody is a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc. In some embodiments, the second antibody binds specifically to the surface protein of the virus.

In some embodiments, the second antibody is a multispecific antibody. In some embodiments, the second multispecific antibody binds specifically to more than one epitope on the surface protein of the virus. In some embodiments, the second antibody comprises an amino acid sequence from at least one complementarity determining region of 80R, H4, 7D10, 1E01, F16, 0304-4A10, 2M-14E5, 9A1, COV2-2021, COV2-2026, COV2-2146, 2M-10B11, C021, C018, BG4-5, BG7-15, or BG10-19. In some embodiments, the second antibody comprises the scFv and an amino acid sequence from at least one complementarity determining region of 80R. In some embodiments, the second antibody comprises the scFv and an amino acid sequence from at least one complementarity determining region of H4. In some embodiments, the second antibody comprises the scFv and an amino acid sequence from at least one complementarity determining region of 7D10. In some embodiments, the second antibody comprises the scFv and an amino acid sequence from at least one complementarity determining region of COV2-2021. In some embodiments, the second antibody comprises the scFv and an amino acid sequence from at least one complementarity determining region of C021. In some embodiments, the second antibody comprises the scFv and an amino acid sequence from at least one complementarity determining region of C018. In some embodiments, the second antibody neutralizes the virus when expressed within the fusion protein but does not neutralize the virus when expressed as an isolated antibody. In some embodiments, the second antibody neutralizes the virus when expressed within the fusion protein and neutralizes the virus when expressed as an isolated antibody.

In some embodiments, the second antibody neutralizes the virus when displayed as a fusion protein on the surface of the antivirus but does not neutralize the virus when expressed as an isolated antibody. In some embodiments, the second antibody neutralizes the virus when expressed within the fusion protein and neutralizes the virus when expressed as an isolated antibody.

In some embodiments, the second fusion protein further comprises an oligomerization domain. In some embodiments, the oligomerization domain is a dimerization domain. In some embodiments, the oligomerization domain is a trimerization domain. In some embodiments, the oligomerization domain comprises the D4 trimerization domain of VSV-G protein.

In some embodiments, the antibody comprises the scFv and an amino acid sequence from at least one complementarity determining region of COV2-2021. In some embodiments, the antibody comprises the scFv and an amino acid sequence from at least one complementarity determining region of C021. In some embodiments, the antibody comprises the scFv and an amino acid sequence from at least one complementarity determining region of C018. In some embodiments, the antibody comprises the scFv and an amino acid sequence from at least one complementarity determining region of H4 and the oligomerization domain comprises the D4 trimerization domain of VSV-G protein. In some embodiments, the antibody comprises the scFv and an amino acid sequence from at least one complementarity determining region of 0304-4A10, and the oligomerization domain comprises the D4 trimerization domain of VSV-G protein. In some embodiments, the antibody comprises the scFv and an amino acid sequence from at least one complementarity determining region of 2M-14E5, and the oligomerization domain comprises the D4 trimerization domain of VSV-G protein. In some embodiments, the antibody comprises the scFv and an amino acid sequence from at least one complementarity determining region of 9A1, and the oligomerization domain comprises the D4 trimerization domain of VSV-G protein. In some embodiments, the antibody comprises the scFv and an amino acid sequence from at least one complementarity determining region of COV2-2021, and the oligomerization domain comprises the D4 trimerization domain of VSV-G protein. In some embodiments, the antibody comprises the scFv and an amino acid sequence from at least one complementarity determining region of COV2-2026, and the oligomerization domain comprises the D4 trimerization domain of VSV-G protein. In some embodiments, the antibody comprises the scFv and an amino acid sequence from at least one complementarity determining region of COV2-2146, and the oligomerization domain comprises the D4 trimerization domain of VSV-G protein. In some embodiments, the antibody comprises the scFv and an amino acid sequence from at least one complementarity determining region of 2M-10B11, and the oligomerization domain comprises the D4 trimerization domain of VSV-G protein. In some embodiments, the antibody comprises the scFv and an amino acid sequence from at least one complementarity determining region of C021, and the oligomerization domain comprises the D4 trimerization domain of VSV-G protein. In some embodiments, the antibody comprises the scFv and an amino acid sequence from at least one complementarity determining region of C018, and the oligomerization domain comprises the D4 trimerization domain of VSV-G protein. In some embodiments, the antibody comprises the scFv and an amino acid sequence from at least one complementarity determining region of BG4-5, and the oligomerization domain comprises the D4 trimerization domain of VSV-G protein. In some embodiments, the antibody comprises the scFv and an amino acid sequence from at least one complementarity determining region of BG7-15, and the oligomerization domain comprises the D4 trimerization domain of VSV-G protein. In some embodiments, the antibody comprises the scFv and an amino acid sequence from at least one complementarity determining region of BG10-19, and the oligomerization domain comprises the D4 trimerization domain of VSV-G protein.

In some embodiments, the second antibody is a multispecific antibody. In some embodiments, the second multispecific antibody binds specifically to more than one epitope on the surface protein of the virus. In some embodiments, the multispecific antibody that comprises a tandem scFv format binds to a Neuraminidase active site and a Hemagglutinin stem of influenza virus and the fusion protein oligomerization domain comprises the D4 trimerization domain of VSV-G protein. In some embodiments, the multispecific antibody that comprises a tandem scFv format binds to a Spike NTD and a Spike RBD of SARS COV-2 and the fusion protein oligomerization domain comprises the D4 trimerization domain of VSV-G protein. In some embodiments, the multispecific antibody that comprises a tandem scFv format comprises an amino acid sequence from at least one complementarity determining region of CoV2-2021 and an amino acid sequence from at least one complementarity determining region of C012 and the fusion protein oligomerization domain comprises the D4 trimerization domain of VSV-G protein. In some embodiments, the antibody comprises the scFv and an amino acid sequence from at least one complementarity determining region of 0304-4A10, and the oligomerization domain comprises the D4 trimerization domain of VSV-G protein, and the second antibody comprises the scFv and an amino acid sequence from at least one complementarity determining region of C018.

In some embodiments, the second antibody comprises amino acid sequence of at least 75% sequence identity to an amino acid sequence according to any one of SEQ ID NOs: 1-28 and 81-86. In some embodiments, the second antibody comprises amino acid sequence of at least 80% sequence identity to an amino acid sequence according to any one of SEQ ID NOs: 1-28 and 81-86. In some embodiments, the second antibody comprises amino acid sequence of at least 85% sequence identity to an amino acid sequence according to any one of SEQ ID NOs: 1-28 and 81-86. In some embodiments, the second antibody comprises amino acid sequence of at least 90% sequence identity to an amino acid sequence according to any one of SEQ ID NOs: 1-28 and 81-86. In some embodiments, the second antibody comprises amino acid sequence of at least 95% sequence identity to an amino acid sequence according to any one of SEQ ID NOs: 1-28 and 81-86. In some embodiments, the second antibody comprises amino acid sequence of at least 98% sequence identity to an amino acid sequence according to any one of SEQ ID NOs: 1-28 and 81-86. In some embodiments, the second antibody comprises amino acid sequence of at least 99% sequence identity to an amino acid sequence according to any one of SEQ ID NOs: 1-28 and 81-86.

In some embodiments, the second antibody comprises amino acid sequence of at least 75% sequence homology to an amino acid sequence according to any one of SEQ ID NOs: 1-28 and 81-86. In some embodiments, the second antibody comprises amino acid sequence of at least 80% sequence homology to an amino acid sequence according to any one of SEQ ID NOs: 1-28 and 81-86. In some embodiments, the second antibody comprises amino acid sequence of at least 85% sequence homology to an amino acid sequence according to any one of SEQ ID NOs: 1-28 and 81-86. In some embodiments, the second antibody comprises amino acid sequence of at least 90% sequence homology to an amino acid sequence according to any one of SEQ ID NOs: 1-28 and 81-86. In some embodiments, the second antibody comprises amino acid sequence of at least 95% sequence homology to an amino acid sequence according to any one of SEQ ID NOs: 1-28 and 81-86. In some embodiments, the second antibody comprises amino acid sequence of at least 98% sequence homology to an amino acid sequence according to any one of SEQ ID NOs: 1-28 and 81-86. In some embodiments, the second antibody comprises amino acid sequence of at least 99% sequence homology to an amino acid sequence according to any one of SEQ ID NOs: 1-28 and 81-86.

In some instances, the second antibody comprises amino acid sequence comprising at least a portion having at least or about 10, 20, 30, 40, 50, 60, 70, 80, 90, 100, 110, or more than 100 amino acids of SEQ ID NOs: 1-28 and 81-86.

In some embodiments, the second antibody comprises a variable domain of heavy chain amino acid sequence of at least 75% sequence identity to an amino acid sequence according to any one of SEQ ID NOs: 1-14, 81, 83 and 85. In some embodiments, the second antibody comprises a variable domain of heavy chain amino acid sequence of at least 80% sequence identity to an amino acid sequence according to any one of SEQ ID NOs: 1-14, 81, 83 and 85. In some embodiments, the second antibody comprises a variable domain of heavy chain amino acid sequence of at least 85% sequence identity to an amino acid sequence according to any one of SEQ ID NOs: 1-14, 81, 83 and 85. In some embodiments, the second antibody comprises a variable domain of heavy chain amino acid sequence of at least 90% sequence identity to an amino acid sequence according to any one of SEQ ID NOs: 1-14, 81, 83 and 85. In some embodiments, the second antibody comprises a variable domain of heavy chain amino acid sequence of at least 95% sequence identity to an amino acid sequence according to any one of SEQ ID NOs: 1-14, 81, 83 and 85. In some embodiments, the second antibody comprises a variable domain of heavy chain amino acid sequence of at least 98% sequence identity to an amino acid sequence according to any one of SEQ ID NOs: 1-14, 81, 83 and 85. In some embodiments, the second antibody comprises a variable domain of heavy chain amino acid sequence of at least 99% sequence identity to an amino acid sequence according to any one of SEQ ID NOs: 1-14, 81, 83 and 85.

In some embodiments, the second antibody comprises a variable domain of heavy chain amino acid sequence of at least 75% sequence homology to an amino acid sequence according to any one of SEQ ID NOs: 1-14, 81, 83 and 85. In some embodiments, the second antibody comprises a variable domain of heavy chain amino acid sequence of at least 80% sequence homology to an amino acid sequence according to any one of SEQ ID NOs: 1-14, 81, 83 and 85. In some embodiments, the second antibody comprises a variable domain of heavy chain amino acid sequence of at least 85% sequence homology to an amino acid sequence according to any one of SEQ ID NOs: 1-14, 81, 83 and 85. In some embodiments, the second antibody comprises a variable domain of heavy chain amino acid sequence of at least 90% sequence homology to an amino acid sequence according to any one of SEQ ID NOs: 1-14, 81, 83 and 85. In some embodiments, the second antibody comprises a variable domain of heavy chain amino acid sequence of at least 95% sequence homology to an amino acid sequence according to any one of SEQ ID NOs: 1-14, 81, 83 and 85. In some embodiments, the second antibody comprises a variable domain of heavy chain amino acid sequence of at least 98% sequence homology to an amino acid sequence according to any one of SEQ ID NOs: 1-14, 81, 83 and 85. In some embodiments, the second antibody comprises a variable domain of heavy chain amino acid sequence of at least 99% sequence homology to an amino acid sequence according to any one of SEQ ID NOs: 1-14, 81, 83 and 85.

In some instances, the second antibody comprises a variable domain of heavy chain amino acid sequence comprising at least a portion having at least or about 10, 20, 30, 40, 50, 60, 70, 80, 90, 100, 110, or more than 100 amino acids of SEQ ID NOs: 1-14, 81, 83 and 85.

In some embodiments, the second antibody comprises a variable domain of light chain amino acid sequence of at least 75% sequence identity to an amino acid sequence according to any one of SEQ ID NOs: 15-28, 82, 84 and 86. In some embodiments, the second antibody comprises a variable domain of light chain amino acid sequence of at least 80% sequence identity to an amino acid sequence according to any one of SEQ ID NOs: 15-28, 82, 84 and 86. In some embodiments, the second antibody comprises a variable domain of light chain amino acid sequence of at least 85% sequence identity to an amino acid sequence according to any one of SEQ ID NOs: 15-28, 82, 84 and 86. In some embodiments, the second antibody comprises a variable domain of light chain amino acid sequence of at least 90% sequence identity to an amino acid sequence according to any one of SEQ ID NOs: 15-28, 82, 84 and 86. In some embodiments, the second antibody comprises a variable domain of light chain amino acid sequence of at least 95% sequence identity to an amino acid sequence according to any one of SEQ ID NOs: 15-28, 82, 84 and 86. In some embodiments, the second antibody comprises a variable domain of light chain amino acid sequence of at least 98% sequence identity to an amino acid sequence according to any one of SEQ ID NOs: 15-28, 82, 84 and 86. In some embodiments, the second antibody comprises a variable domain of light chain amino acid sequence of at least 99% sequence identity to an amino acid sequence according to any one of SEQ ID NOs: 15-28, 82, 84 and 86.

In some embodiments, the second antibody comprises a variable domain of light chain amino acid sequence of at least 75% sequence homology to an amino acid sequence according to any one of SEQ ID NOs: 15-28, 82, 84 and 86. In some embodiments, the second antibody comprises a variable domain of light chain amino acid sequence of at least 80% sequence homology to an amino acid sequence according to any one of SEQ ID NOs: 15-28, 82, 84 and 86. In some embodiments, the second antibody comprises a variable domain of light chain amino acid sequence of at least 85% sequence homology to an amino acid sequence according to any one of SEQ ID NOs: 15-28, 82, 84 and 86. In some embodiments, the second antibody comprises a variable domain of light chain amino acid sequence of at least 90% sequence homology to an amino acid sequence according to any one of SEQ ID NOs: 15-28, 82, 84 and 86. In some embodiments, the second antibody comprises a variable domain of light chain amino acid sequence of at least 95% sequence homology to an amino acid sequence according to any one of SEQ ID NOs: 15-28, 82, 84 and 86. In some embodiments, the second antibody comprises a variable domain of light chain amino acid sequence of at least 98% sequence homology to an amino acid sequence according to any one of SEQ ID NOs: 15-28, 82, 84 and 86. In some embodiments, the second antibody comprises a variable domain of light chain amino acid sequence of at least 99% sequence homology to an amino acid sequence according to any one of SEQ ID NOs: 15-28, 82, 84 and 86.

In some instances, the second antibody comprises a variable domain of light chain amino acid sequence comprising at least a portion having at least or about 10, 20, 30, 40, 50, 60, 70, 80, 90, 100, or more than 100 amino acids of SEQ ID NOs: 15-28, 82, 84 and 86.

In some embodiments, the second antibody neutralizes the virus when displayed as a fusion protein on the surfaces of the antivirus but does not neutralize the virus when expressed as an isolated antibody. In some embodiments, the second antibody neutralizes the virus when expressed within the fusion protein and neutralizes the virus when expressed as an isolated antibody. In some embodiments, the multispecific antibody comprises the scFv and an amino acid sequence from at least one complementarity determining region of 0304-4A10, and the oligomerization domain comprises the D4 trimerization domain of VSV-G protein, and the second antibody comprises the scFv and an amino acid sequence from at least one complementarity determining region of C018.

### Transmembrane Polypeptides

Described herein, in some embodiments, are antiviruses comprising a fusion protein that comprises a transmembrane polypeptide and an antibody. In some embodiments, the transmembrane polypeptide comprises the transmembrane domain of a Vesicular Stomatitis virus glycoprotein (VSV-G). In some embodiments, the transmembrane polypeptide comprises the transmembrane domain and cytosolic domain of a Vesicular Stomatitis virus glycoprotein (VSV-G). In some embodiments, the transmembrane polypeptide comprises the transmembrane domain of a Dengue E protein. In some embodiments, the transmembrane polypeptide comprises the transmembrane domain and cytosolic domain of a Dengue E protein. In some embodiments, the transmembrane polypeptide comprises the transmembrane domain of influenza Hemagglutinin (HA). In some embodiments, the transmembrane polypeptide comprises the transmembrane domain and cytosolic domain of influenza Hemagglutinin (HA). In some embodiments, the transmembrane polypeptide comprises the transmembrane domain of HIV surface glycoprotein GP120 or GP41. In some embodiments, the transmembrane polypeptide comprises the transmembrane domain and cytosolic domain of HIV surface glycoprotein GP120 or GP41. In some embodiments, the transmembrane domain comprises the transmembrane polypeptide of measles virus surface glycoprotein hamagglutinin (H) protein. In some embodiments, the transmembrane polypeptide comprises the transmembrane domain and cytosolic domain of measles virus surface glycoprotein hamagglutinin (H) protein. In some embodiments, the transmembrane polypeptide comprises the transmembrane domain of influenza Neuraminidase (NA). In some embodiments, the transmembrane polypeptide comprises the transmembrane domain and cytosolic domain of influenza Neuraminidase (NA).

TABLE 2
Exemplary Transmembrane Domain Sequences

| Domain | Amino Acid Sequence | SEQ ID NO: |
|---|---|---|
| VSV-G | IASFFFIIGLIIGLFLVLRVGI | 44 |
| Transmembrane | | |
| (TM) V1 | | |
| VSV-G | PIELVEGWFSSWKSSIASFFFIIGLIIGLFL | 45 |
| Transmembrane | VLRVGI | |
| (TM) V2 | | |
| VSV-G | DDESLFFGDTGLSKNPIELVEGWFSSWK | 46 |
| Transmembrane | SSIASFFFIIGLIIGLFLVLRVGIH | |
| (TM) V3 | | |
| VSV-G | GMLDSDLHLSSKAQVFEHPHIQDAASQL | 47 |
| Transmembrane | PDDESLFFGDTGLSKNPIELVEGWFSSW | |
| TM) V4 | KSSIASFFFIIGLIIGLFLVLRVGI | |
| VSV-G | HLCIKLKHTKKRQIYTDIEMNRLGK | 48 |
| Cytosolic Tail | | |
| (CT) | | |
| Influenza | IITIGSVCMTIGMANLILQIGNI | 49 |
| Neuraminidase | | |
| TM (N1) | | |
| Influenza | LAIYSTVASSLVLVVSLGAISFW | 50 |
| Hemagglutinin | | |
| TM (H1) | | |
| Dengue E | AYGVLFSGVSWTMKIGIGILLTWLGLNS | 51 |
| Protein TM | RSTSLSMTCIAVGMVTLYLGVMVQ | |
| HIV gp TM | FIMIVGGLVGLRIVFAVLSIV | 52 |

In some embodiments, the transmembrane domain comprises an amino acid sequence disclosed in Table 2, or an amino acid sequence that is substantially identical to an amino acid sequence in Table 2 (e.g. 80%, 85%, 90%, 95%, 96%, 97%, 98%, 99% sequence identity). In some instances, the transmembrane domain comprises an amino acid sequence comprising at least a portion having at least or about 10, 20, 30, 40, 50, 60, 70, 80, 90, 100, 110, 120, 130 amino acid sequences of any sequence according to Table 2.

Described herein, in some embodiments, are antiviruses comprising a fusion protein that comprises a transmembrane polypeptide and an antibody. In some embodiments, the transmembrane polypeptide anchors the fusion protein to a bilayer of the antivirus. In some embodiments, the transmembrane polypeptide comprises the transmembrane domain of a Vesicular Stomatitis virus glycoprotein (VSV-G). In some embodiments, the transmembrane polypeptide comprises the transmembrane domain of influenza Hemagglutinin (HA). In some embodiments, the transmembrane polypeptide comprises the transmembrane domain of HIV surface glycoprotein GP120 or GP41. In some embodiments, the transmembrane polypeptide comprises the transmembrane domain of measles virus surface glycoprotein hamagglutinin (H) protein. In some embodiments, the transmembrane polypeptide comprises the transmembrane domain of influenza Neuraminidase (NA).

In some embodiments, the transmembrane polypeptide comprises an amino acid sequence of at least 75% sequence identity to an amino acid sequence according to SEQ ID NO: 29. In some embodiments, the transmembrane polypeptide comprises an amino acid sequence of at least 80% sequence identity to an amino acid sequence according to SEQ ID NO: 29. In some embodiments, the transmembrane polypeptide comprises an amino acid sequence of at least 85% sequence identity to an amino acid sequence according to SEQ ID NO: 29. In some embodiments, the transmembrane polypeptide comprises an amino acid sequence of at least 90% sequence identity to an amino acid sequence according to SEQ ID NO: 29. In some embodiments, the transmembrane polypeptide comprises an amino acid sequence of at least 95% sequence identity to an amino acid sequence according to SEQ ID NO: 29. In some embodiments, the transmembrane polypeptide comprises an amino acid sequence of at least 98% sequence identity to an amino acid sequence according to SEQ ID NO: 29. In some embodiments, the transmembrane polypeptide comprises an amino acid sequence of at least 99% sequence identity to an amino acid sequence according to SEQ ID NO: 29.

In some embodiments, the transmembrane polypeptide comprises an amino acid sequence of at least 75% sequence homology to an amino acid sequence according to SEQ ID NO: 29. In some embodiments, the transmembrane polypeptide comprises an amino acid sequence of at least 80% sequence homology to an amino acid sequence according to SEQ ID NO: 29. In some embodiments, the transmembrane polypeptide comprises an amino acid sequence of at least 85% sequence homology to an amino acid sequence according to SEQ ID NO: 29. In some embodiments, the transmembrane polypeptide comprises an amino acid sequence of at least 90% sequence homology to an amino acid sequence according to SEQ ID NO: 29. In some embodiments, the transmembrane polypeptide comprises an amino acid sequence of at least 95% sequence homology to an amino acid sequence according to SEQ ID NO: 29. In some embodiments, the transmembrane polypeptide comprises an amino acid sequence of at least 98% sequence homology to an amino acid sequence according to SEQ ID NO: 29. In some embodiments, the transmembrane polypeptide comprises an amino acid sequence of at least 99% sequence homology to an amino acid sequence according to SEQ ID NO: 29.

In some instances, the transmembrane polypeptide comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or more than 490 amino acids of SEQ ID NO: 29.

TABLE 3
VSVG Sequence

| | SEQ | | |
|---|---|---|---|
| Name | ID No. | Accession No. | Amino Acid Sequences |
| VSVG | 29 | NP_955548 | KFTIVFPHNQKGNWKNVPSNYHYCPSSSDLNWHNDLI |
| | | | GTALQVKMPKSHKAIQADGWMCHASKWVTTCDFR |
| | | | WYGPKYITHSIRSFTPSVEQCKESIEQTKQGTWLNPGF |
| | | | PPQSCGYATVIDAEAVIVQVTPHHVLVDEYTGEWVD |
| | | | SQFINGKCSNYICPTVHNSTTWHSDYKVKGLCDSNLIS |
| | | | MDITFFSEDGELSSLGKEGTGFRSNYFAYETGGKACK |
| | | | MQYCKHWGVRLPSGVWFEMADKDLFAAARFPECPE |
| | | | GSSISAPSQTSVDVSLIQDVERILDYSLCQETWSKIRAG |
| | | | LPISPVDLSYLAPKNPGTGPAFTIINGTLKYFETRYIRV |
| | | | DIAAPILSRMVGMISGTTTERELWDDWAPYEDVEIGP |
| | | | NGVLRTSSGYKFPLYMIGHGMLDSDLHLSSKAQVFEH |
| | | | PHIQDAASQLPDDESLFFGDTGLSKNPIELVEGWFSSW |
| | | | KSSIASFFFIIGLIIGLFLVLRVGIHLCIKLKHTKKRQIYT |
| | | | DIEMNRLGK |

Described herein, in some embodiments, are antiviruses comprising a fusion protein that comprises a transmembrane polypeptide and an antibody wherein the antiviruses further comprises a second fusion protein that comprises a second transmembrane polypeptide and a second antibody. In some embodiments, the second transmembrane polypeptide anchors the fusion protein to a bilayer of the antivirus. In some embodiments, the second transmembrane polypeptide comprises the transmembrane domain of a Vesicular Stomatitis virus glycoprotein (VSV-G). In some embodiments, the second transmembrane polypeptide comprises the transmembrane domain of influenza Hemagglutinin (HA). In some embodiments, the second transmembrane polypeptide comprises the transmembrane domain of HIV surface glycoprotein GP120 or GP41. In some embodiments, the second transmembrane polypeptide comprises the transmembrane domain of measles virus surface glycoprotein hamagglutinin (H) protein. In some embodiments, the second transmembrane polypeptide comprises the transmembrane domain of influenza Neuraminidase (NA).

In some embodiments, the second transmembrane polypeptide comprises an amino acid sequence of at least 75% sequence identity to an amino acid sequence according to SEQ ID NO: 29. In some embodiments, the second transmembrane polypeptide comprises an amino acid sequence of at least 80% sequence identity to an amino acid sequence according to SEQ ID NO: 29. In some embodiments, the second transmembrane polypeptide comprises an amino acid sequence of at least 85% sequence identity to an amino acid sequence according to SEQ ID NO: 29. In some embodiments, the second transmembrane polypeptide comprises an amino acid sequence of at least 90% sequence identity to an amino acid sequence according to SEQ ID NO: 29. In some embodiments, the second transmembrane polypeptide comprises an amino acid sequence of at least 95% sequence identity to an amino acid sequence according to SEQ ID NO: 29. In some embodiments, the second transmembrane polypeptide comprises an amino acid sequence of at least 98% sequence identity to an amino acid sequence according to SEQ ID NO: 29. In some embodiments, the second transmembrane polypeptide comprises an amino acid sequence of at least 99% sequence identity to an amino acid sequence according to SEQ ID NO: 29.

In some embodiments, the second transmembrane polypeptide comprises an amino acid sequence of at least 75% sequence homology to an amino acid sequence according to SEQ ID NO: 29. In some embodiments, the second transmembrane polypeptide comprises an amino acid sequence of at least 80% sequence homology to an amino acid sequence according to SEQ ID NO: 29. In some embodiments, the second transmembrane polypeptide comprises an amino acid sequence of at least 85% sequence homology to an amino acid sequence according to SEQ ID NO: 29. In some embodiments, the second transmembrane polypeptide comprises an amino acid sequence of at least 90% sequence homology to an amino acid sequence according to SEQ ID NO: 29. In some embodiments, the second transmembrane polypeptide comprises an amino acid sequence of at least 95% sequence homology to an amino acid sequence according to SEQ ID NO: 29. In some embodiments, the second transmembrane polypeptide comprises an amino acid sequence of at least 98% sequence homology to an amino acid sequence according to SEQ ID NO: 29. In some embodiments, the second transmembrane polypeptide comprises an amino acid sequence of at least 99% sequence homology to an amino acid sequence according to SEQ ID NO: 29.

In some instances, the second transmembrane polypeptide comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or more than 490 amino acids of SEQ ID NO: 29.

### Antibody and Transmembrane Polypeptide Combinations

Described herein, in some embodiments, are antiviruses comprising a fusion protein that comprises a transmembrane polypeptide and an antibody which binds to a surface protein of a virus. In some embodiments, the fusion protein comprises an antibody that comprises an amino acid sequence from at least one complementarity determining region of 80R, H4, 7D10, 1E01, F16, 0304-4A10, 2M-14E5, 9A1, COV2-2021, COV2-2026, COV2-2146, 2M-10B11, C021, or C018 and a transmembrane polypeptide that comprises the transmembrane domain of a Vesicular Stomatitis virus glycoprotein (VSV-G), the transmembrane domain of influenza Hemagglutinin (HA), the transmembrane domain of HIV surface glycoprotein GP120 or GP41, the transmembrane domain of measles virus surface glycoprotein hamagglutinin (H) protein, or the transmembrane domain of influenza Neuraminidase (NA).

Described herein, in some embodiments, are antiviruses comprising a fusion protein that comprises a transmembrane polypeptide and an antibody which binds to a surface protein of a virus, wherein the antiviruses further comprise an oligomerization domain.

In some embodiments, the oligomerization domain is a dimerization domain. In some embodiments, the dimerization domain comprises a leucine zipper dimerization domain. In some embodiments, the oligomerization domain is a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the oligomerization domain is a tetramerization domain. In some embodiments, the tetramerization domain comprises an influenza neuraminidase stem domain.

In some embodiments, when the fusion protein is expressed on the surface of the antivirus, the oligomerization domain is outside of the antivirus. In some embodiments, when the fusion protein is expressed on the surface of the antivirus, the oligomerization domain is outside of the antivirus and adjacent to a signal peptide. In some embodiments, when the fusion protein is expressed on the surface of the antivirus, the oligomerization domain is inside of the antivirus. In some embodiments, when the fusion protein is expressed on the surface of the antivirus, the oligomerization domain is inside of the antivirus and adjacent to the transmembrane domain.

In some embodiments, the fusion protein comprises a signal peptide.

In some embodiments, domains of the fusion protein are arranged from the N-terminus to the C-terminus in the following orders: (a) signal peptide, antibody which binds to a surface protein of a virus, oligomerization domain, transmembrane domain, and cytosolic domain; (b) signal peptide, antibody which binds to a surface protein of a virus, transmembrane domain, oligomerization domain, and cytosolic domain; or (c) signal peptide, oligomerization domain, antibody which binds to a surface protein of a virus, transmembrane domain, and cytosolic domain. In some embodiments, domains of the fusion protein are arranged from the N-terminus to the C-terminus in the following order: signal peptide, antibody which binds to a surface protein of a virus, oligomerization domain, transmembrane domain, and cytosolic domain. In some embodiments, domains of the fusion protein are arranged from the N-terminus to the C-terminus in the following order: signal peptide, antibody which binds to a surface protein of a virus, transmembrane domain, oligomerization domain, and cytosolic domain. In some embodiments, domains of the fusion protein are arranged from the N-terminus to the C-terminus in the following order: signal peptide, oligomerization domain, antibody which binds to a surface protein of a virus, transmembrane domain, and cytosolic domain.

Disclosed herein are fusion proteins comprising a transmembrane domain, a cytosolic domain, an antibody which binds to a surface protein of a virus, and an oligomerization domain wherein when the fusion protein is expressed on the surface of an antivirus, the fusion protein is displayed in an oligomeric format.

In some embodiments, the antibody comprises the scFv and an amino acid sequence from at least one complementarity determining region of H4 and the oligomerization domain comprises the D4 trimerization domain of VSV-G protein. In some embodiments, the antibody comprises the scFv and an amino acid sequence from at least one complementarity determining region of 1E01 and the oligomerization domain comprises the D4 trimerization domain of VSV-G protein. In some embodiments, the antibody comprises the scFv and an amino acid sequence from at least one complementarity determining region of FI6 and the oligomerization domain comprises the D4 trimerization domain of VSV-G protein. In some embodiments, the antibody comprises the scFv and an amino acid sequence from at least one complementarity determining region of 0304-4A10, and the oligomerization domain comprises the D4 trimerization domain of VSV-G protein. In some embodiments, the antibody comprises the scFv and an amino acid sequence from at least one complementarity determining region of 2M-14E5, and the oligomerization domain comprises the D4 trimerization domain of VSV-G protein. In some embodiments, the antibody comprises the scFv and an amino acid sequence from at least one complementarity determining region of 9A1, and the oligomerization domain comprises the D4 trimerization domain of VSV-G protein. In some embodiments, the antibody comprises the scFv and an amino acid sequence from at least one complementarity determining region of COV2-2021, and the oligomerization domain comprises the D4 trimerization domain of VSV-G protein. In some embodiments, the antibody comprises the scFv and an amino acid sequence from at least one complementarity determining region of COV2-2026, and the oligomerization domain comprises the D4 trimerization domain of VSV-G protein. In some embodiments, the antibody comprises the scFv and an amino acid sequence from at least one complementarity determining region of COV2-2146, and the oligomerization domain comprises the D4 trimerization domain of VSV-G protein. In some embodiments, the antibody comprises the scFv and an amino acid sequence from at least one complementarity determining region of 10B11, and the oligomerization domain comprises the D4 trimerization domain of VSV-G protein. In some embodiments, the antibody comprises the scFv and an amino acid sequence from at least one complementarity determining region of C021, and the oligomerization domain comprises the D4 trimerization domain of VSV-G protein. In some embodiments, the antibody comprises the scFv and an amino acid sequence from at least one complementarity determining region of C018, and the oligomerization domain comprises the D4 trimerization domain of VSV-G protein. In some embodiments, the antibody comprises the scFv and an amino acid sequence from at least one complementarity determining region of BG4-5, and the oligomerization domain comprises the D4 trimerization domain of VSV-G protein. In some embodiments, the antibody comprises the scFv and an amino acid sequence from at least one complementarity determining region of BG7-15, and the oligomerization domain comprises the D4 trimerization domain of VSV-G protein. In some embodiments, the antibody comprises the scFv and an amino acid sequence from at least one complementarity determining region of BG10-19, and the oligomerization domain comprises the D4 trimerization domain of VSV-G protein.

In some embodiments, the antibody comprises the scFv and an amino acid sequence from at least one complementarity determining region of COV2-2021. In some embodiments, the antibody comprises the scFv and an amino acid sequence from at least one complementarity determining region of C021. In some embodiments, the antibody comprises the scFv and an amino acid sequence from at least one complementarity determining region of C018. In some embodiments, the antibody comprises the scFv and an amino acid sequence from at least one complementarity determining region of H4 and the oligomerization domain comprises the D4 trimerization domain of VSV-G protein. In some embodiments, the antibody comprises the scFv and an amino acid sequence from at least one complementarity determining region of 0304-4A10, and the oligomerization domain comprises the D4 trimerization domain of VSV-G protein. In some embodiments, the antibody comprises the scFv and an amino acid sequence from at least one complementarity determining region of 2M-14E5, and the oligomerization domain comprises the D4 trimerization domain of VSV-G protein. In some embodiments, the antibody comprises the scFv and an amino acid sequence from at least one complementarity determining region of 9A1, and the oligomerization domain comprises the D4 trimerization domain of VSV-G protein. In some embodiments, the antibody comprises the scFv and an amino acid sequence from at least one complementarity determining region of COV2-2021, and the oligomerization domain comprises the D4 trimerization domain of VSV-G protein. In some embodiments, the antibody comprises the scFv and an amino acid sequence from at least one complementarity determining region of COV2-2026, and the oligomerization domain comprises the D4 trimerization domain of VSV-G protein. In some embodiments, the antibody comprises the scFv and an amino acid sequence from at least one complementarity determining region of COV2-2146, and the oligomerization domain comprises the D4 trimerization domain of VSV-G protein. In some embodiments, the antibody comprises the scFv and an amino acid sequence from at least one complementarity determining region of 2M-10B11, and the oligomerization domain comprises the D4 trimerization domain of VSV-G protein. In some embodiments, the antibody comprises the scFv and an amino acid sequence from at least one complementarity determining region of C021, and the oligomerization domain comprises the D4 trimerization domain of VSV-G protein. In some embodiments, the antibody comprises the scFv and an amino acid sequence from at least one complementarity determining region of C018, and the oligomerization domain comprises the D4 trimerization domain of VSV-G protein. In some embodiments, the antibody comprises the scFv and an amino acid sequence from at least one complementarity determining region of BG4-5, and the oligomerization domain comprises the D4 trimerization domain of VSV-G protein. In some embodiments, the antibody comprises the scFv and an amino acid sequence from at least one complementarity determining region of BG7-15, and the oligomerization domain comprises the D4 trimerization domain of VSV-G protein. In some embodiments, the antibody comprises the scFv and an amino acid sequence from at least one complementarity determining region of BG10-19, and the oligomerization domain comprises the D4 trimerization domain of VSV-G protein.

In some embodiments, the multispecific antibody that comprises a tandem scFv format binds to a Neuraminidase active site and a Hemagglutinin stem of influenza virus and the fusion protein oligomerization domain comprises the D4 trimerization domain of VSV-G protein. In some embodiments, the multispecific antibody that comprises a tandem scFv format comprises an amino acid sequence from at least one complementarity determining region of 1E01 and an amino acid sequence from at least one complementarity determining region of F16 and the fusion protein oligomerization domain comprises the D4 trimerization domain of VSV-G protein. In some embodiments, the multispecific antibody that comprises a tandem scFv format binds to a Spike NTD and a Spike RBD of SARS COV-2 and the fusion protein oligomerization domain comprises the D4 trimerization domain of VSV-G protein. In some embodiments, the multispecific antibody that comprises a tandem scFv format comprises an amino acid sequence from at least one complementarity determining region of CoV2-2021 and an amino acid sequence from at least one complementarity determining region of C012 and the fusion protein oligomerization domain comprises the D4 trimerization domain of VSV-G protein.

Described herein, in some embodiments, are antiviruses comprising a fusion protein that comprises a transmembrane polypeptide and an antibody which binds to a surface protein of a virus, wherein the antivirus further comprises a second fusion protein that comprises a second transmembrane polypeptide and a second antibody which binds to a surface protein of the virus. In some embodiments, the second fusion protein comprises a second antibody that comprises an amino acid sequence from at least one complementarity determining region of 80R, H4, 7D10, 1E01, F16, 0304-4A10, 2M-14E5, 9A1, COV2-2021, COV2-2026, COV2-2146, 2M-10B11, C021, C018, BG4-5, BG7-15, or BG10-19 and a second transmembrane polypeptide that comprises the transmembrane domain of a Vesicular Stomatitis virus glycoprotein (VSV-G), the transmembrane domain of influenza Hemagglutinin (HA), the transmembrane domain of HIV surface glycoprotein GP120 or GP41, the transmembrane domain of measles virus surface glycoprotein hamagglutinin (H) protein, or the transmembrane domain of influenza Neuraminidase (NA).

In some embodiments, the second antibody comprises the scFv and an amino acid sequence from at least one complementarity determining region of H4 and the oligomerization domain comprises the D4 trimerization domain of VSV-G protein. In some embodiments, the second antibody comprises the scFv and an amino acid sequence from at least one complementarity determining region of 1E01 and the oligomerization domain comprises the D4 trimerization domain of VSV-G protein. In some embodiments, the second antibody comprises the scFv and an amino acid sequence from at least one complementarity determining region of F16 and the oligomerization domain comprises the D4 trimerization domain of VSV-G protein. In some embodiments, the second antibody comprises the scFv and an amino acid sequence from at least one complementarity determining region of 0304-4A10, and the oligomerization domain comprises the D4 trimerization domain of VSV-G protein. In some embodiments, the second antibody comprises the scFv and an amino acid sequence from at least one complementarity determining region of 2M-14E5, and the oligomerization domain comprises the D4 trimerization domain of VSV-G protein. In some embodiments, the second antibody comprises the scFv and an amino acid sequence from at least one complementarity determining region of 9A1, and the oligomerization domain comprises the D4 trimerization domain of VSV-G protein. In some embodiments, the second antibody comprises the scFv and an amino acid sequence from at least one complementarity determining region of COV2-2021, and the oligomerization domain comprises the D4 trimerization domain of VSV-G protein. In some embodiments, the second antibody comprises the scFv and an amino acid sequence from at least one complementarity determining region of COV2-2026, and the oligomerization domain comprises the D4 trimerization domain of VSV-G protein. In some embodiments, the second antibody comprises the scFv and an amino acid sequence from at least one complementarity determining region of COV2-2146, and the oligomerization domain comprises the D4 trimerization domain of VSV-G protein. In some embodiments, the second antibody comprises the scFv and an amino acid sequence from at least one complementarity determining region of 2M-10B11, and the oligomerization domain comprises the D4 trimerization domain of VSV-G protein. In some embodiments, the second antibody comprises the scFv and an amino acid sequence from at least one complementarity determining region of C021, and the oligomerization domain comprises the D4 trimerization domain of VSV-G protein. In some embodiments, the second antibody comprises the scFv and an amino acid sequence from at least one complementarity determining region of C018, and the oligomerization domain comprises the D4 trimerization domain of VSV-G protein. In some embodiments, the second antibody comprises the scFv and an amino acid sequence from at least one complementarity determining region of BG4-5, and the oligomerization domain comprises the D4 trimerization domain of VSV-G protein. In some embodiments, the second antibody comprises the scFv and an amino acid sequence from at least one complementarity determining region of BG7-15, and the oligomerization domain comprises the D4 trimerization domain of VSV-G protein. In some embodiments, the second antibody comprises the scFv and an amino acid sequence from at least one complementarity determining region of BG10-19, and the oligomerization domain comprises the D4 trimerization domain of VSV-G protein.

Described herein, in some embodiments, are antiviruses comprising a fusion protein that comprises a transmembrane polypeptide and an antibody which binds to a surface protein of a virus, wherein the antivirus further comprises a second fusion protein that comprises a second transmembrane polypeptide and a second antibody which binds to a surface protein of the virus. Described herein, in some embodiments, are antiviruses comprising a fusion protein that comprises an antibody and a transmembrane domain, wherein the antivirus further comprises a second fusion protein. In some embodiments, the antivirus further comprises a second fusion protein that comprises transmembrane polypeptide and a second antibody which binds to a surface protein of the virus, wherein the second antibody comprises a CDR sequence that has less than 100% sequence identity to an equivalent CDR sequence of the antibody. In some embodiments, the fusion protein and the second fusion protein comprise the same transmembrane polypeptide. In some embodiments, the fusion protein and the second fusion protein comprise different transmembrane polypeptides. In some embodiments, the fusion protein and the second fusion protein comprise the same oligomerization polypeptide. In some embodiments, the fusion protein and the second fusion protein comprise different oligomerization polypeptides. In some embodiments, the second antibody binds to the same surface protein as the antibody. In some embodiments, the second antibody binds to a different surface protein as the antibody.

In some embodiments, the second antibody is a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc. In some embodiments, the second antibody binds specifically to the surface protein of the virus.

In some embodiments, the second antibody comprises an amino acid sequence from at least one complementarity determining region of 80R, H4, 7D10, 1E01, F16, 0304-4A10, 2M-14E5, 9A1, COV2-2021, COV2-2026, COV2-2146, 2M-10B11, C021, C018, BG4-5, BG7-15, or BG10-19.

In some embodiments, the second antibody neutralizes the virus when displayed as a fusion protein on the surface of the antivirues but does not neutralize the virus when expressed as an isolated antibody. In some embodiments, the second antibody neutralizes the virus when displayed as a fusion protein on the surface of the antiviruses and neutralizes the virus when expressed as an isolated antibody.

In some embodiments, the second antibody is a multispecific antibody. In some embodiments, the second multispecific antibody binds specifically to more than one epitope on the surface protein of the virus. In some embodiments, the multispecific antibody that comprises a tandem scFv format binds to a Neuraminidase active site and a Hemagglutinin stem of influenza virus and the fusion protein oligomerization domain comprises the D4 trimerization domain of VSV-G protein. In some embodiments, the multispecific antibody that comprises a tandem scFv format binds to a Spike NTD and a Spike RBD of SARS COV-2 and the fusion protein oligomerization domain comprises the D4 trimerization domain of VSV-G protein. In some embodiments, the multispecific antibody that comprises a tandem scFv format comprises an amino acid sequence from at least one complementarity determining region of CoV2-2021 and an amino acid sequence from at least one complementarity determining region of C012 and the fusion protein oligomerization domain comprises the D4 trimerization domain of VSV-G protein. In some embodiments, the antibody comprises the scFv and an amino acid sequence from at least one complementarity determining region of 0304-4A10, and the oligomerization domain comprises the D4 trimerization domain of VSV-G protein, and the second antibody comprises the scFv and an amino acid sequence from at least one complementarity determining region of C018.

In some embodiments, the second multispecific antibody that comprises a tandem scFv format binds to a Neuraminidase active site and a Hemagglutinin stem of influenza virus and the fusion protein oligomerization domain comprises the D4 trimerization domain of VSV-G protein. In some embodiments, the second multispecific antibody that comprises a tandem scFv format comprises an amino acid sequence from at least one complementarity determining region of 1E01 and an amino acid sequence from at least one complementarity determining region of F16 and the fusion protein oligomerization domain comprises the D4 trimerization domain of VSV-G protein. In some embodiments, the second multispecific antibody that comprises a tandem scFv format binds to a Spike NTD and a Spike RBD of SARS COV-2 and the fusion protein oligomerization domain comprises the D4 trimerization domain of VSV-G protein. In some embodiments, the second multispecific antibody that comprises a tandem scFv format comprises an amino acid sequence from at least one complementarity determining region of CoV2-2021 and an amino acid sequence from at least one complementarity determining region of C012 and the fusion protein oligomerization domain comprises the D4 trimerization domain of VSV-G protein.

In some embodiments, a fusion protein disclosed herein comprises an amino acid sequence of at least 75% sequence homology to an amino acid sequence according to any one of SEQ ID NOs: 53-72, 74-76 and 78. In some embodiments, a fusion protein disclosed herein comprises an amino acid sequence of at least 80% sequence homology to an amino acid sequence according to any one of SEQ ID NOs: 53-72, 74-76 and 78. In some embodiments, a fusion protein disclosed herein comprises an amino acid sequence of at least 85% sequence homology to an amino acid sequence according to any one of SEQ ID NOs: 53-72, 74-76 and 78. In some embodiments, a fusion protein disclosed herein comprises an amino acid sequence of at least 90% sequence homology to an amino acid sequence according to any one of SEQ ID NOs: 53-72, 74-76 and 78. In some embodiments, a fusion protein disclosed herein comprises an amino acid sequence of at least 95% sequence homology to an amino acid sequence according to any one of SEQ ID NOs: 53-72, 74-76 and 78. In some embodiments, a fusion protein disclosed herein comprises an amino acid sequence of at least 98% sequence homology to an amino acid sequence according to any one of SEQ ID NOs: 53-72, 74-76 and 78. In some embodiments, a fusion protein disclosed herein comprises an amino acid sequence of at least 99% sequence homology to an amino acid sequence according to any one of SEQ ID NOs: 53-72, 74-76 and 78.

In some embodiments, the first fusion protein comprises an amino acid sequence of at least 75% sequence homology to an amino acid sequence according to any one of SEQ ID NOS: 53-72, 74-76 and 78. In some embodiments, the first fusion protein comprises an amino acid sequence of at least 80% sequence homology to an amino acid sequence according to any one of SEQ ID NOs: 53-72, 74-76 and 78. In some embodiments, the first fusion protein comprises an amino acid sequence of at least 85% sequence homology to an amino acid sequence according to any one of SEQ ID NOs: 53-72, 74-76 and 78. In some embodiments, the first fusion protein comprises an amino acid sequence of at least 90% sequence homology to an amino acid sequence according to any one of SEQ ID NOs: 53-72, 74-76 and 78. In some embodiments, the first fusion protein comprises an amino acid sequence of at least 95% sequence homology to an amino acid sequence according to any one of SEQ ID NOs: 53-72, 74-76 and 78. In some embodiments, the first fusion protein comprises an amino acid sequence of at least 98% sequence homology to an amino acid sequence according to any one of SEQ ID NOs: 53-72, 74-76 and 78. In some embodiments, the first fusion protein comprises an amino acid sequence of at least 99% sequence homology to an amino acid sequence according to any one of SEQ ID NOs: 53-72, 74-76 and 78.

In some embodiments, the second fusion protein comprises an amino acid sequence of at least 75% sequence homology to an amino acid sequence according to any one of SEQ ID NOS: 53-72, 74-76 and 78. In some embodiments, the second fusion protein comprises an amino acid sequence of at least 80% sequence homology to an amino acid sequence according to any one of SEQ ID NOs: 53-72, 74-76 and 78. In some embodiments, the second fusion protein comprises an amino acid sequence of at least 85% sequence homology to an amino acid sequence according to any one of SEQ ID NOs: 53-72, 74-76 and 78. In some embodiments, the second fusion protein comprises an amino acid sequence of at least 90% sequence homology to an amino acid sequence according to any one of SEQ ID NOs: 53-72, 74-76 and 78. In some embodiments, the second fusion protein comprises an amino acid sequence of at least 95% sequence homology to an amino acid sequence according to any one of SEQ ID NOs: 53-72, 74-76 and 78. In some embodiments, the second fusion protein comprises an amino acid sequence of at least 98% sequence homology to an amino acid sequence according to any one of SEQ ID NOs: 53-72, 74-76 and 78. In some embodiments, the second fusion protein comprises an amino acid sequence of at least 99% sequence homology to an amino acid sequence according to any one of SEQ ID NOs: 53-72, 74-76 and 78.

In some embodiments, the antibody comprises a scFv antibody comprising a heavy chain variable domain comprising an amino acid sequence with at least 90% sequence identity to SEQ ID No.: 1 and a light chain variable domain comprising an amino acid sequence with at least 90% sequence identity to SEQ ID No.: 15, and the transmembrane polypeptide comprises a transmembrane domain comprising an amino acid sequence with at least 90% sequence identity to any of SEQ ID NOs: 44-48. In some embodiments, the antibody comprises a scFv antibody comprising a heavy chain variable domain comprising an amino acid sequence with at least 90% sequence identity to SEQ ID No.: 2 and a light chain variable domain comprising an amino acid sequence with at least 90% sequence identity to SEQ ID No.: 16, and the transmembrane polypeptide comprises a transmembrane domain comprising an amino acid sequence with at least 90% sequence identity to any of SEQ ID NOs: 44-48. In some embodiments, the antibody comprises a scFv antibody comprising a heavy chain variable domain comprising an amino acid sequence with at least 90% sequence identity to SEQ ID No.: 2 and a light chain variable domain comprising an amino acid sequence with at least 90% sequence identity to SEQ ID No.: 16, the transmembrane polypeptide comprises a transmembrane domain comprising an amino acid sequence with at least 90% sequence identity to any of SEQ ID NOs: 44-48, and the oligomerization domain comprises an amino acid sequence with at least 90% sequence identity to any of SEQ ID NOs: 30-34. In some embodiments, the antibody comprises a scFv antibody comprising a heavy chain variable domain comprising an amino acid sequence with at least 90% sequence identity to SEQ ID No.: 3 and a light chain variable domain comprising an amino acid sequence with at least 90% sequence identity to SEQ ID No.: 17, and the transmembrane polypeptide comprises a transmembrane domain comprising an amino acid sequence with at least 90% sequence identity to any of SEQ ID NOs: 44-48. In some embodiments, the antibody comprises a scFv antibody comprising a heavy chain variable domain comprising an amino acid sequence with at least 90% sequence identity to SEQ ID No.: 4 and a light chain variable domain comprising an amino acid sequence with at least 90% sequence identity to SEQ ID No.: 18, the transmembrane polypeptide comprises a transmembrane domain comprising an amino acid sequence with at least 90% sequence identity to any of SEQ ID NOs: 44-48, and the oligomerization domain comprises an amino acid sequence with at least 90% sequence identity to any of SEQ ID NOs: 30-34. In some embodiments, the antibody comprises a scFv antibody comprising a heavy chain variable domain comprising an amino acid sequence with at least 90% sequence identity to SEQ ID No.: 5 and a light chain variable domain comprising an amino acid sequence with at least 90% sequence identity to SEQ ID No.: 19, the transmembrane polypeptide comprises a transmembrane domain comprising an amino acid sequence with at least 90% sequence identity to any of SEQ ID NOs: 44-48, and the oligomerization domain comprises an amino acid sequence with at least 90% sequence identity to any of SEQ ID NOs: 30-34. In some embodiments, the antibody comprises a scFv antibody comprising a heavy chain variable domain comprising an amino acid sequence with at least 90% sequence identity to SEQ ID No.: 6 and a light chain variable domain comprising an amino acid sequence with at least 90% sequence identity to SEQ ID No.: 20, the transmembrane polypeptide comprises a transmembrane domain comprising an amino acid sequence with at least 90% sequence identity to any of SEQ ID NOs: 44-48, and the oligomerization domain comprises an amino acid sequence with at least 90% sequence identity to any of SEQ ID NOs: 30-34. In some embodiments, the antibody comprises a scFv antibody comprising a heavy chain variable domain comprising an amino acid sequence with at least 90% sequence identity to SEQ ID No.: 7 and a light chain variable domain comprising an amino acid sequence with at least 90% sequence identity to SEQ ID No.: 21, the transmembrane polypeptide comprises a transmembrane domain comprising an amino acid sequence with at least 90% sequence identity to any of SEQ ID NOs: 44-48, and the oligomerization domain comprises an amino acid sequence with at least 90% sequence identity to any of SEQ ID NOs: 30-34. In some embodiments, the antibody comprises a scFv antibody comprising a heavy chain variable domain comprising an amino acid sequence with at least 90% sequence identity to SEQ ID No.: 8 and a light chain variable domain comprising an amino acid sequence with at least 90% sequence identity to SEQ ID No.: 22, the transmembrane polypeptide comprises a transmembrane domain comprising an amino acid sequence with at least 90% sequence identity to any of SEQ ID NOs: 44-48, and the oligomerization domain comprises an amino acid sequence with at least 90% sequence identity to any of SEQ ID NOs: 30-34. In some embodiments, the antibody comprises a scFv antibody comprising a heavy chain variable domain comprising an amino acid sequence with at least 90% sequence identity to SEQ ID No.: 9 and a light chain variable domain comprising an amino acid sequence with at least 90% sequence identity to SEQ ID No.: 23, and the transmembrane polypeptide comprises a transmembrane domain comprising an amino acid sequence with at least 90% sequence identity to any of SEQ ID NOs: 44-48. In some embodiments, the antibody comprises a scFv antibody comprising a heavy chain variable domain comprising an amino acid sequence with at least 90% sequence identity to SEQ ID No.: 9 and a light chain variable domain comprising an amino acid sequence with at least 90% sequence identity to SEQ ID No.: 23, the transmembrane polypeptide comprises a transmembrane domain comprising an amino acid sequence with at least 90% sequence identity to any of SEQ ID NOs: 44-48, and the oligomerization domain comprises an amino acid sequence with at least 90% sequence identity to any of SEQ ID NOs: 30-34. In some embodiments, the antibody comprises a scFv antibody comprising a heavy chain variable domain comprising an amino acid sequence with at least 90% sequence identity to SEQ ID No.: 10 and a light chain variable domain comprising an amino acid sequence with at least 90% sequence identity to SEQ ID No.: 24, the transmembrane polypeptide comprises a transmembrane domain comprising an amino acid sequence with at least 90% sequence identity to any of SEQ ID NOs: 44-48, and the oligomerization domain comprises an amino acid sequence with at least 90% sequence identity to any of SEQ ID NOs: 30-34. In some embodiments, the antibody comprises a scFv antibody comprising a heavy chain variable domain comprising an amino acid sequence with at least 90% sequence identity to SEQ ID No.: 11 and a light chain variable domain comprising an amino acid sequence with at least 90% sequence identity to SEQ ID No.: 25, the transmembrane polypeptide comprises a transmembrane domain comprising an amino acid sequence with at least 90% sequence identity to any of SEQ ID NOs: 44-48, and the oligomerization domain comprises an amino acid sequence with at least 90% sequence identity to any of SEQ ID NOs: 30-34. In some embodiments, the antibody comprises a scFv antibody comprising a heavy chain variable domain comprising an amino acid sequence with at least 90% sequence identity to SEQ ID No.: 12 and a light chain variable domain comprising an amino acid sequence with at least 90% sequence identity to SEQ ID No.: 26, the transmembrane polypeptide comprises a transmembrane domain comprising an amino acid sequence with at least 90% sequence identity to any of SEQ ID NOs: 44-48, and the oligomerization domain comprises an amino acid sequence with at least 90% sequence identity to any of SEQ ID NOs: 30-34. In some embodiments, the antibody comprises a scFv antibody comprising a heavy chain variable domain comprising an amino acid sequence with at least 90% sequence identity to SEQ ID No.: 13 and a light chain variable domain comprising an amino acid sequence with at least 90% sequence identity to SEQ ID No.: 27, and the transmembrane polypeptide comprises a transmembrane domain comprising an amino acid sequence with at least 90% sequence identity to any of SEQ ID NOs: 44-48. In some embodiments, the antibody comprises a scFv antibody comprising a heavy chain variable domain comprising an amino acid sequence with at least 90% sequence identity to SEQ ID No.: 13 and a light chain variable domain comprising an amino acid sequence with at least 90% sequence identity to SEQ ID No.: 27, the transmembrane polypeptide comprises a transmembrane domain comprising an amino acid sequence with at least 90% sequence identity to any of SEQ ID NOs: 44-48, and the oligomerization domain comprises an amino acid sequence with at least 90% sequence identity to any of SEQ ID NOs: 30-34. In some embodiments, the antibody comprises a scFv antibody comprising a heavy chain variable domain comprising an amino acid sequence with at least 90% sequence identity to SEQ ID No.: 14 and a light chain variable domain comprising an amino acid sequence with at least 90% sequence identity to SEQ ID No.: 28, and the transmembrane polypeptide comprises a transmembrane domain comprising an amino acid sequence with at least 90% sequence identity to any of SEQ ID NOs: 44-48. In some embodiments, the antibody comprises a scFv antibody comprising a heavy chain variable domain comprising an amino acid sequence with at least 90% sequence identity to SEQ ID No.: 14 and a light chain variable domain comprising an amino acid sequence with at least 90% sequence identity to SEQ ID No.: 28, the transmembrane polypeptide comprises a transmembrane domain comprising an amino acid sequence with at least 90% sequence identity to any of SEQ ID NOs: 44-48, and the oligomerization domain comprises an amino acid sequence with at least 90% sequence identity to any of SEQ ID NOs: 30-34. In some embodiments, the antibody comprises a tandem bispecific scFv antibody comprising: a first scFv antibody comprising a first heavy chain variable domain comprising an amino acid sequence with at least 90% sequence identity to SEQ ID No.: 5 and a first light chain variable domain comprising an amino acid sequence with at least 90% sequence identity to SEQ ID No.: 19, and a second scFv antibody comprising a second heavy chain variable domain comprising an amino acid sequence with at least 90% sequence identity to SEQ ID No.: 4 and a second light chain variable domain comprising an amino acid sequence with at least 90% sequence identity to SEQ ID No.: 18, the transmembrane polypeptide comprises a transmembrane domain comprising an amino acid sequence with at least 90% sequence identity to any of SEQ ID NOs: 44-48, and the oligomerization domain comprises an amino acid sequence with at least 90% sequence identity to any of SEQ ID NOs: 30-34. In some embodiments, the antibody comprises a tandem bispecific scFv antibody comprising: a first scFv antibody comprising a first heavy chain variable domain comprising an amino acid sequence with at least 90% sequence identity to SEQ ID No.: 13 and a first light chain variable domain comprising an amino acid sequence with at least 90% sequence identity to SEQ ID No.: 27, and a second scFv antibody comprising a second heavy chain variable domain comprising an amino acid sequence with at least 90% sequence identity to SEQ ID No.: 9 and a second light chain variable domain comprising an amino acid sequence with at least 90% sequence identity to SEQ ID No.: 23, the transmembrane polypeptide comprises a transmembrane domain comprising an amino acid sequence with at least 90% sequence identity to any of SEQ ID NOs: 44-48, and the oligomerization domain comprises an amino acid sequence with at least 90% sequence identity to any of SEQ ID NOs: 30-34. In some embodiments, the first fusion protein comprises: a first scFv antibody comprising a first heavy chain variable domain comprising an amino acid sequence with at least 90% sequence identity to SEQ ID No.: 14 and a first light chain variable domain comprising an amino acid sequence with at least 90% sequence identity to SEQ ID No.: 28, and a first transmembrane polypeptide comprising a transmembrane domain comprising an amino acid sequence with at least 90% sequence identity to any of SEQ ID NOs: 44-48; and the second fusion protein comprises: a second scFv antibody comprising a second heavy chain variable domain comprising an amino acid sequence with at least 90% sequence identity to SEQ ID No.: 10 and a second light chain variable domain comprising an amino acid sequence with at least 90% sequence identity to SEQ ID No.: 24, a second transmembrane polypeptide comprising a transmembrane domain comprising an amino acid sequence with at least 90% sequence identity to any of SEQ ID NOs: 44-48, and a second oligomerization domain comprising an amino acid sequence with at least 90% sequence identity to any of SEQ ID NOs: 30-34. In some embodiments, the antibody comprises a scFv antibody comprising a heavy chain variable domain comprising an amino acid sequence with at least 90% sequence identity to SEQ ID No.: 81 and a light chain variable domain comprising an amino acid sequence with at least 90% sequence identity to SEQ ID No.: 82, and the transmembrane polypeptide comprises a transmembrane domain comprising an amino acid sequence with at least 90% sequence identity to any of SEQ ID NOs: 44-48. In some embodiments, the antibody comprises a scFv antibody comprising a heavy chain variable domain comprising an amino acid sequence with at least 90% sequence identity to SEQ ID No.: 83 and a light chain variable domain comprising an amino acid sequence with at least 90% sequence identity to SEQ ID No.: 84, the transmembrane polypeptide comprises a transmembrane domain comprising an amino acid sequence with at least 90% sequence identity to any of SEQ ID NOs: 44-48, and the oligomerization domain comprises an amino acid sequence with at least 90% sequence identity to any of SEQ ID NOs: 30-34. In some embodiments, the antibody comprises a scFv antibody comprising a heavy chain variable domain comprising an amino acid sequence with at least 90% sequence identity to SEQ ID No.: 85 and a light chain variable domain comprising an amino acid sequence with at least 90% sequence identity to SEQ ID No.: 86, the transmembrane polypeptide comprises a transmembrane domain comprising an amino acid sequence with at least 90% sequence identity to any of SEQ ID NOs: 44-48, and the oligomerization domain comprises an amino acid sequence with at least 90% sequence identity to any of SEQ ID NOs: 30-34. In some embodiments, the first fusion protein comprises: a first scFv antibody comprising a first heavy chain variable domain comprising an amino acid sequence with at least 90% sequence identity to SEQ ID No.: 9 and a first light chain variable domain comprising an amino acid sequence with at least 90% sequence identity to SEQ ID No.: 23, and a first transmembrane polypeptide comprising a transmembrane domain comprising an amino acid sequence with at least 90% sequence identity to any of SEQ ID NOs: 44-48; and the second fusion protein comprises: a second scFv antibody comprising a second heavy chain variable domain comprising an amino acid sequence with at least 90% sequence identity to SEQ ID No.: 85 and a second light chain variable domain comprising an amino acid sequence with at least 90% sequence identity to SEQ ID No.: 86, a second transmembrane polypeptide comprising a transmembrane domain comprising an amino acid sequence with at least 90% sequence identity to any of SEQ ID NOs: 44-48, and a second oligomerization domain comprising an amino acid sequence with at least 90% sequence identity to any of SEQ ID NOs: 30-34. In some embodiments, the first fusion protein comprises: a first scFv antibody comprising a first heavy chain variable domain comprising an amino acid sequence with at least 90% sequence identity to SEQ ID No.: 10 and a first light chain variable domain comprising an amino acid sequence with at least 90% sequence identity to SEQ ID No.: 24, and a first transmembrane polypeptide comprising a transmembrane domain comprising an amino acid sequence with at least 90% sequence identity to any of SEQ ID NOs: 44-48; and the second fusion protein comprises: a second scFv antibody comprising a second heavy chain variable domain comprising an amino acid sequence with at least 90% sequence identity to SEQ ID No.: 85 and a second light chain variable domain comprising an amino acid sequence with at least 90% sequence identity to SEQ ID No.: 86, a second transmembrane polypeptide comprising a transmembrane domain comprising an amino acid sequence with at least 90% sequence identity to any of SEQ ID NOs: 44-48, and a second oligomerization domain comprising an amino acid sequence with at least 90% sequence identity to any of SEQ ID NOs: 30-34. In some embodiments, the first fusion protein comprises: a first scFv antibody comprising a first heavy chain variable domain comprising an amino acid sequence with at least 90% sequence identity to SEQ ID No.: 81 and a first light chain variable domain comprising an amino acid sequence with at least 90% sequence identity to SEQ ID No.: 82, and a first transmembrane polypeptide comprising a transmembrane domain comprising an amino acid sequence with at least 90% sequence identity to any of SEQ ID NOs: 44-48; and the second fusion protein comprises: a second scFv antibody comprising a second heavy chain variable domain comprising an amino acid sequence with at least 90% sequence identity to SEQ ID No.: 85 and a second light chain variable domain comprising an amino acid sequence with at least 90% sequence identity to SEQ ID No.: 86, a second transmembrane polypeptide comprising a transmembrane domain comprising an amino acid sequence with at least 90% sequence identity to any of SEQ ID NOs: 44-48, and a second oligomerization domain comprising an amino acid sequence with at least 90% sequence identity to any of SEQ ID NOs: 30-34.

### Compositions and Methods for Generation of Antiviruses

Described herein, in some embodiments, are compositions comprising an antivirus comprising an enveloped particle that displays an antibody on a surface of the antivirus. Described herein, in some embodiments, are compositions comprising an antivirus comprising a fusion protein that comprises an antibody and a transmembrane polypeptide. In some embodiments, are compositions comprising an antivirus comprising a fusion protein that comprises an antibody, oligomerization domain, and a transmembrane polypeptide. In some embodiments, the compositions comprise a first nucleic acid sequence encoding antivirus described herein. Further described herein, in some embodiments, are methods for producing a virus neutralizing composition from a non-neutralizing antibody that binds specifically to a viral protein; the method comprising expressing the non-neutralizing antibody that binds to the viral protein as a fusion protein with a transmembrane polypeptide on a surface of an antivirus at a valency of at least about 10 copies of the fusion protein on the surface of the antivirus.

Compositions and methods for generating antiviruses, in some embodiments, further comprise a second nucleic acid sequence that encodes one or more packaging viral proteins. In some embodiments, the one or more packaging viral proteins is a lentiviral protein, a retroviral protein, an adenoviral protein, or combinations thereof. In some embodiments, the one or more packaging viral proteins comprises gag, pol, pre, tat, rev, or combinations thereof.

Compositions and methods for generating antiviruses, in some embodiments, further comprise a third nucleic acid sequence that encodes a reporter, a therapeutic molecule, or combinations thereof.

In some embodiments, the reporter protein is a fluorescent protein or an enzyme. Exemplary reporter genes include, but are not limited to, acetohydroxyacid synthase (AHAS), alkaline phosphatase (AP), beta galactosidase (LacZ), beta glucuronidase (GUS), chloramphenicol acetyltransferase (CAT), green fluorescent protein (GFP), red fluorescent protein (RFP), yellow fluorescent protein (YFP), cyan fluorescent protein (CFP), cerulean fluorescent protein, citrine fluorescent protein, orange fluorescent protein, cherry fluorescent protein, turquoise fluorescent protein, blue fluorescent protein, horseradish peroxidase (HRP), luciferase (Luc), nopaline synthase (NOS), octopine synthase (OCS), luciferase, and derivatives thereof. Methods to determine modulation of a reporter gene are well known in the art, and include, but are not limited to, fluorometric methods (e.g. fluorescence spectroscopy, Fluorescence Activated Cell Sorting (FACS), fluorescence microscopy), and antibiotic resistance determination. In some embodiments, the reporter is a fluorescent protein. In some embodiments, the fluorescent protein is green fluorescent protein. In some embodiments, the reporter protein emits green fluorescence, yellow fluorescence, or red fluorescence. In some embodiments, the reporter is an enzyme. In some embodiments, the enzyme is β-galactosidase, alkaline phosphatase, β-lactamase, or luciferase.

In some embodiments, the therapeutic molecule is an immune modulating protein, a cellular signal modulating molecule, a proliferation modulating molecule, a cell death modulating molecule, or combinations thereof. In some embodiments, the therapeutic molecule is an immune checkpoint molecule. Exemplary immune checkpoint molecules include, but are not limited to, CTLA4, PD1, OX40, and CD28. In some embodiments, the therapeutic molecule is an inflammatory cytokine. In some embodiments, the inflammatory cytokine comprises IL-1, IL-12, IL-18, TNF-alpha, or TNF-beta. In some embodiments, the therapeutic molecule is a proliferation cytokine. In some embodiments, the proliferation cytokine comprises IL-2, IL-4, IL-7, or IL-15. In some embodiments, the cell death molecule comprises Fas or a death receptor.

Compositions and methods for generating multivalent particles, in some embodiments, further comprise a fourth nucleic acid sequence encoding a second fusion protein that comprises a second antibody and a second transmembrane polypeptide as described herein.

In some embodiments, the first nucleic acid sequence, the second nucleic acid sequence, and the third nucleic acid sequence are within a same vector. In some embodiments, the first nucleic acid sequence, the second nucleic acid sequence, and the third nucleic acid sequence are within different vectors. In some embodiments, the first nucleic acid sequence, the second nucleic acid sequence, the third nucleic acid sequence, and the fourth nucleic acid sequence are within a same vector. In some embodiments, the first nucleic acid sequence, the second nucleic acid sequence, third nucleic acid sequence, and the fourth nucleic acid sequence are within different vectors.

Various vectors, in some embodiments, are used herein. In some embodiments, the vector is a eukaryotic or prokaryotic vector. In some embodiments, the vector is a viral vector. In some embodiments, the vector is a lentivirus vector, an adenovirus vector, or an adeno-associated virus vector. Exemplary vectors include, without limitation, mammalian expression vectors: pSF-CMV-NEO-NH2-PPT-3XFLAG, pSF-CMV-NEO-COOH-3XFLAG, pSF-CMV-PURO-NH2-GST-TEV, pSF-OXB20-COOH-TEV-FLAG(R)-6His, pCEP4 pDEST27, pSF-CMV-Ub-KrYFP, pSF-CMV-FMDV-daGFP, pEF1a-mCherry-N1 Vector, pEF1a-tdTomato Vector, pSF-CMV-FMDV-Hygro, pSF-CMV-PGK-Puro, pMCP-tag(m), and pSF-CMV-PURO-NH2-CMYC; bacterial expression vectors: pSF-OXB20-BetaGa1, pSF-OXB20-Fluc, pSF-OXB20, and pSF-Tac; plant expression vectors: pRI 101-AN DNA and pCambia2301; and yeast expression vectors: pTYB21 and pKLAC2, and insect vectors: pAc5.1/V5-His A and pDEST8.

### Compositions and Pharmaceutical Compositions

Described herein, in some embodiments, are compositions comprising an antivirus comprising an enveloped particle that displays an antibody on a surface of the antivirus. Described herein, in some embodiments, are compositions comprising antiviruses comprising a fusion protein that comprises an antibody and a transmembrane polypeptide. Described herein, in some embodiments, are pharmaceutical compositions comprising antiviruses comprising a fusion protein that comprises an antibody and a transmembrane polypeptide.

For administration to a subject, the antiviruses as disclosed herein, may be provided in a pharmaceutical composition together with one or more pharmaceutically acceptable carriers or excipients. In some embodiments, the antiviruses as disclosed herein, may be provided in a composition together with one or more carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, intravenous, or inhalation) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

### Methods of Use

Antiviruses described herein, in some embodiments, are used to treat a viral infection. Described herein, in certain embodiments, are methods of treating a viral disease in a subject in need thereof comprising administering to the subject an antivirus comprising a fusion protein that comprises a transmembrane polypeptide and an antibody which binds to a surface protein of a virus of the viral disease wherein the fusion protein is expressed at a valency of at least about 10 copies on a surface of the antivirus, and the antivirus neutralizes the virus when the fusion protein is bound to the surface protein of the virus. In some instances, the viral infection is caused by SARS-COV-1. In some instances, the viral infection is caused by SARS-COV-2. In some instances, the viral infection is caused by MERS-COV. In some instances, the viral infection is caused by influenza.

In some instances, the subject is a mammal. In some instances, the subject is a mouse, rabbit, dog, pig, cattle, or human. Subjects treated by methods described herein may be infants, adults, or children. Pharmaceutical compositions or compositions comprising multivalent particles as described herein may be administered intravenously, subcutaneously, or inhalation. In some embodiments, the antivirus is administered intravenously. In some embodiments, the antivirus is administered through inhalation. In some embodiments, the antivirus is administered by an intraperitoneal injection. In some embodiments, the antivirus is administered by a subcutaneous injection.

Described herein, in some embodiments, are antiviruses, wherein the antivirus induces T cell mediated cytotoxicity against viral infected cells. In some embodiments, the antivirus induces immune cell-mediated virus clearance. In some embodiments, the administering to the subject of the antivirus is sufficient to reduce or eliminate the viral disease as compared to a baseline measurement of the viral disease taken from the subject prior to the administering of the antivirus. In some embodiments, the reduction is at least about 1-fold, 5-fold, 10-fold, 20-fold, 40-fold, 60-fold, 80-fold, or up to about 100-fold.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Generation of Ab-Antiviruses by Selective Display of Single-Chain Variable Fragments (scFv) on Viral-Like Particles (VLPs)

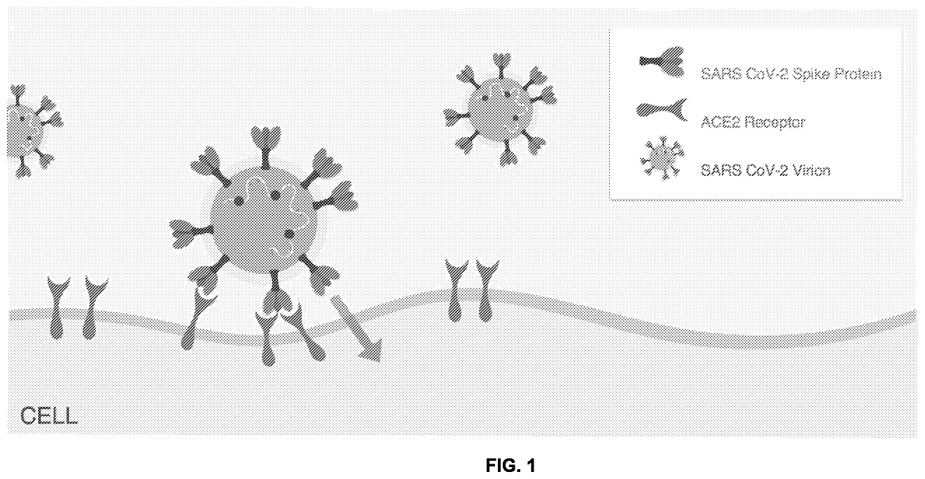

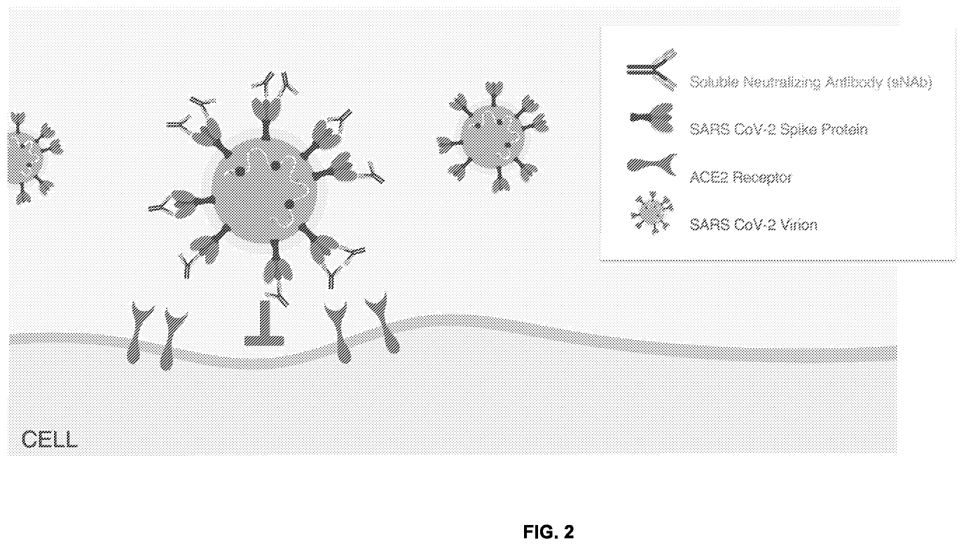

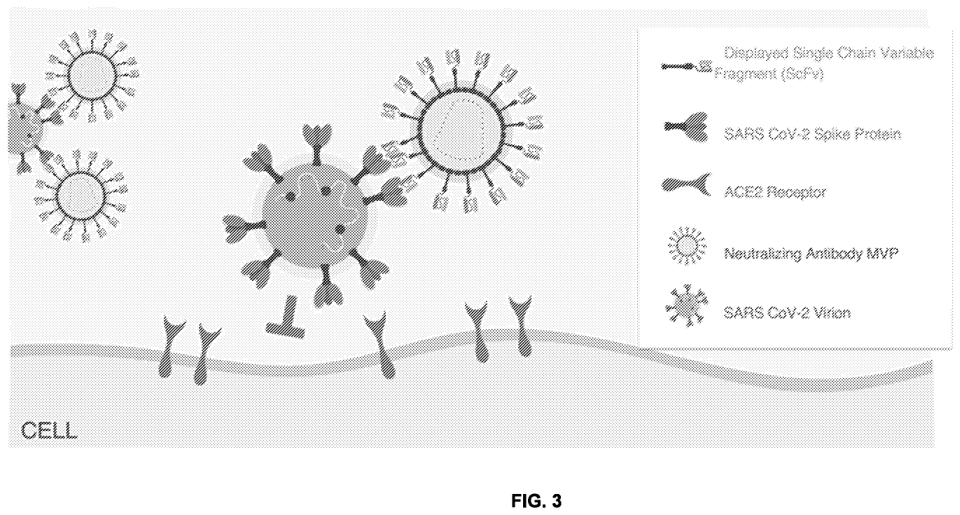

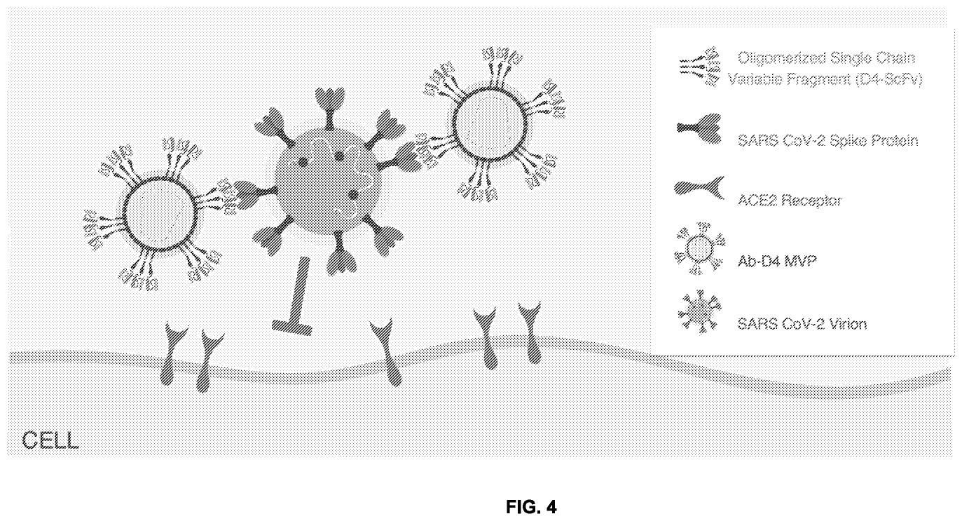

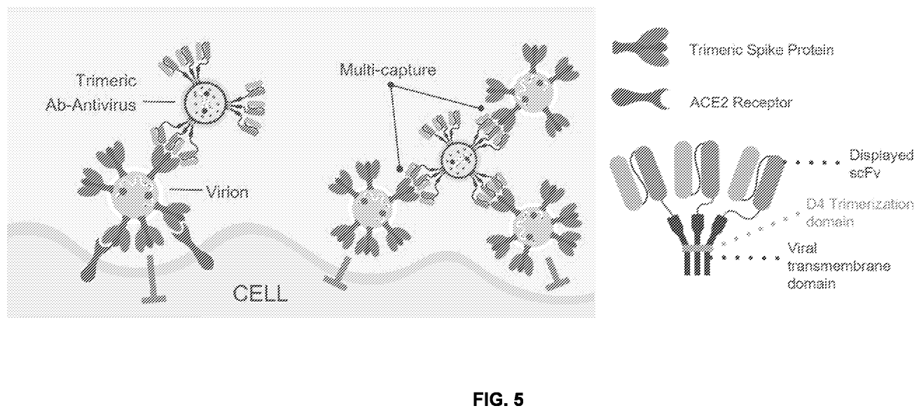

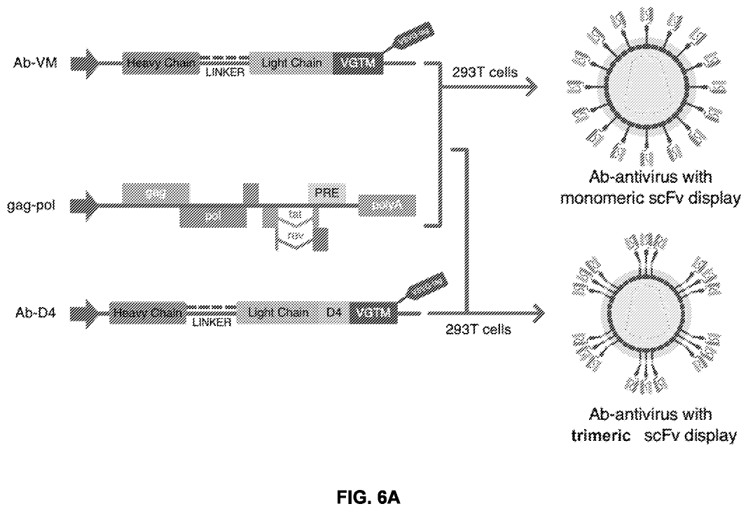

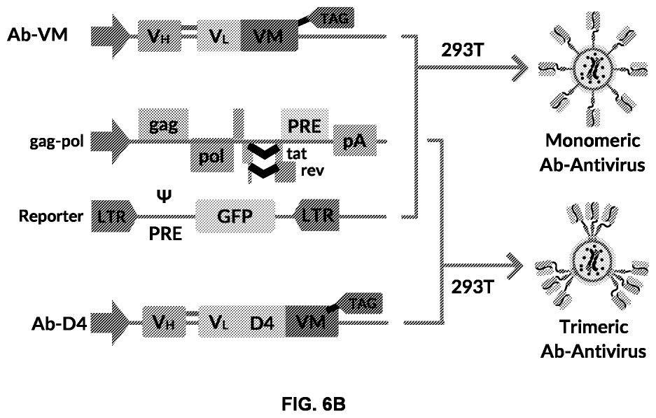

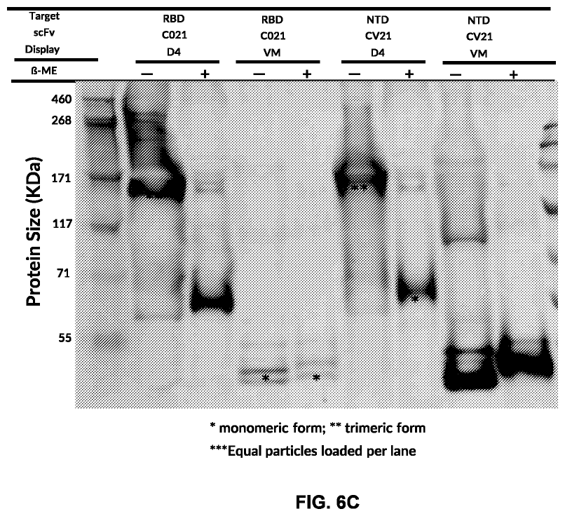

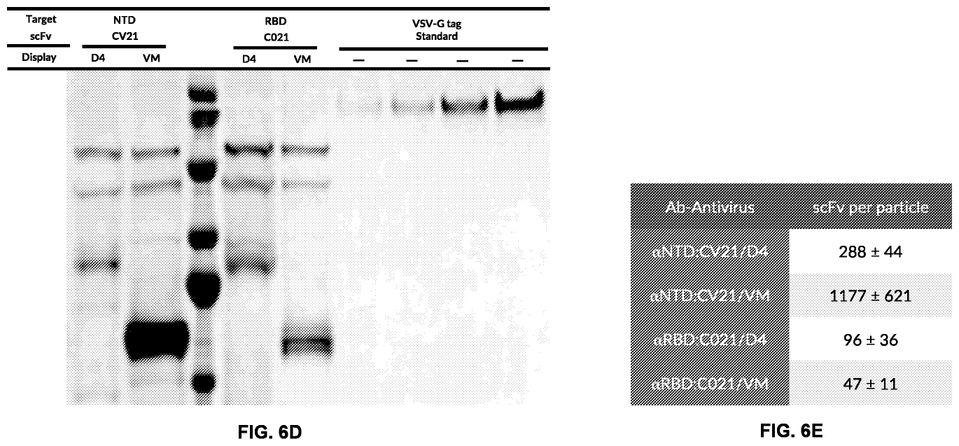

SARS COV-2 virions enter host cells through multivalent interactions with host receptors (FIG. 1). SARS COV-2 entry can be blocked by neutralizing antibodies (FIG. 2). Ab-MVP avidity was optimized by mimicking viral spike oligomerization in order to test that multivalent antibody particles can more effectively block SARS COV-2 infection, (FIG. 3 and FIG. 4). SARS COV-2 virions enter host cells through multivalent interactions with host receptors (FIG. 5). SARS COV-2 entry can be blocked by neutralizing antibodies multivalent antibody particles (MVPs) that were optimized by mimicking viral spike oligomerization (FIG. 5). Ab-MVP can more effectively block SARS COV-2 infection. For example, one Ab-MVP may interact and neutralize multiple SARS COV-2 virions (FIG. 5). Ab-antiviruses were generated by pseudotyping lentiviral VLPs with scFv fusion peptides to be displayed in patterns that mimic the ENV/spike protein distribution on virions. Each scFv-display fusion peptide was comprised of a scFv joined to a display peptide derived from a viral envelope protein. Single chain variable fragments were designed by linking published antibody heavy and light chain sequences with a flexible linker sequence (4GGGGS). Bi-specific, tandem scFvs were designed by joining two scFvs with an additional flexible linker sequence. Human codon-optimized scFv sequences were synthesized (Twist) and fused to a truncated VSV-G sequence encoding the transmembrane and cytoplasmic tail domains and the VSV-G epitope tag. A modified truncated VSV-G sequence containing the D4 trimerization domain as well was used to generate scFv-D4-fusion constructs. Full scFv fusion sequences were then cloned into a mammalian expression vector under the control of a CMV promoter. A lentiviral genome transfer vector encoding a reporter gene, such as green fluorescent protein (GFP), may be used in the generation of lentiviral VLPs. FIG. 6A and FIG. 6B represent the results from display vectors generated using transmembrane and intracellular anchors originating from vesicular stomatitis virus. Ab-Antiviruses were generated via co-transfection of 293T cells with an Ab display construct, lentiviral packaging constructs encoding structural components, with or without a lentiviral genome transfer vector encoding a GFP reporter. Ab-Antivirus particles were later purified, and concentrations were determined by P24 ELISA analysis. Display copy numbers and oligomeric configurations of Ab fusion proteins on Ab-Antivirus particles were determined via quantitative western blot and PAGE analysis, respectively.

Since ENV/spike proteins may contain specific signals required for selective display on enveloped particles, display vectors using transmembrane and intracellular anchors from ENV/spike proteins originating from various enveloped viruses, including measles virus, SARS CoV-2 and influenza virus were generated.

Non-replicative lenti-SARS-COV-1, lenti-SARS-COV-2 and lenti-MERS-COV pseudovirus were generated as follows: Genes encoding human codon-optimized SARS COV-1 (AY278741.1), wild type CoV-2 (MN908947.3), D614G mutant CoV-2 (MW079429.1) and MERS CoV (KT225476.2) spike proteins were synthesized (Twist) and cloned into a mammalian cell expression plasmid. In preparation for transfection, 7.5×10⁶ HEK293T cells (ATCC CRL-3216) were seeded overnight in 10-cm dishes containing DMEM media with glucose, L-glutamine and sodium pyruvate (Corning) supplemented with 10% fetal bovine serum (Sigma) and 1% Penicillin Streptomycin (Life Technologies), referred to as “293T Growth Media.” Cells reached≈90% confluence the next day at time for transfection. The following day, transfection mixture containing 1.25 μg spike expression plasmid, 5 μg psPAX2 lentiviral structural protein vector and with or without 7.5 μg lentiviral firefly luciferase reporter transfer vector expression plasmid, along with 27.5 μg polyethylenimine (PEI) in OPTI-MEM reduced serum medium (Gibco) was prepared. Transfection mixture was incubated at room temperature for 15 minutes before being added to cells, which were then incubated at 37ºC in 5% CO₂. 6 hours post-transfection, 293T Growth Media was changed to 293T Growth Media supplemented with 0.1% sodium butyrate (referred to as “Transfection Media”) before being returned to incubation. After incubating for 24 hours at 37° C. with 5% CO₂ in Transfection Media, supernatant containing pseudovirus was collected, centrifuged at 1680 rpm for 5 minutes to remove cellular debris and mixed with 1× polyethylene glycol 8000 solution (PEG, Hampton Research), before being stored at 4° C. for 24 hours to allow fractionation. Cells were replenished with fresh Transfection Media, and a second pseudovirus supernatant collection was performed at 48 hours. Supernatant collections were then pooled, PEG precipitated and purified by size exclusion chromatography using Sephacryl S-300 High Resolution Beads (Sigma Aldrich).

Non-replicative lenti-H5N1-influenza pseudovirus was generated as follows. Genes encoding human codon-optimized H5 hemagglutinin (HM006759) and N1 Neuraminidase (HM006761), as well as M2 influenza ion channel protein (EU014145.1), were synthesized (Twist) and cloned into a mammalian cell expression plasmid. H5 and N1 were cloned into a single, dual promoter mammalian expression plasmid called pVITRO2-hygro-mcs (InvivoGen). Preparation and transfection protocols were identical to lentiviral pseudotyping of coronaviruses, however, transfection mixture ratio was modified to contain 1.25 μg of H5/N1 dual expression plasmid, 1.25 μg M2 expression plasmid, 5 μg psPAX2 lentiviral structural protein vector and with or without 7.5 μg lentiviral firefly luciferase reporter transfer vector. Incubation of transfected cells, pseudovirus supernatant collections, PEG precipitation and pseudovirus purification were performed as described in coronavirus pseudotyping.

Briefly, Ab-antiviruses were generated via co-transfection of 293T cells with a scFv-display construct, lentiviral packaging constructs encoding structural components without a lentiviral genome transfer vector. (FIG. 6A). For example, to produce antiviruses, 293T cells were transfected with 1.25 μg scFv fusion expression construct and 5 μg psPAX2 lentiviral structural protein vector, following the PEI transfection protocol described above. Incubation of transfected cells, antivirus supernatant collections, PEG precipitation and antivirus purification and concentration were performed as described above.

Ab-antivirus particles were purified, and their concentrations were determined by p24 ELISA analysis. P24 concentrations in antivirus samples of pseudotyped coronaviruses, influenza viruses and antivirus particles were determined using an HIV p24 SimpleStep ELISA kit (Abcam) per the manufacturer's protocol. Concentrations of antivirus or pseudovirus particles were extrapolated from the assumption that each lentiviral particle contains approximately 2000 molecules of p24, or 1.25×10⁴ antivirus particles per picogram of p24 protein.

Antivirus concentrations determined via p24 ELISA were corroborated by tunable resistive pulse sensing (TRPS, qNano, IZON). Purified antivirus collections were diluted in 0.2 μm filtered PBS with 0.03% Tween-20 (Thermo Fisher Scientific) prior to qNano analysis. Concentration and size distributions of pseudotyped particles were then determined using an NP200 nanopore at a 45.5 mm stretch, and applied voltages between 0.5 and 0.7V were used to achieve a stable current of 130 nA through the nanopore. Measurements for each antivirus sample were taken at pressures of 3, 5 and 8 mbar, and considered valid if at least 500 events were recorded, particle rate was linear and root mean squared signal noise was maintained below 10 pA. Antivirus concentrations were then determined by comparison to a standardized multi-pressure calibration using CPC200 (mode diameter: 200 nm) (IZON) carboxylated polystyrene beads diluted 1:200 in 0.2 μM filtered PBS from their original concentration of 7.3×10¹¹ particles per/mL. Measurements were analyzed using IZON Control Suite 3.4 software to determine original sample concentrations.

Expression of scFv fusion constructs on multivalent antibody particles was confirmed via western blot analysis of purified particles. Copy numbers and oligomeric configurations of scFv fusion proteins on Ab-antiviruses were determined via quantitative western blot and PAGE analysis, respectively. Briefly, samples of purified antivirus were lysed at 4° C. for 10 minutes with cell lysis buffer (Cell Signaling) before being mixed with NuPage LDS sample buffer (Thermo Fisher Scientific) and boiled at 95° C. for 5 minutes. Differences in oligomerization were determined by running samples in reducing and non-reducing conditions. Under reducing conditions, 5% 2-Mercaptoethanol (Thermo Fisher Scientific) was added to samples to dissociate oligomerized scFv-D4 fusion constructs. Protein samples were then separated on NuPAGE 4-12% Bis-Tris gels (Thermo Fisher Scientific) and transferred onto a polyvinylidene fluoride (PVDF) membrane (Life Technologies). PVDF membranes were blocked with TRIS-buffered saline with Tween-20 (TBST) and 5% skim milk (Research Products International) for 1 hour, prior to overnight incubation with primary antibody diluted in 5% milk. For scFv fusion constructs expressing VSVG-tag, an anti-VSV-G epitope tag rabbit polyclonal antibody (BioLegend, Poly29039) was used at a 1:2000 dilution. The following day, the PVDF membrane was washed 3 times with 1×TBST and stained with a goat-anti-rabbit secondary antibody (IRDye 680) at a 1:5000 dilution for 60 minutes in 5% milk. Post-secondary antibody staining, the PVDF membrane was again washed 3 times with TBST before imaging on a Licor Odyssey scanner.

Quantitative Western blot analyses were performed to determine the copies of scFv fusion constructs displayed per particle. Purified antivirus samples were processed and analyzed via western blot under reducing conditions as described above. A reference decoy-MVP with a known display copy number was used to generate a standard curve, from which copy numbers of displayed scFvs on respective Ab-MVPs was determined. All western blots were validated using an automated Simple Western size-based protein assay (Protein Simple) following the manufacturer's protocols. Unless otherwise mentioned, all reagents used here are from Protein Simple. Concentrated samples were lysed as described above, before being diluted 1:10 in 0.1× sample buffer for loading on capillaries. ScFv fusion construct expression levels were identified using the same primary rabbit polyclonal antibody at a 1:400 dilution and an HRP conjugated anti-rabbit secondary antibody (Protein Simple). Chemiluminescence signal analysis and absolute quantitation were performed using Compass software (Protein Simple).

Two kinds of scFv-display constructs derived from VSV glycoprotein (VSV-G) were highly effective in displaying scFv antibodies. The Ab-VM display construct, which expresses a scFv fused to the VSV-G transmembrane and intracellular domains, was designed to pseudotype VLPs with primarily monomeric scFv fusion proteins (termed monomeric Ab-antivirus, FIG. 6A and FIG. 6B). The Ab-D4 display construct, which expresses a scFv fused to the D4 trimerization domain on top of the VSVG transmembrane and intracellular domains, was designed to pseudotype VLPs with trimeric scFv fusion proteins (designated trimeric Ab-antivirus, FIG. 6A and FIG. 6B). These constructs were used to display scFv chains derived from two non-neutralizing antibodies: αRBD:C021 (anti-Spike Receptor-binding domain, clone C021) and αNTD:CV21 (anti-Spike N-terminal domain, clone CV21). As illustrated in FIGS. 6C-6E, Ab-antiviruses were produced using a transfer vector and therefore contained viral genetic materials. Ab-antiviruses pseudotyped with αRBD:C021/VM and αNTD:CV21/VM vectors displayed scFv peptides in monomeric form, whereas Ab-antiviruses pseudotyped with αRBD:C021/D4 and αNTD:CV21/D4 vectors displayed scFv peptides in trimers or higher degree oligomers, as indicated by PAGE/Western blot analyses under non-reducing conditions (FIG. 6C). Further quantitative Western-blot analyses revealed that αNTD:CV21 and αRBD:C021 scFv peptides were displayed at 1177±621 or 47±11 copies/particle in the monomeric form, or at 288±44 or 96±36 copies/particle in the trimeric form, respectively (FIG. 6D and FIG. 6E, Table 4 and Table 5). The copies of displayed scFv antibody on each particle appear to vary widely and have no apparent correlation with monomeric or trimeric display format (Table 4 and Table 5). Display frequency is likely dependent on scFv amino acid sequences and/or codon usage of the coding gene. More detailed information about these antibodies can be found in Table 4, Table 5 and Table 6 and Table 7.

TABLE 4

| | | | | | | Antivirus vs |
|---|---|---|---|---|---|---|
| Ab-Antivirus | | | | | Ab Copies per | Pseudovirus IC50 |
| Name | Ab | Valency | OD¹ | TD² | Antivirus | (pM) |
| αRBD:80R/VM | scFv | Monomer | — | VSV-G TM | 22 | 0.6 ± 0.4 |
| | | | | (VM) | | |
| αRBD:H4/VM | scFv | Monomer | — | VM | 119 ± 80 | 0.77 ± 0.17 |
| αRBD:H4/D4 | scFv | Trimer | D4 | VM | 8 ± 2 | 2.2 ± 1.6 |
| αNTD:7D10/VM | scFv | Monomer | — | VM | 337 | 12 ± 5 |
| αNA:1E01/D4 | scFv | Trimer | D4 | VM | N/A | 5.86 ± 0.07 |
| αHA:FI6/D4 | scFv | Trimer | D4 | VM | N/A | 0.8 ± 1.1 |
| αS2:4A10/D4 | scFv | Trimer | D4 | VM | 335 ± 8 | 0.28 ± 0.26 |
| αS2:14E5/D4 | scFv | Trimer | D4 | VM | 19 ± 6 | 2.6 ± 2.0 |
| αS2:9A1/D4 | scFv | Trimer | D4 | VM | 25 ± 8 | 6 ± 4 |
| αNTD:CV21/VM | scFv | Monomer | — | VM | 682 | 0.8 ± 0.6 |
| αNTD:CV21/D4 | scFv | Trimer | D4 | VM | 288 ± 44 | 0.19 ± 0.05 |
| αNTD:CV26/D4 | scFv | Trimer | D4 | VM | 74 ± 28 | 4.3 ± 2.4 |
| αNTD:CV46/D4 | scFv | Trimer | D4 | VM | 67 ± 21 | 6 ± 3 |
| αRBD:B11/D4 | scFv | Trimer | D4 | VM | 46 ± 27 | 2.3 ± 0.5 |
| αRBD:C021/VM | scFv | Monomer | — | VM | 47 ± 11 | 2.6 ± 2.2 |
| αRBD:C021/D4 | scFv | Trimer | D4 | VM | 96 ± 36 | 0.27 ± 0.23 |
| αRBD:C018/VM | scFv | Monomer | — | VM | N/A | 7 ± 6 |
| αRBD:C018/D4 | scFv | Trimer | D4 | VM | 356 ± 196 | 1.6 ± 1.2 |
| αRBD:BG4-5/VM | scFv | Monomer | — | VM | N/A | 2.8 ± 2.7 |
| αRBD:BG7-15/D4 | scFv | Trimer | D4 | VM | N/A | 1.6 ± 0.8 |
| αRBD:BG10-19/D4 | scFv | Trimer | D4 | VM | N/A | 76 ± 5 |

OD¹: Oligomerization Domain
TD²: Transmembrane Domain

TABLE 5

| Ab-Antivirus | | | Binding | mAb Neutralizing | mAb Binding |
|---|---|---|---|---|---|
| Name | Ab | Target | Locale | Potential | Affinity (ng/mL) |
| αRBD: 80R/VM | 80R | SARS | Spike RBD | Neutralizing | — |
| | | CoV-1 | | | |
| αRBD: H4/VM | H4 | SARS | Spike RBD | Neutralizing | — |
| | | CoV-2 | | | |
| αRBD: H4/D4 | H4 | SARS | Spike RBD | Neutralizing | — |
| | | CoV-2 | | | |
| αNTD: 7D10/VM | 7D10 | MERS | Spike NTD | Neutralizing | — |
| | | CoV | | | |
| αNA: 1E01/D4 | 1E01 | Influenza | NA active | Neutralizing | — |
| | | | site | | |
| αHA: FI6/D4 | FI6 | Influenza | HA stem | Neutralizing | — |
| αRBD: BG7-15 | BG7-15 | SARS | Spike RBD | Neutralizing | — |
| | | CoV-2 | | | |
| αRBD: BG10-19 | BG10-19 | SARS | Spike RBD | Neutralizing | — |
| | | CoV-2 | | | |
| αS2: 4A10/D4 | 0304-4A10 | SARS | Spike S2 | Non-neutralizing | 3 |
| | | CoV-2 | | | |
| αS2: 14E5/D4 | 2M-14E5 | SARS | Spike S2 | Non-neutralizing | 7 |
| | | CoV-2 | | | |
| αS2: 9A1/D4 | 9A1 | SARS | Spike S2 | Non-neutralizing | 5 |
| | | CoV-2 | | | |
| αNTD: CV21/VM | COV2-2021 | SARS | Spike NTD | Non-neutralizing | — |
| | | CoV-2 | | | |
| αNTD: CV21/D4 | COV2-2021 | SARS | Spike NTD | Non-neutralizing | — |
| | | CoV-2 | | | |
| αNTD: CV26/D4 | COV2-2026 | SARS | Spike NTD | Non-neutralizing | — |
| | | CoV-2 | | | |
| αNTD: CV46/D4 | COV2-2146 | SARS | Spike NTD | Non-neutralizing | — |
| | | CoV-2 | | | |
| αRBD: B11/D4 | 2M-10B11 | SARS | Spike RBD | Non-neutralizing | 5 |
| | | CoV-2 | | | |
| αRBD: C021/VM | C021 | SARS | Spike RBD | Non-neutralizing | 1.25 |
| | | CoV-2 | | | |
| αRBD: C021/D4 | C021 | SARS | Spike RBD | Non-neutralizing | 1.25 |
| | | CoV-2 | | | |
| αRBD: C018/VM | C018 | SARS | Spike RBD | Non-neutralizing | 1.53 |
| | | CoV-2 | | | |
| αRBD: C018/D4 | C018 | SARS | Spike RBD | Non-neutralizing | 1.53 |
| | | CoV-2 | | | |
| αRBD: BG4-5/VM | BG4-5 | SARS | Spike RBD | Non-neutralizing | — |
| | | CoV-2 | | | |

TABLE 6

| Multi-specific | | | | | | |
|---|---|---|---|---|---|---|
| Ab-Antivirus | | Antibody | Target | Binding | Ab Display | |
| Name | Constituent Abs | Name | Virus | Locale | Format | Valency |
| αHA-NA-D4 | αNA:1E01 | 1E01 | Influenza | Neuraminidase | Tandem scFv | Trimer |
| | | | | active site | | |
| | αHA:FI6 | FI6 | | Hemagglutinin | | |
| | | | | stem | | |
| αRBD-NTD- | αNTD:CV21 | COV2- | SARS | Spike NTD | Tandem scFv | Trimer |
| D4 | | 2021 | CoV-2 | | | |
| | αRBD:C021 | C021 | | Spike RBD | | |
| αRBD- | αNTD:CV26/D4 | COV2- | SARS | Spike NTD | scFv | Trimer |
| VM&NTD-D4 | | 2026 | CoV-2 | | | |
| | αRBD:C018/VM | C018 | | Spike RBD | scFv | Monomer |
| (αRBD:BG10- | αRBD:BG10-19/D4 | BG10-19 | SARS | Spike RBD | scFv | Trimer |
| 19/D4) | αNTD:CV21/VM | COV2- | CoV-2 | Spike NTD | scFv | Monomer |
| &(αNTD:CV21/ | | 2021 | | | | |
| VM) | | | | | | |
| (αRBD:BG10- | αRBD:BG10-19/D4 | BG10-19 | SARS | Spike RBD | scFv | Trimer |
| 19/D4) & | αNTD:CV26/VM | COV2- | CoV-2 | Spike NTD | scFv | Monomer |
| (αNTD:CV26/ | | 2026 | | | | |
| VM) | | | | | | |
| (αRBD:BG10- | αRBD:BG10-19/D4 | BG10-19 | SARS | Spike RBD | scFv | Trimer |
| 19/D4) & | αRBD:BG4-5/VM | BG4-5 | CoV-2 | Spike RBD | scFv | Monomer |
| (αRBD:BG4- | | | | | | |
| 5/VM) | | | | | | |

TABLE 7

| Multi-specific | | | | Antivirus vs | mAb | mAb Binding |
|---|---|---|---|---|---|---|
| Ab-Antivirus | | | Ab Copies per | Pseudovirus | Neutralizing | Affinity |
| Name | OD¹ | TD² | Particle | IC50 (pM) | Potential | (ng/mL) |
| αHA-NA-D4 | D4 | VM | N/A | 0.47 ± 0.22 | Neutralizing | — |
| | | | | | Neutralizing | — |
| αRBD-NTD- | D4 | VM | N/A | 0.32 ± 0.27 | Non- | — |
| D4 | — | | | | neutralizing | |
| | | | | | Non- | 1.25 |
| | | | | | neutralizing | |
| αRBD- | D4 | VM | N/A | 3.06 ± 0.23 | Non- | — |
| VM&NTD- | — | | | | neutralizing | |
| D4 | | VM | N/A | | Non- | 1.53 |
| | | | | | neutralizing | |
| (αRBD:BG10- | D4 | VM | N/A | 0.4 ± 0.3 | Neutralizing | — |
| 19/D4) | — | VM | N/A | | Non- | — |
| &(αNTD:CV21/VM) | | | | | neutralizing | |
| (αRBD:BG10- | D4 | VM | N/A | 0.63 ± 0.12 | Neutralizing | — |
| 19/D4) & | — | VM | N/A | | Non- | — |
| (αNTD:CV26/VM) | | | | | neutralizing | |
| (αRBD:BG10- | D4 | VM | N/A | 0.5 ± 0.3 | Neutralizing | |
| 19/D4) & | — | VM | N/A | | Non- | |
| (αRBD:BG4- | | | | | neutralizing | |
| 5/VM) | | | | | | |

OD¹: Oligomerization Domain
TD²: Transmembrane Domain

### Example 2: Conversion of Non-Neutralizing Antibodies into Potent Inhibitors of CoV-2 by Multivalent Display on Antivirus Particles

A primary antibody library of binding Abs specific for a viral surface protein was constructed. A secondary library of fusion Ab-display constructs was derived from the primary library. Respective Ab-MVPs were produced using constructs from this secondary display construct library, before being screened in binding assays and pseudovirus neutralization to isolate potent clones.

In order to determine optimal target infection cells for pseudoviruses, various cell lines were screened and TCID₅₀ titration of pseudotyped coronaviruses and influenza viruses was conducted. Briefly, target cells were seeded in 120 μL of respective growth media at 25×10⁴ cells per well in 96-well, flat bottom, tissue-culture-treated plates (Thermo Fisher Scientific) with 6 μg/mL Hexadimethrine bromide (polybrene, Sigma) and a saturating dose of pseudovirus. Plates were then centrifuged at 800×g, 25° C. for 60 minutes to enhance infection efficiency. 48 hours post-infection, cells were lysed with Firefly Luciferase Lysis Buffer (Biotium) and lysis was transferred to 96-well, white assay plates (costar) before luciferase activity was analyzed via GLOMAX multi-detection system (Promega). Cells yielding infection signals more than 103-fold above background were considered viable infection cell lines. H1560 cells (ATCC CRL-5883), H1573 cells (ATCC CRL-5877) and 293T/17 cells (ATCC CRL-11268) were identified as optimal infection cell lines for MERS CoV, SARS COV-1 and CoV-2, and H5N1 pseudovirus, respectively.

To further boost the infection efficiency of SARS COV-1 and CoV-2 pseudovirus, ACE2 expression in H1573 cells was amplified. Lenti-ACE2/GFP pseudovirus was produced via PEI transfection of 293T cells with 1.25 μg VSV-G expression plasmid, 5 μg psPAX2 lentiviral structural protein vector and 7.5 μg of a lentiviral genome transfer vector encoding ACE2 and GFP. H1573 cells in 15 cm plates were then infected with lenti-ACE2/GFP pseudovirus. 48 hours post-infection, cells were trypsinized and sorted for GFP expression via fluorescence activated cell sorting (FACS) on an SH800S cell sorter (Sony Biotechnology) and the sorted population of cells overexpressing ACE2 was cultured (referred to as H1573/ACE2 cells).

The 50% tissue culture infective dose (TCID₅₀) for each pseudovirus was determined by titration of pseudovirus infection in each respective target cell line. Target cells were seeded as described during pseudovirus infection screening and infected in technical duplicates by 9 2-fold serial dilutions of pseudovirus, diluted in corresponding target cell growth media. Luciferase activity of infected cells was analyzed 48 hours post-infection, and data was fitted using a 4-parameter logistic curve to determine the TCID₅₀ pseudovirus concentration (GraphPad Prism 9.0.0). These concentrations were used during pseudovirus neutralization assays to measure the inhibitory potential of multivalent antibody particles.

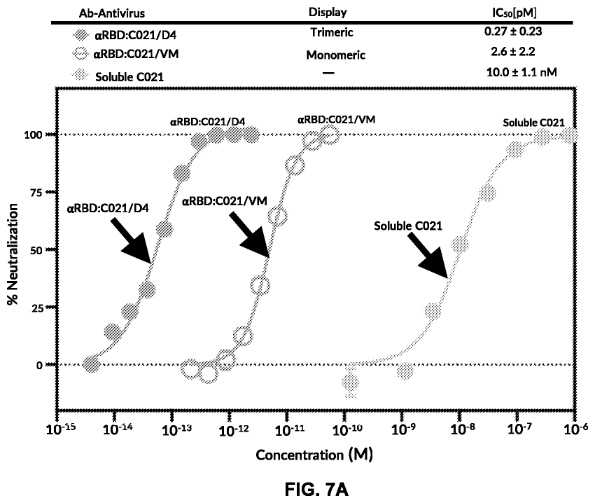

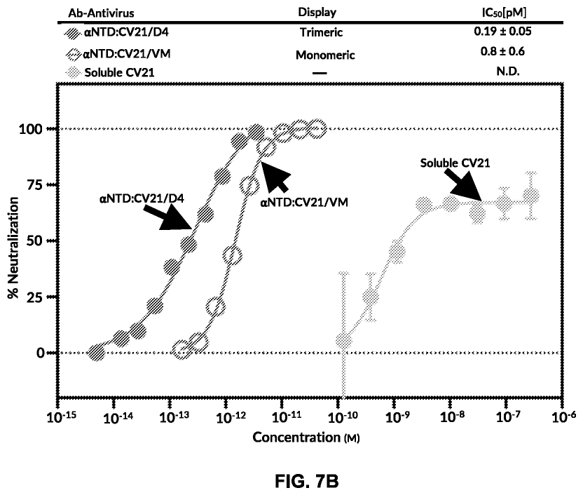

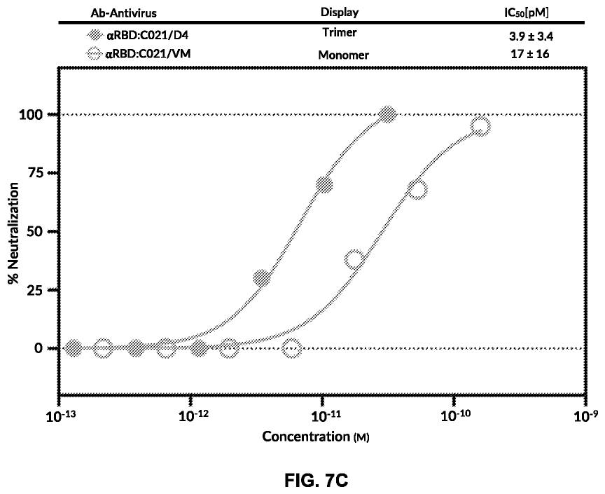

As illustrated in FIGS. 7A-7C, Ab-antiviruses were produced using a transfer vector and therefore contained viral genetic materials. The neutralizing activity of both trimeric and monomeric Ab-antiviruses pseudotyped with αRBD:C021 and αNTD:CV21 scFv antibodies was tested against SARS COV-2 in a pseudovirus neutralization assay (PNA), using H1573/ACE2 cells as target cells (FIG. 7A and FIG. 7B). In this assay, respective target cells were seeded in 96-well, flat-bottom, clear, tissue-culture treated plates (Thermo Fisher Scientific) at 25,000 cells/well with 6 μg/mL polybrene (Sigma) in the appropriate base medium supplemented with 10% fetal bovine serum (Sigma) and 1% Penicillin Streptomycin (Life Technologies). RPMI media with glucose, HEPES Buffer, L-Glutamine, sodium bicarbonate and sodium pyruvate (gibco) served as base medium for H1573/ACE2 cells and H1650 cells, while 293T Growth Media was used as base medium for 293T/17 cells. Pseudovirus was then added to wells at TCID₅₀ concentrations, along with titrated antivirus in 9×2-fold serial dilutions, yielding a 10-point dilution curve. In delayed pseudovirus neutralization assays, pseudovirus was added to wells in TCID₅₀ concentrations and incubated with cells for 60 minutes prior to the addition of titrated anti-virus. Plates containing cells, pseudovirus and antivirus were then centrifuged at 800×g, 25° C. for 60 minutes to maximize infection efficiency. 48 hours post-infection, cells were lysed using Firefly Luciferase Lysis Buffer (Biotium) and lysis was transferred to 96-well, white assay plates (costar) before luciferase activity was analyzed via GLOMAX multi-detection system (Promega). Titrated infection data was then plotted and fitted to a 4-parameter, logistic curve (GraphPad Prism 9.0.0) in order to calculate the half maximal inhibitory concentration (IC₅₀) of various antiviruses neutralizing their respective pseudoviruses.

The neutralizing activity of both monomeric and trimeric Ab-Antiviruses pseudotyped with αRBD:C021 and αNTD:CV21 scFvs was tested against wild type SARS COV-2 (strain USA-WA1/2020) in pseudovirus neutralization assays. Ab-Antiviruses pseudotyped with trimeric αRBD:C021 and αNTD:CV21 are both highly potent inhibitors, neutralizing pseudovirus at 50% inhibitory concentrations (IC50s) of 0.27±0.23 pM and 0.19±0.05 pM, respectively (FIG. 7A and FIG. 7B). In contrast, Ab-Antiviruses pseudotyped with monomeric αRBD:C021 and αNTD:CV21 had IC50s of 2.6±2.2 pM and 0.8±0.6 pM, respectively, a more than 4-fold increase in comparison to the corresponding trimeric Ab-Antivirus (FIG. 7A and FIG. 7B). As neither αRBD:C021 nor αNTD:CV21 antibodies demonstrated significant neutralizing activity in soluble form (FIG. 7A and FIG. 7B), these results demonstrated that non-neutralizing antibodies can be converted into potent, neutralizing Ab-Antiviruses through multivalent display. Importantly, both trimeric Ab-Antiviruses were more potent than the monomeric Ab-Antiviruses displaying identical scFvs, despite lower or comparable scFv display frequencies, illustrating that thorough mimicry of trimeric spike display on pathogenic viruses further enhances Ab-Antivirus neutralizing potency.

Furthermore, both monomeric and trimeric αRBD: C021 Ab-antiviruses had IC₅₀s of 17±16 pM and 3.9±3.4 pM, respectively, against live SARS COV-2 virus in Plaque Reduction Neutralization Test (PRNT) assays (FIG. 7C), which is comparable to their activities in pseudovirus neutralization assays. Briefly, in the PRNT assay, Vero E6 cells (ATCC: CRL-1586) were seeded at 175,000 cells/well using DMEM media (Thermo Fisher) supplemented with 10% fetal bovine serum (FBS) and Gentamicin (Quality Biological) in 24-well, tissue-culture treated plates. Cells were then incubated overnight at 37° C. in 5% CO₂ until reaching 80-100% confluence the next day. The following day, antivirus samples in serum were heat inactivated at 56° C. for 30 minutes before preparing serial dilutions. All dilutions were made using DMEM supplemented with 2% FBS and Gentamicin (referred to as “diluent”). Antivirus serial dilutions, to a total volume of 300 μL, were made using diluent, and 300 μL empty diluent served as a virus positive control. Next, 300 μL diluent containing SARS COV-2 (30 PFU/well) was added to antivirus serial dilutions and to the virus-only positive control, to a final volume of 600 μL. Mixtures of antivirus and SARS CoV-2 were incubated at 37° C. in 5.0% CO₂ for 60 minutes, before serial dilutions and virus positive control were added to cells. Cells were incubated with mixtures for 1 hour to allow for infection, and virus titers for each serial dilution were then determined by plaque assay. Percent neutralization data was plotted and a 4-parameter logistic curve was fitted to data to determine the 50% plaque reduction neutralization titer (PRNT₅₀) of various antiviruses neutralizing live SARS CoV-2 virus (GraphPad Prism 9.0.0).

In summary, non-neutralizing antibodies can be effectively converted into highly potent, neutralizing Ab-antiviruses through multivalent display. More importantly, trimeric Ab-antiviruses can be more potent inhibitors than monomeric Ab-antiviruses despite lower or comparable scFv display frequencies, illustrating that mimicking the trimeric spike patterns of pathogenic viruses can be an effective strategy for designing more effective antiviruses.

### Example 3: Neutralization of SARS COV-2 by Ab-Antiviruses Displaying Diverse scFv Chains

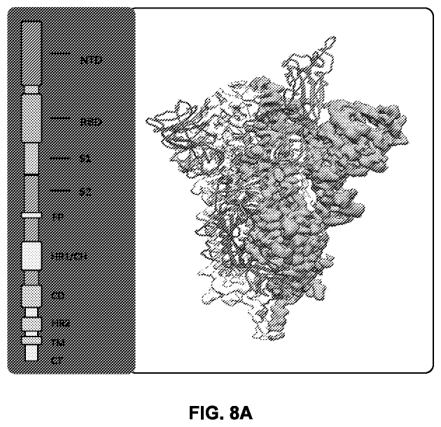

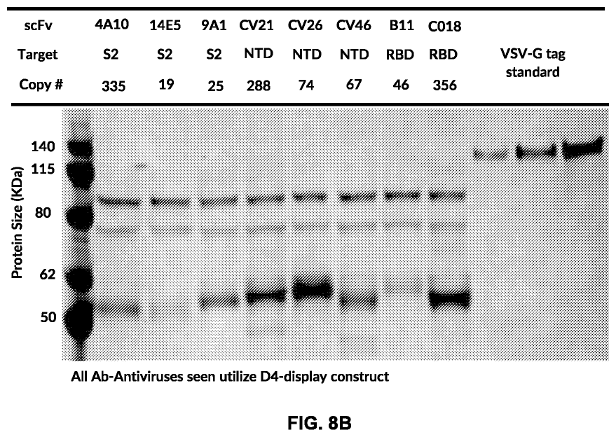

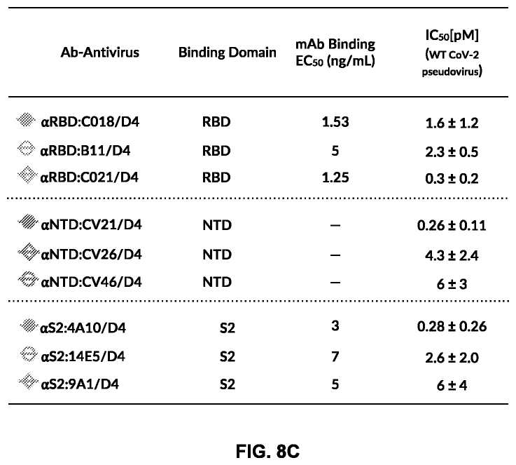

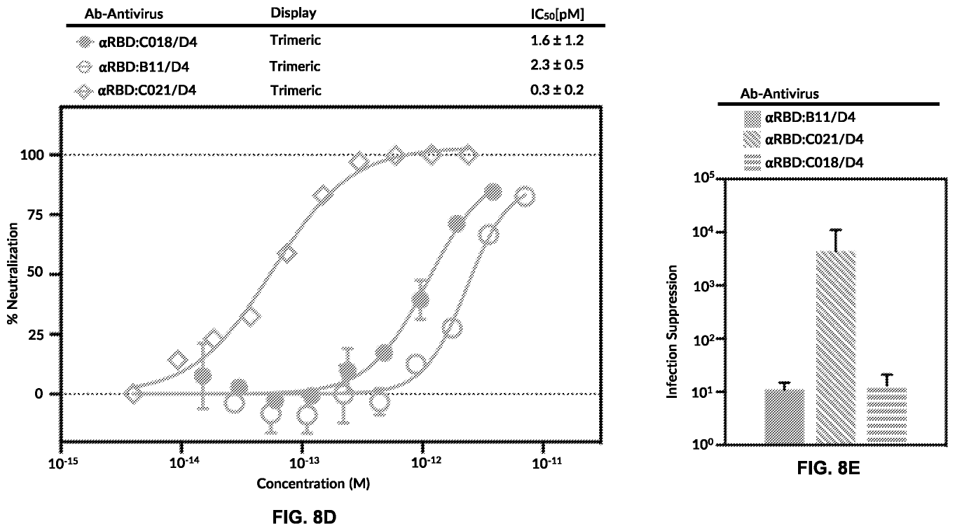

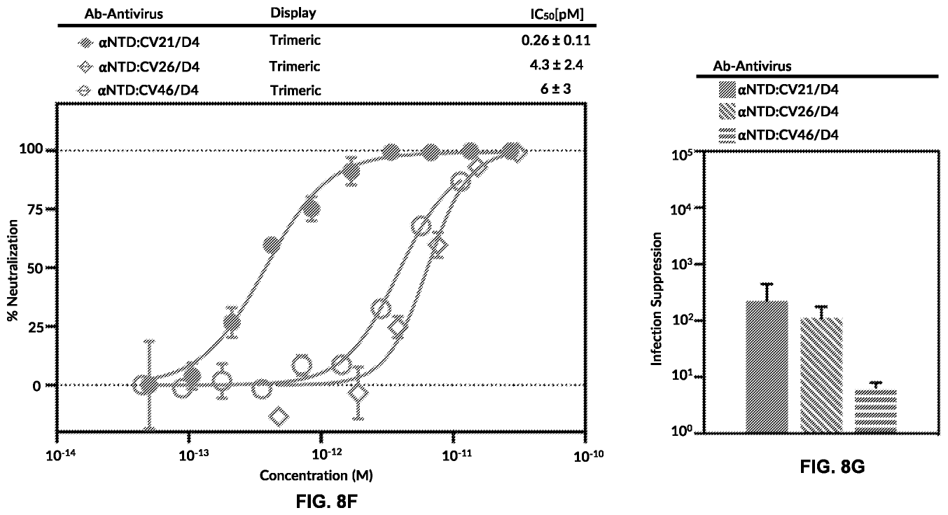

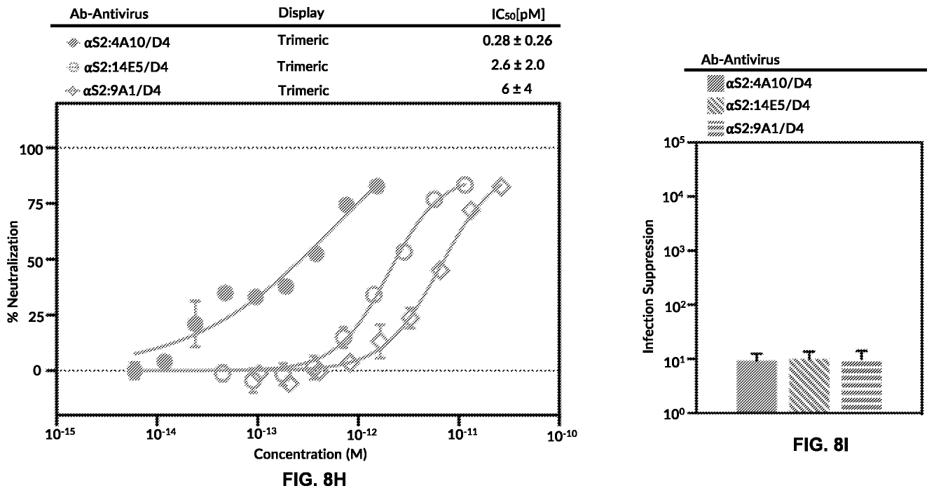

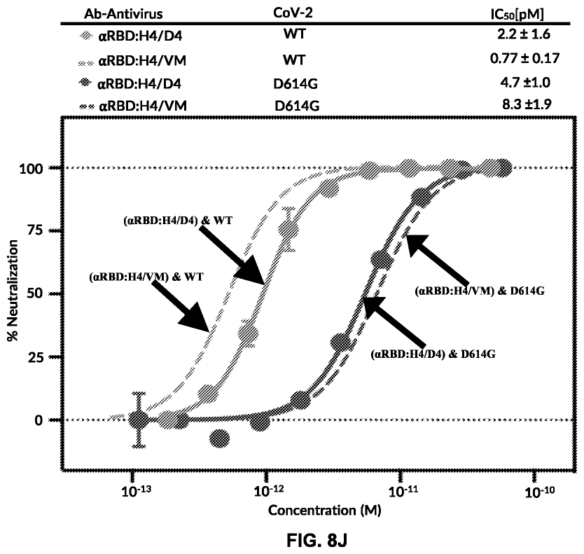

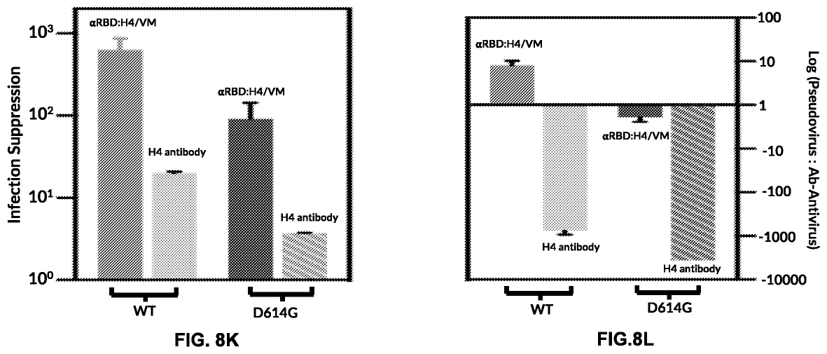

Trimeric Ab-antiviruses using seven non-neutralizing antibodies targeting distinct regions of the SARS COV-2 spike protein were generated: the RBD, NTD, and S2 domains (FIG. 8A, FIG. 8B, and FIG. 8C). As illustrated in FIGS. 8B-8L, Ab-antiviruses were produced using a transfer vector and therefore contained viral genetic materials. These Ab-antiviruses exhibited a range of display frequencies, from 19 copies/particle to 300 copies/particles (Table 4 and Table 5 above), as also seen in terms of protein expression by quantitative western-blot analyses (FIG. 8B). All 8 Ab-antiviruses demonstrated neutralizing activity in the low or sub-picomolar IC₅₀ range against SARS COV-2 pseudovirus (FIG. 8C, FIG. 8D, FIG. 8F, and FIG. 8H) indicating that antibodies targeting distinct spike regions can be used to generate Ab-antiviruses. Ab-antiviruses targeting the spike NTD or RBD domains achieved near 100-fold or over 1000-fold maximum infection suppression, respectively, while the Ab-antiviruses targeting the S2 domain of the spike protein, which lies below the exposed RBD and NTD domains, achieved a maximum of 10-fold infection suppression, measured as a function of the fold decrease in pseudovirus luciferase signal (FIG. 8E, FIG. 8G, and FIG. 8I). Several Ab-Antiviruses targeting the exposed spike NTD and RBD domains achieved at least 100-fold maximum infection suppression (FIG. 8E and FIG. 8G). These results demonstrated that diverse antibodies targeting multiple spike regions can be used to generate Ab-Antiviruses. The steric availability of the antibody binding site may impact the strength of multivalent interactions between Ab-Antivirus and target virions.

Next, the efficacy of Ab-antiviruses displaying neutralizing antibodies for SARS COV-2 was examined. In order to test whether multivalent display would significantly enhance the potency of moderately neutralizing antibodies, monomeric and trimeric Ab-antiviruses displaying approximately 119 and 8 copies of αRBD: H4 scFv per particle were generated. αRBD:H4 is a neutralizing antibody with ˜4.5 nM binding affinity. The αRBD:H4 antiviruses were tested for infection against wild type and D614G mutant SARS COV-2 using H1573/ACE2 cells in PNA assays (described in Example 2 above). Monomeric (αRBD:H4/VM) and trimeric (αRBD:H4/D4) antiviruses neutralized wild type SARS COV-2 at IC₅₀s of 0.77 and 2.2 pM, respectively (FIG. 8J). Bivalent αRBD:H4 in soluble form neutralized live SARS COV-2 at an IC₅₀ of 5.97 nM, indicating that both monomeric and trimeric Ab-antiviruses were approximately 1000-fold more potent than soluble αRBD:H4 antibody. Furthermore, monomeric αRBD: H4 antivirus exhibits at least 10-fold greater maximum infection suppression compared to its bivalent antibody, against both wild type and D614G CoV-2 pseudovirus (FIG. 8K and FIG. 8L). Stoichiometric neutralization ratios between monomeric αRBD:H4 antivirus and pseudovirus particles at IC₅₀ concentrations via P24 ELISA showed that a single monomeric αRBD:H4 antivirus particle neutralizes nearly 8 wild type SARS COV-2 pseudovirus particles, whereas approximately 700 molecules of bivalent αRBD:H4 are required to neutralize a single pseudovirus particle (FIG. 8K and FIG. 8L), demonstrating that antibodies with different targets and intrinsic neutralizing qualities can be used in the generation of highly potent Ab-antivirus.

### Example 4: Generation of Ab-Antiviruses Against Evolving Coronaviruses and Influenza Virus

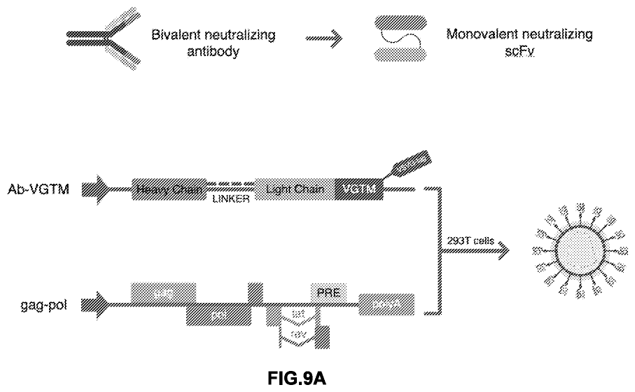

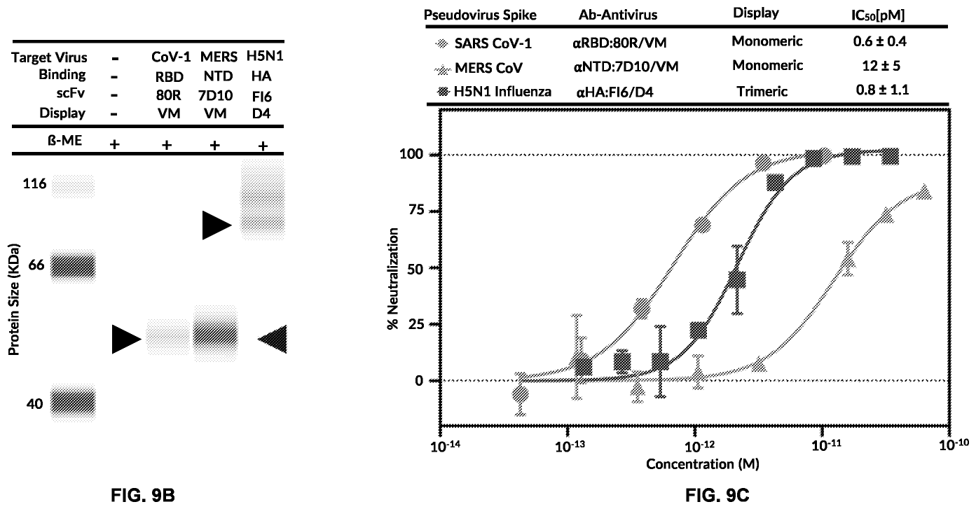

To demonstrate the broad applicability of the methods described herein, Ab-antiviruses displaying antibodies targeting the spike proteins of SARS COV-1, MERS COV and H5N1 influenza hemagglutinin, αCoV1:80R/VM, αMERS:7D10/VM, and αHA:FI6/D4, respectively, were generated. (FIG. 9A and FIG. 9B). As illustrated in FIGS. 9B-9C, Ab-antiviruses were produced using a transfer vector and therefore contained viral genetic materials. The neutralizing activity of these antiviruses against pseudovirus in microneutralization assays were evaluated using 293T/ACE2 cells, H1650 cells and 293T/17 cells as target cells, respectively. The αCoV1:80R/VM, αMERS:7D10/VM and αHA:FI6/D4 antiviruses neutralize their respective pseudoviruses at IC₅₀s of 0.6±0.40, 12±5.0, and 0.8±1.1 pM, respectively (FIG. 9C). Multivalent display on antiviruses drastically improved neutralizing efficacy for all three antibodies; in the instance of αMERS:7D10, soluble 7D10 neutralizes MERS CoV with an IC₅₀ of 1.2 nM, i.e., αMERS:7D10 antiviruses were 100-fold more potent than their bivalent counterpart.

### Example 5: Ab-Antiviruses Inhibit Pseudovirus Infection Pre- and Post-Attachment

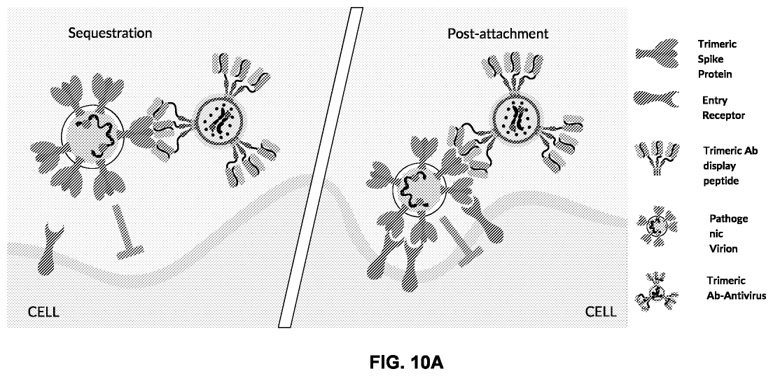

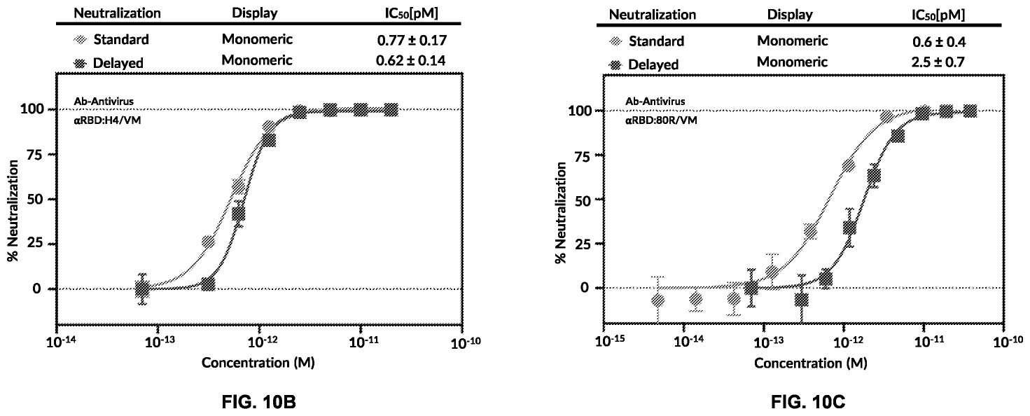

In order to test the mechanism by which antibody multivalent antibody particles stop viral infection, delayed inhibition microneutralization assays were conducted to test whether Ab-antiviruses can prevent virus infection post pseudovirus attachment to target cells (FIG. 10A). As illustrated in FIGS. 10B-10C, Ab-antiviruses were produced using a transfer vector and therefore contained viral genetic materials. In a standard microneutralization assay, Ab-antiviruses and pseudovirus are incubated together before being added to cells, allowing antiviruses to potentially sequester pseudovirus and preventing infection pre-attachment. In the delayed inhibition microneutralization assays, pseudovirus was incubated with target cells prior to the introduction of antivirus, whereby subsequent inhibition of pseudovirus infection is attributable to post-attachment neutralization by antiviruses. αRBD:H4/VM and αRBD:80R/VM antiviruses neutralized SARS CoV-1 and CoV-2 pseudovirus in delayed inhibition assays at IC₅₀s of 0.62±0.14 and 2.5±0.7 pM (FIG. 10B and FIG. 10C), respectively, with only a minimal loss of neutralizing potential in delayed pseudovirus inhibition implying that multivalent contact between the two entities can disrupt post-attachment processes of viral infection, such as fusion with target cells.

### Example 6: Genetic Programming of Ab-Antiviruses for Multi-Specific Recognition

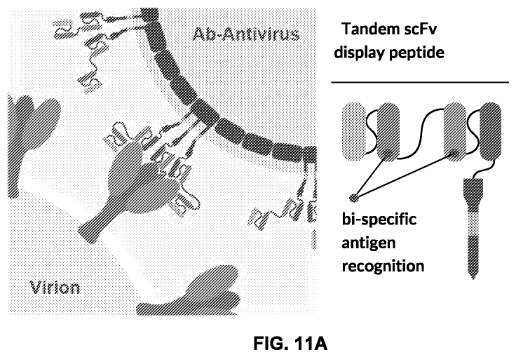

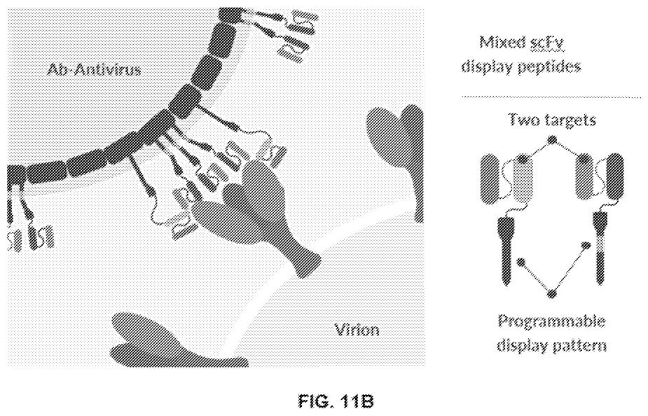

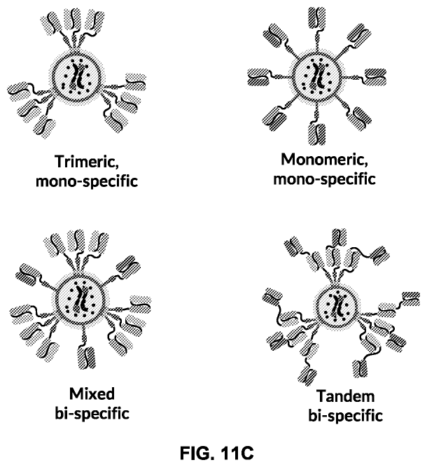

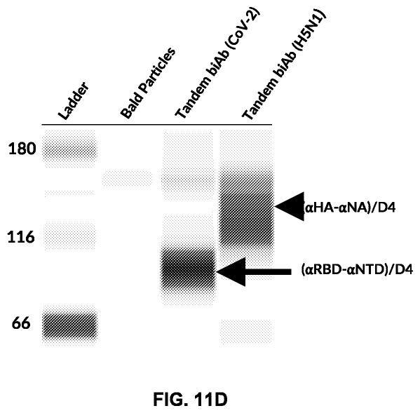

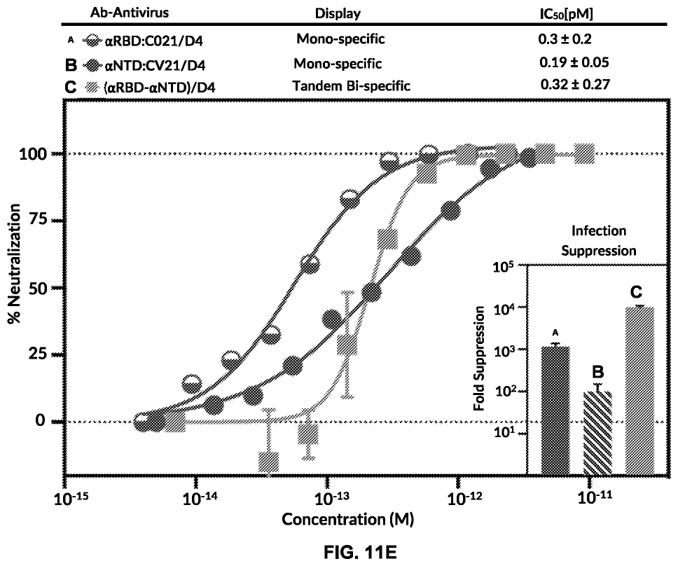

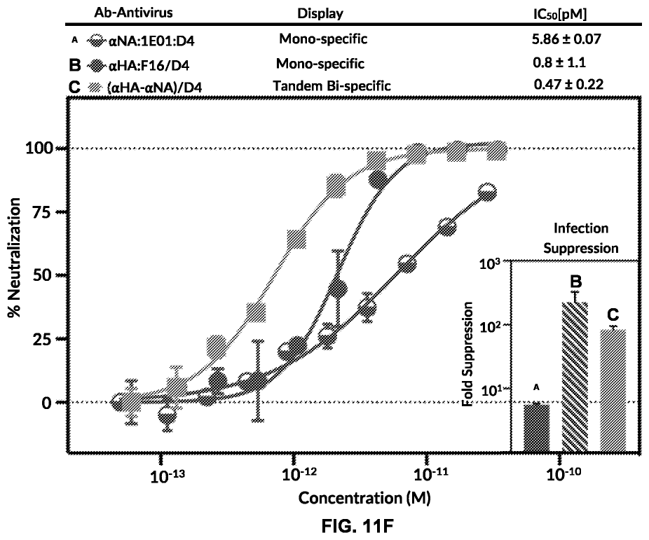

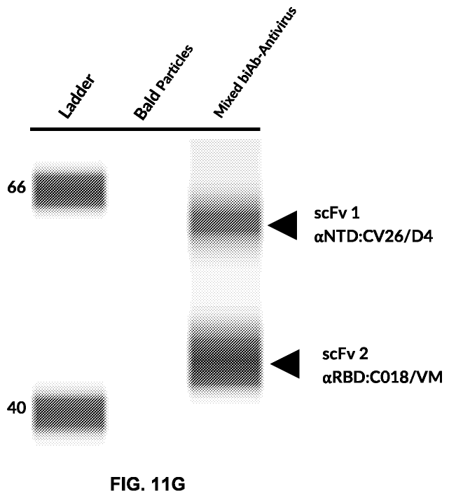

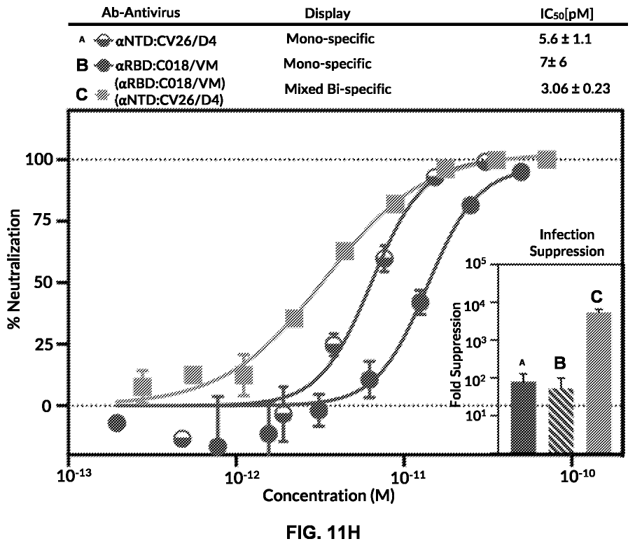

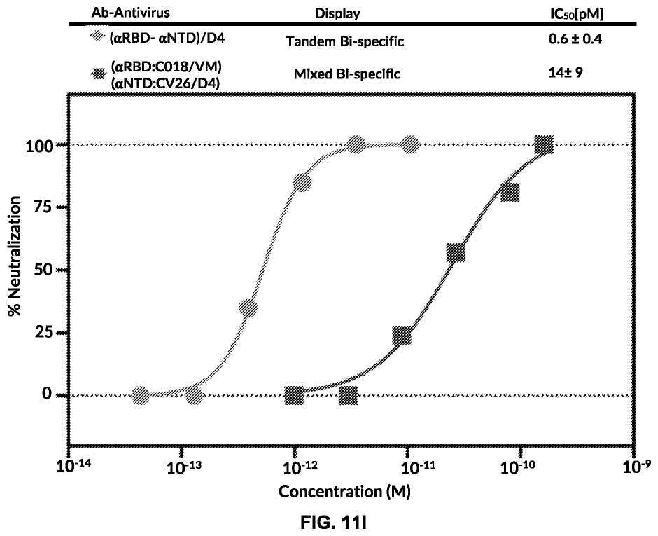

In order to further enhance the neutralizing potency of Ab-antiviruses against pandemic viruses and mitigate the effects of typical spike escape mutagenesis on neutralizing antibodies, Ab-antiviruses were genetically programmed to recognize multiple binding sites on the Env/spike protein or multiple surface proteins on the pandemic viruses by displaying combinations of distinct scFv antibodies. Two different approaches were taken to endow Ab-antiviruses with multiple specificities: (1) by displaying tandem scFvs recognizing two distinct epitopes; (2) by displaying two independent scFv chains on the surface of Ab-antiviruses (FIG. 11A, FIG. 11B and FIG. 11C). As illustrated in FIGS. 11D-11I, Ab-antiviruses were produced using a transfer vector and therefore contained viral genetic materials. For the first approach, bi-specific Ab-antiviruses (biAb-antiviruses) displaying trimeric tandem scFv chains for SARS COV-2, (αRBD-αNTD)/D4 biAb-antiviruses, and for H5N1 influenza, (αHA-αNA)/D4 biAb-antivirus, were designed and generated (FIG. 11D). The (αRBD-αNTD)/D4 biAb-antivirus was designed to target both the RBD and NTD regions of SARS COV-2 spike protein, while the (αHA-αNA)/D4 biAb-antivirus was designed to target both the stem region of hemagglutinin and the active site of neuraminidase on influenza H5N1. For the second approach, a bispecific Ab-antivirus (“biAb-antiviruses”) targeting the RBD and NTD regions of CoV-2 spike protein by co-displaying monomeric αRBD:C18/VM and trimeric αNTD:CV26/D4 antibodies on the same antivirus was designed and generated (FIG. 11G).

These biAb-antiviruses were tested against their respective target viruses in pseudovirus neutralization assays. Tandem (αRBD-αNTD)/D4 biAb-antiviruses had an IC₅₀ of 0.32±0.27 pM in pseudovirus neutralization assay which was comparable to the IC₅₀ of the mono-specific Ab-antiviruses displaying αRBD:C021 antibody or αNTD:CV21 (FIG. 11E). Tandem (αHA-αNA)/D4 biAb-antiviruses had an IC₅₀ of 0.47±0.22 pM in pseudovirus neutralization assays, a more than 2-fold increase in potency from mono-specific Ab-antiviruses displaying only αNA:1E01/D4 or αHA:FI6/D4 antibodies (FIG. 11F). Mixed (αRBD:C018/VM)(αNTD:CV26/D4) biAb-antiviruses had an IC₅₀ of 3.06±0.23 pM in pseudovirus neutralization assays, a more than 2 to 3-fold increase in potency as compared to Ab-antiviruses with individual αNTD:CV26/D4 or αRBD:C018/VM antibody displayed (FIG. 11H). Mixed biAb-antiviruses also suppressed pseudovirus infection by approximately 10,000-fold, more than 100-fold more than their respective mono-specific Ab-antiviruses (FIG. 11H). Tandem scFv (αRBD-αNTD)/D4 bi-specific Ab-antivirus was also able to neutralize SARS COV-2 in Plaque Reduction Neutralization Test (PRNT) assays (FIG. 11I). Mixed antivirus co-displaying αRBD:C018/VM and αNTD:CV26/D4 was also able to neutralize SARS COV-2 in Plaque Reduction Neutralization Test (PRNT) assays (FIG. 11I).

### Example 7: Mitigation of Spike Protein Escape Mutagenesis by Multivalent and Multi-Specific Ab-antiviruses

Multi-specific and multivalent Ab-antiviruses were tested against SARS COV-2 pseudovirus displaying spike proteins with circulating mutations known to increase virulence. For example, pseudoviruses displaying D614G, N439K/ΔH69/ΔV70, or N501Y/ΔH69/ΔV70/Δ144Υ mutant spike proteins were generated and tested against a panel of Ab-antiviruses in pseudovirus neutralization assays using H1573/ACE2 cells for infection.

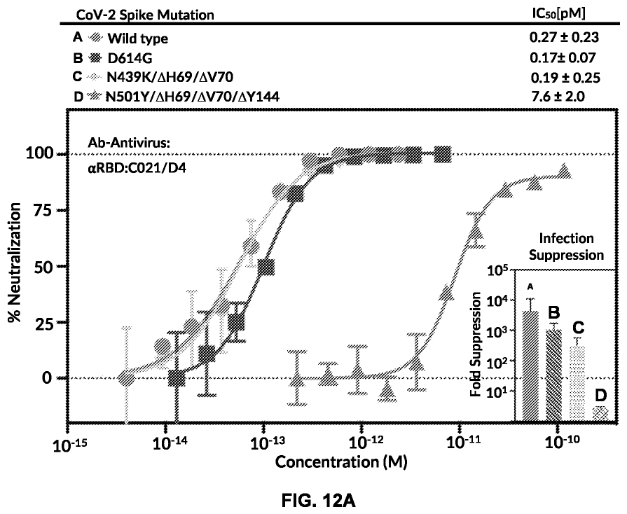

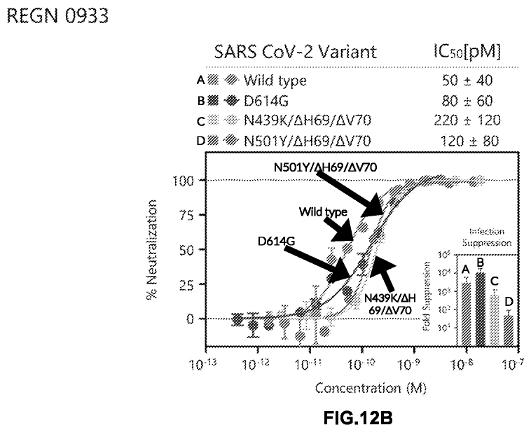

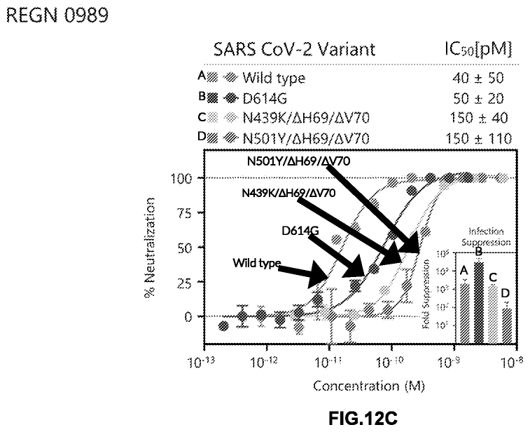

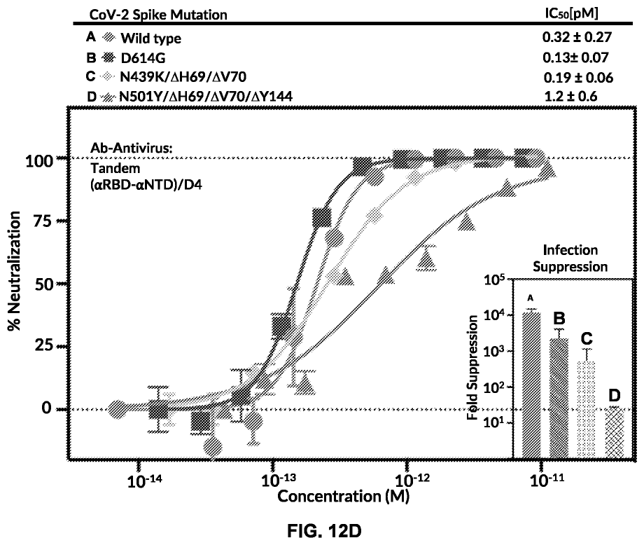

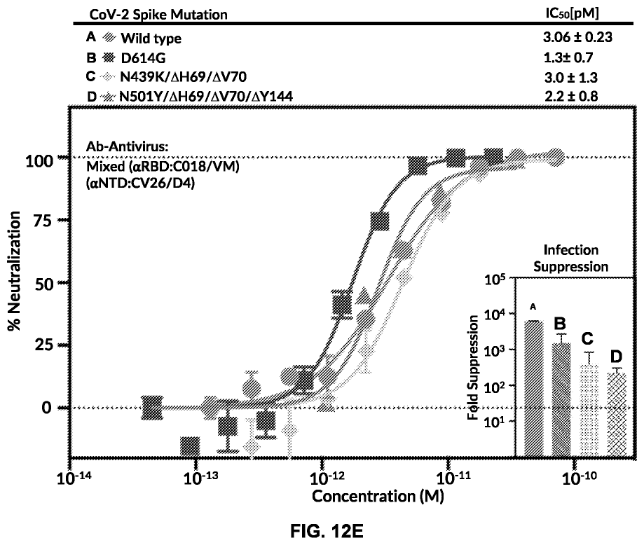

Mono-specific αRBD:C021/D4 retained sub-picomolar IC₅₀ neutralizing activity against the D614G and N439K escape mutants, while losing more than 10-fold potency against the N501Y spike mutant (FIG. 12A). As illustrated in FIGS. 12A-12E, Ab-antiviruses were produced using a transfer vector and therefore contained viral genetic materials. Two bivalent neutralizing antibodies, REGN 0933 and REGN 0989, used in Regeneron's antibody cocktail were also evaluated (FIG. 12B and FIG. 12C). Both tandem (αRBD-αNTD)/D4 biAb-antiviruses and mixed (αRBD:C018/VM)(αNTD:CV26/D4) biAb-antiviruses were resilient to all 3 spike mutations, demonstrating negligible changes in IC₅₀ (FIG. 12D and FIG. 12E). (αRBD-αNTD)/D4, which displayed a tandem scFv composed of αRBD:C021 and αNTD:CV21, was approximately 6 times more potent than mono-specific αRBD:C021/D4 against the N501Y spike mutant, demonstrating the potency-rescuing effect of multi-specificity on mono-specific Ab-Antiviruses impacted by escape mutations. These results indicated that multi-specificity and multivalence cushion Ab-Antiviruses from the effect of viral escape mutagenesis, providing the binding redundancy to retain avidity and potent neutralization despite loss of individual spike-scFv affinity.

### Example 8: Ab-Antiviruses Targeting Circulating SARS COV-2 Variants

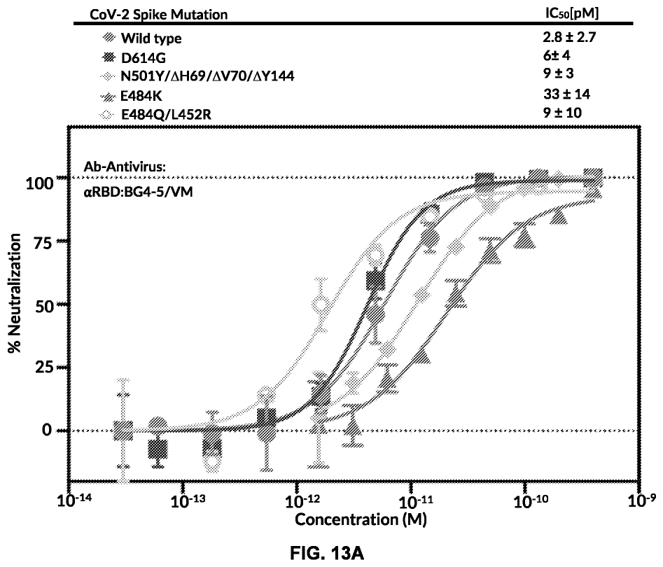

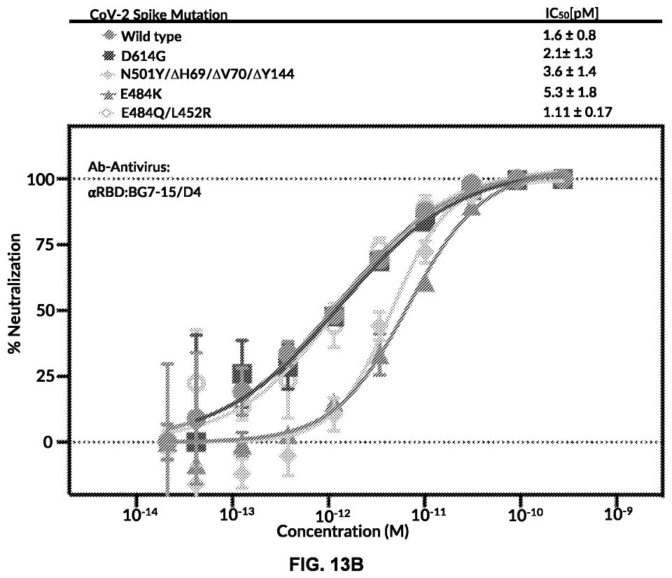

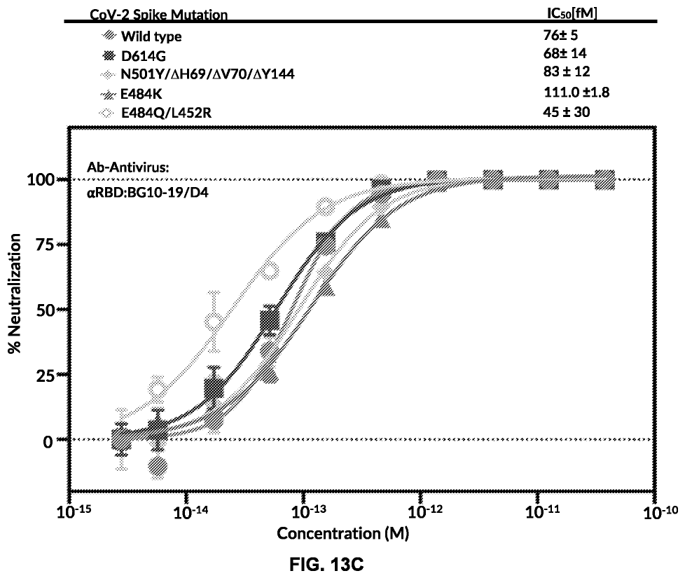

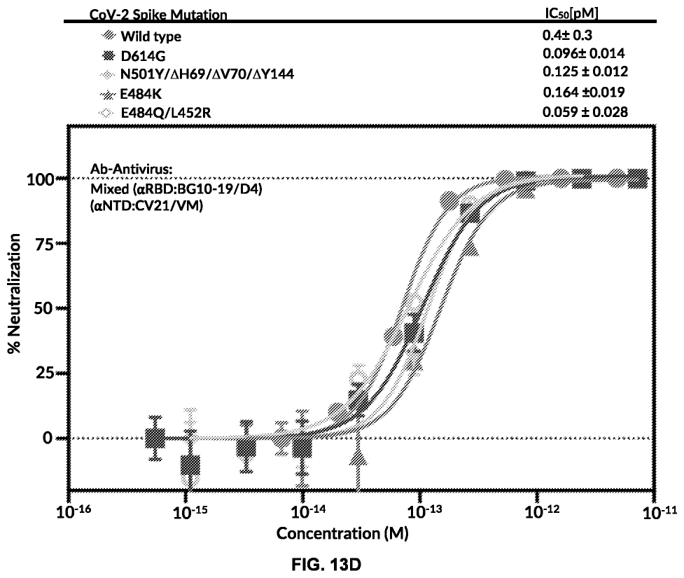

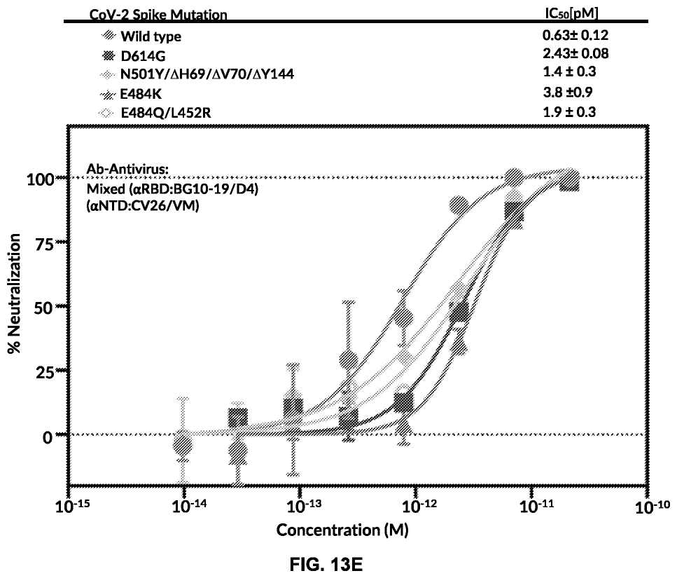

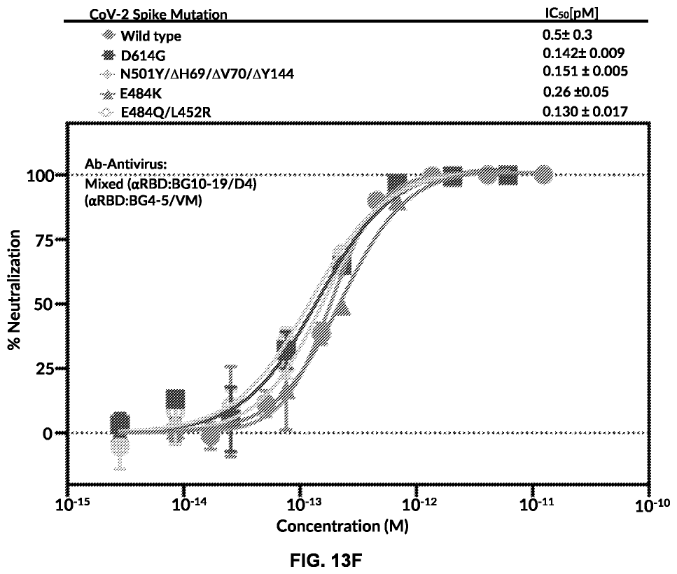

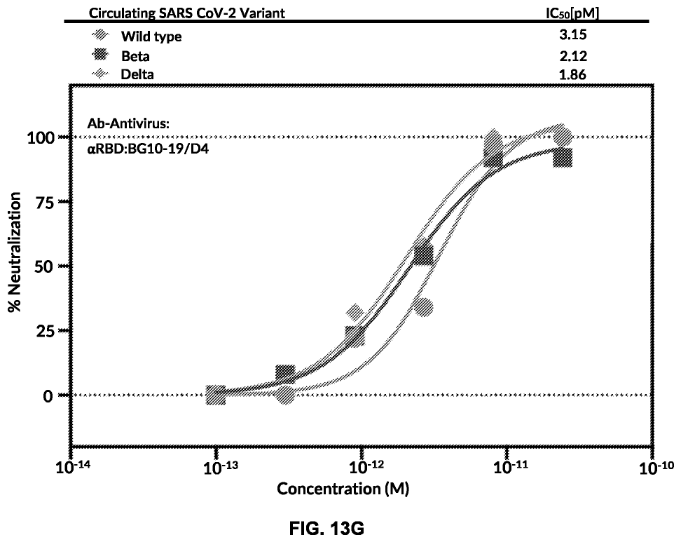

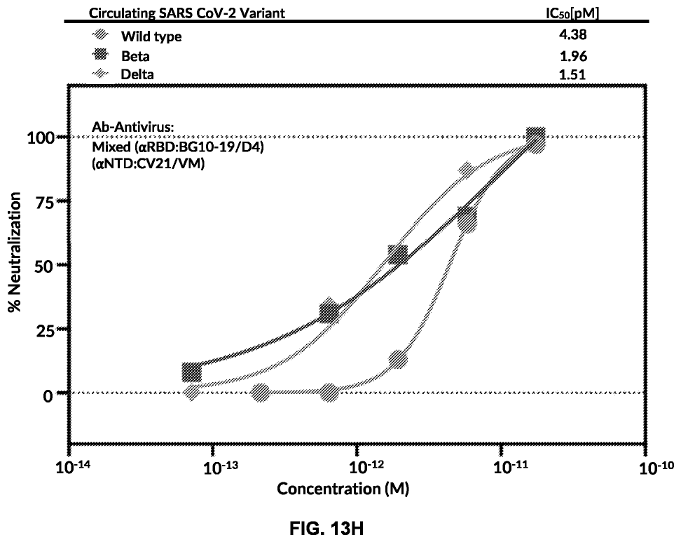

Additional mono and bi-specific Ab-Antiviruses were generated and evaluated in pseudovirus neutralization assays against an expanded panel of SARS COV-2 spike mutants, D614G, N501Y/ΔH69/ΔV70/ΔΥ144, Ε484K, and E484Q/L452R and the wild type. As illustrated in FIGS. 13A-13I, Ab-antiviruses were produced using a transfer vector and therefore contained viral genetic materials. All 3 mono-specific Ab-Antiviruses, αRBD:BG4-5/VM (FIG. 13A), αRBD:BG7-15/D4 (FIG. 13B) and αRBD:BG10-19/D4 (FIG. 13C), neutralized all spike mutants at low or sub-picomolar IC₅₀s, and one in particular, αRBD:BG10-19/D4 (FIG. 13C), neutralized all mutants in the mid-femtomolar range. Furthermore, all three mixed biAb-antiviruses, (αRBD: BG10-19/D4)(αNTD:CV21/VM) (FIG. 13D), (αRBD:BG10-19/D4)(αNTD:CV26/VM) (FIG. 13E), and (αRBD:BG10-19/D4)(αRBD:BG4-5/VM) (FIG. 13F), neutralized the mutant pseudoviruses at sub-picomolar IC₅₀s. Also, αRBD:BG10-19/D4 and the two derivative biAb-Antiviruses neutralized wild type,

Also, mono-specific multivalent Ab-antivirus, αRBD:BG10-19/D4 (FIG. 13G), and bispecific multivalent Ab-antiviruses, (αRBD:BG10-19/D4)(αNTD:CV21/VM) (FIG. 13H) and (αRBD:BG10-19/D4)(αRBD:BG4-5/VM) (FIG. 13I), neutralized the circulating SARS COV-2 variants, Beta variant and Delta, as well as the wild type SARS COV-2, at low or sub-picomolar IC₅₀s. These results demonstrated that multivalent, multi-specific Ab-Antiviruses can cushion soluble antibodies from viral escape mutagenesis and significantly enhance neutralizing potency.

### Example 9: In Vivo Inhibition of SARS COV-2 Infection in Mice by Ab-Antiviruses

SARS COV-2 infection in mice caused lethality and induced symptoms and pathology recapitulating many defining features of COVID-19 in humans. High viral titer in the lungs and infection progression to the brain and other organs were observed in infected mice, coinciding with upregulation of inflammatory cytokines and lymphocyte infiltration in affected organs. The in vivo therapeutic efficacy of Ab-Antiviruses against SARS COV-2 was evaluated in mice.

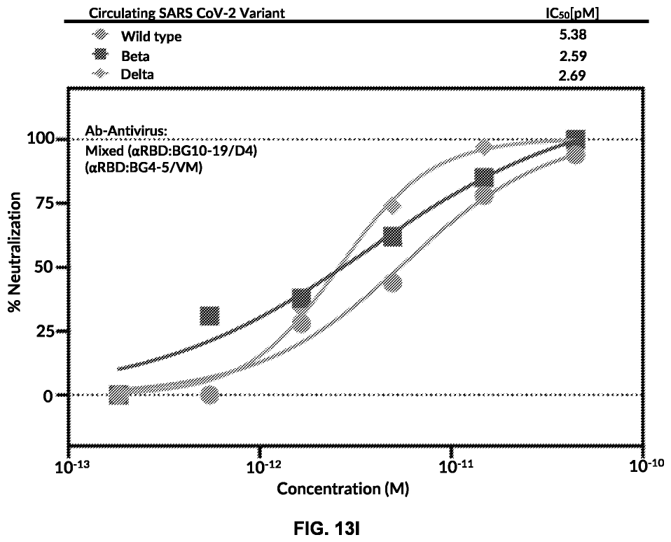

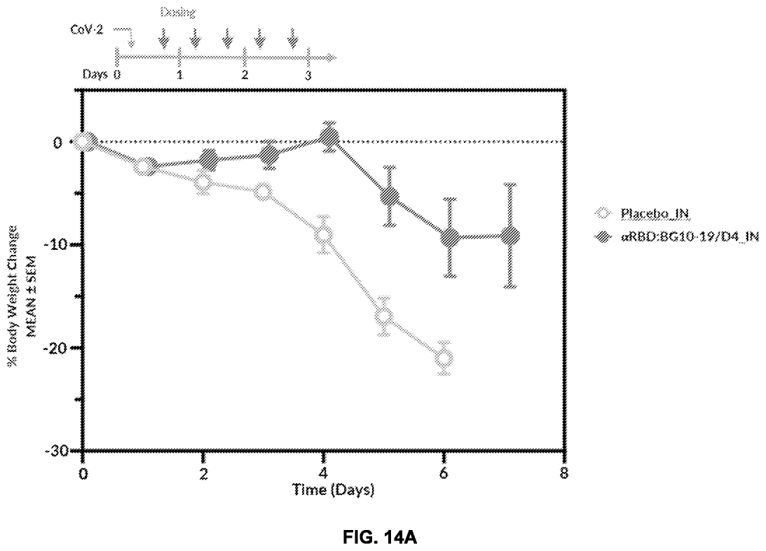

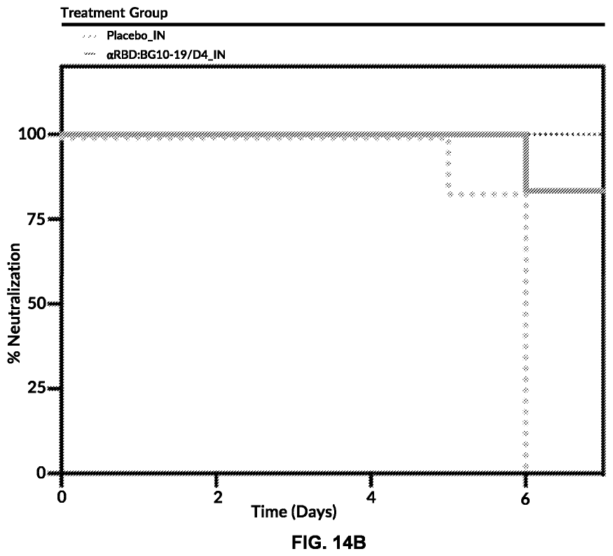

In vivo live virus neutralization efficacy of Ab-Antiviruses was conducted in in hACE2 mice. Briefly, K18-hACE2 mice were challenged with 2800 PFU of the SARS COV-2 (Strain USA-WA1/2020, BEA resource, NR-52281), and then treated with total 5 doses of intranasal delivered αRBD:BG10-19/D4 Ab-Antiviruses (1×10¹¹ particles per dose) (FIG. 14A and FIG. 14B). As illustrated in FIGS. 14A-14B, Ab-antiviruses were produced using a transfer vector and therefore contained viral genetic materials. A placebo was given to the mice in the control group (FIG. 14A and FIG. 14B). The first dose was given at 4 hours after infection, and subsequent doses were given twice a day at day 1 and day 2 post-infection. Animals were monitored twice daily for signs of COVID-19 disease phenotype (ruffled fur, hunched posture, labored breathing) and survival during the study period. Body weights were measured once daily during the study period, and lung tissue was collected and sampled for viral load assays by PRNT. Tissues were stored at 80° C. for histology and viral load analysis by qPCR and PRNT analysis. The mice treated with the αRBD:BG10-19/D4 Ab-Antiviruses exhibited post-infection neutralization of SARS COV-2 (FIG. 14A). Mice treated with αRBD:BG10-19/D4 Ab-Antiviruses exhibited less weight loss (FIG. 14B) and survived the challenge, while all mice in the placebo group exhibited typical symptomatic disease progression and succumbed around day 6 post-infection (FIG. 14A and FIG. 14B). The results demonstrated that Ab-Antiviruses can serve as viable therapeutics for SARS COV-2 at a fraction of the efficacious dose used in monoclonal antibody treatments.

### Example 10: Design of Enveloped Particle Display Vector

To mimic multivalent protein expression on cell surface, two different types of enveloped particle display vectors for efficient protein display on viral-like particle (VLP) and extracellular vesicles (EV) are designed.

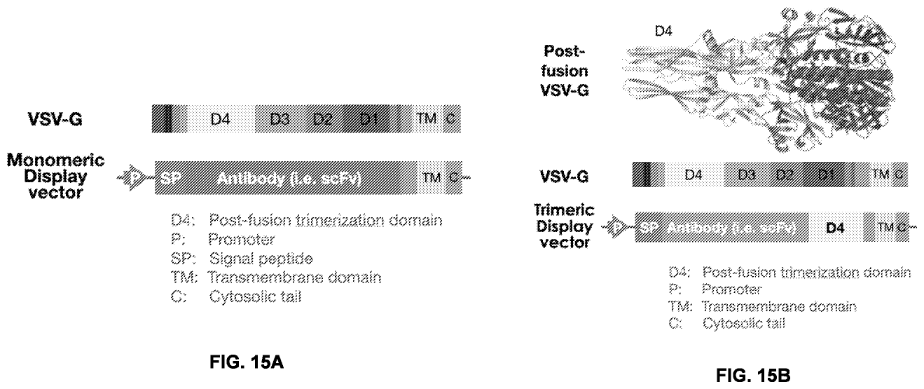

A monomeric display vector expressing a fusion protein consisting of an antiviral protein (i.e. spike-binding scFv) linked to the VSVG transmembrane and intracellular domains is designed as shown in FIG. 15A to display hundreds of copies of monomeric proteins on the surface of VLPs and EVs. Aside from the use of monomeric formats that are suited to form high avidity interactions with similarly displayed patterned proteins on cell surfaces, enveloped particles are made to match oligomeric display formats to enhance avidity at the level of individual oligomeric binding partners. To this end, a trimeric display vector expressing a fusion protein consisting of an antiviral protein (i.e., spike-binding scFv) linked to the D4 post-fusion trimerization domain of VSVG, followed by the transmembrane and intracellular domains of VSVG is designed as shown in FIG. 15B. The vector is used to display hundreds of copies of trimeric proteins on the surface of VLPs and EVs and are well suited to form high avidity interactions with similarly oligomeric proteins on the surface of cells.

### Example 11. Generation of Monomeric Antivirus

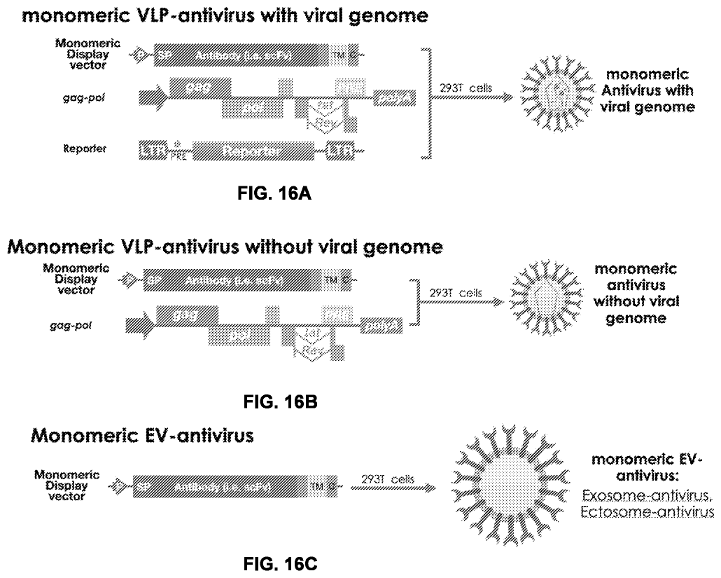

Multivalent spike-binding antibodies are displayed as monomers on the surface of a viral-like particle (VLP) and an extracellular vesicle using a monomeric display vector. The monomeric VLP-based enveloped particle is produced with viral RNA genomes in which the monomeric peptide display construct with a lentiviral packaging construct expresses essential packaging components including Gag-Pol and Rev proteins and a viral genome transfer encoding a GFP/luciferase reporter as shown in FIG. 16A. The monomeric VLP-based enveloped particle without RNA genome is produced by co-transfecting displaying vector with only a lentiviral packaging construct but not the viral genome transfer vector as shown in FIG. 16B. The monomeric EV-based antivirus which includes exosome-antivirus and ectosome-antivirus is produced by transfecting only monomeric peptide displaying vector in 293T cells as shown in FIG. 16C.

Codon-optimized display peptide sequences are synthesized (Twist) and cloned into a display construct to create fusion peptides consisting of the extracellular domain of display peptide and a display anchoring protein. To generate enveloped particles displaying monomeric peptides the extracellular domains were fused to a synthetic VSV-G sequence encoding the transmembrane and cytoplasmic tail domains.

Enveloped particles based on VLPs or EVs can be produced from transfected 293T cells. To produce lentiviral-VLP based MVPs with viral genomes, surface display construct, lentiviral packaging vector (i.e. psPAX2), and lentiviral genome transfer vector are co-transfected into 293T cells. To produce lentiviral-VLP based MVPs without viral genomes, display peptides displaying construct and lentiviral packaging vector (i.e. psPAX2) are co-transfected into 293T cells. Finally, to produce EV-based MVPs, only display peptide displaying construct is transfected into 293T cells.

Specifically, in preparation for transfection, 7.5×106 HEK293T cells (ATCC CRL-3216) are seeded overnight in 10-cm dishes containing DMEM media with glucose, L-glutamine and sodium pyruvate (Corning) supplemented with 10% fetal bovine serum (Sigma) and 1% Penicillin Streptomycin (Life Technologies), referred to as “293T Growth Media.” Cells should reach ≈90% confluence the next day at time of transfection. The following day, transfection DNA mixture along with polyethylenimine (PEI) in OPTI-MEM reduced serum medium (gibco) are prepared. Transfection mixture is incubated at room temperature for 15 minutes before being added to cells, which are then incubated at 37° C. in 5% CO2. 6 hours post-transfection, 293T Growth Media is changed to 293T Growth Media supplemented with 0.1% sodium butyrate (referred to as “Transfection Media”) before being returned to incubation. After incubating for 24 hours at 37° C. with 5% CO2 in Transfection Media, supernatant containing pseudovirus is collected, centrifuged at 1680 rpm for 5 minutes to remove cellular debris and mixed with 1× polyethylene glycol 8000 solution (PEG, Hampton Research), before being stored at 4° C. for 24 hours to allow fractionation. Cells are replenished with fresh Transfection Media, and a second pseudovirus supernatant collection are performed at 48 hours. Supernatant collections are then pooled, PEG precipitated and purified by size exclusion chromatography using Sephacryl S-300 High Resolution Beads (Sigma Aldrich).

### Example 12. Generation of Trimeric Antivirus

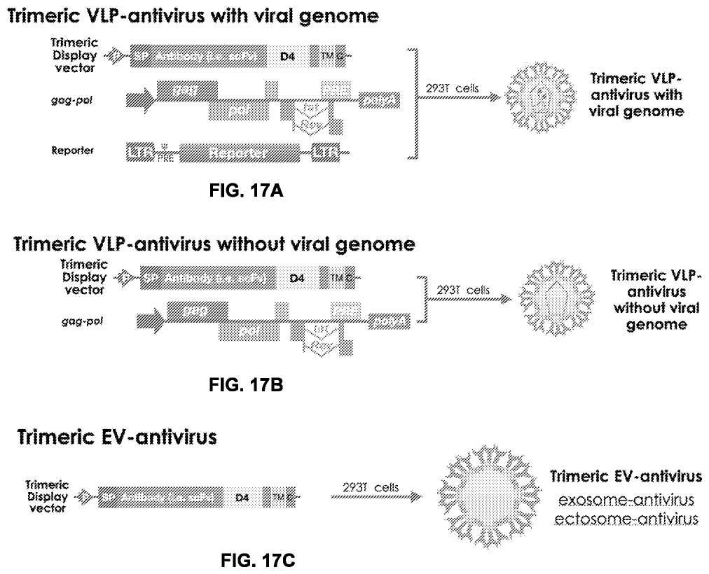

Multivalent spike-binding antibodies are displayed as trimers on the surface of a viral-like particle (VLP) and an extracellular vesicle using a trimeric display vector. The trimeric VLP-based enveloped particle is produced with viral RNA genomes in which the trimeric peptide display construct with a lentiviral packaging construct expresses essential packaging components including Gag-Pol and Rev proteins and a viral genome transfer encoding a GFP/luciferase reported as shown in FIG. 17A. The trimeric VLP-based enveloped particle without RNA genome is produced by co-transfecting displaying vector together with only a lentiviral packaging construct but not the viral genome transfer vector as shown in FIG. 17B. The trimeric EV-based antivirus which includes exosome-antivirus and ectosome-antivirus is produced by transfecting only the trimeric peptide displaying vector in 293T cells as shown in FIG. 17C.

Codon-optimized display peptide sequences are synthesized (Twist) and cloned into a display construct to create fusion peptides consisting of the extracellular domain of display peptide and a display anchoring protein. To generate enveloped particles displaying trimeric peptides the extracellular domains are fused to a synthetic VSV-G sequence encoding the D4 post-fusion trimerization domain and the transmembrane and cytoplasmic tail domains.

Methods for production of VLP-based or EV-based enveloped particles are according to methods as described in Example 11.

### Example 13: Generation of Mixed Monomeric and Trimeric Antivirus

Enveloped particles displaying mixed monomeric and trimeric spike-binding antibodies are generated by co-transfecting monomeric and trimeric peptide display constructs. Such design is used to increase the density of the displayed peptide or to create combinatorial of distinct displayed peptides. Mixed monomeric and trimeric enveloped particles are built with VLPs and EVs by co-transfecting monomeric and trimeric display vectors.

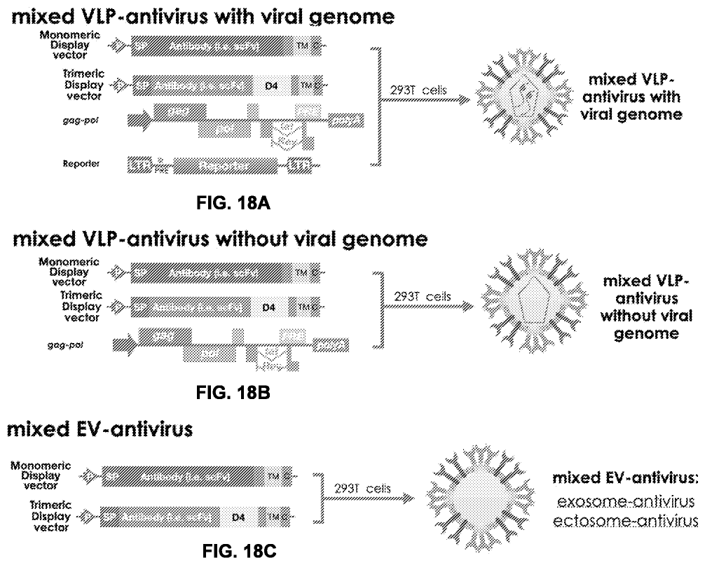

To produce mixed VLPs with viral RNA genomes, the mixed monomeric and trimeric display peptides fusion constructs are co-transfected with a lentiviral packaging construct expressing essential packaging components, such as Gag-Pol and Rec proteins, and viral genome transfer vector encoding a GFP/luciferase reported as shown in FIG. 18A. The mixed VLP-based enveloped particle without RNA genome are produced by co-transfecting the mixed monomeric and trimeric display vector together with only a lentiviral packaging construct but not the viral genome transfer vector as shown in FIG. 18B. The mixed EV-based antivirus which includes mixed exosome-antivirus and ectosome-antivirus is produced by transfecting the mixed monomeric and trimeric display peptide constructs into 293T cells as shown FIG. 18C.

Methods for production of VLP-based or EV-based enveloped particles are according to methods as described in Example 11.

### Example 14. Protein Display Configurations on Enveloped Particles

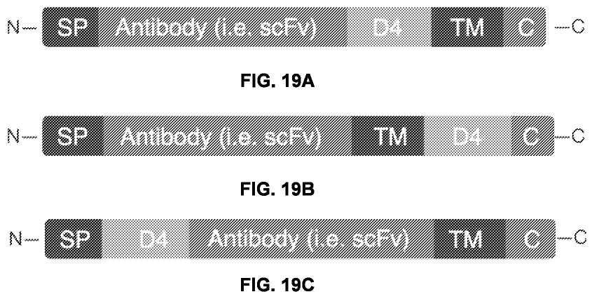

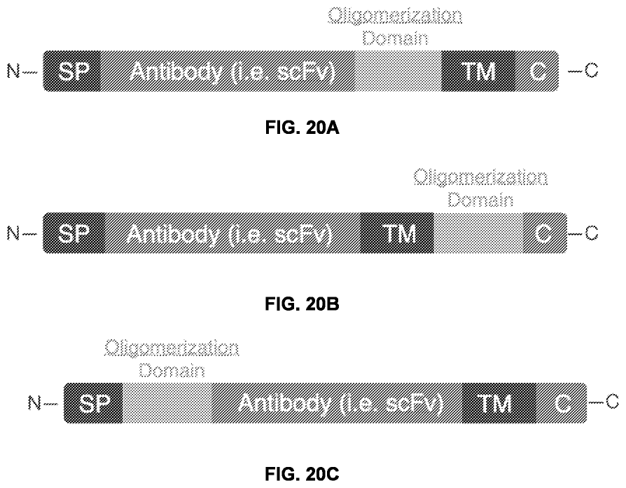

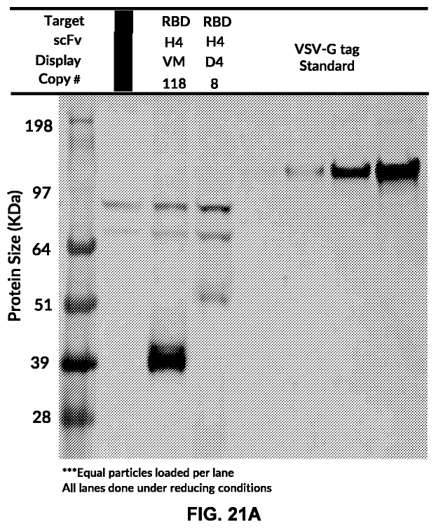

Enveloped particles are genetically programmed to display peptides of interest in various configurations by modifying the display vector as shown in FIGS. 19A-19C and FIGS. 20A-20C. The VSVG D4 trimerization domain can be placed at various positions of the fusion peptide: (1) extracellular and juxtaposed to the transmembrane domain; (2) intracellular and juxtaposed to the transmembrane domain; (3) extracellular and after the signal peptide (FIGS. 19A-19C). Furthermore, various oligomerization domains may be used for distinct surface display patterns that are suitable for the function of displayed molecules (FIGS. 20A-20C). In addition to the VSVG D4 trimerization domain, the Dengue E protein fusion domain or a foldon domain are used to create trimeric display patterns on the surface of VLPs and EVs. Leucine zipper domains and the influenza neuraminidase stem domain are used to create dimeric and tetrameric display patterns on the surface of VLPs and EVs, respectively. Exemplary oligomerization domains and valence are summarized in Table 8. With these display configurations, it is possible to program combinatorial MVPs with mixed monomeric, dimeric, trimeric, and tetrameric display patterns optimized to their function in target cell regulation or virus neutralization.

TABLE 8
Exemplary oligomerization domains and valence

| Oligomerization Domain | Valence |
|---|---|
| VSV-G protein D4 | Trimer |
| Dengue E protein fusion protein | Trimer |
| Foldon | Trimer |
| Leucine Zipper | Dimer |
| Influenza Neuraminidase stem | Tetramer |

### Example 15: Characterization of Proteins Displayed on Enveloped Particles

The concentration of VLP- or EV-based enveloped particles are measured by P24 ELISA or tunable resistive pulse sensing (TRPS, qNano), respectively. Then, copies of displayed peptides on enveloped particles are determined by quantitative Western-blot analyses. Finally, the oligomerization patterns of displayed peptides on the enveloped particles were discerned by non-reducing PAGE analyses. Enveloped particles are expected to display at least 10 copies of protein molecules on surface of VLPs and EVs with monomeric or trimeric configurations.

### Lentiviral Particle Quantification by p24 ELISA and Tunable Resistive Pulse Sensing

P24 concentrations in pseudovirus samples of pseudotyped coronaviruses, influenza viruses and antibody-based antivirus particles are determined using an HIV p24 SimpleStep ELISA kit (Abcam) per the manufacturer's protocol. Concentrations of lentiviral pseudovirus particles are extrapolated from the assumption that each lentiviral particle contains approximately 2000 molecules of p24, or 1.25×104 pseudovirus particles per picogram of p24 protein.

Pseudovirus concentrations determined via p24 ELISA are corroborated by tunable resistive pulse sensing (TRPS, qNano, IZON). Purified pseudovirus collections are diluted in 0.2 μm filtered PBS with 0.03% Tween-20 (Thermo Fisher Scientific) prior to qNano analysis. Concentration and size distributions of pseudotyped particles are then determined using an NP200 nanopore at a 45.5 mm stretch, and applied voltages between 0.5 and 0.7V are used to achieve a stable current of 130 nA through the nanopore. Measurements for each pseudovirus sample are taken at pressures of 3, 5 and 8 mbar, and considered valid if at least 500 events were recorded, particle rate was linear and root mean squared signal noise was maintained below 10 pA. Pseudovirus concentrations are then determined by comparison to a standardized multi-pressure calibration using CPC200 (mode diameter: 200 nm) (IZON) carboxylated polystyrene beads diluted 1:200 in 0.2 μM filtered PBS from their original concentration of 7.3×10¹¹ particles per/mL. Measurements are analyzed using IZON Control Suite 3.4 software to determine original sample concentrations.

### Quantify Lentiviral VIP-Based MVPs

P24 concentrations of the MVP samples are determined by using the Abcam HIV P24 SimpleStep ELISA kit following manufacturer's instruction. The concentrations of lentiviral pseudovirion particles are derived based on the assumption that each lentiviral particle contains about ˜2000 molecules of P24 or 1.25×10⁴ viral particles/picogram of P24 protein.

### Quantify EV-Based MVPs

The sizes and concentrations of extracellular vesicle-based MVPs are determined by tunable resistive pulse sensing (TRPS, qNano, IZON). Purified pseudovirus collections are diluted in 0.2 μm filtered PBS with 0.03% Tween-20 (Thermo Fisher Scientific) prior to qNano analysis. Concentration and size distributions of MVP-ICs are then determined using an NP200 nanopore at a 45.5 mm stretch, and applied voltages between 0.5 and 0.7V were used to achieve a stable current of 130 nA through the nanopore. Measurements for each pseudovirus sample are taken at pressures of 3, 5 and 8 mbar, and considered valid if at least 500 events were recorded, particle rate was linear and root mean squared signal noise was maintained below 10 pA. MVPs concentrations are then determined by comparison to a standardized multi-pressure calibration using CPC200 (mode diameter: 200 nm) (IZON) carboxylated polystyrene beads diluted 1:200 in 0.2 μM filtered PBS from their original concentration of 7.3×1011 particles per/mL. Measurements are analyzed using IZON Control Suite 3.4 software to determine original sample concentrations.

### Western Blot Analysis of MVPs

Expression of fusion proteins on MVPs are confirmed via western blot analysis of purified particles. Samples of purified MVPs are lysed at 4° C. for 10 minutes with cell lysis buffer (Cell Signaling) before being mixed with NuPage LDS sample buffer (Thermo Fisher Scientific) and boiled at 95° C. for 5 minutes. Differences in oligomerization are determined by running samples in reducing and non-reducing conditions. Under reducing conditions, 5% 2-Mercaptoethanol (Thermo Fisher Scientific) are added to samples to dissociate oligomerized MVP-ICs. Protein samples are then separated on NuPAGE 4-12% Bis-Tris gels (Thermo Fisher Scientific) and transferred onto a polyvinylidene fluoride (PVDF) membrane (Life Technologies). PVDF membranes are blocked with TRIS-buffered saline with Tween-20 (TBST) and 5% skim milk (Research Products International) for 1 hour, prior to overnight incubation with primary antibody diluted in 5% milk. For display fusion constructs expressing VSVG-tag, an anti-VSV-G epitope tag rabbit polyclonal antibody (BioLegend, Poly29039) are used at a 1:2000 dilution. The following day, the PVDF membrane are washed 3 times with 1×TBST and stained with a goat-anti-rabbit secondary antibody (IRDye 680) at a 1:5000 dilution for 60 minutes in 5% milk. Post-secondary antibody staining, the PVDF membrane are again washed 3 times with TBST before imaging on a Licor Odyssey scanner.

Alternatively, western blot analyses are performed using an automated Simple Western size-based protein assay (Protein Simple) following the manufacturer's protocols. Unless otherwise mentioned, all reagents used here are from Protein Simple. Concentrated samples are lysed as described above, before being diluted 1:10 in 0.1× sample buffer for loading on capillaries. Displayed fusion protein expression levels are identified using the same primary rabbit polyclonal antibody at a 1:400 dilution and an HRP conjugated anti-rabbit secondary antibody (Protein Simple). Chemiluminescence signal analysis and absolute quantitation are performed using Compass software (Protein Simple).

### Quantitative Western Blot Analyses

Quantitative Western blot analyses are performed to determine the copies of fusion protein displayed per particle. P24 ELISA or TRPS (qNano) assays are used to determine the MVP sample concentrations. Purified MVP samples are processed and analyzed via western blot under reducing conditions as described above. A reference decoy-MVP with a known display copy number are used to generate a standard curve, from which copy numbers of displayed protein on respective particles are determined.

### Example 16: Exemplary Sequences

TABLE 9
Exemplary Antibody Sequences

| | SEQ | | SEQ | |
|---|---|---|---|---|
| | ID | | ID | |
| Name | NO. | VH Amino Acid Sequence | NO. | VL Amino Acid Sequence |
| 80R | 1 | EVQLVQSGGGVVQPGK | 15 | ETTLTQSPATLSLSPGERATLSC |
| | | SLRLSCAASGFAFSSYA | | RASQSVRSNLAWYQQKPGQAP |
| | | MHWVRQAPGKGLEWV | | RPLIYDASTRATGIPDRFSGSGS |
| | | AVISYDGSNKYYADSVK | | GTDFTLTISRLEPEDFAVYYCQQ |
| | | GRFTISRDNSKNTLYLQ | | RSNWPPTFGQGTKVEVKSGLVP |
| | | MNSLRAEDTAVYYCAR | | R |
| | | DRSYYLDYWGQGTLVT | | |
| | | VSS | | |
| H4 | 2 | QVQLVQSGAEVKKPGA | 16 | DIQMTQSPLSLPVTPGEPASISCR |
| | | SVKVSCKASGYTFTGYY | | SSQSLLDSDDGNTYLDWYLQKP |
| | | MHWVRQAPGQGLEWM | | GQSPQLLIYTLSYRASGVPDRFS |
| | | GRINPNSGGTNYAQKFQ | | GSGSGTDFTLKISRVEAEDVGV |
| | | GRVTMTRDTSISTAYME | | YYCMQRIEFPLTFGGGTKVEIK |
| | | LSRLRSDDTAVYYCARV | | |
| | | PYCSSTSCHRDWYFDL | | |
| | | WGRGTLVTVSS | | |
| 7D10 | 3 | EVQLVESGAEVVKPGAS | 17 | DIVMTQSPASLTVSLGQRATISC |
| | | VKMSCKASGYPFTSYNI | | RASKSVSASGYNYLHWYQQRP |
| | | HWIKQTPGQGLEWIGAI | | GQPPKLLIYLAFNLESGVPARFN |
| | | YPGNGDTSYTQKFKVK | | GSGSGTDFTLNIHPVEEEDAATY |
| | | ATLTSDKSSSTAYMQLS | | YCQHSRDLPFTFGSGTKLEIK |
| | | SLTSEDSAVYFCARYGN | | |
| | | YPSYAMDYWGQGTSVT | | |
| | | VSS | | |
| 1E01 | 4 | EVQLVESGGRVVRPGGS | 18 | DIQLTQSPSFLSASVGDRVTITCR |
| | | LRLSCAASGFTEDDYGM | | ASQDISSYLAWYQQKPGNAPKL |
| | | SWVRQPPGKGLEFVSGL | | LIYAASLLQSGVPSRFSAFGSGT |
| | | NWNGDITAFTDSVKGRF | | EFTLTISSLQPEDFATYYCQHLK |
| | | TISRDNVKSSLYLQMNS | | SYPLFTFGPGTKVDIK |
| | | LRADDTAFYYCARVRT | | |
| | | WGDYTTGEEIINSWYFD | | |
| | | LWGRGTLVTVSS | | |
| FI6 | 5 | QVQLVESGGGVVQPGR | 19 | DIVMTQSPDSLAVSLGERATINC |
| | | SLRLSCAASGFTFSTYA | | KSSQSVTFNYKNYLAWYQQKP |
| | | MHWVRQAPGKGLEWV | | GQPPKLLIYWASTRESGVPDRFS |
| | | AVISYDANYKYYADSV | | GSGSGTDFTLTISSLQAEDVAVY |
| | | KGRFTISRDNSKNTLYL | | YCQQHYRTPPTFGQGTKVEIKR |
| | | QMNSLRAEDTAVYYCA | | TVAAPSVFIFPPSDEQLKSGTAS |
| | | KDSQLRSLLYFEWLSQG | | VVCLLNNFYPREAKVQWKVDN |
| | | YFDYWGQGTLVTVSSA | | ALQSGNSQESVTEQDSKDSTYS |
| | | STKGPSVFPLAPSSGGTA | | LSSTLTLSKADYEKHKVYACEV |
| | | ALGCLVKDYFPEPVTVS | | THQGLSSPVTKSFNRGEC |
| | | WNSGALTSGVHTFPAVL | | |
| | | QSSGLYSLSSVVTVPSSS | | |
| | | LGTQTYICNVNHKPSNT | | |
| | | KVDKRVEPK | | |
| 0304-4A10 | 6 | EVQLVESGGGLVQPGGS | 20 | EIVLTQSPDSLAVSLGERATINC |
| | | LRLSCAASGFTFSTYAM | | RSSQSVLYSSNNKNYLAWYQQ |
| | | HWVRQAPGKGLEYVSG | | KPGQPPKVLIYWASTRESGVPD |
| | | ISSNGGSTYYANSVKGR | | RFSGSGSGTDFTLTISSLQAEDV |
| | | FTISRDNSKNTLYIQMGS | | AVYYCQQYYSSPYAFGPGTKV |
| | | LRAEDMAVYYCARSSS | | DIK |
| | | RGFDYWGQGTLVTVSS | | |
| 2M-14E5 | 7 | EVQLVESGGGVVQPGRS | 21 | AIRMTQSPSFLSVSVGDRVTITC |
| | | LRLSCAASGFTFSTYSM | | RASQGISSYLAWYQQKPGKAPK |
| | | HWVRQAPGKGLEWVA | | LLIYAASTLQSGVPSRFSGSGSG |
| | | VISYDGSNKYYADSVKG | | TEFTLTISSLQPEDFATYYCQQL |
| | | RFTISRDNSKNTLYLQV | | NSYVTFGGGTKVEIK |
| | | NSLRAEDTAVYYCARSG | | |
| | | GGSYRGPFDYWGQGTL | | |
| | | VTVSS | | |
| 9A1 | 8 | EVQLLESGGGVVQPGRS | 22 | DIVMTQSPATLSASPGERVTLSC |
| | | LRLSCVVSGFTFNNYGM | | RASQNIRNNLAWYQQKPGQAP |
| | | HWVRQAPGKGLEWVA | | RLLIHGASTRAAGAPARFSGSGS |
| | | VISYEGSVKYYGDHVDG | | DTQFTLTVSSLQSEDFAVYYCH |
| | | RFTISRDPFKNTLYLHM | | QYSKWPVTFGGGTKVDIK |
| | | NNLRPDDTAVYYCAKV | | |
| | | SAIFWLGQGLSPIDVWG | | |
| | | QGTTVTVSS | | |
| COV2- | 9 | QVQLVQSGAEVKKPGA | 23 | QSVLTQPPSASGTPGQRVTISCS |
| 2021 | | SVKVSCKVSGYTLIELSI | | GSSSNIGSNYVYWYQQLPGTAP |
| | | HWVRQAPGKGLEWMG | | KLLIYRNNQRPSGVPDRFSGSKS |
| | | GFDPEDVETIYAQKFQG | | GTSASLAISGLRSEDEADYYCA |
| | | RVTMTEDTSTDTAYME | | AWDASLSGHVVFGGGTKLTVL |
| | | LSSLTSEDRAVYYCATQ | | |
| | | PAAIGGTPPYYWGQGTL | | |
| | | VTVSS | | |
| COV2- | 10 | QVQLVQSGAEVKKPGA | 24 | DIQMTQSPSSLSASVGDRVTITC |
| 2026 | | SVKVSCKVSGYTLTELSI | | RASQGITNYLAWFQQKPGKAPK |
| | | HWVRQAPGKGLEWMG | | SLIYAVSSLQSGVPSKFSGSGSG |
| | | GFDPEDGETVYAQKFQG | | TDFTLTISSLQPEDFATYYCQQY |
| | | RVTMTEDTSSDTAYMEL | | NSYPWTFGQGTKVEIK |
| | | SSLRSEDTAVYYCATSFP | | |
| | | IRGDPSYYYYYYGMDV | | |
| | | WGQGTTVTVSS | | |
| COV2- | 11 | EVQLVESGGGLVQPGGS | 25 | DIQLTQSPSFLSASVGDRVTITCR |
| 2146 | | LRLSCEASGFTFSSSEIN | | ASQGISSYLAWYQQKPGKAPKL |
| | | WVRQAPGKGLEWVSHI | | LIYAASTLQSGVPSRFSGSGSGT |
| | | SSSGSIIYYADSVKGRFTI | | EFTLTISSLQPEDFATYYCQQLN |
| | | SRDNAKNSLYLQMNSL | | SYPVTFGQGTKVEIK |
| | | RAEDTAVYYCARRSYRS | | |
| | | SWYYYYGMDVWGQGT | | |
| | | TVTVSS | | |
| 2M-10B11 | 12 | EVQLVESGGGLVQPGGS | 26 | QSALTQPHSVSESPGKTVTISCT |
| | | LRLSCAASGFTVSSNYM | | GSSGSIASNYVQWYQQRPGSAP |
| | | SWVRQAPGKGLEWVSV | | TTVIYEDNQRPSGVPDRFSGSID |
| | | IYSGGSTYYADSVKGRF | | SSSNSASLTISGLKTEDEADYYC |
| | | TISRDNSKNTLYLQMNS | | QSYDSSNHWVFGGGTKLTVL |
| | | LRAEDTAVYYCARATW | | |
| | | LRGVMDVWGQGTTVTV | | |
| | | SS | | |
| C021 | 13 | QVQLQESGPGLVKPSQT | 27 | DIVMTQSPLSLPVTPGEPASISCR |
| | | LSLTCTVSGGSISSGGYY | | SSQSLLHSNGYNYLDWYLQKPG |
| | | WSWIRQHPGKGLEWIG | | QSPQLLIYLGSNRASGVPDRFSG |
| | | YIYYSGSTYYNPSLKSR | | SGSGTDFTLKISRVEAEDVGVY |
| | | VTISVDTSKNQFSLKLSS | | YCMQALQTPFTFGPGTKVDIK |
| | | VTAADTAVYYCARVWQ | | |
| | | YYDSSGSFDYWGQGTL | | |
| | | VTVSS | | |
| C018 | 14 | EVQLVESGGGVVQPGRS | 28 | DIQLTQSPSSLSASVGDRVTITCR |
| | | LRLSCAASGFTFSNYAIH | | ASQSIRSYLNWYQQKPGKAPKL |
| | | WVRQAPGKGLEWVAVI | | LIYAASSLQSGVPSRFSGSGSGT |
| | | SYDGSNKYYADSVKGR | | DFTLTISSLQPDDFATYYCQQSY |
| | | FTISRDNSKNTLYLQMN | | STPPATFGQGTKLEIK |
| | | SLRAEDTAVYYCARDFD | | |
| | | DSSFWAFDYWGQGTLV | | |
| | | TVSS | | |
| BG4-5 | 81 | ATGVHSQVQLMQSGAE | 82 | TGSWAQSVLTQPPSASGTPGQR |
| | | VKKPGASVKVSCKASG | | VTVSCSGSSSNILNNYVYWYQQ |
| | | DSFTDYYVHWVRQAPG | | LPGTAPKLLIYRNNQRPSGVPDR |
| | | QGLEWMGWISPYSGGT | | FSGSKSGTSASLAISGLRSEDEA |
| | | NYAPKFRGRVTMTRDTS | | DYYCAAWDDSLFVVFGGGTKL |
| | | ITTAHMELGRLRSDDTA | | TVLGQPKAAPSVTLFPPS |
| | | VYFCARSVGYDAFDVW | | |
| | | GPGTMVTVSSAST | | |
| BG7-15 | 83 | ATGVHSQVQLVQSGAE | 84 | ATGVHSEIVLTQSPGTLSLSPGE |
| | | VKKPGASVKVSCKASG | | RATLSCRASQSVSGTFLAWYQQ |
| | | YTFTSYAISWVRQAPGQ | | KPGQAPRLLISGASSRATGIPDR |
| | | GLEWMGWVSAYNGNT | | FSGSGSGTDFTLTISRLEPEDFAV |
| | | NYAQKLQGRVTMTTDT | | YYCQQYGSSRPTFGQGTKLEIK |
| | | STNTAYMELRSLRSDDT | | RT |
| | | AIYYCAIPYSSVTFDCW | | |
| | | GQGTLVTVSSAST | | |
| BG10-19 | 85 | ATGVHSEVQLVQSGAE | 86 | ATGSWAQSVLTQPPSASGTPGQ |
| | | VKKPGESLKISCKGSGY | | RVTISCSGSSSNIGSDHVYWYQQ |
| | | SFTSYWIGWVRQMPGK | | LPGTAPKLFIYRNNQRPSGVPDR |
| | | GLEWMGVIYPGDSDTR | | FSGSKSGTSASLAISGLRSEDEA |
| | | YSPSFQGQVTISADKSIS | | DYYCAAWDASLSGYVFGTGTK |
| | | TAYLQWSSLKASDTAM | | VTVLGQPKANPTVTLFPPS |
| | | YYCARTQWGYNYGSHF | | |
| | | FYMDVWGKGTTVTVSS | | |
| | | AST | | |

TABLE 10
Exemplary Antibody Fusion Protein Sequences

| | SEQ | |
|---|---|---|
| | ID | |
| Name | NO. | Amino Acid Sequences |
| αRBD:80R/VM | 53 | MEFGLSWVFLVALFRGVQSEVQLVQSGGGVVQPGKSLRLSCAASGFAFSS |
| | | YAMHWVRQAPGKGLEWVAVISYDGSNKYYADSVKGRFTISRDNSKNTLY |
| | | LQMNSLRAEDTAVYYCARDRSYYLDYWGQGTLVTVSSGGGGSGGGGSG |
| | | GGGSETTLTQSPATLSLSPGERATLSCRASQSVRSNLAWYQQKPGQAPRPL |
| | | IYDASTRATGIPDRFSGSGSGTDFTLTISRLEPEDFAVYYCQQRSNWPPTFG |
| | | QGTKVEVKSGLVPRRSGMLDSDLHLSSKAQVFEHPHIQDAASQLPDDESL |
| | | FFGDTGLSKNPIELVEGWFSSWKSSIASFFFIIGLIIGLFLVLRVGIHLCIKLK |
| | | HTKKRQIYTDIEMNRLGK |
| αRBD:H4/VM | 54 | MEFGLSWVFLVALFRGVQSQVQLVQSGAEVKKPGASVKVSCKASGYTFT |
| | | GYYMHWVRQAPGQGLEWMGRINPNSGGTNYAQKFQGRVTMTRDTSIST |
| | | AYMELSRLRSDDTAVYYCARVPYCSSTSCHRDWYFDLWGRGTLVTVSSG |
| | | GGGSGGGGSGGGGSDIQMTQSPLSLPVTPGEPASISCRSSQSLLDSDDGNT |
| | | YLDWYLQKPGQSPQLLIYTLSYRASGVPDRFSGSGSGTDFTLKISRVEAED |
| | | VGVYYCMQRIEFPLTFGGGTKVEIKSRGMLDSDLHLSSKAQVFEHPHIQDA |
| | | ASQLPDDESLFFGDTGLSKNPIELVEGWFSSWKSSIASFFFIIGLIIGLFLVLR |
| | | VGIHLCIKLKHTKKRQIYTDIEMNRLGK |
| αRBD:H4/D4 | 55 | MEFGLSWVFLVALFRGVQSQVQLVQSGAEVKKPGASVKVSCKASGYTFT |
| | | GYYMHWVRQAPGQGLEWMGRINPNSGGTNYAQKFQGRVTMTRDTSIST |
| | | AYMELSRLRSDDTAVYYCARVPYCSSTSCHRDWYFDLWGRGTLVTVSSG |
| | | GGGSGGGGSGGGGSDIQMTQSPLSLPVTPGEPASISCRSSQSLLDSDDGNT |
| | | YLDWYLQKPGQSPQLLIYTLSYRASGVPDRFSGSGSGTDFTLKISRVEAED |
| | | VGVYYCMQRIEFPLTFGGGTKVEIKDIIQADGWMCHASKWVTTCDFRWY |
| | | GPKYITHSIRSFTPSVEQCKESIEQTKQGTWLNPGFPPQSCGYATVIDAEAV |
| | | IVQVTPHHVLVDEYTGEWVDSQFINGKCSNYICPTVHNSTTWHSDYKVKG |
| | | LCDSNLGMLDSDLHLSSKAQVFEHPHIQDAASQLPDDESLFFGDTGLSKNP |
| | | IELVEGWFSSWKSSIASFFFIIGLIIGLFLVLRVGIHLCIKLKHTKKRQIYTDIE |
| | | MNRLGK |
| αNTD:7D10/VM | 56 | MEFGLSWVFLVALFRGVQSDIVMTQSPASLTVSLGQRATISCRASKSVSAS |
| | | GYNYLHWYQQRPGQPPKLLIYLAFNLESGVPARFNGSGSGTDFTLNIHPVE |
| | | EEDAATYYCQHSRDLPFTFGSGTKLEIKGGGSGGGSGGGSEVQLVESGAE |
| | | VVKPGASVKMSCKASGYPFTSYNIHWIKQTPGQGLEWIGAIYPGNGDTSY |
| | | TQKFKVKATLTSDKSSSTAYMQLSSLTSEDSAVYFCARYGNYPSYAMDY |
| | | WGQGTSVTVSSSRGMLDSDLHLSSKAQVFEHPHIQDAASQLPDDESLFFG |
| | | DTGLSKNPIELVEGWFSSWKSSIASFFFIIGLIIGLFLVLRVGIHLCIKLKHTK |
| | | KRQIYTDIEMNRLGK |
| αNA:1E01/D4 | 57 | MEFGLSWVFLVALFRGVQSEVQLVESGGRVVRPGGSLRLSCAASGFTFDD |
| | | YGMSWVRQPPGKGLEFVSGLNWNGDITAFTDSVKGRFTISRDNVKSSLYL |
| | | QMNSLRADDTAFYYCARVRTWGDYTTGEEIINSWYFDLWGRGTLVTVSS |
| | | GGGGSGGGGSGGGGSGGGGSDIQLTQSPSFLSASVGDRVTITCRASQDISS |
| | | YLAWYQQKPGNAPKLLIYAASLLQSGVPSRFSAFGSGTEFTLTISSLQPEDF |
| | | ATYYCQHLKSYPLFTFGPGTKVDIKDIIQADGWMCHASKWVTTCDFRWY |
| | | GPKYITHSIRSFTPSVEQCKESIEQTKQGTWLNPGFPPQSCGYATVTDAEAV |
| | | IVQVTPHHVLVDEYTGEWVDSQFINGKCSNYICPTVHNSTTWHSDYKVKG |
| | | LCDSNLGMLDSDLHLSSKAQVFEHPHIQDAASQLPDDESLFFGDTGLSKNP |
| | | IELVEGWFSSWKSSIASFFFIIGLIIGLFLVLRVGIHLCIKLKHTKKRQIYTDIE |
| | | MNRLGK |
| αHA:FI6/D4 | 58 | MEFGLSWVFLVALFRGVQSQVQLVESGGGVVQPGRSLRLSCAASGFTFST |
| | | YAMHWVRQAPGKGLEWVAVISYDANYKYYADSVKGRFTISRDNSKNTL |
| | | YLQMNSLRAEDTAVYYCAKDSQLRSLLYFEWLSQGYFDYWGQGTLVTVS |
| | | SASTKGPSVFPLAPSSGGTAALGCLVKDYFPEPVTVSWNSGALTSGVHTFP |
| | | AVLQSSGLYSLSSVVTVPSSSLGTQTYICNVNHKPSNTKVDKRVEPKGGGG |
| | | SGGGGSGGGGSGGGGSDIVMTQSPDSLAVSLGERATINCKSSQSVTFNYK |
| | | NYLAWYQQKPGQPPKLLIYWASTRESGVPDRFSGSGSGTDFTLTISSLQAE |
| | | DVAVYYCQQHYRTPPTFGQGTKVEIKRTVAAPSVFIFPPSDEQLKSGTASV |
| | | VCLLNNFYPREAKVQWKVDNALQSGNSQESVTEQDSKDSTYSLSSTLTLS |
| | | KADYEKHKVYACEVTHQGLSSPVTKSFNRGECDIIQADGWMCHASKWVT |
| | | TCDFRWYGPKYITHSIRSFTPSVEQCKESIEQTKQGTWLNPGFPPQSCGYAT |
| | | VTDAEAVIVQVTPHHVLVDEYTGEWVDSQFINGKCSNYICPTVHNSTTWH |
| | | SDYKVKGLCDSNLGMLDSDLHLSSKAQVFEHPHIQDAASQLPDDESLFFG |
| | | DTGLSKNPIELVEGWFSSWKSSIASFFFIIGLIIGLFLVLRVGIHLCIKLKHTK |
| | | KRQIYTDIEMNRLGK |
| αS2:4A10/D4 | 59 | MEFGLSWVFLVALFRGVQSEVQLVESGGGLVQPGGSLRLSCAASGFTFST |
| | | YAMHWVRQAPGKGLEYVSGISSNGGSTYYANSVKGRFTISRDNSKNTLYI |
| | | QMGSLRAEDMAVYYCARSSSRGFDYWGQGTLVTVSSGGGGSGGGGSGG |
| | | GGSGGGGSEIVLTQSPDSLAVSLGERATINCRSSQSVLYSSNNKNYLAWYQ |
| | | QKPGQPPKVLIYWASTRESGVPDRFSGSGSGTDFTLTISSLQAEDVAVYYC |
| | | QQYYSSPYAFGPGTKVDIKDIIQADGWMCHASKWVTTCDFRWYGPKYIT |
| | | HSIRSFTPSVEQCKESIEQTKQGTWLNPGFPPQSCGYATVTDAEAVIVQVTP |
| | | HHVLVDEYTGEWVDSQFINGKCSNYICPTVHNSTTWHSDYKVKGLCDSN |
| | | LGMLDSDLHLSSKAQVFEHPHIQDAASQLPDDESLFFGDTGLSKNPIELVE |
| | | GWFSSWKSSIASFFFIIGLIIGLFLVLRVGIHLCIKLKHTKKRQIYTDIEMNRL |
| | | GK |
| αS2:14E5/D4 | 60 | MEFGLSWVFLVALFRGVQSEVQLVESGGGVVQPGRSLRLSCAASGFTFST |
| | | YSMHWVRQAPGKGLEWVAVISYDGSNKYYADSVKGRFTISRDNSKNTLY |
| | | LQVNSLRAEDTAVYYCARSGGGSYRGPFDYWGQGTLVTVSSGGGGSGGG |
| | | GSGGGGSGGGGSAIRMTQSPSFLSVSVGDRVTITCRASQGISSYLAWYQQK |
| | | PGKAPKLLIYAASTLQSGVPSRFSGSGSGTEFTLTISSLQPEDFATYYCQQL |
| | | NSYVTFGGGTKVEIKDIIQADGWMCHASKWVTTCDFRWYGPKYITHSIRS |
| | | FTPSVEQCKESIEQTKQGTWLNPGFPPQSCGYATVTDAEAVIVQVTPHHVL |
| | | VDEYTGEWVDSQFINGKCSNYICPTVHNSTTWHSDYKVKGLCDSNLGML |
| | | DSDLHLSSKAQVFEHPHIQDAASQLPDDESLFFGDTGLSKNPIELVEGWFSS |
| | | WKSSIASFFFIIGLIIGLFLVLRVGIHLCIKLKHTKKRQIYTDIEMNRLGK |
| aS2:9A1/D4 | 61 | MEFGLSWVFLVALFRGVQSEVQLLESGGGVVQPGRSLRLSCVVSGFTFNN |
| | | YGMHWVRQAPGKGLEWVAVISYEGSVKYYGDHVDGRFTISRDPFKNTLY |
| | | LHMNNLRPDDTAVYYCAKVSAIFWLGQGLSPIDVWGQGTTVTVSSGGGG |
| | | SGGGGSGGGGSGGGGSDIVMTQSPATLSASPGERVTLSCRASQNIRNNLA |
| | | WYQQKPGQAPRLLIHGASTRAAGAPARFSGSGSDTQFTLTVSSLQSEDFAV |
| | | YYCHQYSKWPVTFGGGTKVDIKDIIQADGWMCHASKWVTTCDFRWYGP |
| | | KYITHSIRSFTPSVEQCKESIEQTKQGTWLNPGFPPQSCGYATVTDAEAVIV |
| | | QVTPHHVLVDEYTGEWVDSQFINGKCSNYICPTVHNSTTWHSDYKVKGL |
| | | CDSNLGMLDSDLHLSSKAQVFEHPHIQDAASQLPDDESLFFGDTGLSKNPI |
| | | ELVEGWFSSWKSSIASFFFIIGLIIGLFLVLRVGIHLCIKLKHTKKRQIYTDIE |
| | | MNRLGK |
| αNTD:CV21/VM | 62 | MEFGLSWVFLVALFRGVQSQVQLVQSGAEVKKPGASVKVSCKVSGYTLIE |
| | | LSIHWVRQAPGKGLEWMGGFDPEDVETIYAQKFQGRVTMTEDTSTDTAY |
| | | MELSSLTSEDRAVYYCATQPAAIGGTPPYYWGQGTLVTVSSGGGGSGGG |
| | | GSGGGGSGGGGSQSVLTQPPSASGTPGQRVTISCSGSSSNIGSNYVYWYQQ |
| | | LPGTAPKLLIYRNNQRPSGVPDRFSGSKSGTSASLAISGLRSEDEADYYCAA |
| | | WDASLSGHVVFGGGTKLTVLRGMLDSDLHLSSKAQVFEHPHIQDAASQLP |
| | | DDESLFFGDTGLSKNPIELVEGWFSSWKSSIASFFFIIGLIIGLFLVLRVGIHL |
| | | CIKLKHTKKRQIYTDIEMNRLGK |
| αNTD:CV21/D4 | 63 | MEFGLSWVFLVALFRGVQSQVQLVQSGAEVKKPGASVKVSCKVSGYTLIE |
| | | LSIHWVRQAPGKGLEWMGGFDPEDVETIYAQKFQGRVTMTEDTSTDTAY |
| | | MELSSLTSEDRAVYYCATQPAAIGGTPPYYWGQGTLVTVSSGGGGSGGG |
| | | GSGGGGSGGGGSQSVLTQPPSASGTPGQRVTISCSGSSSNIGSNYVYWYQQ |
| | | LPGTAPKLLIYRNNQRPSGVPDRFSGSKSGTSASLAISGLRSEDEADYYCAA |
| | | WDASLSGHVVFGGGTKLTVLDIIQADGWMCHASKWVTTCDFRWYGPKYI |
| | | THSIRSFTPSVEQCKESIEQTKQGTWLNPGFPPQSCGYATVTDAEAVIVQVT |
| | | PHHVLVDEYTGEWVDSQFINGKCSNYICPTVHNSTTWHSDYKVKGLCDS |
| | | NLGMLDSDLHLSSKAQVFEHPHIQDAASQLPDDESLFFGDTGLSKNPIELV |
| | | EGWFSSWKSSIASFFFIIGLIIGLFLVLRVGIHLCIKLKHTKKRQIYTDIEMNR |
| | | LGK |
| αNTD:CV26/D4 | 64 | MEFGLSWVFLVALFRGVQSQVQLVQSGAEVKKPGASVKVSCKVSGYTLT |
| | | ELSIHWVRQAPGKGLEWMGGFDPEDGETVYAQKFQGRVTMTEDTSSDTA |
| | | YMELSSLRSEDTAVYYCATSFPIRGDPSYYYYYYGMDVWGQGTTVTVSS |
| | | GGGGSGGGGSGGGGSGGGGSDIQMTQSPSSLSASVGDRVTITCRASQGITN |
| | | YLAWFQQKPGKAPKSLIYAVSSLQSGVPSKFSGSGSGTDFTLTISSLQPEDF |
| | | ATYYCQQYNSYPWTFGQGTKVEIKDIIQADGWMCHASKWVTTCDFRWY |
| | | GPKYITHSIRSFTPSVEQCKESIEQTKQGTWLNPGFPPQSCGYATVTDAEAV |
| | | IVQVTPHHVLVDEYTGEWVDSQFINGKCSNYICPTVHNSTTWHSDYKVKG |
| | | LCDSNLGMLDSDLHLSSKAQVFEHPHIQDAASQLPDDESLFFGDTGLSKNP |
| | | IELVEGWFSSWKSSIASFFFIIGLIIGLFLVLRVGIHLCIKLKHTKKRQIYTDIE |
| | | MNRLGK |
| αNTD:CV46/D4 | 65 | MEFGLSWVFLVALFRGVQSEVQLVESGGGLVQPGGSLRLSCEASGFTFSSS |
| | | EINWVRQAPGKGLEWVSHISSSGSIIYYADSVKGRFTISRDNAKNSLYLQM |
| | | NSLRAEDTAVYYCARRSYRSSWYYYYGMDVWGQGTTVTVSSGGGGSGG |
| | | GGSGGGGSGGGGSDIQLTQSPSFLSASVGDRVTITCRASQGISSYLAWYQQ |
| | | KPGKAPKLLIYAASTLQSGVPSRFSGSGSGTEFTLTISSLQPEDFATYYCQQ |
| | | LNSYPVTFGQGTKVEIKDIIQADGWMCHASKWVTTCDFRWYGPKYITHSI |
| | | RSFTPSVEQCKESIEQTKQGTWLNPGFPPQSCGYATVTDAEAVIVQVTPHH |
| | | VLVDEYTGEWVDSQFINGKCSNYICPTVHNSTTWHSDYKVKGLCDSNLG |
| | | MLDSDLHLSSKAQVFEHPHIQDAASQLPDDESLFFGDTGLSKNPIELVEGW |
| | | FSSWKSSIASFFFIIGLIIGLFLVLRVGIHLCIKLKHTKKRQIYTDIEMNRLGK |
| αRBD:B11/D4 | 66 | MEFGLSWVFLVALFRGVQSEVQLVESGGGLVQPGGSLRLSCAASGFTVSS |
| | | NYMSWVRQAPGKGLEWVSVIYSGGSTYYADSVKGRFTISRDNSKNTLYL |
| | | QMNSLRAEDTAVYYCARATWLRGVMDVWGQGTTVTVSSGGGGSGGGG |
| | | SGGGGSGGGGSQSALTQPHSVSESPGKTVTISCTGSSGSIASNYVQWYQQR |
| | | PGSAPTTVIYEDNQRPSGVPDRFSGSIDSSSNSASLTISGLKTEDEADYYCQS |
| | | YDSSNHWVFGGGTKLTVLDIIQADGWMCHASKWVTTCDFRWYGPKYITH |
| | | SIRSFTPSVEQCKESIEQTKQGTWLNPGFPPQSCGYATVTDAEAVIVQVTPH |
| | | HVLVDEYTGEWVDSQFINGKCSNYICPTVHNSTTWHSDYKVKGLCDSNL |
| | | GMLDSDLHLSSKAQVFEHPHIQDAASQLPDDESLFFGDTGLSKNPIELVEG |
| | | WFSSWKSSIASFFFIIGLIIGLFLVLRVGIHLCIKLKHTKKRQIYTDIEMNRLG |
| | | K |
| αRBD:C021/VM | 67 | MEFGLSWVFLVALFRGVQSQVQLQESGPGLVKPSQTLSLTCTVSGGSISSG |
| | | GYYWSWIRQHPGKGLEWIGYIYYSGSTYYNPSLKSRVTISVDTSKNQFSLK |
| | | LSSVTAADTAVYYCARVWQYYDSSGSFDYWGQGTLVTVSSGGGGSGGG |
| | | GSGGGGSGGGGSDIVMTQSPLSLPVTPGEPASISCRSSQSLLHSNGYNYLD |
| | | WYLQKPGQSPQLLIYLGSNRASGVPDRFSGSGSGTDFTLKISRVEAEDVGV |
| | | YYCMQALQTPFTFGPGTKVDIKRGMLDSDLHLSSKAQVFEHPHIQDAASQ |
| | | LPDDESLFFGDTGLSKNPIELVEGWFSSWKSSIASFFFIIGLIIGLFLVLRVGI |
| | | HLCIKLKHTKKRQIYTDIEMNRLGK |
| αRBD:C021/D4 | 68 | MEFGLSWVFLVALFRGVQSQVQLQESGPGLVKPSQTLSLTCTVSGGSISSG |
| | | GYYWSWIRQHPGKGLEWIGYIYYSGSTYYNPSLKSRVTISVDTSKNQFSLK |
| | | LSSVTAADTAVYYCARVWQYYDSSGSFDYWGQGTLVTVSSGGGGSGGG |
| | | GSGGGGSGGGGSDIVMTQSPLSLPVTPGEPASISCRSSQSLLHSNGYNYLD |
| | | WYLQKPGQSPQLLIYLGSNRASGVPDRFSGSGSGTDFTLKISRVEAEDVGV |
| | | YYCMQALQTPFTFGPGTKVDIKDIIQADGWMCHASKWVTTCDFRWYGPK |
| | | YITHSIRSFTPSVEQCKESIEQTKQGTWLNPGFPPQSCGYATVIDAEAVIVQ |
| | | VTPHHVLVDEYTGEWVDSQFINGKCSNYICPTVHNSTTWHSDYKVKGLC |
| | | DSNLGMLDSDLHLSSKAQVFEHPHIQDAASQLPDDESLFFGDTGLSKNPIE |
| | | LVEGWFSSWKSSIASFFFIIGLIIGLFLVLRVGIHLCIKLKHTKKRQIYTDIEM |
| | | NRLGK |
| αRBD:C018/VM | 69 | MEFGLSWVFLVALFRGVQSEVQLVESGGGVVQPGRSLRLSCAASGFTFSN |
| | | YAIHWVRQAPGKGLEWVAVISYDGSNKYYADSVKGRFTISRDNSKNTLY |
| | | LQMNSLRAEDTAVYYCARDFDDSSFWAFDYWGQGTLVTVSSGGGGSGG |
| | | GGSGGGGSGGGGSDIQLTQSPSSLSASVGDRVTITCRASQSIRSYLNWYQQ |
| | | KPGKAPKLLIYAASSLQSGVPSRFSGSGSGTDFTLTISSLQPDDFATYYCQQ |
| | | SYSTPPATFGQGTKLEIKSRGMLDSDLHLSSKAQVFEHPHIQDAASQLPDD |
| | | ESLFFGDTGLSKNPIELVEGWFSSWKSSIASFFFIIGLIIGLFLVLRVGIHLCIK |
| | | LKHTKKRQIYTDIEMNRLGK |
| αRBD:C018/D4 | 70 | MEFGLSWVFLVALFRGVQSEVQLVESGGGVVQPGRSLRLSCAASGFTFSN |
| | | YAIHWVRQAPGKGLEWVAVISYDGSNKYYADSVKGRFTISRDNSKNTLY |
| | | LQMNSLRAEDTAVYYCARDFDDSSFWAFDYWGQGTLVTVSSGGGGSGG |
| | | GGSGGGGSGGGGSDIQLTQSPSSLSASVGDRVTITCRASQSIRSYLNWYQQ |
| | | KPGKAPKLLIYAASSLQSGVPSRFSGSGSGTDFTLTISSLQPDDFATYYCQQ |
| | | SYSTPPATFGQGTKLEIKDIIQADGWMCHASKWVTTCDFRWYGPKYITHSI |
| | | RSFTPSVEQCKESIEQTKQGTWLNPGFPPQSCGYATVTDAEAVIVQVTPHH |
| | | VLVDEYTGEWVDSQFINGKCSNYICPTVHNSTTWHSDYKVKGLCDSNLG |
| | | MLDSDLHLSSKAQVFEHPHIQDAASQLPDDESLFFGDTGLSKNPIELVEGW |
| | | FSSWKSSIASFFFIIGLIIGLFLVLRVGIHLCIKLKHTKKRQIYTDIEMNRLGK |
| (αHA:FI6- | 71 | MEFGLSWVFLVALFRGVQSEVQLVESGGRVVRPGGSLRLSCAASGFTFDD |
| αNA:IE01)/D4 | | YGMSWVRQPPGKGLEFVSGLNWNGDITAFTDSVKGRFTISRDNVKSSLYL |
| | | QMNSLRADDTAFYYCARVRTWGDYTTGEEIINSWYFDLWGRGTLVTVSS |
| | | GGGGSGGGGSGGGGSGGGGSDIQLTQSPSFLSASVGDRVTITCRASQDISS |
| | | YLAWYQQKPGNAPKLLIYAASLLQSGVPSRFSAFGSGTEFTLTISSLQPEDF |
| | | ATYYCQHLKSYPLFTFGPGTKVDIKGGGGSGGGGSGGGGSGGGGSQVQL |
| | | VESGGGVVQPGRSLRLSCAASGFTFSTYAMHWVRQAPGKGLEWVAVISY |
| | | DANYKYYADSVKGRFTISRDNSKNTLYLQMNSLRAEDTAVYYCAKDSQL |
| | | RSLLYFEWLSQGYFDYWGQGTLVTVSSASTKGPSVFPLAPSSGGTAALGC |
| | | LVKDYFPEPVTVSWNSGALTSGVHTFPAVLQSSGLYSLSSVVTVPSSSLGT |
| | | QTYICNVNHKPSNTKVDKRVEPKGGGGSGGGGSGGGGSGGGGSDIVMTQ |
| | | SPDSLAVSLGERATINCKSSQSVTFNYKNYLAWYQQKPGQPPKLLIYWAS |
| | | TRESGVPDRFSGSGSGTDFTLTISSLQAEDVAVYYCQQHYRTPPTFGQGTK |
| | | VEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGECIIQADGWMCHASKWVTTCDFRWYGPKYITHSIRSFTPSVEQC |
| | | KESIEQTKQGTWLNPGFPPQSCGYATVTDAEAVIVQVTPHHVLVDEYTGE |
| | | WVDSQFINGKCSNYICPTVHNSTTWHSDYKVKGLCDSNLGMLDSDLHLSS |
| | | KAQVFEHPHIQDAASQLPDDESLFFGDTGLSKNPIELVEGWFSSWKSSIASF |
| | | FFIIGLIIGLFLVLRVGIHLCIKLKHTKKRQIYTDIEMNRLGK |
| (αRBD:C021- | 72 | MEFGLSWVFLVALFRGVQSQVQLVQSGAEVKKPGASVKVSCKVSGYTLIE |
| αNTD:CV21)/ | | LSIHWVRQAPGKGLEWMGGFDPEDVETIYAQKFQGRVTMTEDTSTDTAY |
| D4 | | MELSSLTSEDRAVYYCATQPAAIGGTPPYYWGQGTLVTVSSGGGGSGGG |
| | | GSGGGGSGGGGSQSVLTQPPSASGTPGQRVTISCSGSSSNIGSNYVYWYQQ |
| | | LPGTAPKLLIYRNNQRPSGVPDRFSGSKSGTSASLAISGLRSEDEADYYCAA |
| | | WDASLSGHVVFGGGTKLTVLGGGGSGGGGSGGGGSGGGGSQVQLQESGP |
| | | GLVKPSQTLSLTCTVSGGSISSGGYYWSWIRQHPGKGLEWIGYIYYSGSTY |
| | | YNPSLKSRVTISVDTSKNQFSLKLSSVTAADTAVYYCARVWQYYDSSGSF |
| | | DYWGQGTLVTVSSGGGGSGGGGSGGGGSGGGGSDIVMTQSPLSLPVTPG |
| | | EPASISCRSSQSLLHSNGYNYLDWYLQKPGQSPQLLIYLGSNRASGVPDRF |
| | | SGSGSGTDFTLKISRVEAEDVGVYYCMQALQTPFTFGPGTKVDIKIIQADG |
| | | WMCHASKWVTTCDFRWYGPKYITHSIRSFTPSVEQCKESIEQTKQGTWLN |
| | | PGFPPQSCGYATVTDAEAVIVQVTPHHVLVDEYTGEWVDSQFINGKCSNY |
| | | ICPTVHNSTTWHSDYKVKGLCDSNLGMLDSDLHLSSKAQVFEHPHIQDAA |
| | | SQLPDDESLFFGDTGLSKNPIELVEGWFSSWKSSIASFFFIIGLIIGLFLVLRV |
| | | GIHLCIKLKHTKKRQIYTDIEMNRLGK |
| (αRBD:C018/VM) | 73 | SEQ ID Nos. 69 & 64 |
| & | | |
| (αNTD:CV26/D4) | | |
| αRBD:BG4-5/ | 74 | MEFGLSWVFLVALFRGVQSATGVHSQVQLMQSGAEVKKPGASVKVSCK |
| VM | | ASGDSFTDYYVHWVRQAPGQGLEWMGWISPYSGGTNYAPKFRGRVTMT |
| | | RDTSITTAHMELGRLRSDDTAVYFCARSVGYDAFDVWGPGTMVTVSSAS |
| | | TGGGGSGGGGSGGGGSGGGGSTGSWAQSVLTQPPSASGTPGQRVTVSCS |
| | | GSSSNILNNYVYWYQQLPGTAPKLLIYRNNQRPSGVPDRFSGSKSGTSASL |
| | | AISGLRSEDEADYYCAAWDDSLFVVFGGGTKLTVLGQPKAAPSVTLFPPS |
| | | RGMLDSDLHLSSKAQVFEHPHIQDAASQLPDDESLFFGDTGLSKNPIELVE |
| | | GWFSSWKSSIASFFFIIGLIIGLFLVLRVGIHLCIKLKHTKKRQIEQKLISEEDL |
| αRBD:BG7-15/ | 75 | MEFGLSWVFLVALFRGVQSATGVHSQVQLVQSGAEVKKPGASVKVSCKA |
| D4 | | SGYTFTSYAISWVRQAPGQGLEWMGWVSAYNGNTNYAQKLQGRVTMTT |
| | | DTSTNTAYMELRSLRSDDTAIYYCAIPYSSVTFDCWGQGTLVTVSSASTGG |
| | | GGSGGGGSGGGGSGGGGSATGVHSEIVLTQSPGTLSLSPGERATLSCRASQ |
| | | SVSGTFLAWYQQKPGQAPRLLISGASSRATGIPDRFSGSGSGTDFTLTISRL |
| | | EPEDFAVYYCQQYGSSRPTFGQGTKLEIKRTIIQADGWMCHASKWVTTCD |
| | | FRWYGPKYITHSIRSFTPSVEQCKESIEQTKQGTWLNPGFPPQSCGYATVTD |
| | | AEAVIVQVTPHHVLVDEYTGEWVDSQFINGKCSNYICPTVHNSTTWHSDY |
| | | KVKGLCDSNLGMLDSDLHLSSKAQVFEHPHIQDAASQLPDDESLFFGDTG |
| | | LSKNPIELVEGWFSSWKSSIASFFFIIGLIIGLFLVLRVGIHLCIKLKHTKKRQI |
| | | YTDIEMNRLGK |
| αRBD:BG10-19/ | 76 | MEFGLSWVFLVALFRGVQSATGVHSEVQLVQSGAEVKKPGESLKISCKGS |
| D4 | | GYSFTSYWIGWVRQMPGKGLEWMGVIYPGDSDTRYSPSFQGQVTISADKS |
| | | ISTAYLQWSSLKASDTAMYYCARTQWGYNYGSHFFYMDVWGKGTTVTV |
| | | SSASTGGGGSGGGGSGGGGSGGGGSATGSWAQSVLTQPPSASGTPGQRVT |
| | | ISCSGSSSNIGSDHVYWYQQLPGTAPKLFIYRNNQRPSGVPDRFSGSKSGTS |
| | | ASLAISGLRSEDEADYYCAAWDASLSGYVFGTGTKVTVLGQPKANPTVTL |
| | | FPPSIIQADGWMCHASKWVTTCDFRWYGPKYITHSIRSFTPSVEQCKESIEQ |
| | | TKQGTWLNPGFPPQSCGYATVIDAEAVIVQVTPHHVLVDEYTGEWVDSQ |
| | | FINGKCSNYICPTVHNSTTWHSDYKVKGLCDSNLGMLDSDLHLSSKAQVF |
| | | EHPHIQDAASQLPDDESLFFGDTGLSKNPIELVEGWFSSWKSSIASFFFIIGLI |
| | | IGLFLVLRVGIHLCIKLKHTKKRQIYTDIEMNRLGK |
| (αRBD:BG10- | 77 | SEQ ID Nos. 76 & 62 |
| 19/D4) & | | |
| (αNTD:CV21/VM) | | |
| αNTD:CV26/VM | 78 | MEFGLSWVFLVALFRGVQSQVQLVQSGAEVKKPGASVKVSCKVSGYTLT |
| | | ELSIHWVRQAPGKGLEWMGGFDPEDGETVYAQKFQGRVTMTEDTSSDTA |
| | | YMELSSLRSEDTAVYYCATSFPIRGDPSYYYYYYGMDVWGQGTTVTVSS |
| | | GGGGSGGGGSGGGGSGGGGSDIQMTQSPSSLSASVGDRVTITCRASQGITN |
| | | YLAWFQQKPGKAPKSLIYAVSSLQSGVPSKFSGSGSGTDFTLTISSLQPEDF |
| | | ATYYCQQYNSYPWTFGQGTKVEIKSRGMLDSDLHLSSKAQVFEHPHIQDA |
| | | ASQLPDDESLFFGDTGLSKNPIELVEGWFSSWKSSIASFFFIIGLIIGLFLVLR |
| | | VGIHLCIKLKHTKKRQIYTDIEMNRLGK |
| (αRBD:BG10- | 79 | SEQ ID Nos. 76 & 78 |
| 19/D4) & | | |
| (αNTD:CV26/VM) | | |
| (αRBD:BG10- | 80 | SEQ ID Nos. 76 & 74 |
| 19/D4) & | | |
| (αRBD:BG4-5/VM) | | |

### Example 17: Exemplary Ab-Antiviruses

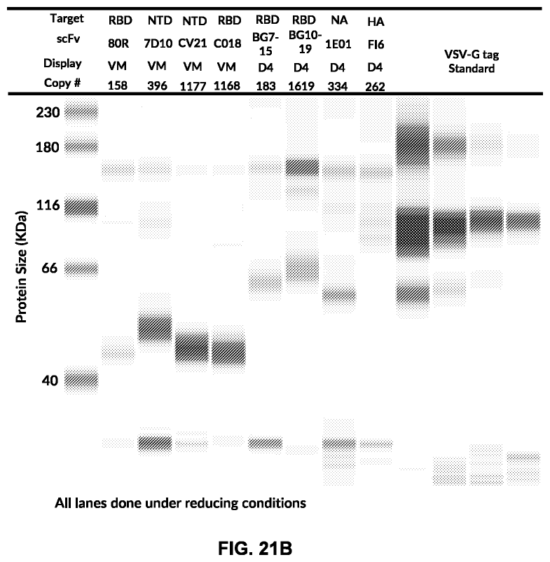

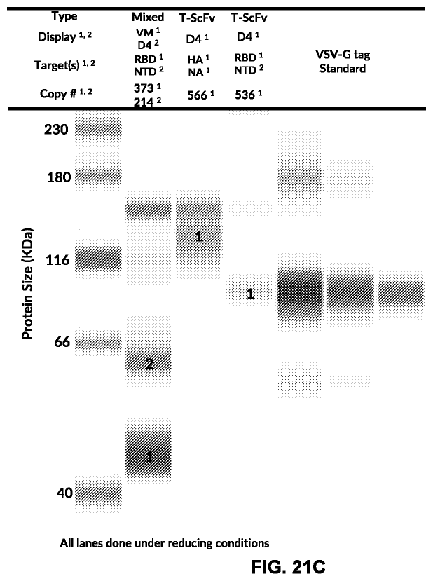

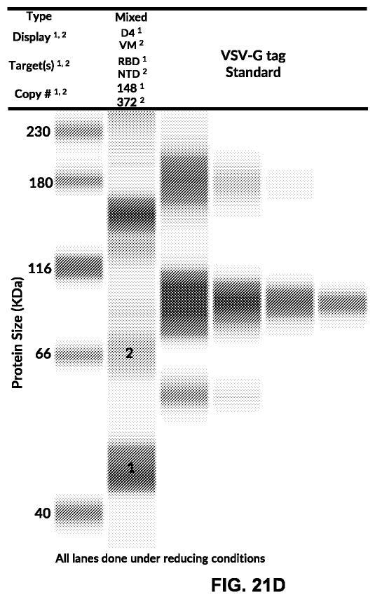

This example illustrates additional Ab-antiviruses produced according to methods disclosed herein, for example, such as methods disclosed in examples 1-16. As illustrated in FIGS. 21A-21D, Ab-antiviruses were produced using a transfer vector and therefore contained viral genetic materials. Expression of scFv fusion constructs on multivalent antibody particles was confirmed via western blot analysis of purified particles. Copy numbers and oligomeric configurations of scFv fusion proteins on Ab-antiviruses were determined via quantitative western blot and PAGE analysis, respectively. Ab-antiviruses pseudotyped with αRBD:H4/VM vector displayed scFv peptides in monomeric form, whereas Ab-antiviruses pseudotyped with αRBD:H4/D4 vector displayed scFv peptides in trimers or higher degree oligomers, as indicated by PAGE/Western blot analyses (FIG. 21A). Further quantitative Western-blot analyses revealed that αRBD:H4/VM was displayed at 118 copies/particle in the monomeric form, and αRBD:H4/D4 scFv peptides was displayed at 8 copies/particle in the trimeric form (FIG. 21A).

Ab-antiviruses pseudotyped with αRBD:80R/VM, αNTD:7D10/VM, αNTD:CV21/VM, or αRBD:C018/VM vectors displayed scFv peptides in monomeric form, whereas Ab-antiviruses pseudotyped with αRBD:BG7-15/D4, αRBD:BG10-19/D4, αNA:1E01/D4, or αHA:FI6/D4 vectors displayed scFv peptides in trimers or higher degree oligomers (FIG. 21B). Further quantitative Western-blot analyses revealed that αRBD:80R/VM, αNTD:7D10/VM, αNTD:CV21/VM, and αRBD:C018/VM scFv peptides were displayed at 158, 396, 1177, or 1168, copies/particle, respectively, in the monomeric form, while αRBD:BG7-15/D4, αRBD:BG10-19/D4, αNA:1E01/D4, or αHA:FI6/D4 scFv peptides were displayed at 183, 1619, 334, and 262, copies/particle, respectively, in the trimeric form (FIG. 21B).

Furthermore, biAb-antiviruses pseudotyped with mixed (αRBD/VM)(αNTD/D4) or (αRBD/D4)(αNTD/VM) scFv antibody vectors displayed mixed scFv peptides, while their αRBD/VM, αNTD/D4, αRBD/D4, and αNTD/VM scFv peptides were displayed at 373, 214, 148 and 372, copies/particle, respectively (FIG. 21C and FIG. 21D). BiAb-antiviruses pseudotyped with tandem (αHA-αNA/D4) or (αRBD-αNTD/D4) scFV antibody vectors displayed tandem scFv peptides in trimers or higher degree oligomers, while their αHA-αNA/D4 and αRBD-αNTD/D4 tandem scFv peptides were displayed at 566 and 536 copies/particle in the trimeric form, respectively (FIG. 21C).

### Example 18: In Vivo Live Virus Neutralization Efficacy of Ab-Antiviruses in hACE2 Mice

This example illustrates a method for evaluation of in vivo live virus neutralization efficacy of Ab-Antiviruses in hACE2 mice. 6 AC70-human ACE2 transgenic mice, all male, 8-10 weeks old were used in each cohort. Animals were weighed prior to the start of the study. Animals were challenged with 55 PFU of authentic SARS COV-2 (strain ITA/INMI1/2020) through intranasal (IN) administration of 50 μL of viral inoculum per nostril. Mice were treated with 5 doses of Ab-Antiviruses (1×10¹¹ particles per dose) via IN delivery, beginning 4 hours post-infection and twice a day on day 1 and day 2 post-infection. Animals were monitored twice daily for signs of COVID-19 disease phenotype (ruffled fur, hunched posture, labored breathing) and survival during the study period. Body weights were measured once daily during the study period, and lung tissue was collected and sampled for viral load assays by PRNT. Tissues were stored at 80° C. for histology and viral load analysis by qPCR and PRNT analysis.

### Example 19: Generation of Monomeric Ab-Antiviruses without Viral Genomes that Express Neutralizing Antibody

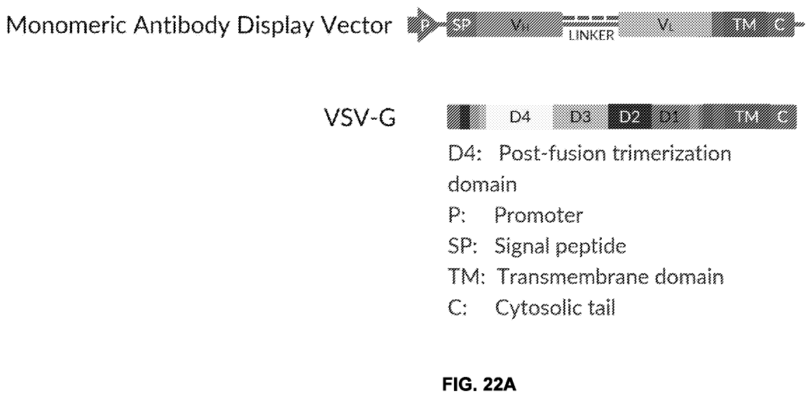

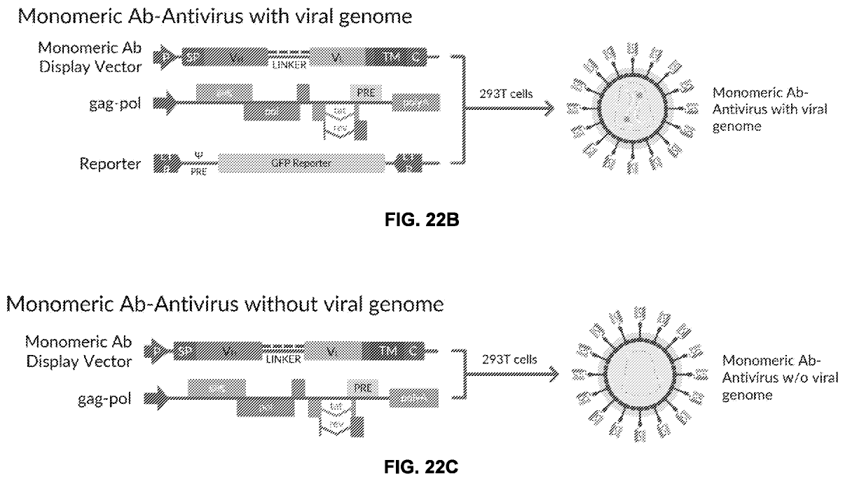

This example illustrates the production and characterization of monomeric Ab-Antiviruses that do not contain viral genetic material and express a neutralizing antibody. FIG. 22A shows the design and composition of monomeric antibody display vector in which monomeric scFv fusion display constructs were designed by fusing the desired scFv sequence to the transmembrane and cytosolic tail regions of VSV-G. FIGS. 22B and 22C show schematic production of monomeric Ab-Antiviruses with viral genomes (FIG. 22B) or without viral genomes (FIG. 22C). Monomeric Ab-Antiviruses in the form of VLPs containing viral RNA genomes were produced via co-transfection of S293 cells with monomeric antibody display constructs along with a lentiviral packaging construct expressing essential packaging components, such as Gag-Pol and Rev proteins, and a viral genome transfer vector encoding a GFP/luciferase reporter (FIG. 22B) according to the protocols described in examples 1-7 above. Also, monomeric Ab-Antiviruses in the form of VLPs without viral RNA genomes were produced by co-transfecting S293 cells with antibody-display vectors along with a lentiviral packaging construct but without the viral genome transfer vector (FIG. 22C) according to the protocols described in examples 1-7 above.

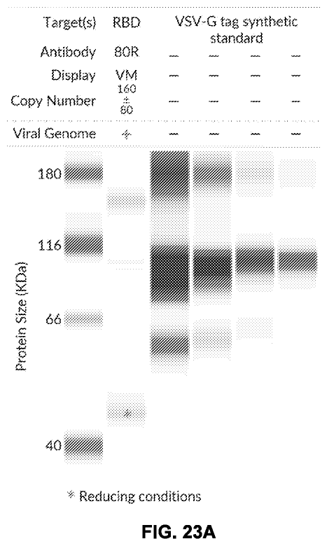

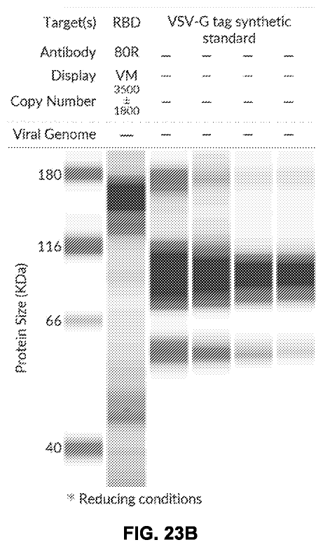

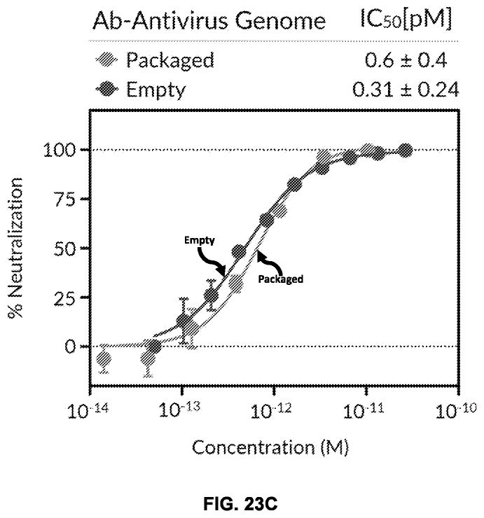

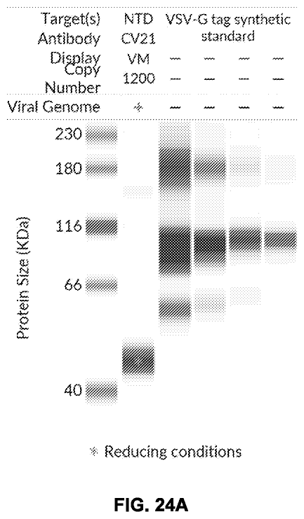

To investigate whether multivalent display on Ab-Antiviruses enhances neutralizing antibodies, monomeric Ab-Antiviruses displaying a neutralizing antibody targeting the spike receptor binding domain (RBD) of SARS COV-1 were generated, namely αCoV1:80R/VM (FIGS. 23A-23C). αCoV1:80R/VM Ab-Antiviruses with (FIG. 23A) and without viral genetic material (FIG. 23B) were produced and characterized. The concentrations of purified Ab-Antiviruses were determined via P24 ELISA assay. Quantitative western blot analysis indicated that αCoV1:80R/VM Ab-Antiviruses with viral genomes, termed packaged Ab-Antiviruses, displayed 160±80 copies of monomeric scFv fusion peptide per particle (FIG. 23A), while the same Ab-Antiviruses produced without viral genomes, termed empty Ab-Antiviruses, displayed 3500±1800 copies of scFv (FIG. 23B), through quantitative western blot analysis. The removal of a viral genome transfer vector did not impact the antibody display efficiency of αCoV1:80R/VM Ab-Antiviruses.

To confirm whether both the packaged and empty versions of αCoV1:80R/VM Ab-Antiviruses displayed functional scFv that retained binding specificity, a pseudovirus neutralization assay against SARS COV-1 was carried out using H1573/ACE2 cells for mock infection according to the protocols described in examples 1-7 above. The results showed that αCoV1:80R/VM Ab-Antiviruses, with and without viral genomes, neutralized CoV-1 pseudovirus at IC50s of 0.6±0.4 and 0.31±0.24, respectively (FIG. 23C). Both versions of αCoV1:80R/VM Ab-Antiviruses were more than 10,000-fold more potent than soluble 80R scFv, which had an IC50 of 7.43 nM. These results demonstrated that monomeric Ab-Antiviruses with and without genetic material can effectively display functional, neutralizing antibodies at high copy numbers. In addition, multivalent antibody display on Ab-Antiviruses enhanced neutralizing antibodies by orders of magnitude.

### Example 20: Generation of Monomeric Ab-Antiviruses without Viral Genome that Express Non-Neutralizing Antibody

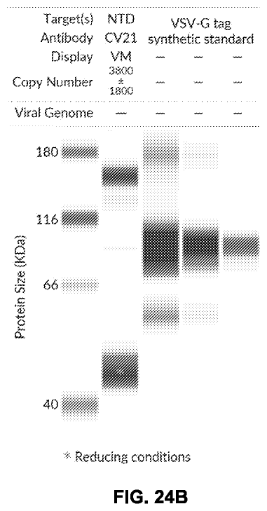

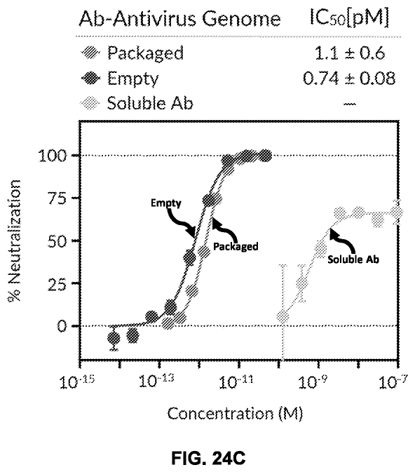

This example illustrates the production and characterization of monomeric Ab-Antiviruses that do not contain viral genetic material and express a non-neutralizing antibody. Ab-Antiviruses capturing virions using multivalent interactions may neutralize viruses via a mechanism independent of the nature of displayed antibody. Monomeric Ab-Antiviruses αNTD:CV21/VM displaying a non-neutralizing antibody, CV21, that binds to the N-terminal domain (NTD) of the SARS COV-2 spike protein, were generated with and without viral genome transfer vectors according to the protocols described in examples 1-7 above. The concentrations of purified Ab-Antivirus were determined via P24 ELISA. It was found that αNTD:CV21/VM Ab-Antiviruses packaging viral genomes displayed 1200±1600 copies of scFv fusion peptide per particle (FIG. 24A), while those without viral genomes displayed 3800±1800 copies per particle (FIG. 24B), through quantitative western blot analysis. These results demonstrated that the removal of a viral genome transfer vector does not interfere with efficient, monomeric antibody display on αNTD:CV21/VM Ab-Antiviruses.

To confirm that both the packaged and empty versions of αNTD:CV21/VM Ab-Antivirus displayed functional scFv that retained binding specificity, both versions were tested in a pseudovirus neutralization assay against SARS COV-2, using H1573/ACE2 cells for mock infection. Packaged and empty versions of αNTD:CV21/VM Ab-Antivirus neutralize CoV-2 pseudovirus at IC50s of 1.1±0.6 and 0.74±0.08 pM, respectively (FIG. 24C). Soluble CV21 did not exhibit significant neutralizing activity against SARS COV-2 pseudovirus (FIG. 24C), demonstrating that potently neutralizing Ab-Antiviruses can be derived from non-neutralizing, binding antibodies. These results indicated that monomeric Ab-Antiviruses with and without genetic material can effectively display functional, non-neutralizing antibodies at high copy numbers. The absence of viral genome transfer vector did not hinder the display efficiency or neutralizing potency of Ab-Antiviruses. In addition, multivalent antibody display on monomeric Ab-Antiviruses converted non-neutralizing antibodies into potently neutralizing molecules.

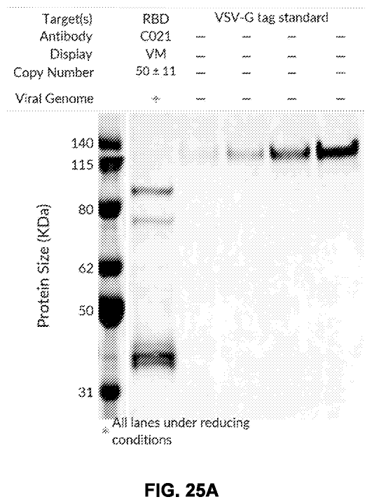

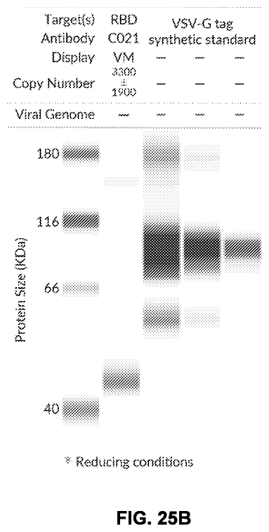

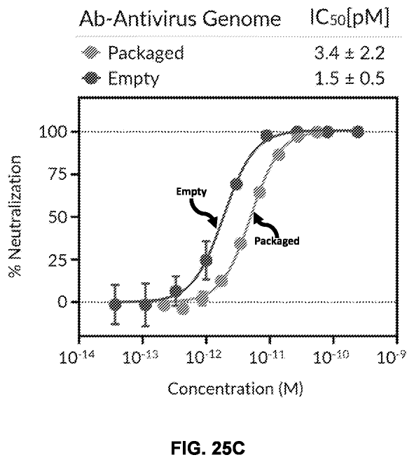

Next, it was examined whether neutralizing Ab-Antiviruses can be designed from antibodies targeting distinct domains on the target virus spike protein. Monomeric Ab-Antiviruses αRBD:C021/VM displaying scFv derived from the non-neutralizing antibody C021, which binds to the SARS COV-2 spike RBD, were generated with and without viral genome transfer vectors. The concentrations of purified Ab-Antiviruses were determined via P24 ELISA. It was found that αRBD:C021/VM Ab-Antiviruses packaging viral genomes displayed 47±11 copies of scFv fusion peptide per particle (FIG. 25A), while the empty versions displayed 3300±1900 copies per particle (FIG. 25B), through quantitative western blot analysis. These results demonstrated that the removal of viral genome transfer vector did not interfere with efficient, monomeric antibody display on αRBD:C021/VM Ab-Antiviruses.

To determine whether these RBD-specific Ab-Antiviruses displayed functional antibodies retaining binding specificity, both packaged and empty αRBD:C021/VM Ab-Antiviruses were analyzed in a pseudovirus neutralization assay against SARS COV-2, using H1573/ACE2 cells for mock infection according to the protocols described in examples 1-7 above. Packaged and empty versions of αRBD:C021/VM Ab-Antivirus neutralized CoV-2 pseudovirus at IC50s of 3.4±2.2 and 1.5±0.5 pM, respectively (FIG. 25C). Soluble C021 did not demonstrate significant neutralizing activity against SARS COV-2 pseudovirus, again demonstrating that potently neutralizing Ab-Antiviruses can be derived from non-neutralizing binding antibodies. These results demonstrated that monomeric Ab-Antiviruses with and without genetic material can effectively display functional, non-neutralizing antibodies at high copy numbers. The absence of viral genome transfer vector did not hinder the display efficiency or neutralizing potency of Ab-Antiviruses. Furthermore, as illustrated, Ab-Antiviruses can take advantage of binding antibodies targeting diverse spike regions, circumventing the target domain restrictions of neutralizing antibodies, which rely on blocking the spike RBD.

### Example 21: Generation of Trimeric Ab-Antiviruses without Viral Genome

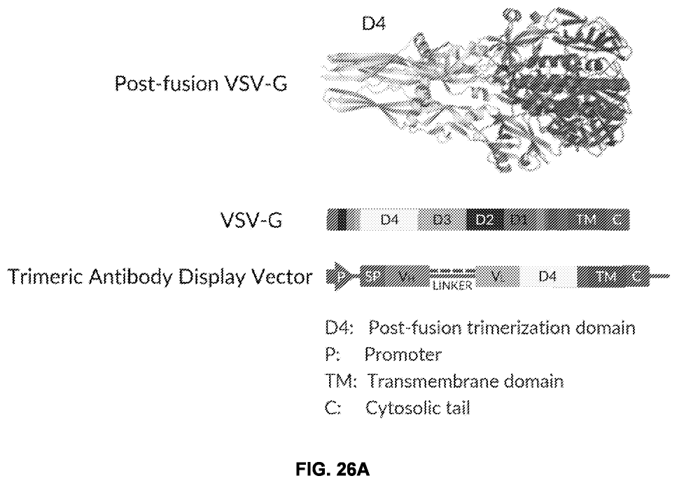

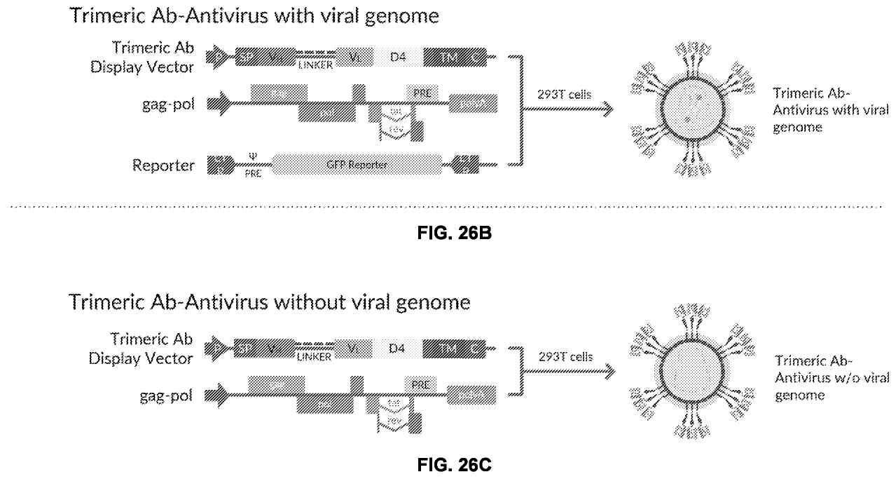

This example illustrates the production and characterization of trimeric Ab-Antiviruses that do not contain viral genetic material. Trimeric scFv fusion display constructs were designed by fusing the desired scFv sequence to the D4 post-fusion trimerization domain of VSV-G protein, followed by the transmembrane and cytosolic tail regions of VSV-G (FIG. 26A). Trimeric Ab-Antiviruses in the form of VLPs containing viral RNA genomes were produced according to the protocols described in examples 1-7 above via co-transfection of S293 cells with trimeric antibody display constructs along with a lentiviral packaging construct expressing essential packaging components, such as Gag-Pol and Rev proteins, and a viral genome transfer vector encoding a GFP/luciferase reporter (FIG. 26B). Alternatively, trimeric Ab-Antiviruses in the form of VLPs without viral genomes were produced according to the protocols described in examples 1-7 above by co-transfecting S293 cells with antibody-display vectors along with a lentiviral packaging construct, without a viral genome transfer vector (FIG. 26C).

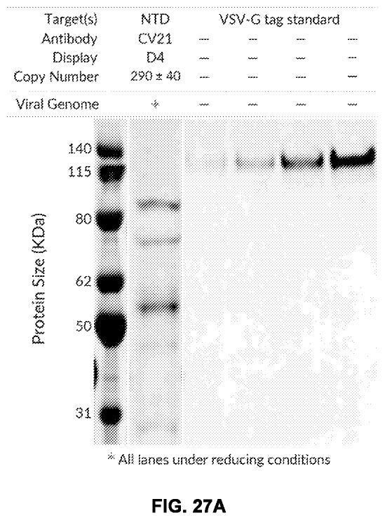

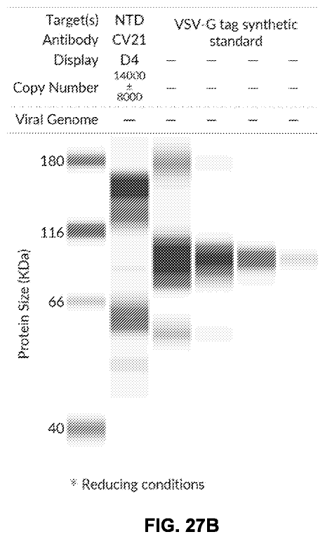

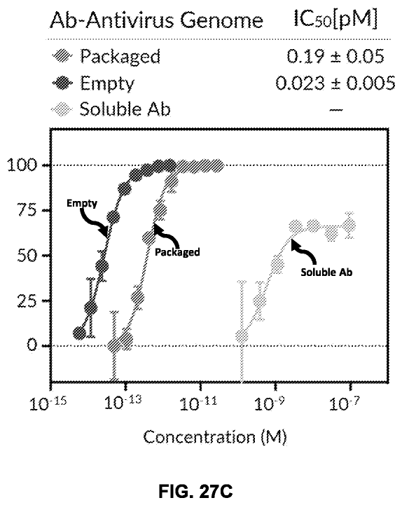

Next, whether trimeric Ab-Antiviruses that mimic spike display oligomerization demonstrated increased antiviral potency compared to their monomeric counterparts were examined. Trimeric Ab-Antiviruses displaying scFv fusion peptides derived from the non-neutralizing antibody CV21, which binds to the SARS COV-2 spike NTD, termed αNTD:CV21/D4 Ab-Antiviruses, were generated. Packaged and empty αNTD:CV21/D4 Ab-Antiviruses were produced and the concentration of each purified Ab-Antivirus was determined via P24 ELISA. It was found that packaged αNTD:CV21/D4 Ab-Antiviruses displayed 290±40 copies of scFv fusion peptide per particle (FIG. 27A), while the empty αNTD:CV21/D4 Ab-Antiviruses displayed 14000±8000 copies per particle (FIG. 27B), through quantitative western blot analysis. These results demonstrated that the removal of viral genome transfer vector did not interfere with efficient, trimeric antibody display on αNTD:CV21/D4 Ab-Antiviruses.

To determine whether these trimeric Ab-Antiviruses displayed functional antibodies retaining binding specificity, both packaged and empty αNTD:CV21/D4 Ab-Antiviruses were analyzed in a pseudovirus neutralization assay against SARS COV-2, using H1573/ACE2 cells for mock infection according to the protocols described in examples 1-7 above. Packaged and empty versions of αNTD:CV21/D4 Ab-Antivirus neutralized CoV-2 pseudovirus at IC50s of 0.19±0.05 and 0.023±0.005 pM, respectively (FIG. 27C). Soluble CV21 did not demonstrate significant neutralizing activity against SARS COV-2 pseudovirus, again demonstrating that potently neutralizing Ab-Antiviruses can be derived from non-neutralizing, binding antibodies (FIG. 27C). Both the packaged and empty versions of trimeric αNTD:CV21/D4 were more than 10-fold more potent than their monomeric counterparts as determined by PNA IC50 (FIG. 24C). This increase in potency can be attributed to increased valency as well as trimeric antibody display mirroring SARS CoV-2 spike display, allowing for increased local multivalent binding between virus and antivirus. These results demonstrated that trimeric Ab-Antiviruses with and without genetic material can effectively display functional, non-neutralizing antibodies at high copy numbers. The absence of viral genome transfer vector did not hinder the display efficiency or neutralizing potency of Ab-Antiviruses.

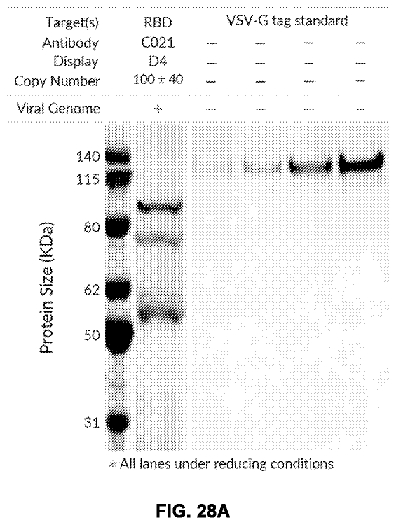

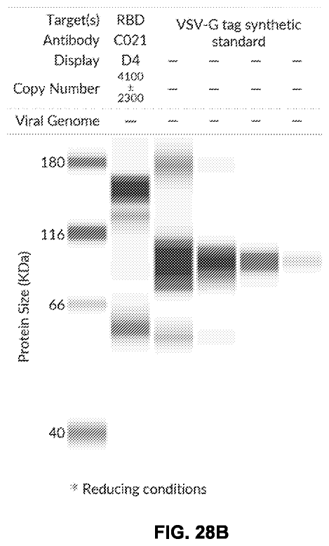

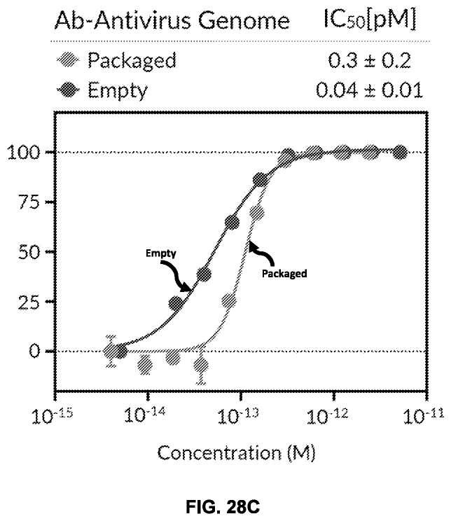

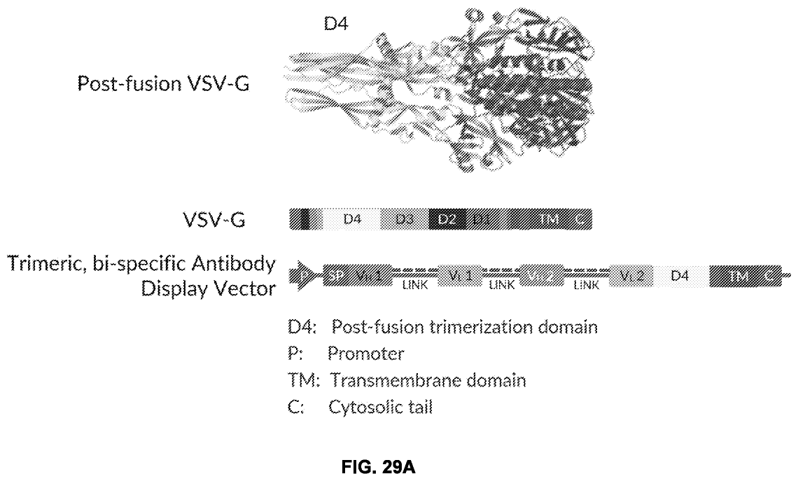

Next, it was examined whether trimeric Ab-Antiviruses can similarly be generated displaying antibodies with various spike target domains. To this end, trimeric Ab-Antiviruses displaying scFv fusion peptides derived from the non-neutralizing antibody C021, which binds to the SARS COV-2 spike NTD, termed αRBD:C021/D4 Ab-Antiviruses, were generated. Packaged αRBD:C021/D4 containing viral genomes and empty αRBD:C021/D4 Ab-Antiviruses without genomes, were produced and the concentration of each purified Ab-Antivirus was determined via P24 ELISA. It was found that packaged αRBD:C021/D4 Ab-Antiviruses displayed 100±40 copies of scFv fusion peptide per particle (FIG. 28A), while the empty version displays 4100±2300 copies per particle (FIG. 28B), through quantitative western blot analysis. These results demonstrated that the removal of viral genome transfer vector did not interfere with efficient, trimeric antibody display on αRBD:C021/D4 Ab-Antiviruses.

To determine whether these trimeric Ab-Antiviruses displayed functional antibodies retaining binding specificity, both packaged and empty αRBD:C021/D4 Ab-Antiviruses were analyzed in a pseudovirus neutralization assay against SARS COV-2, using H1573/ACE2 cells for mock infection according to the protocols described in examples 1-7 above. Packaged and empty versions of αRBD:C021/D4 Ab-Antivirus neutralized CoV-2 pseudovirus at IC50s of 0.3±0.2 and 0.04±0.01 pM, respectively (FIG. 28C). Both the packaged and empty versions of trimeric αRBD:C021/D4 antiviruses were more than 10-fold more potent than their monomeric counterparts as determined by PNA IC50 (FIG. 25C). This increase in potency can be attributed to increased valency as well as trimeric antibody display mirroring SARS COV-2 spike display, allowing for increased local multivalent binding between virus and antivirus. These results demonstrated that trimeric Ab-Antiviruses with and without genetic material can effectively display functional, non-neutralizing antibodies at high copy numbers. The absence of viral genome transfer vector did not hinder the display efficiency or neutralizing potency of Ab-Antiviruses. Furthermore, as demonstrated, Ab-Antiviruses can take advantage of binding antibodies targeting diverse spike regions, circumventing the target domain restrictions of most neutralizing antibodies, which rely on blocking the spike RBD.

### Example 22: Generation of Trimeric Multispecific Ab-Antiviruses without Viral Genome

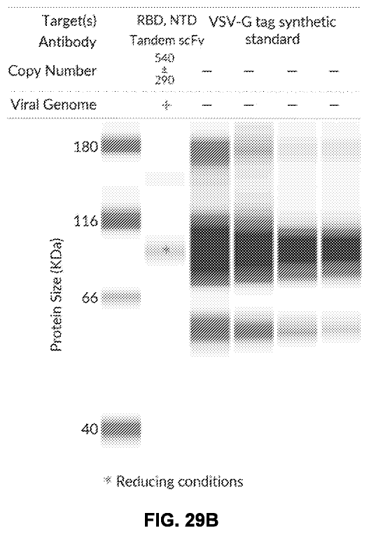

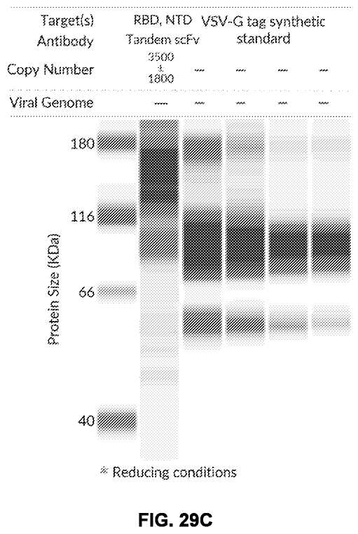

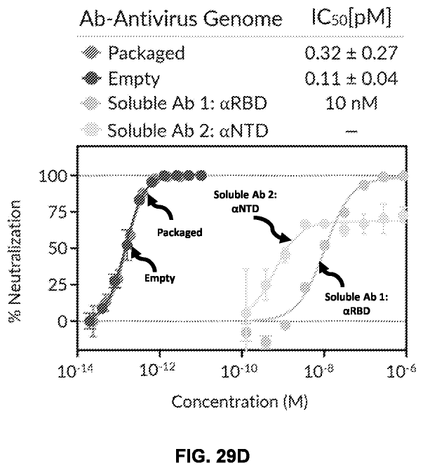

This example illustrates the production and characterization of bi-specific Ab-Antiviruses and whether Ab-Antivirus neutralizing potency is enhanced by multi-specificity. Bi-specific, trimeric antibody display constructs were designed by fusing a bi-specific antibody to the D4 post-fusion trimerization domain of VSV-G, followed by the transmembrane and cytosolic tail domains of VSV-G (FIG. 29A). A bi-specific, tandem scFv was constructed derived from the non-neutralizing antibodies CV21 and C021, which bind to the SARS COV-2 spike NTD and RBD domains, respectively. Using this bi-specific antibody display construct, packaged (αRBD-αNTD)/D4 biAb-Antiviruses containing viral genomes and empty (αRBD-αNTD)/D4 biAb-Antiviruses without viral genomes were generated according to the protocols described in examples 1-7 above, and the concentration of each purified Ab-Antivirus was determined via P24 ELISA. It was found that packaged (αRBD-αNTD)/D4 displayed 540±290 copies of scFv fusion peptide per particle (FIG. 29B), while the empty version displayed 3500±1800 copies per particle (FIG. 29C), through quantitative western blot analysis. These results demonstrated that the removal of viral genome transfer vector did not interfere with efficient, trimeric bi-specific antibody display on (αRBD-αNTD)/D4 biAb-Antiviruses.

To determine whether these trimeric biAb-Antiviruses displayed functional bi-specific antibodies retaining binding specificity, both packaged and empty (αRBD-αNTD)/D4 biAb-Antiviruses were analyzed in a pseudovirus neutralization assay against SARS COV-2, using H1573/ACE2 cells for mock infection according to the protocols described in examples 1-7 above (FIG. 29D). Packaged and empty versions of (αRBD-αNTD)/D4 biAb-Antiviruses neutralized CoV-2 pseudovirus at IC50s of 0.32±0.27 and 0.11±0.04 pM, respectively (FIG. 29D). As neither of the soluble antibodies from which the bi-specific display construct was derived display significant neutralizing activity against SARS COV-2, these results again demonstrated that potently neutralizing, bi-specific Ab-Antiviruses can be derived from non-neutralizing, binding antibodies. BiAb-Antiviruses are likely to exhibit increased viral escape mutation resistance, as a result of multi-epitope binding redundance, further enhancing antivirus multivalent binding. Collectively, the results demonstrated that Ab-Antiviruses displaying monomeric or trimeric antibodies with varying spike targets can be generated with and without genetic material. These Ab-Antiviruses displayed high copy numbers of spike-specific antibodies, and can convert non-neutralizing antibodies into potently neutralizing molecules. Furthermore, Ab-Antiviruses can be genetically programmed to display antibodies in various formats, including bi-specific antibodies. The absence of viral genome transfer vector did not hinder the display efficiency or neutralizing potency of Ab-Antiviruses. A summary of characterization of the display copy number and pseudovirus neutralization IC₅₀ of Ab-Antiviruses with and without viral genomes is listed in Table 11 below.

TABLE 11
Summary of Ab-Antiviruses with and without viral genomes

| | | | | Pseudovirus | Pseudovirus |
|---|---|---|---|---|---|
| | | Copy Number | Copy Number | IC₅₀ (M) | IC₅₀ (M) |
| | | (with viral | (without viral | (with viral | (without viral |
| Ab-Antivirus | Target Virus | genome) | genome) | genome) | genome) |
| αNTD: CV21/VM | SARS CoV-2 | 1200 ± 600 | 3800 ± 1800 | 1.1 ± 0.6 | 0.74 ± 0.08 |
| αNTD: CV21/D4 | SARS CoV-2 | 290 ± 40 | 14000 ± 8000 | 0.19 ± 0.05 | 0.023 ± 0.005 |
| αNTD: C021/VM | SARS CoV-2 | 47 ± 11 | 3300 ± 1900 | 3.4 ± 2.2 | 1.5 ± 0.5 |
| αRBD: C021/D4 | SARS CoV-2 | 100 ± 40 | 4100 ± 2300 | 0.3 ± 0.2 | 0.04 ± 0.01 |
| αRBD: 80R/VM | SARS CoV-1 | 160 ± 80 | 3500 ± 1800 | 0.6 ± 0.4 | 0.31 ± 0.24 |
| (αRBD-αNTD)/D4 | SARS CoV-2 | 540 ± 290 | 3500 ± 1800 | 0.32 ± 0.27 | 0.11 ± 0.04 |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EMBODIMENTS

The following non-limiting embodiments provide illustrative examples of the invention, but do not limit the scope of the invention.
Embodiment 1. An antivirus comprising a fusion protein that comprises a transmembrane polypeptide and an antibody which binds to a surface protein of a virus wherein the fusion protein is expressed at a valency of at least about 10 copies on a surface of the antivirus, wherein the antibody neutralizes the virus when expressed within the fusion protein on the surface of the antivirus, but does not neutralize the virus when expressed as an isolated antibody.
Embodiment 2. The antivirus of embodiment 1, wherein the fusion protein further comprises an oligomerization domain.
Embodiment 3. The antivirus of embodiment 2, wherein the oligomerization domain is a dimerization domain.
Embodiment 4. The antivirus of embodiment 3, wherein the dimerization domain comprises a leucine zipper dimerization domain.
Embodiment 5. The antivirus of embodiment 2, wherein the oligomerization domain is a trimerization domain.
Embodiment 6. The antivirus of embodiment 5, wherein the trimerization domain comprises a post-fusion oligomerization domain of viral surface protein.
Embodiment 7. The antivirus of embodiment 5, wherein the trimerization domain comprises a D4 post-fusion trimerization domain of VSV-G protein.
Embodiment 8. The antivirus of embodiment 5, wherein the trimerization domain comprises a Dengue E protein post-fusion trimerization domain.
Embodiment 9. The antivirus of embodiment 5, wherein the trimerization domain comprises a foldon trimerization domain.
Embodiment 10. The antivirus of embodiment 2, wherein the oligomerization domain is a tetramerization domain.
Embodiment 11. The antivirus of embodiment 10, wherein the tetramerization domain comprises an influenza neuraminidase stem domain.
Embodiment 12. The antivirus of embodiment 2, wherein the oligomerization domain comprises an amino acid sequence that has at least 95% sequence identity to an amino acid sequence according to SEQ ID NOs: 30-43.
Embodiment 13. The antivirus of any one of embodiments 2-12, wherein when the fusion protein is expressed on the surface of the antivirus, the oligomerization domain is outside of the antivirus.
Embodiment 14. The antivirus of any one of embodiments 2-12, wherein when the fusion protein is expressed on the surface of the antivirus, the oligomerization domain is outside of the antivirus and adjacent to a signal peptide.
Embodiment 15. The antivirus of any one of embodiments 2-12, wherein when the fusion protein is expressed on the surface of the antivirus, the oligomerization domain is inside of the antivirus.
Embodiment 16. The antivirus of any one of embodiments 2-12, wherein when the fusion protein is expressed on the surface of the antivirus, the oligomerization domain is inside of the antivirus and adjacent to the transmembrane polypeptide.
Embodiment 17. The antivirus of any one of embodiments 1-15, wherein the fusion protein comprises a signal peptide.
Embodiment 18. The antivirus of any one of embodiments 1-15, wherein domains of the fusion protein are arranged from the N-terminus to the C-terminus in the following orders:
   (a) signal peptide, antibody which binds to a surface protein of a virus, oligomerization domain, transmembrane polypeptide, and cytosolic domain;
   (b) signal peptide, antibody which binds to a surface protein of a virus, transmembrane polypeptide, oligomerization domain, and cytosolic domain; or
   (c) signal peptide, oligomerization domain, antibody which binds to a surface protein of a virus, transmembrane polypeptide, and cytosolic domain.
Embodiment 19. The antivirus of any one of embodiments 1-18, wherein the antibody comprises a single chain variable fragment (scFv), a single domain antibody, an Fv, a VH domain, a VL domain, a Fab fragment, a monoclonal antibody, F(ab′), F(ab′)2, single chain antibodies, diabodies, or a scFv-Fc.
Embodiment 20. The antivirus of any one of embodiments 1-18, wherein the antibody binds specifically to the surface protein of the virus.
Embodiment 21. The antivirus of any one of embodiments 1-18, wherein the antibody is a multispecific antibody.
Embodiment 22. The antivirus of embodiment 21, wherein the multispecific antibody binds specifically to more than one epitope on the surface protein of the virus.
Embodiment 23. The antivirus of embodiment 21, wherein the multispecific antibody comprises a tandem scFv format.
Embodiment 24. The antivirus of any one of embodiments 1-23, wherein the virus comprises SARS COV-1, CoV-2, influenza, or MERS CoV virus.
Embodiment 25. The antivirus of any one of embodiments 1-24, wherein the antibody comprises an amino acid sequence from at least one complementarity determining region of 0304-4A10, 2M-14E5, 9A1, COV2-2021, COV2-2026, COV2-2146, 2M-10B11, C021, C018, and BG4-5.
Embodiment 26. The antivirus of any one of embodiments 1-24, wherein the antibody comprises an amino acid sequence according to any one of SEQ ID NOs: 6-14, 20-28, and 81-82.
Embodiment 27. The antivirus of any one of embodiments 1-26, wherein the transmembrane polypeptide anchors the fusion protein to a bilayer of the antivirus.
Embodiment 28. The antivirus of any one of embodiments 1-26, wherein the transmembrane polypeptide comprises the transmembrane domain of a Vesicular Stomatitis virus glycoprotein (VSV-G).
Embodiment 29. The antivirus of any one of embodiments 1-26, wherein the transmembrane polypeptide comprises the transmembrane domain of influenza Hemagglutinin (HA).
Embodiment 30. The antivirus of any one of embodiments 1-26, wherein the transmembrane polypeptide comprises the transmembrane domain of HIV surface glycoprotein GP120 or GP41.
Embodiment 31. The antivirus of any one of embodiments 1-26, wherein the transmembrane polypeptide comprises the transmembrane domain of measles virus surface glycoprotein hamagglutinin (H) protein.
Embodiment 32. The antivirus of any one of embodiment 1-26, wherein the transmembrane polypeptide comprises the transmembrane domain of influenza Neuraminidase (NA).
Embodiment 33. The antivirus of any one of embodiments 1-26, wherein the transmembrane polypeptide comprises an amino acid sequence at least about 90% identical to that set forth in SEQ ID NO: 29.
Embodiment 34. The antivirus of any one of embodiments 1-33, wherein the fusion protein is expressed at a valency of about 10 copies on a surface of the antivirus.
Embodiment 35. The antivirus of any one of embodiments 1-33, wherein the fusion protein is expressed at a valency of about 10 to 15 copies on a surface of the antivirus.
Embodiment 36. The antivirus of any one of embodiments 1-33, wherein the fusion protein is expressed at a valency of at least about 25 copies on a surface of the antivirus.
Embodiment 37. The antivirus of any one of embodiments 1-33, wherein the fusion protein is expressed at a valency of at least about 50 copies on a surface of the antivirus.
Embodiment 38. The antivirus of any one of embodiments 1-33, wherein the fusion protein is expressed at a valency of at least about 100 copies on a surface of the antivirus.
Embodiment 39. The antivirus of any one of embodiments 1-33, wherein the fusion protein is expressed at a valency of at least about 200 copies on a surface of the antivirus.
Embodiment 40. The antivirus of any one of embodiments 1-33, wherein the fusion protein is expressed at a valency of at least about 400 copies on a surface of the antivirus.
Embodiment 41. The antivirus of any one of embodiments 1-33, wherein the fusion protein is expressed at a valency of at least about 600 copies on a surface of the antivirus.
Embodiment 42. The antivirus of any one of embodiments 1-33, wherein the fusion protein is expressed at a valency of at least about 1000 copies on a surface of the antivirus.
Embodiment 43. The antivirus of any one of embodiments 1-42, wherein the antivirus is an enveloped particle.
Embodiment 44. The antivirus of any one of embodiments 1-42, wherein the antivirus is not a lentiviral particle.
Embodiment 45. The antivirus of any one of embodiments 1-42, wherein the antivirus does not comprise viral genetic material.
Embodiment 46. The antivirus of any one of embodiments 1-42, wherein the antivirus comprises a lipid bilayer.
Embodiment 47. The antivirus of any one of embodiments 1-42, wherein the antivirus is a virus.
Embodiment 48. The antivirus of any one of embodiments 1-42, wherein the antivirus is a replication incompetent virus.
Embodiment 49. The antivirus of any one of embodiments 1-42, wherein the antivirus is a replication competent virus.
Embodiment 50. The antivirus of any one of embodiments 1-42, wherein the antivirus is a viral-like particle.
Embodiment 51. The antivirus of any one of embodiments 1-42, wherein the antivirus is an extracellular vesicle.
Embodiment 52. The antivirus of any one of embodiments 1-42, wherein the antivirus is an exosome.
Embodiment 53. The antivirus of any one of embodiments 1-52, wherein the antivirus further comprises a second fusion protein that comprises transmembrane polypeptide and a second antibody which binds to a surface protein of the virus, wherein the second antibody comprises a CDR sequence that has less than 100% sequence identity to an equivalent CDR sequence of the antibody.
Embodiment 54. The antivirus of embodiment 53, wherein the fusion protein and the second fusion protein comprise the same transmembrane polypeptide.
Embodiment 55. The antivirus of embodiment 53, wherein the fusion protein and the second fusion protein comprise different transmembrane polypeptides.
Embodiment 56. The antivirus of embodiment 46, wherein the second antibody binds to the same surface protein as the antibody.
Embodiment 57. The antivirus of embodiment 53, wherein the second antibody binds to a different surface protein as the antibody.
Embodiment 58. The antivirus of embodiment 53, wherein the second antibody is a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc.
Embodiment 59. The antivirus of embodiment 53, wherein the second antibody binds specifically to the surface protein of the virus.
Embodiment 60. The antivirus of embodiment 53, wherein the second antibody is a multispecific antibody.
Embodiment 61. The antivirus of embodiment 53, wherein the second multispecific antibody binds specifically to more than one epitope on the surface protein of the virus.
Embodiment 62. The antivirus of embodiment 53, wherein the second antibody comprises an amino acid sequence from at least one complementarity determining region of 80R, H4, 7D10, 1E01, F16, 0304-4A10, 2M-14E5, 9A1, COV2-2021, COV2-2026, COV2-2146, 2M-10B11, C021, C018, BG4-5, BG7-15, and BG10-19.
Embodiment 63. The antivirus of embodiment 53, wherein the second antibody comprises an amino acid sequence according to any one of SEQ ID NOs: 1-28 and 81-86.
Embodiment 64. The antivirus of embodiment 53, wherein the second antibody neutralizes the virus when expressed within the fusion protein but does not neutralize the virus when expressed as an isolated antibody.
Embodiment 65. The antivirus of embodiment 53, wherein the second antibody neutralizes the virus when expressed within the fusion protein and neutralizes the virus when expressed as an isolated antibody.
Embodiment 66. The antivirus of embodiment 53, wherein the second fusion protein further comprises an oligomerization domain.
Embodiment 67. The antivirus of embodiment 66, wherein the oligomerization domain is a dimerization domain.
Embodiment 68. The antivirus of embodiment 66, wherein the oligomerization domain is a trimerization domain.
Embodiment 69. The antivirus of embodiment 66, wherein the oligomerization domain comprises the D4 trimerization domain of VSV-G protein.
Embodiment 70. The antivirus of any one of embodiments 1-69, wherein the antibody comprises the scFv and an amino acid sequence from at least one complementarity determining region of COV2-2021.
Embodiment 71. The antivirus of any one of embodiments 1-69, wherein the antibody comprises the scFv and an amino acid sequence from at least one complementarity determining region of C021.
Embodiment 72. The antivirus of any one of embodiments 1-69, wherein the antibody comprises the scFv and an amino acid sequence from at least one complementarity determining region of C018.
Embodiment 73. The antivirus of any one of embodiments 1-69, wherein the antibody comprises the scFv and an amino acid sequence from at least one complementarity determining region of H4 and the oligomerization domain comprises the D4 trimerization domain of VSV-G protein.
Embodiment 74. The antivirus of any one of embodiments 1-69, wherein the antibody comprises the scFv and an amino acid sequence from at least one complementarity determining region of 0304-4A10, and the oligomerization domain comprises the D4 trimerization domain of VSV-G protein.
Embodiment 75. The antivirus of any one of embodiments 1-69, wherein the antibody comprises the scFv and an amino acid sequence from at least one complementarity determining region of 2M-14E5, and the oligomerization domain comprises the D4 trimerization domain of VSV-G protein.
Embodiment 76. The antivirus of any one of embodiments 1-69, wherein the antibody comprises the scFv and an amino acid sequence from at least one complementarity determining region of 9A1, and the oligomerization domain comprises the D4 trimerization domain of VSV-G protein.
Embodiment 77. The antivirus of any one of embodiments 1-69, wherein the antibody comprises the scFv and an amino acid sequence from at least one complementarity determining region of COV2-2021, and the oligomerization domain comprises the D4 trimerization domain of VSV-G protein.
Embodiment 78. The antivirus of any one of embodiments 1-69, wherein the antibody comprises the scFv and an amino acid sequence from at least one complementarity determining region of COV2-2026, and the oligomerization domain comprises the D4 trimerization domain of VSV-G protein.
Embodiment 79. The antivirus of any one of embodiments 1-69, wherein the antibody comprises the scFv and an amino acid sequence from at least one complementarity determining region of COV2-2146, and the oligomerization domain comprises the D4 trimerization domain of VSV-G protein.
Embodiment 80. The antivirus of any one of embodiments 1-69, wherein the antibody comprises the scFv and an amino acid sequence from at least one complementarity determining region of 2M-10B11, and the oligomerization domain comprises the D4 trimerization domain of VSV-G protein.
Embodiment 81. The antivirus of any one of embodiments 1-69, wherein the antibody comprises the scFv and an amino acid sequence from at least one complementarity determining region of C021, and the oligomerization domain comprises the D4 trimerization domain of VSV-G protein.
Embodiment 82. The antivirus of any one of embodiments 1-69, wherein the antibody comprises the scFv and an amino acid sequence from at least one complementarity determining region of C018, and the oligomerization domain comprises the D4 trimerization domain of VSV-G protein.
Embodiment 83. The antivirus of any one of embodiments 21-69, wherein the multispecific antibody that comprises a tandem scFv format binds to a Neuraminidase active site and a Hemagglutinin stem of influenza virus and the fusion protein oligomerization domain comprises the D4 trimerization domain of VSV-G protein.
Embodiment 84. The antivirus of any one of embodiments 21-69, wherein the multispecific antibody that comprises a tandem scFv format binds to a Spike NTD and a Spike RBD of SARS COV-2 and the fusion protein oligomerization domain comprises the D4 trimerization domain of VSV-G protein.
Embodiment 85. The antivirus of any one of embodiments 21-69, wherein the multispecific antibody that comprises a tandem scFv format comprises an amino acid sequence from at least one complementarity determining region of CoV2-2021 and an amino acid sequence from at least one complementarity determining region of C012 and the fusion protein oligomerization domain comprises the D4 trimerization domain of VSV-G protein.
Embodiment 86. The antivirus of any one of embodiments 21-69, wherein the antibody comprises the scFv and an amino acid sequence from at least one complementarity determining region of 0304-4A10, and the oligomerization domain comprises the D4 trimerization domain of VSV-G protein, and the second antibody comprises the scFv and an amino acid sequence from at least one complementarity determining region of C018.
Embodiment 87. An antivirus comprising a fusion protein that comprises a transmembrane polypeptide, an oligomerization domain, and an antibody which binds to a surface protein of a virus wherein the fusion protein is expressed at a valency of at least about 10 copies on a surface of the antivirus, and the antivirus neutralizes the virus when the fusion protein is bound to the surface protein of the virus.
Embodiment 88. An antivirus comprising
   (a) a first fusion protein that comprises a first transmembrane polypeptide and a first antibody which binds to a surface protein of a virus wherein the first fusion protein is expressed at a valency of at least about 10 copies on a surface of the antivirus,
   (b) a second fusion protein that comprises a second transmembrane polypeptide and second antibody which binds to a surface protein of the virus, wherein the second antibody comprises a CDR sequence that has less than 100% sequence identity to an equivalent CDR sequence of the first antibody,
   wherein the antivirus neutralizes the virus when either the first fusion protein or second fusion protein is bound to the surface protein of the virus.
Embodiment 89. An antivirus comprising a fusion protein that comprises a transmembrane polypeptide and a multispecific antibody which binds to a surface protein of a virus wherein the fusion protein is expressed at a valency of at least about 10 copies on a surface of the antivirus, and the antivirus neutralizes the virus when the fusion protein is bound to the surface protein of the virus.
Embodiment 90. A pharmaceutical composition comprising the antivirus of any one of embodiments 1-89 and a pharmaceutically acceptable excipient.
Embodiment 91. A composition comprising a nucleic acid sequence that encodes the fusion protein of any one of embodiments 1-89.
Embodiment 92. A composition comprising a nucleic acid sequence that encodes the fusion protein of any one of embodiments 1-89 and the second fusion protein of any one of embodiments 53-89.
Embodiment 93. The composition of embodiment 92, wherein the composition further comprises a second nucleic acid sequence that encodes one or more packaging viral proteins.
Embodiment 94. The composition of embodiment 93, wherein the one or more packaging viral proteins is a lentiviral protein, a retroviral protein, an adenoviral protein, or combinations thereof.
Embodiment 95. The composition of embodiment 93, wherein the one or more packaging viral proteins comprises gag, pol, pre, tat, rev, or combinations thereof.
Embodiment 96. The composition of any one of embodiments 92-95, further comprising a third nucleic acid sequence that encodes a reporter, a therapeutic molecule, or combinations thereof.
Embodiment 97. The composition of embodiment 96, wherein the reporter is a fluorescent protein or luciferase.
Embodiment 98. The composition of embodiment 97, wherein the fluorescent protein is green fluorescent protein.
Embodiment 99. The composition of embodiment 96, wherein the therapeutic molecule is an immune modulating protein, a cellular signal modulating molecule, a proliferation modulating molecule, a cell death modulating molecule, or combinations thereof.
Embodiment 100. The composition of any one of embodiments 92-99, wherein the nucleic acid sequence that encodes the fusion protein or the nucleic acid sequence that encodes the fusion protein and the second fusion protein and the second nucleic acid sequence and the third nucleic acid sequence are within a same vector.
Embodiment 101. The composition of any one of embodiments 92-99, wherein the nucleic acid sequence that encodes the fusion protein or the nucleic acid sequence that encodes the fusion protein and the second fusion protein and the second nucleic acid sequence and the third nucleic acid sequence are within different vectors.
Embodiment 102. The composition of any one of embodiments 100-101, wherein the vector is a lentivirus vector, an adenovirus vector, or an adeno-associated virus vector.
Embodiment 103. A method for producing a virus neutralizing composition from a non-neutralizing antibody that binds specifically to a viral protein; the method comprising expressing the non-neutralizing antibody that binds to the viral protein as a fusion protein with a transmembrane polypeptide on a surface of an antivirus at a valency of at least about 10 copies of the fusion protein on the surface of the antivirus.
Embodiment 104. A method of treating a viral disease in a subject in need thereof comprising administering to the subject an antivirus comprising a fusion protein that comprises a transmembrane polypeptide and an antibody which binds to a surface protein of a virus of the viral disease wherein the fusion protein is expressed at a valency of at least about 10 copies on a surface of the antivirus, and the antivirus neutralizes the virus when the fusion protein is bound to the surface protein of the virus.
Embodiment 105. The method of any one of embodiments 103-104, wherein the fusion protein further comprises an oligomerization domain.
Embodiment 106. The method of embodiment 105, wherein the oligomerization domain is a dimerization domain.
Embodiment 107. The method of embodiment 106, wherein the dimerization domain comprises a leucine zipper dimerization domain.
Embodiment 108. The method of embodiment 105, wherein the oligomerization domain is a trimerization domain.
Embodiment 109. The method of embodiment 108, wherein the trimerization domain comprises a post-fusion oligomerization domain of viral surface protein.
Embodiment 110. The method of embodiment 108, wherein the trimerization domain comprises a D4 post-fusion trimerization domain of VSV-G protein.
Embodiment 111. The method of embodiment 108, wherein the trimerization domain comprises a Dengue E protein post-fusion trimerization domain.
Embodiment 112. The method of embodiment 108, wherein the trimerization domain comprises a foldon trimerization domain.
Embodiment 113. The method of embodiment 105, wherein the oligomerization domain is a tetramerization domain.
Embodiment 114. The method of embodiment 113, wherein the tetramerization domain comprises an influenza neuraminidase stem domain.
Embodiment 115. The method of embodiment 105, wherein the oligomerization domain comprises an amino acid sequence that has at least 95% sequence identity to an amino acid sequence according to SEQ ID NOs: 30-43
Embodiment 116. The method of any one of embodiments 105-115, wherein when the fusion protein is expressed on the surface of the antivirus, the oligomerization domain is outside of the antivirus.
Embodiment 117. The method of any one of embodiments 105-115, wherein when the fusion protein is expressed on the surface of the antivirus, the oligomerization domain is outside of the antivirus and adjacent to a signal peptide.
Embodiment 118. The method of any one of embodiments 105-115, wherein when the fusion protein is expressed on the surface of the antivirus, the oligomerization domain is inside of the antivirus.
Embodiment 119. The method of any one of embodiments 105-115, wherein when the fusion protein is expressed on the surface of the antivirus, the oligomerization domain is inside of the antivirus and adjacent to the transmembrane polypeptide.
Embodiment 120. The method of any one of embodiments 105-118, wherein the fusion protein comprises a signal peptide.
Embodiment 121. The method of any one of embodiments 105-118, wherein domains of the fusion protein are arranged from the N-terminus to the C-terminus in the following orders:
   (a) signal peptide, antibody which binds to a surface protein of a virus, oligomerization domain, transmembrane polypeptide, and cytosolic domain;
   (b) signal peptide, antibody which binds to a surface protein of a virus, transmembrane polypeptide, oligomerization domain, and cytosolic domain; or
   (c) signal peptide, oligomerization domain, antibody which binds to a surface protein of a virus, transmembrane polypeptide, and cytosolic domain.
Embodiment 122. The method of any one of embodiments 103-121, wherein the antibody comprises a single chain variable fragment (scFv), a single domain antibody, an Fv, a VH domain, a VL domain, a Fab fragment, a monoclonal antibody, F(ab′), F(ab′)2, single chain antibodies, diabodies, or a scFv-Fc.
Embodiment 123. The method of any one of embodiments 103-121, wherein the antibody binds specifically to the surface protein of the virus.
Embodiment 124. The method of any one of embodiments 103-121, wherein the antibody is a multispecific antibody.
Embodiment 125. The method of embodiment 124, wherein the multispecific antibody binds specifically to more than one epitope on the surface protein of the virus.
Embodiment 126. The method of embodiment 124, wherein the multispecific antibody comprises a tandem scFv format.
Embodiment 127. The method of any one of embodiments 103-126, wherein the virus comprises SARS COV-1, CoV-2, influenza, or MERS CoV virus.
Embodiment 128. The method of any one of embodiments 103-127, wherein the antibody comprises an amino acid sequence from at least one complementarity determining region of 80R, H4, 7D10, 1E01, F16, 0304-4A10, 2M-14E5, 9A1, COV2-2021, COV2-2026, COV2-2146, 2M-10B11, C021, C018, BG4-5, BG7-15, and BG10-19.
Embodiment 129. The method of any one of embodiments 103-127, wherein the antibody comprises an amino acid sequence according to any one of SEQ ID NOs: 1-28 and 81-86.
Embodiment 130. The method of any one of embodiments 103-127, wherein the antibody neutralizes the virus when expressed within the fusion protein but does not neutralize the virus when expressed as an isolated antibody.
Embodiment 131. The method of any one of embodiments 103-127, wherein the antibody neutralizes the virus when expressed within the fusion protein and neutralizes the virus when expressed as an isolated antibody.
Embodiment 132. The method of any one of embodiments 103-131, wherein the transmembrane polypeptide anchors the fusion protein to a bilayer of the antivirus.
Embodiment 133. The method of any one of embodiments 103-131, wherein the transmembrane polypeptide comprises the transmembrane domain of a Vesicular Stomatitis virus glycoprotein (VSV-G).
Embodiment 134. The method of any one of embodiments 103-131, wherein the transmembrane polypeptide comprises the transmembrane domain of influenza Hemagglutinin (HA).
Embodiment 135. The method of any one of embodiments 103-131, wherein the transmembrane polypeptide comprises the transmembrane domain of HIV surface glycoprotein GP120 or GP41.
Embodiment 136. The method of any one of embodiments 103-131, wherein the transmembrane polypeptide comprises the transmembrane domain of measles virus surface glycoprotein hamagglutinin (H) protein.
Embodiment 137. The method of any one of embodiments 103-131, wherein the transmembrane polypeptide comprises the transmembrane domain of influenza Neuraminidase (NA).
Embodiment 138. The method of any one of embodiments 103-131, wherein the transmembrane polypeptide comprises an amino acid sequence at least about 90% identical to that set forth in SEQ ID NO: 29.
Embodiment 139. The method of any one of embodiments 103-138, wherein the fusion protein is expressed at a valency of about 10 copies on a surface of the antivirus.
Embodiment 140. The method of any one of embodiments 103-138, wherein the fusion protein is expressed at a valency of about 10 to 15 copies on a surface of the antivirus.
Embodiment 141. The method of any one of embodiments 103-138, wherein the fusion protein is expressed at a valency of at least about 25 copies on a surface of the antivirus.
Embodiment 142. The method of any one of embodiments 103-138, wherein the fusion protein is expressed at a valency of at least about 50 copies on a surface of the antivirus.
Embodiment 143. The method of any one of embodiments 103-138, wherein the fusion protein is expressed at a valency of at least about 100 copies on a surface of the antivirus.
Embodiment 144. The method of any one of embodiments 103-138, wherein the fusion protein is expressed at a valency of at least about 200 copies on a surface of the antivirus.
Embodiment 145. The method of any one of embodiment s 103-138, wherein the fusion protein is expressed at a valency of at least about 400 copies on a surface of the antivirus.
Embodiment 146. The method of any one of embodiments 103-138, wherein the fusion protein is expressed at a valency of at least about 600 copies on a surface of the antivirus.
Embodiment 147. The method of any one of embodiments 103-138, wherein the fusion protein is expressed at a valency of at least about 1000 copies on a surface of the antivirus.
Embodiment 148. The method of any one of embodiments 103-147, wherein the antivirus is an enveloped particle.
Embodiment 149. The method of any one of embodiments 103-147, wherein the antivirus is not a lentiviral particle.
Embodiment 150. The method of any one of embodiments 103-147, wherein the antivirus does not comprise viral genetic material.
Embodiment 151. The method of any one of embodiments 103-147, wherein the antivirus comprises a lipid bilayer.
Embodiment 152. The method of any one of embodiments 103-147, wherein the antivirus is a virus.
Embodiment 153. The method of any one of embodiments 103-147, wherein the antivirus is a replication incompetent virus.
Embodiment 154. The method of any one of embodiments 103-147, wherein the antivirus is a replication competent virus.
Embodiment 155. The method of any one of embodiments 103-147, wherein the antivirus is a viral-like particle.
Embodiment 156. The method of any one of embodiments 103-147, wherein the antivirus is an extracellular vesicle.
Embodiment 157. The method of any one of embodiments 103-147, wherein the antivirus is an exosome.
Embodiment 158. The method of any one of embodiments 103-157, wherein the antivirus further comprises a second fusion protein that comprises transmembrane polypeptide and a second antibody which binds to a surface protein of the virus, wherein the second antibody comprises a CDR sequence that has less than 100% sequence identity to an equivalent CDR sequence of the antibody.
Embodiment 159. The method of embodiment 158, wherein the fusion protein and the second fusion protein comprise the same transmembrane polypeptide.
Embodiment 160. The method of embodiment 158, wherein the fusion protein and the second fusion protein comprise different transmembrane polypeptides.
Embodiment 161. The method of embodiment 158, wherein the second antibody binds to the same surface protein as the antibody.
Embodiment 162. The method of embodiment 158, wherein the second antibody binds to a different surface protein as the antibody.
Embodiment 163. The method of embodiment 158, wherein the second antibody is a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc.
Embodiment 164. The method of embodiment 158, wherein the second antibody binds specifically to the surface protein of the virus.
Embodiment 165. The method of embodiment 158, wherein the second antibody is a multispecific antibody.
Embodiment 166. The method of embodiment 158, wherein the second multispecific antibody binds specifically to more than one epitope on the surface protein of the virus.
Embodiment 167. The method of embodiment 158, wherein the second antibody comprises an amino acid sequence from at least one complementarity determining region of 80R, H4, 7D10, 1E01, F16, 0304-4A10, 2M-14E5, 9A1, COV2-2021, COV2-2026, COV2-2146, 2M-10B11, C021, C018, BG4-5, BG7-15, and BG10-19.
Embodiment 168. The method of embodiment 158, wherein the second antibody comprises an amino acid sequence according to any one of SEQ ID NOs: 1-28 and 81-86.
Embodiment 169. The method of embodiment 158, wherein the second antibody neutralizes the virus when expressed within the fusion protein but does not neutralize the virus when expressed as an isolated antibody.
Embodiment 170. The method of embodiment 158, wherein the second antibody neutralizes the virus when expressed within the fusion protein and neutralizes the virus when expressed as an isolated antibody.
Embodiment 171. The method of embodiment 158, wherein the second fusion protein further comprises an oligomerization domain.
Embodiment 172. The method of embodiment 171, wherein the oligomerization domain is a dimerization domain.
Embodiment 173. The method of embodiment 171, wherein the oligomerization domain is a trimerization domain.
Embodiment 174. The method of embodiment 171, wherein the oligomerization domain comprises the D4 trimerization domain of VSV-G protein.
Embodiment 175. The method of any one of embodiments 103-174, wherein the antibody comprises the scFv and an amino acid sequence from at least one complementarity determining region of 80R.
Embodiment 176. The method of any one of embodiments 103-174, wherein the antibody comprises the scFv and an amino acid sequence from at least one complementarity determining region of H4.
Embodiment 177. The method of any one of embodiments 103-174, wherein the antibody comprises the scFv and an amino acid sequence from at least one complementarity determining region of 7D10.
Embodiment 178. The method of any one of embodiments 103-174, wherein the antibody comprises the scFv and an amino acid sequence from at least one complementarity determining region of COV2-2021.
Embodiment 179. The method of any one of embodiments 103-174, wherein the antibody comprises the scFv and an amino acid sequence from at least one complementarity determining region of C021.
Embodiment 180. The method of any one of embodiments 103-174, wherein the antibody comprises the scFv and an amino acid sequence from at least one complementarity determining region of C018.
Embodiment 181. The method of any one of embodiments 103-174, wherein the antibody comprises the scFv and an amino acid sequence from at least one complementarity determining region of H4 and the oligomerization domain comprises the D4 trimerization domain of VSV-G protein.
Embodiment 182. The method of any one of embodiments 103-174, wherein the antibody comprises the scFv and an amino acid sequence from at least one complementarity determining region of 1E01 and the oligomerization domain comprises the D4 trimerization domain of VSV-G protein.
Embodiment 183. The method of any one of embodiments 103-174, wherein the antibody comprises the scFv and an amino acid sequence from at least one complementarity determining region of F16 and the oligomerization domain comprises the D4 trimerization domain of VSV-G protein.
Embodiment 184. The method of any one of embodiments 103-174, wherein the antibody comprises the scFv and an amino acid sequence from at least one complementarity determining region of 0304-4A10, and the oligomerization domain comprises the D4 trimerization domain of VSV-G protein.
Embodiment 185. The method of any one of embodiments 103-174, wherein the antibody comprises the scFv and an amino acid sequence from at least one complementarity determining region of 2M-14E5, and the oligomerization domain comprises the D4 trimerization domain of VSV-G protein.
Embodiment 186. The method of any one of embodiments 103-174, wherein the antibody comprises the scFv and an amino acid sequence from at least one complementarity determining region of 9A1, and the oligomerization domain comprises the D4 trimerization domain of VSV-G protein.
Embodiment 187. The method of any one of embodiments 103-174, wherein the antibody comprises the scFv and an amino acid sequence from at least one complementarity determining region of COV2-2021, and the oligomerization domain comprises the D4 trimerization domain of VSV-G protein.
Embodiment 188. The method of any one of embodiments 103-174, wherein the antibody comprises the scFv and an amino acid sequence from at least one complementarity determining region of COV2-2026, and the oligomerization domain comprises the D4 trimerization domain of VSV-G protein.
Embodiment 189. The method of any one of embodiments 103-174, wherein the antibody comprises the scFv and an amino acid sequence from at least one complementarity determining region of COV2-2146, and the oligomerization domain comprises the D4 trimerization domain of VSV-G protein.
Embodiment 190. The method of any one of embodiments 103-174, wherein the antibody comprises the scFv and an amino acid sequence from at least one complementarity determining region of 2M-10B11, and the oligomerization domain comprises the D4 trimerization domain of VSV-G protein.
Embodiment 191. The method of any one of embodiments 103-174, wherein the antibody comprises the scFv and an amino acid sequence from at least one complementarity determining region of C021, and the oligomerization domain comprises the D4 trimerization domain of VSV-G protein.
Embodiment 192. The method of any one of embodiments 103-174, wherein the antibody comprises the scFv and an amino acid sequence from at least one complementarity determining region of C018, and the oligomerization domain comprises the D4 trimerization domain of VSV-G protein.
Embodiment 193. The method of any one of embodiments 124-174, wherein the multispecific antibody that comprises a tandem scFv format binds to a Neuraminidase active site and a Hemagglutinin stem of influenza virus and the fusion protein oligomerization domain comprises the D4 trimerization domain of VSV-G protein.
Embodiment 194. The method of any one of embodiments 124-174, wherein the multispecific antibody that comprises a tandem scFv format comprises an amino acid sequence from at least one complementarity determining region of 1E01 and an amino acid sequence from at least one complementarity determining region of F16 and the fusion protein oligomerization domain comprises the D4 trimerization domain of VSV-G protein.
Embodiment 195. The method of any one of embodiments 124-174, wherein the multispecific antibody that comprises a tandem scFv format binds to a Spike NTD and a Spike RBD of SARS COV-2 and the fusion protein oligomerization domain comprises the D4 trimerization domain of VSV-G protein.
Embodiment 196. The method of any one of embodiments 124-174, wherein the multispecific antibody that comprises a tandem scFv format comprises an amino acid sequence from at least one complementarity determining region of CoV2-2021 and an amino acid sequence from at least one complementarity determining region of C012 and the fusion protein oligomerization domain comprises the D4 trimerization domain of VSV-G protein.
Embodiment 197. The method of any one of embodiments 103-174, wherein the antibody comprises the scFv and an amino acid sequence from at least one complementarity determining region of 0304-4A10, and the oligomerization domain comprises the D4 trimerization domain of VSV-G protein, and the second antibody comprises the scFv and an amino acid sequence from at least one complementarity determining region of C018.
Embodiment 198. The method of any one of embodiments 103-197, wherein the antivirus is administered to the subject intravenously.
Embodiment 199. The method of any one of embodiments 103-197, wherein the antivirus is administered to the subject through inhalation.
Embodiment 200. The method of any one of embodiments 307-396, wherein the antivirus is administered to the subject through intranasal delivery.
Embodiment 201. The method of any one of embodiments 307-396, wherein the antivirus is administered to the subject through intratracheal delivery.
Embodiment 202. The method of any one of embodiments 103-197, wherein the antivirus is administered to the subject by an intraperitoneal injection.
Embodiment 203. The method of any one of embodiments 103-197, wherein the antivirus is administered to the subject by an subcutaneous injection.
Embodiment 204. The method of any one of embodiments 103-197, wherein the antivirus induces T cell mediated cytotoxicity against viral infected cells.
Embodiment 205. The method of any one of embodiments 103-197, wherein the administering to the subject of the antivirus is sufficient to reduce or eliminate the viral disease as compared to a baseline measurement of the viral disease taken from the subject prior to the administering of the antivirus.
Embodiment 206. The method of embodiment 205, wherein the reduction is at least about 1-fold, 5-fold, 10-fold, 20-fold, 40-fold, 60-fold, 80-fold, or up to about 100-fold.
Embodiment 207. An antivirus comprising a fusion protein that comprises a transmembrane polypeptide and an antibody which binds to a surface protein of a virus wherein the fusion protein is expressed at a valency of at least about 10 copies on a surface of the antivirus, and wherein the antivirus does not comprise viral genetic material and the antivirus neutralizes the virus when the fusion protein is bound to the surface protein of the virus.
Embodiment 208. The antivirus of embodiment 207, wherein the fusion protein further comprises an oligomerization domain.
Embodiment 209. The antivirus of embodiment 208, wherein the oligomerization domain is a dimerization domain.
Embodiment 210. The antivirus of embodiment 209, wherein the dimerization domain comprises a leucine zipper dimerization domain.
Embodiment 211. The antivirus of embodiment 208, wherein the oligomerization domain is a trimerization domain.
Embodiment 212. The antivirus of embodiment 211, wherein the trimerization domain comprises a post-fusion oligomerization domain of viral surface protein.
Embodiment 213. The antivirus of embodiment 211, wherein the trimerization domain comprises a D4 post-fusion trimerization domain of VSV-G protein.
Embodiment 214. The antivirus of embodiment 211, wherein the trimerization domain comprises a Dengue E protein post-fusion trimerization domain.
Embodiment 215. The antivirus of embodiment 211, wherein the trimerization domain comprises a foldon trimerization domain.
Embodiment 216. The antivirus of embodiment 211, wherein the oligomerization domain is a tetramerization domain.
Embodiment 217. The antivirus of embodiment 216, wherein the tetramerization domain comprises an influenza neuraminidase stem domain.
Embodiment 218. The antivirus of embodiment 208, wherein the oligomerization domain comprises an amino acid sequence that has at least 95% sequence identity to an amino acid sequence according to SEQ ID NOs: 30-43.
Embodiment 219. The antivirus of any one of embodiments 208-218, wherein when the fusion protein is expressed on the surface of the antivirus, the oligomerization domain is outside of the antivirus.
Embodiment 220. The antivirus of any one of embodiments 208-218, wherein when the fusion protein is expressed on the surface of the antivirus, the oligomerization domain is outside of the antivirus and adjacent to a signal peptide.
Embodiment 221. The antivirus of any one of embodiments 208-218, wherein when the fusion protein is expressed on the surface of the antivirus, the oligomerization domain is inside of the antivirus.
Embodiment 222. The antivirus of any one of embodiments 208-218, wherein when the fusion protein is expressed on the surface of the antivirus, the oligomerization domain is inside of the antivirus and adjacent to the transmembrane polypeptide.
Embodiment 223. The antivirus of any one of embodiments 207-221, wherein the fusion protein comprises a signal peptide.
Embodiment 224. The antivirus of any one of embodiments 207-221, wherein domains of the fusion protein are arranged from the N-terminus to the C-terminus in the following orders:
   (a) signal peptide, antibody which binds to a surface protein of a virus, oligomerization domain, transmembrane polypeptide, and cytosolic domain;
   (b) signal peptide, antibody which binds to a surface protein of a virus, transmembrane polypeptide, oligomerization domain, and cytosolic domain; or
   (c) signal peptide, oligomerization domain, antibody which binds to a surface protein of a virus, transmembrane polypeptide, and cytosolic domain.
Embodiment 225. The antivirus of any one of embodiments 207-224, wherein the antibody comprises a single chain variable fragment (scFv), a single domain antibody, an Fv, a VH domain, a VL domain, a Fab fragment, a monoclonal antibody, F(ab′), F(ab′)2, single chain antibodies, diabodies, or a scFv-Fc.
Embodiment 226. The antivirus of embodiment 225, wherein the antibody binds specifically to the surface protein of the virus.
Embodiment 227. The antivirus of any one of embodiments 207-224, wherein the antibody is a multispecific antibody.
Embodiment 228. The antivirus of embodiment 227, wherein the multispecific antibody binds specifically to more than one epitope on the surface protein of the virus.
Embodiment 229. The antivirus of embodiment 227, wherein the multispecific antibody comprises a tandem scFv format.
Embodiment 230. The antivirus of any one of embodiments 207-229, wherein the virus comprises SARS COV-1, CoV-2, influenza, or MERS CoV virus.
Embodiment 231. The antivirus of any one of embodiments 207-229, wherein the antibody comprises an amino acid sequence from at least one complementarity determining region of 80R, H4, 7D10, 1E01, F16, 0304-4A10, 2M-14E5, 9A1, COV2-2021, COV2-2026, COV2-2146, 2M-10B11, C021, C018, BG4-5, BG7-15, and BG10-19.
Embodiment 232. The antivirus of any one of embodiments 207-231, wherein the antibody comprises an amino acid sequence according to any one of SEQ ID NOs: 1-28 and 81-86.
Embodiment 233. The antivirus of any one of embodiments 207-232, wherein the antibody neutralizes the virus when expressed within the fusion protein but does not neutralize the virus when expressed as an isolated antibody.
Embodiment 234. The antivirus of any one of embodiments 207-232, wherein the antibody neutralizes the virus when expressed within the fusion protein and neutralizes the virus when expressed as an isolated antibody.
Embodiment 235. The antivirus of any one of embodiments 207-233, wherein the transmembrane polypeptide anchors the fusion protein to a bilayer of the antivirus.
Embodiment 236. The antivirus of any one of embodiments 207-233, wherein the transmembrane polypeptide comprises the transmembrane domain of a Vesicular Stomatitis virus glycoprotein (VSV-G).
Embodiment 237. The antivirus of any one of embodiments 207-233, wherein the transmembrane polypeptide comprises the transmembrane domain of influenza Hemagglutinin (HA).
Embodiment 238. The antivirus of any one of embodiments 207-233, wherein the transmembrane polypeptide comprises the transmembrane domain of HIV surface glycoprotein GP120 or GP41.
Embodiment 239. The antivirus of any one of embodiments 207-233, wherein the transmembrane polypeptide comprises the transmembrane domain of measles virus surface glycoprotein hamagglutinin (H) protein.
Embodiment 240. The antivirus of any one of embodiments 207-233, wherein the transmembrane polypeptide comprises the transmembrane domain of influenza Neuraminidase (NA).
Embodiment 241. The antivirus of any one of embodiments 207-233, wherein the transmembrane polypeptide comprises an amino acid sequence at least about 90% identical to that set forth in SEQ ID NO: 29.
Embodiment 242. The antivirus of any one of embodiments 207-241, wherein the fusion protein is expressed at a valency of about 10 copies on a surface of the antivirus.
Embodiment 243. The antivirus of any one of embodiments 207-241, wherein the fusion protein is expressed at a valency of about 10 to 15 copies on a surface of the antivirus.
Embodiment 244. The antivirus of any one of embodiments 207-241, wherein the fusion protein is expressed at a valency of at least about 25 copies on a surface of the antivirus.
Embodiment 245. The antivirus of any one of embodiments 207-241, wherein the fusion protein is expressed at a valency of at least about 50 copies on a surface of the antivirus.
Embodiment 246. The antivirus of any one of embodiments 207-241, wherein the fusion protein is expressed at a valency of at least about 100 copies on a surface of the antivirus.
Embodiment 247. The antivirus of any one of embodiments 207-241, wherein the fusion protein is expressed at a valency of at least about 200 copies on a surface of the antivirus.
Embodiment 248. The antivirus of any one of embodiments 207-241, wherein the fusion protein is expressed at a valency of at least about 400 copies on a surface of the antivirus.
Embodiment 249. The antivirus of any one of embodiments 207-241, wherein the fusion protein is expressed at a valency of at least about 600 copies on a surface of the antivirus.
Embodiment 250. The antivirus of any one of embodiments 207-241, wherein the fusion protein is expressed at a valency of at least about 1000 copies on a surface of the antivirus.
Embodiment 251. The antivirus of any one of embodiments 207-250, wherein the antivirus is an enveloped particle.
Embodiment 252. The antivirus of any one of embodiments 207-250, wherein the antivirus is not a lentiviral particle.
Embodiment 253. The antivirus of any one of embodiments 207-250, wherein the antivirus comprises a lipid bilayer.
Embodiment 254. The antivirus of any one of embodiments 207-250, wherein the antivirus is a viral-like particle.
Embodiment 255. The antivirus of any one of embodiments 207-250, wherein the antivirus is an extracellular vesicle.
Embodiment 256. The antivirus of any one of embodiments 207-250, wherein the antivirus is an exosome.
Embodiment 257. The antivirus of any one of embodiments 207-250, wherein the antivirus is an ectosome.
Embodiment 258. The antivirus of any one of embodiments 207-256, wherein the antivirus further comprises a second fusion protein that comprises a second transmembrane polypeptide and a second antibody which binds to a surface protein of the virus, wherein the second antibody comprises a CDR sequence that has less than 100% sequence identity to an equivalent CDR sequence of the antibody.
Embodiment 259. The antivirus of embodiment 258, wherein the fusion protein and the second fusion protein comprise the same transmembrane polypeptide.
Embodiment 260. The antivirus of embodiment 258, wherein the fusion protein and the second fusion protein comprise different transmembrane polypeptides.
Embodiment 261. The antivirus of embodiment 258, wherein the second antibody binds to the same surface protein as the antibody.
Embodiment 262. The antivirus of embodiment 258, wherein the second antibody binds to a different surface protein as the antibody.
Embodiment 263. The antivirus of embodiment 258, wherein the second antibody is a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc.
Embodiment 264. The antivirus of embodiment 258, wherein the second antibody binds specifically to the surface protein of the virus.
Embodiment 265. The antivirus of embodiment 258, wherein the second antibody is a multispecific antibody.
Embodiment 266. The antivirus of embodiment 258, wherein the second multispecific antibody binds specifically to more than one epitope on the surface protein of the virus.
Embodiment 267. The antivirus of embodiment 258, wherein the second antibody comprises an amino acid sequence from at least one complementarity determining region of 80R, H4, 7D10, 1E01, F16, 0304-4A10, 2M-14E5, 9A1, COV2-2021, COV2-2026, COV2-2146, 2M-10B11, C021, C018, BG4-5, BG7-15, and BG10-19.
Embodiment 268. The antivirus of embodiment 258, wherein the second antibody comprises an amino acid sequence according to any one of SEQ ID NOs: 1-28 and 81-86.
Embodiment 269. The antivirus of embodiment 258, wherein the second antibody neutralizes the virus when expressed within the fusion protein but does not neutralize the virus when expressed as an isolated antibody.
Embodiment 270. The antivirus of embodiment 258, wherein the second antibody neutralizes the virus when expressed within the fusion protein and neutralizes the virus when expressed as an isolated antibody.
Embodiment 271. The antivirus of embodiment 258, wherein the second fusion protein further comprises an oligomerization domain.
Embodiment 272. The antivirus of embodiment 271, wherein the oligomerization domain is a dimerization domain.
Embodiment 273. The antivirus of embodiment 271, wherein the oligomerization domain is a trimerization domain.
Embodiment 274. The antivirus of embodiment 271, wherein the oligomerization domain comprises the D4 trimerization domain of VSV-G protein.
Embodiment 275. The antivirus of any one of embodiments 207-274, wherein the antibody comprises the scFv and an amino acid sequence from at least one complementarity determining region of 80R.
Embodiment 276. The antivirus of any one of embodiments 207-274, wherein the antibody comprises the scFv and an amino acid sequence from at least one complementarity determining region of H4.
Embodiment 277. The antivirus of any one of embodiments 207-274, wherein the antibody comprises the scFv and an amino acid sequence from at least one complementarity determining region of 7D10.
Embodiment 278. The antivirus of any one of embodiments 207-274, wherein the antibody comprises the scFv and an amino acid sequence from at least one complementarity determining region of COV2-2021.
Embodiment 279. The antivirus of any one of embodiments 207-274, wherein the antibody comprises the scFv and an amino acid sequence from at least one complementarity determining region of C021.
Embodiment 280. The antivirus of any one of embodiments 207-274, wherein the antibody comprises the scFv and an amino acid sequence from at least one complementarity determining region of C018.
Embodiment 281. The antivirus of any one of embodiments 207-274, wherein the antibody comprises the scFv and an amino acid sequence from at least one complementarity determining region of H4 and the oligomerization domain comprises the D4 trimerization domain of VSV-G protein.
Embodiment 282. The antivirus of any one of embodiments 207-274, wherein the antibody comprises the scFv and an amino acid sequence from at least one complementarity determining region of 1E01 and the oligomerization domain comprises the D4 trimerization domain of VSV-G protein.
Embodiment 283. The antivirus of any one of embodiments 207-274, wherein the antibody comprises the scFv and an amino acid sequence from at least one complementarity determining region of F16 and the oligomerization domain comprises the D4 trimerization domain of VSV-G protein.
Embodiment 284. The antivirus of any one of embodiments 207-274, wherein the antibody comprises the scFv and an amino acid sequence from at least one complementarity determining region of 0304-4A10, and the oligomerization domain comprises the D4 trimerization domain of VSV-G protein.
Embodiment 285. The antivirus of any one of embodiments 207-274, wherein the antibody comprises the scFv and an amino acid sequence from at least one complementarity determining region of 2M-14E5, and the oligomerization domain comprises the D4 trimerization domain of VSV-G protein.
Embodiment 286. The antivirus of any one of embodiments 207-274, wherein the antibody comprises the scFv and an amino acid sequence from at least one complementarity determining region of 9A1, and the oligomerization domain comprises the D4 trimerization domain of VSV-G protein.
Embodiment 287. The antivirus of any one of embodiments 207-274, wherein the antibody comprises the scFv and an amino acid sequence from at least one complementarity determining region of COV2-2021, and the oligomerization domain comprises the D4 trimerization domain of VSV-G protein.
Embodiment 288. The antivirus of any one of embodiments 207-274, wherein the antibody comprises the scFv and an amino acid sequence from at least one complementarity determining region of COV2-2026, and the oligomerization domain comprises the D4 trimerization domain of VSV-G protein.
Embodiment 289. The antivirus of any one of embodiments 207-274, wherein the antibody comprises the scFv and an amino acid sequence from at least one complementarity determining region of COV2-2146, and the oligomerization domain comprises the D4 trimerization domain of VSV-G protein.
Embodiment 290. The antivirus of any one of embodiments 207-274, wherein the antibody comprises the scFv and an amino acid sequence from at least one complementarity determining region of 2M-10B11, and the oligomerization domain comprises the D4 trimerization domain of VSV-G protein.
Embodiment 291. The antivirus of any one of embodiments 207-274, wherein the antibody comprises the scFv and an amino acid sequence from at least one complementarity determining region of C021, and the oligomerization domain comprises the D4 trimerization domain of VSV-G protein.
Embodiment 292. The antivirus of any one of embodiments 207-274, wherein the antibody comprises the scFv and an amino acid sequence from at least one complementarity determining region of C018, and the oligomerization domain comprises the D4 trimerization domain of VSV-G protein.
Embodiment 293. The antivirus of any one of embodiments 207-274, wherein the multispecific antibody that comprises a tandem scFv format binds to a Neuraminidase active site and a Hemagglutinin stem of influenza virus and the fusion protein oligomerization domain comprises the D4 trimerization domain of VSV-G protein.
Embodiment 294. The antivirus of any one of embodiments 207-274, wherein the multispecific antibody that comprises a tandem scFv format comprises an amino acid sequence from at least one complementarity determining region of 1E01 and an amino acid sequence from at least one complementarity determining region of F16 and the fusion protein oligomerization domain comprises the D4 trimerization domain of VSV-G protein.
Embodiment 295. The antivirus of any one of embodiments 207-274, wherein the multispecific antibody that comprises a tandem scFv format binds to a Spike NTD and a Spike RBD of SARS COV-2 and the fusion protein oligomerization domain comprises the D4 trimerization domain of VSV-G protein.
Embodiment 296. The antivirus of any one of embodiments 207-274, wherein the multispecific antibody that comprises a tandem scFv format comprises an amino acid sequence from at least one complementarity determining region of CoV2-2021 and an amino acid sequence from at least one complementarity determining region of C012 and the fusion protein oligomerization domain comprises the D4 trimerization domain of VSV-G protein.
Embodiment 297. The antivirus of any one of embodiments 207-274, wherein the antibody comprises the scFv and an amino acid sequence from at least one complementarity determining region of 0304-4A10, and the oligomerization domain comprises the D4 trimerization domain of VSV-G protein, and the second antibody comprises the scFv and an amino acid sequence from at least one complementarity determining region of C018.
Embodiment 298. A pharmaceutical composition comprising the antivirus of any one of embodiments 207-297 and a pharmaceutically acceptable excipient.
Embodiment 299. A composition comprising a nucleic acid sequence that encodes the fusion protein of any one of embodiments 207-297.
Embodiment 300. A composition comprising a nucleic acid sequence that encodes the fusion protein of any one of embodiments 207-256 and the second fusion protein of any one of embodiments 258-297.
Embodiment 301. The composition of embodiment 300, wherein the composition further comprises a second nucleic acid sequence that encodes one or more packaging viral proteins.
Embodiment 302. The composition of embodiment 301, wherein the one or more packaging viral proteins is a lentiviral protein, a retroviral protein, an adenoviral protein, or combinations thereof.
Embodiment 303. The composition of embodiment 301, wherein the one or more packaging viral proteins comprises gag, pol, pre, tat, rev, or combinations thereof.
Embodiment 304. The composition of embodiment 301, wherein the nucleic acid sequence that encodes the fusion protein or the nucleic acid sequence that encodes the fusion protein and the second fusion protein and the second nucleic acid sequence are within a same vector.
Embodiment 305. The composition of embodiment 301, wherein the nucleic acid sequence that encodes the fusion protein or the nucleic acid sequence that encodes the fusion protein and the second fusion protein and the second nucleic acid sequence are within different vectors.
Embodiment 306. The composition of any one of embodiment s 304-305, wherein the vector is a lentivirus vector, an adenovirus vector, or an adeno-associated virus vector.
Embodiment 307. A method of treating a viral disease in a subject in need thereof comprising administering to the subject an antivirus comprising a fusion protein that comprises a transmembrane polypeptide and an antibody which binds to a surface protein of a virus of the viral disease wherein the fusion protein is expressed at a valency of at least about 10 copies on a surface of the antivirus, and wherein the antivirus does not comprise viral genetic material, and the antivirus neutralizes the virus when the fusion protein is bound to the surface protein of the virus.
Embodiment 308. The method of embodiment 307, wherein the fusion protein further comprises an oligomerization domain.
Embodiment 309. The method of embodiment 308, wherein the oligomerization domain is a dimerization domain.
Embodiment 310. The method of embodiment 309, wherein the dimerization domain comprises a leucine zipper dimerization domain.
Embodiment 311. The method of embodiment 308, wherein the oligomerization domain is a trimerization domain.
Embodiment 312. The method of embodiment 311, wherein the trimerization domain comprises a post-fusion oligomerization domain of viral surface protein.
Embodiment 313. The method of Embodiment 311, wherein the trimerization domain comprises a D4 post-fusion trimerization domain of VSV-G protein.
Embodiment 314. The method of embodiment 311, wherein the trimerization domain comprises a Dengue E protein post-fusion trimerization domain.
Embodiment 315. The method of embodiment 311, wherein the trimerization domain comprises a foldon trimerization domain.
Embodiment 316. The method of embodiment 308, wherein the oligomerization domain is a tetramerization domain.
Embodiment 317. The method of embodiment 316, wherein the tetramerization domain comprises an influenza neuraminidase stem domain.
Embodiment 318. The method of embodiment 308, wherein the oligomerization domain comprises an amino acid sequence that has at least 95% sequence identity to an amino acid sequence according to SEQ ID NOs: 30-43.
Embodiment 319. The method of any one of embodiments 308-318, wherein when the fusion protein is expressed on the surface of the antivirus, the oligomerization domain is outside of the antivirus.
Embodiment 320. The method of any one of embodiments 308-318, wherein when the fusion protein is expressed on the surface of the antivirus, the oligomerization domain is outside of the antivirus and adjacent to a signal peptide.
Embodiment 321. The method of any one of embodiments 308-318, wherein when the fusion protein is expressed on the surface of the antivirus, the oligomerization domain is inside of the antivirus.
Embodiment 322. The method of any one of embodiments 308-318, wherein when the fusion protein is expressed on the surface of the antivirus, the oligomerization domain is inside of the antivirus and adjacent to the transmembrane polypeptide.
Embodiment 323. The method of any one of embodiments 307-321, wherein the fusion protein comprises a signal peptide.
Embodiment 324. The method of any one of embodiments 307-321, wherein domains of the fusion protein are arranged from the N-terminus to the C-terminus in the following orders:
   (a) signal peptide, antibody which binds to a surface protein of a virus, oligomerization domain, transmembrane polypeptide, and cytosolic domain;
   (b) signal peptide, antibody which binds to a surface protein of a virus, transmembrane polypeptide, oligomerization domain, and cytosolic domain; or
   (c) signal peptide, oligomerization domain, antibody which binds to a surface protein of a virus, transmembrane polypeptide, and cytosolic domain.
Embodiment 325. The method of any one of embodiments 307-324Error! Reference source not found, wherein the antibody comprises a single chain variable fragment (scFv), a single domain antibody, an Fv, a VH domain, a VL domain, a Fab fragment, a monoclonal antibody, F(ab′), F(ab′)2, single chain antibodies, diabodies, or a scFv-Fc.
Embodiment 326. The method of any one of embodiments 307-324, wherein the antibody binds specifically to the surface protein of the virus.
Embodiment 327. The method of any one of embodiments 307-324, wherein the antibody is a multispecific antibody.
Embodiment 328. The method of any one of embodiments 307-324, wherein the multispecific antibody binds specifically to more than one epitope on the surface protein of the virus.
Embodiment 329. The method of any one of embodiments 307-324, wherein the multispecific antibody comprises a tandem scFv format.
Embodiment 330. The method of any one of embodiments 307-324, wherein the virus comprises SARS COV-1, CoV-2, influenza, or MERS CoV virus.
Embodiment 331. The method of any one of embodiments 307-324, wherein the antibody comprises an amino acid sequence from at least one complementarity determining region of 80R, H4, 7D10, 1E01, F16, 0304-4A10, 2M-14E5, 9A1, COV2-2021, COV2-2026, COV2-2146, 2M-10B11, C021, C018, BG4-5, BG7-15, and BG10-19.
Embodiment 332. The method of any one of embodiments 307-324, wherein the antibody comprises an amino acid sequence according to any one of SEQ ID NOs: 1-28 and 81-86.
Embodiment 333. The method of any one of embodiments 307-324, wherein the antibody neutralizes the virus when expressed within the fusion protein but does not neutralize the virus when expressed as an isolated antibody.
Embodiment 334. The method of any one of embodiments 307-324, wherein the antibody neutralizes the virus when expressed within the fusion protein and neutralizes the virus when expressed as an isolated antibody.
Embodiment 335. The method of any one of embodiments 307-334, wherein the transmembrane polypeptide anchors the fusion protein to a bilayer of the antivirus.
Embodiment 336. The method of any one of embodiments 307-334, wherein the transmembrane polypeptide comprises the transmembrane domain of a Vesicular Stomatitis virus glycoprotein (VSV-G).
Embodiment 337. The method of any one of embodiments 307-334, wherein the transmembrane polypeptide comprises the transmembrane domain of influenza Hemagglutinin (HA).
Embodiment 338. The method of any one of embodiments 307-334, wherein the transmembrane polypeptide comprises the transmembrane domain of HIV surface glycoprotein GP120 or GP41.
Embodiment 339. The method of any one of embodiments 307-334, wherein the transmembrane polypeptide comprises the transmembrane domain of measles virus surface glycoprotein hamagglutinin (H) protein.
Embodiment 340. The method of any one of embodiments 307-334, wherein the transmembrane polypeptide comprises the transmembrane domain of influenza Neuraminidase (NA).
Embodiment 341. The method of any one of embodiments 307-334, wherein the transmembrane polypeptide comprises an amino acid sequence at least about 90% identical to that set forth in any one of SEQ ID NOs: 44-52.
Embodiment 342. The method of any one of embodiments 307-341, wherein the fusion protein is expressed at a valency of about 10 copies on a surface of the antivirus.
Embodiment 343. The method of any one of embodiments 307-341, wherein the fusion protein is expressed at a valency of about 10 to 15 copies on a surface of the antivirus.
Embodiment 344. The method of any one of embodiments 307-341, wherein the fusion protein is expressed at a valency of at least about 25 copies on a surface of the antivirus.
Embodiment 345. The method of any one of embodiments 307-341, wherein the fusion protein is expressed at a valency of at least about 50 copies on a surface of the antivirus.
Embodiment 346. The method of any one of embodiments 307-341, wherein the fusion protein is expressed at a valency of at least about 100 copies on a surface of the antivirus.
Embodiment 347. The method of any one of embodiments 307-341, wherein the fusion protein is expressed at a valency of at least about 200 copies on a surface of the antivirus.
Embodiment 348. The method of any one of embodiments 307-341, wherein the fusion protein is expressed at a valency of at least about 400 copies on a surface of the antivirus.
Embodiment 349. The method of any one of embodiments 307-341, wherein the fusion protein is expressed at a valency of at least about 600 copies on a surface of the antivirus.
Embodiment 350. The method of any one of embodiments 307-341, wherein the fusion protein is expressed at a valency of at least about 1000 copies on a surface of the antivirus.
Embodiment 351. The method of any one of embodiments 307-350, wherein the antivirus is an enveloped particle.
Embodiment 352. The method of any one of embodiments 307-350, wherein the antivirus is not a lentiviral particle.
Embodiment 353. The method of any one of embodiments 307-350, wherein the antivirus comprises a lipid bilayer.
Embodiment 354. The method of any one of embodiments 307-350, wherein the antivirus is a viral-like particle with no viral genome.
Embodiment 355. The method of any one of embodiments 307-350, wherein the antivirus is an extracellular vesicle.
Embodiment 356. The method of any one of embodiments 307-350, wherein the antivirus is an exosome.
Embodiment 357. The method of any one of embodiments 307-356, wherein the antivirus further comprises a second fusion protein that comprises transmembrane polypeptide and a second antibody which binds to a surface protein of the virus, wherein the second antibody comprises a CDR sequence that has less than 100% sequence identity to an equivalent CDR sequence of the antibody.
Embodiment 358. The method of embodiment 357, wherein the fusion protein and the second fusion protein comprise the same transmembrane polypeptide.
Embodiment 359. The method of embodiment 357, wherein the fusion protein and the second fusion protein comprise different transmembrane polypeptides.
Embodiment 360. The method of embodiment 357, wherein the second antibody binds to the same surface protein as the antibody.
Embodiment 361. The method of embodiment 357, wherein the second antibody binds to a different surface protein as the antibody.
Embodiment 362. The method of embodiment 357, wherein the second antibody is a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc.
Embodiment 363. The method of embodiment 357, wherein the second antibody binds specifically to the surface protein of the virus.
Embodiment 364. The method of embodiment 357, wherein the second antibody is a multispecific antibody.
Embodiment 365. The method of embodiment 357, wherein the second multispecific antibody binds specifically to more than one epitope on the surface protein of the virus.
Embodiment 366. The method of embodiment 357, wherein the second antibody comprises an amino acid sequence from at least one complementarity determining region of 80R, H4, 7D10, 1E01, F16, 0304-4A10, 2M-14E5, 9A1, COV2-2021, COV2-2026, COV2-2146, 2M-10B11, C021, C018, BG4-5, BG7-15, and BG10-19.
Embodiment 367. The method of embodiment 357, wherein the second antibody comprises an amino acid sequence according to any one of SEQ ID NOs: 1-28 and 81-86.
Embodiment 368. The method of embodiment 357, wherein the second antibody neutralizes the virus when expressed within the fusion protein but does not neutralize the virus when expressed as an isolated antibody.
Embodiment 369. The method of embodiment 357, wherein the second antibody neutralizes the virus when expressed within the fusion protein and neutralizes the virus when expressed as an isolated antibody.
Embodiment 370. The method of embodiment 357, wherein the second fusion protein further comprises an oligomerization domain.
Embodiment 371. The method of embodiment 370, wherein the oligomerization domain is a dimerization domain.
Embodiment 372. The method of embodiment 370, wherein the oligomerization domain is a trimerization domain.
Embodiment 373. The method of embodiment 370, wherein the oligomerization domain comprises the D4 trimerization domain of VSV-G protein.
Embodiment 374. The method of any one of embodiment s 307-373, wherein the antibody comprises the scFv and an amino acid sequence from at least one complementarity determining region of 80R.
Embodiment 375. The method of any one of embodiments 307-373, wherein the antibody comprises the scFv and an amino acid sequence from at least one complementarity determining region of H4.
Embodiment 376. The method of any one of embodiments 307-373, wherein the antibody comprises the scFv and an amino acid sequence from at least one complementarity determining region of 7D10.
Embodiment 377. The method of any one of embodiments 307-373, wherein the antibody comprises the scFv and an amino acid sequence from at least one complementarity determining region of COV2-2021.
Embodiment 378. The method of any one of embodiments 307-373, wherein the antibody comprises the scFv and an amino acid sequence from at least one complementarity determining region of C021.
Embodiment 379. The method of any one of embodiments 307-373, wherein the antibody comprises the scFv and an amino acid sequence from at least one complementarity determining region of C018.
Embodiment 380. The method of any one of embodiments 307-373, wherein the antibody comprises the scFv and an amino acid sequence from at least one complementarity determining region of H4 and the oligomerization domain comprises the D4 trimerization domain of VSV-G protein.
Embodiment 381. The method of any one of embodiments 307-373, wherein the antibody comprises the scFv and an amino acid sequence from at least one complementarity determining region of 1E01 and the oligomerization domain comprises the D4 trimerization domain of VSV-G protein.
Embodiment 382. The method of any one of embodiments 307-373, wherein the antibody comprises the scFv and an amino acid sequence from at least one complementarity determining region of F16 and the oligomerization domain comprises the D4 trimerization domain of VSV-G protein.
Embodiment 383. The method of any one of embodiments 307-373, wherein the antibody comprises the scFv and an amino acid sequence from at least one complementarity determining region of 0304-4A10, and the oligomerization domain comprises the D4 trimerization domain of VSV-G protein.
Embodiment 384. The method of any one of embodiments 307-373, wherein the antibody comprises the scFv and an amino acid sequence from at least one complementarity determining region of 2M-14E5, and the oligomerization domain comprises the D4 trimerization domain of VSV-G protein.
Embodiment 385. The method of any one of embodiments 307-373, wherein the antibody comprises the scFv and an amino acid sequence from at least one complementarity determining region of 9A1, and the oligomerization domain comprises the D4 trimerization domain of VSV-G protein.
Embodiment 386. The method of any one of embodiments 307-373, wherein the antibody comprises the scFv and an amino acid sequence from at least one complementarity determining region of COV2-2021, and the oligomerization domain comprises the D4 trimerization domain of VSV-G protein.
Embodiment 387. The method of any one of embodiments 307-373, wherein the antibody comprises the scFv and an amino acid sequence from at least one complementarity determining region of COV2-2026, and the oligomerization domain comprises the D4 trimerization domain of VSV-G protein.
Embodiment 388. The method of any one of embodiments 307-373, wherein the antibody comprises the scFv and an amino acid sequence from at least one complementarity determining region of COV2-2146, and the oligomerization domain comprises the D4 trimerization domain of VSV-G protein.
Embodiment 389. The method of any one of embodiments 307-373, wherein the antibody comprises the scFv and an amino acid sequence from at least one complementarity determining region of 2M-10B11, and the oligomerization domain comprises the D4 trimerization domain of VSV-G protein.
Embodiment 390. The method of any one of embodiments 307-373, wherein the antibody comprises the scFv and an amino acid sequence from at least one complementarity determining region of C021, and the oligomerization domain comprises the D4 trimerization domain of VSV-G protein.
Embodiment 391. The method of any one of embodiments 307-373, wherein the antibody comprises the scFv and an amino acid sequence from at least one complementarity determining region of C018, and the oligomerization domain comprises the D4 trimerization domain of VSV-G protein.
Embodiment 392. The method of any one of embodiments 307-373, wherein the multispecific antibody that comprises a tandem scFv format binds to a Neuraminidase active site and a Hemagglutinin stem of influenza virus and the fusion protein oligomerization domain comprises the D4 trimerization domain of VSV-G protein.
Embodiment 393. The method of any one of embodiments 307-373, wherein the multispecific antibody that comprises a tandem scFv format comprises an amino acid sequence from at least one complementarity determining region of 1E01 and an amino acid sequence from at least one complementarity determining region of F16 and the fusion protein oligomerization domain comprises the D4 trimerization domain of VSV-G protein.
Embodiment 394. The method of any one of embodiments 307-373, wherein the multispecific antibody that comprises a tandem scFv format binds to a Spike NTD and a Spike RBD of SARS COV-2 and the fusion protein oligomerization domain comprises the D4 trimerization domain of VSV-G protein.
Embodiment 395. The method of any one of embodiments 307-373, wherein the multispecific antibody that comprises a tandem scFv format comprises an amino acid sequence from at least one complementarity determining region of CoV2-2021 and an amino acid sequence from at least one complementarity determining region of C012 and the fusion protein oligomerization domain comprises the D4 trimerization domain of VSV-G protein.
Embodiment 396. The method of any one of embodiments 307-373, wherein the antibody comprises the scFv and an amino acid sequence from at least one complementarity determining region of 0304-4A10, and the oligomerization domain comprises the D4 trimerization domain of VSV-G protein, and the second antibody comprises the scFv and an amino acid sequence from at least one complementarity determining region of C018.
Embodiment 397. The method of any one of embodiments 307-396, wherein the antivirus is administered to the subject intravenously.
Embodiment 398. The method of any one of embodiments 307-396, wherein the antivirus is administered to the subject through inhalation.
Embodiment 399. The method of any one of embodiments 307-396, wherein the antivirus is administered to the subject through intranasal delivery.
Embodiment 400. The method of any one of embodiments 307-396, wherein the antivirus is administered to the subject through intratracheal delivery.
Embodiment 401. The method of any one of embodiments 307-396, wherein the antivirus is administered to the subject by an intraperitoneal injection.
Embodiment 402. The method of any one of embodiments 307-396, wherein the antivirus is administered to the subject by a subcutaneous injection.
Embodiment 403. The method of any one of embodiments 307-396, wherein the antivirus induces T cell mediated cytotoxicity against viral infected cells.
Embodiment 404. The method of any one of embodiments 307-396, wherein the administering to the subject of the antivirus is sufficient to reduce or eliminate the viral disease as compared to a baseline measurement of the viral disease taken from the subject prior to the administering of the antivirus.
Embodiment 405. The method of any one of embodiments 307-396, wherein the reduction is at least about 1-fold, 5-fold, 10-fold, 20-fold, 40-fold, 60-fold, 80-fold, or up to about 100-fold.
Embodiment 406. An antivirus comprising a fusion protein that comprises an amino acid sequence at least about 90% identical to that set forth in SEQ ID NOs: 53-72, 74-76, or 78, wherein the fusion protein is expressed at a valency of at least about 10 copies on a surface of the antivirus, wherein the fusion protein neutralizes a virus when the fusion protein is bound to a surface protein of the virus.
Embodiment 407. A composition comprising an antibody-based antivirus wherein the antibody-based antivirus comprises an enveloped particle that displays at least about 10 copies of an antibody on a surface of the antivirus, wherein the antibody binds to at least one surface protein of a virus, wherein one of the at least one surface protein of the virus comprises an oligomerized format, wherein the antibody neutralizes the virus when expressed on the surface of the antivirus, but does not neutralize the virus when expressed as an isolated antibody.
Embodiment 408. A composition comprising an antibody-based antivirus wherein the antibody-based antivirus comprises an enveloped particle that displays at least about 10 copies of an antibody on a surface of the antivirus, wherein the antibody comprises an oligomerized format and binds to at least one surface protein of a virus, wherein one of the at least one surface protein of the virus comprises an oligomerized format, wherein the antibody neutralizes the virus when expressed as an isolated antibody.

What is claimed is:

1. An antivirus comprising a fusion protein that comprises a transmembrane polypeptide and an antibody which binds to a surface protein of a virus wherein the fusion protein is expressed at a valency of at least about 10 copies on a surface of the antivirus, wherein the antibody neutralizes the virus when expressed within the fusion protein on the surface of the antivirus, but does not neutralize the virus when expressed as an isolated antibody.

2. The antivirus of claim 1, wherein the fusion protein further comprises an oligomerization domain.

3. The antivirus of claim 2, wherein the oligomerization domain is a dimerization domain, a trimerization domain, or a tetramerization domain.

4. The antivirus of claim 3, wherein the dimerization domain comprises a leucine zipper dimerization domain.

5. The antivirus of claim 3, wherein the trimerization domain comprises a post-fusion oligomerization domain of viral surface protein.

6. The antivirus of claim 3, wherein the trimerization domain comprises a D4 post-fusion trimerization domain of VSV-G protein.

7. The antivirus of claim 3, wherein the trimerization domain comprises a Dengue E protein post-fusion trimerization domain.

8. The antivirus of claim 3, wherein the trimerization domain comprises a foldon trimerization domain.

9. The antivirus of claim 3, wherein the tetramerization domain comprises an influenza neuraminidase stem domain.

10. The antivirus of claim 2, wherein the oligomerization domain comprises an amino acid sequence that is at least about 80%, 85%, 90%, 95%, or 99% identical to any one of SEQ ID NOs: 30-43.

11. The antivirus of claim 2, wherein when the fusion protein is expressed on the surface of the antivirus, the oligomerization domain is outside of the antivirus.

12. The antivirus of claim 2, wherein when the fusion protein is expressed on the surface of the antivirus, the oligomerization domain is outside of the antivirus and adjacent to a signal peptide.

13. The antivirus of claim 2, wherein when the fusion protein is expressed on the surface of the antivirus, the oligomerization domain is inside of the antivirus.

14. The antivirus of claim 2, wherein when the fusion protein is expressed on the surface of the antivirus, the oligomerization domain is inside of the antivirus and adjacent to the transmembrane polypeptide.

15. The antivirus of claim 1, wherein the fusion protein comprises a signal peptide.

16. The antivirus of claim 1, wherein domains of the fusion protein are arranged from the N-terminus to the C-terminus in the following orders:
(a) signal peptide, antibody which binds to a surface protein of a virus, oligomerization domain, transmembrane polypeptide, and cytosolic domain;
(b) signal peptide, antibody which binds to a surface protein of a virus, transmembrane polypeptide, oligomerization domain, and cytosolic domain; or
(c) signal peptide, oligomerization domain, antibody which binds to a surface protein of a virus, transmembrane polypeptide, and cytosolic domain.

17. The antivirus of claim 1, wherein the antibody comprises a single chain variable fragment (scFv), a single domain antibody, an Fv, a VH domain, a VL domain, a Fab fragment, a monoclonal antibody, F(ab′), F(ab′)2, single chain antibodies, diabodies, or a scFv-Fc.

18. The antivirus of claim 1, wherein the antibody binds specifically to the surface protein of the virus.

19. The antivirus of claim 1, wherein the antibody is a multispecific antibody.

20. The antivirus of claim 19, wherein the multispecific antibody binds specifically to more than one epitope on the surface protein of the virus.

21. The antivirus of claim 19, wherein the multispecific antibody comprises a tandem scFv format.

22. The antivirus of claim 1, wherein the virus comprises SARS COV-1, SARS COV-2, influenza, or MERS CoV virus.

23. The antivirus of claim 1, wherein the antibody comprises an amino acid sequence from at least one complementarity determining region of 0304-4A10, 2M-14E5, 9A1, COV2-2021, COV2-2026, COV2-2146, 2M-10B11, C021, C018 and BG4-5.

24. The antivirus of claim 1, wherein the antibody comprises an amino acid sequence at least about 80%, 85%, 90%, 95%, or 99% identical to any one of SEQ ID NOs: 6-14, 20-28 and 81-82.

25. The antivirus of claim 1, wherein the transmembrane polypeptide anchors the fusion protein to a bilayer of the antivirus.

26. The antivirus of claim 1, wherein the transmembrane polypeptide comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA).

27. The antivirus of claim 1, wherein the transmembrane polypeptide comprises a transmembrane domain comprising an amino acid sequence at least about 80%, 85%, 90%, 95%, or 99% identical to any one of SEQ ID NOs.: 44-52.

28. The antivirus of claim 1, wherein the transmembrane polypeptide comprises an amino acid sequence at least about 80%, 85%, 90%, 95%, or 99% identical to that set forth in SEQ ID NO: 29.

29. The antivirus of claim 1, wherein the fusion protein is expressed at a valency of about 10 copies on a surface of the antivirus.

30. The antivirus of claim 1, wherein the fusion protein is expressed at a valency of about 10 to 15 copies on a surface of the antivirus.

31. The antivirus of claim 1, wherein the fusion protein is expressed at a valency of at least about 25 copies on a surface of the antivirus.

32. The antivirus of claim 1, wherein the fusion protein is expressed at a valency of at least about 50 copies on a surface of the antivirus.

33. The antivirus of claim 1, wherein the fusion protein is expressed at a valency of at least about 100 copies on a surface of the antivirus.

34. The antivirus of claim 1, wherein the fusion protein is expressed at a valency of at least about 200 copies on a surface of the antivirus.

35. The antivirus of claim 1, wherein the fusion protein is expressed at a valency of at least about 400 copies on a surface of the antivirus.

36. The antivirus of claim 1, wherein the fusion protein is expressed at a valency of at least about 600 copies on a surface of the antivirus.

37. The antivirus of claim 1, wherein the fusion protein is expressed at a valency of at least about 1000 copies on a surface of the antivirus.

38. The antivirus of claim 1, wherein the antivirus is an enveloped particle.

39. The antivirus of claim 1, wherein the antivirus does not comprise viral genetic material.

40. The antivirus of claim 1, wherein the antivirus comprises a lipid bilayer.

41. The antivirus of claim 1, wherein the antivirus is a virus.

42. The antivirus of claim 1, wherein the antivirus is a replication incompetent virus.

43. The antivirus of claim 1, wherein the antivirus is a replication competent virus.

44. The antivirus of claim 1, wherein the antivirus is a viral-like particle.

45. The antivirus of claim 1, wherein the fusion protein comprises an amino acid sequence at least about 80%, 85%, 90%, 95%, or 99% identical to any one of SEQ ID NOs: 59-70, 72, 74, and 78.

46. The antivirus of claim 1, wherein the antivirus further comprises a second fusion protein that comprises transmembrane polypeptide and a second antibody which binds to a surface protein of the virus, wherein the second antibody comprises a CDR sequence that has less than 100% sequence identity to an equivalent CDR sequence of the antibody.

47. The antivirus of claim 46, wherein the fusion protein and the second fusion protein comprise the same transmembrane polypeptide.

48. The antivirus of claim 46, wherein the fusion protein and the second fusion protein comprise different transmembrane polypeptides.

49. The antivirus of claim 46, wherein the second antibody binds to the same surface protein as the antibody.

50. The antivirus of claim 46, wherein the second antibody binds to a different surface protein as the antibody.

51. The antivirus of claim 46, wherein the second antibody is a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc.

52. The antivirus of claim 46, wherein the second antibody binds specifically to the surface protein of the virus.

53. The antivirus of claim 46, wherein the second antibody is a multispecific antibody.

54. The antivirus of claim 53, wherein the second multispecific antibody binds specifically to more than one epitope on the surface protein of the virus.

55. The antivirus of claim 46, wherein the second antibody comprises an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15 and BG10-19.

56. The antivirus of claim 46, wherein the second antibody comprises an amino acid sequence at least about 80%, 85%, 90%, 95%, or 99% identical to any one of SEQ ID NOs: 1-28, and 81-86.

57. The antivirus of claim 46, wherein the second antibody neutralizes the virus when expressed within the fusion protein but does not neutralize the virus when expressed as an isolated antibody.

58. The antivirus of claim 46, wherein the second antibody neutralizes the virus when expressed within the fusion protein and neutralizes the virus when expressed as an isolated antibody.

59. The antivirus of claim 46, wherein the second fusion protein further comprises an oligomerization domain.

60. The antivirus of claim 59, wherein the oligomerization domain is a dimerization domain or a trimerization domain.

61. The antivirus of claim 59, wherein the oligomerization domain comprises the D4 trimerization domain of VSV-G protein.

62. The antivirus of claim 11, wherein the antibody comprises a single chain variable fragment (scFv) and an amino acid sequence from at least one complementarity determining region of 0304-4A10, 2M-14E5, 9A1, COV2-2021, COV2-2026, COV2-2146, 2M-10B11, C021, C018, and BG4-5.

63. The antivirus of claim 46, wherein the second fusion protein comprises an amino acid sequence at least about 80%, 85%, 90%, 95%, or 99% identical to any one of SEQ ID NOs: 53-72, 74-76, or 78.

64. The antivirus of claim 1, wherein:
(a) the antibody comprises a scFv and an amino acid sequence from at least one complementarity determining region of 0304-4A10, 2M-14E5, 9A1, COV2-2021, COV2-2026, COV2-2146, 2M-10B11, C021, C018 and BG4-5; and
(b) the transmembrane polypeptide comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA).

65. The antivirus of claim 1, wherein:
(a) the antibody comprises a scFv and an amino acid sequence from at least one complementarity determining region of 0304-4A10, 2M-14E5, 9A1, COV2-2021, COV2-2026, COV2-2146, 2M-10B11, C021, C018 and BG4-5;
(b) the transmembrane polypeptide comprises a transmembrane domain comprising an amino acid sequence at least about 80%, 85%, 90%, 95%, or 99% identical to one of SEQ ID NOs.: 44-52.

66. The antivirus of claim 1, wherein:
(a) the antibody comprises a scFv and an amino acid sequence at least about 80%, 85%, 90%, 95%, or 99% identical to any one of SEQ ID NOs: 6-14, 20-28 and 81-82; and
(b) the transmembrane polypeptide comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA).

67. The antivirus of claim 2, wherein:
(a) the antibody comprises a scFv and an amino acid sequence from at least one complementarity determining region of 0304-4A10, 2M-14E5, 9A1, COV2-2021, COV2-2026, COV2-2146, 2M-10B11, C021, C018 and BG4-5; and
(b) the oligomerization domain comprises a leucine zipper dimerization domain, a D4 trimerization domain of VSV-G protein, a Dengue E protein post-fusion trimerization domain, a foldon trimerization domain, or an influenza neuraminidase stem tetramerization domain.

68. The antivirus of claim 2, wherein:
(a) the antibody comprises a single chain variable fragment (scFv) and an amino acid sequence from at least one complementarity determining region of 0304-4A10, 2M-14E5, 9A1, COV2-2021, COV2-2026, COV2-2146, 2M-10B11, C021, C018 and BG4-5; and
(b) the oligomerization domain comprises an amino acid sequence at least about 80%, 85%, 90%, 95%, or 99% identical to any one of SEQ ID NOs: 30-43.

69. The antivirus of claim 2, wherein:
(a) the antibody comprises a scFv and an amino acid sequence at least about 80%, 85%, 90%, 95%, or 99% identical to any one of SEQ ID NOs: 6-14, 20-28 and 81-82; and
(b) the oligomerization domain comprises a leucine zipper dimerization domain, a D4 trimerization domain of VSV-G protein, a Dengue E protein post-fusion trimerization domain, a foldon trimerization domain, or an influenza neuraminidase stem tetramerization domain.

70. The antivirus of claim 2, wherein:
(a) the antibody comprises a scFv and an amino acid sequence from at least one complementarity determining region of 0304-4A10, 2M-14E5, 9A1, COV2-2021, COV2-2026, COV2-2146, 2M-10B11, C021, C018 and BG4-5; and
(b) the transmembrane polypeptide comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA); and
(c) the oligomerization domain comprises a leucine zipper dimerization domain, a D4 trimerization domain of VSV-G protein, a Dengue E protein post-fusion trimerization domain, a foldon trimerization domain, or an influenza neuraminidase stem tetramerization domain.

71. The antivirus of claim 2, wherein:
(a) the antibody comprises a scFv and an amino acid sequence from at least one complementarity determining region of 0304-4A10, 2M-14E5, 9A1, COV2-2021, COV2-2026, COV2-2146, 2M-10B11, C021, C018 and BG4-5;
(b) the transmembrane polypeptide comprises a transmembrane domain comprising an amino acid sequence at least about 80%, 85%, 90%, 95%, or 99% identical to one of SEQ ID NOs.: 44-52; and
(c) the oligomerization domain comprises a leucine zipper dimerization domain, a D4 trimerization domain of VSV-G protein, a Dengue E protein post-fusion trimerization domain, a foldon trimerization domain, an influenza neuraminidase stem tetramerization domain.

72. The antivirus of claim 2, wherein:
(a) the antibody comprises a scFv and an amino acid sequence from at least one complementarity determining region of 0304-4A10, 2M-14E5, 9A1, COV2-2021, COV2-2026, COV2-2146, 2M-10B11, C021, C018 and BG4-5;
(b) the transmembrane polypeptide comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA); and
(c) the oligomerization domain comprises an amino acid sequence at least about 80%, 85%, 90%, 95%, or 99% identical to any one of SEQ ID NOs: 30-43.

73. The antivirus of claim 2, wherein:
(a) the antibody comprises a scFv and an amino acid sequence from at least one complementarity determining region of 0304-4A10, 2M-14E5, 9A1, COV2-2021, COV2-2026, COV2-2146, 2M-10B11, C021, C018 and BG4-5;
(b) the transmembrane polypeptide comprises a transmembrane domain comprising an amino acid sequence at least about 80%, 85%, 90%, 95%, or 99% identical to one of SEQ ID NOs.: 44-52; and
(c) the oligomerization domain comprises an amino acid sequence at least about 80%, 85%, 90%, 95%, or 99% identical to any one of SEQ ID NOs: 30-43.

74. The antivirus of claim 2, wherein:
(a) the antibody comprises a scFv and an amino acid sequence at least about 80%, 85%, 90%, 95%, or 99% identical to any one of SEQ ID NOs: 6-14, 20-28 and 81-82;
(b) the transmembrane polypeptide comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA); and
(c) the oligomerization domain comprises a leucine zipper dimerization domain, a D4 trimerization domain of VSV-G protein, a Dengue E protein post-fusion trimerization domain, a foldon trimerization domain, an influenza neuraminidase stem tetramerization domain.

75. The antivirus of claim 2, wherein:
(a) the antibody comprises a scFv and an amino acid sequence at least about 80%, 85%, 90%, 95%, or 99% identical to any one of SEQ ID NOs: 6-14, 20-28 and 81-82;
(b) the transmembrane polypeptide comprises a transmembrane domain comprising an amino acid sequence at least about 80%, 85%, 90%, 95%, or 99% identical to one of SEQ ID NOs.: 44-52; and
(c) the oligomerization domain comprises a leucine zipper dimerization domain, a D4 trimerization domain of VSV-G protein, a Dengue E protein post-fusion trimerization domain, a foldon trimerization domain, an influenza neuraminidase stem tetramerization domain.

76. The antivirus of claim 2, wherein:
(a) the antibody comprises a scFv and an amino acid sequence at least about 80%, 85%, 90%, 95%, or 99% identical to any one of SEQ ID NOs: 6-14, 20-28 and 81-82;
(b) the transmembrane polypeptide comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA); and
(c) the oligomerization domain comprises an amino acid sequence at least about 80%, 85%, 90%, 95%, or 99% identical to any one of SEQ ID NOs: 30-43.

77. The antivirus of claim 46, wherein:
(a) the second antibody comprises a scFv and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19; and
(b) the transmembrane polypeptide of the second fusion protein comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA).

78. The antivirus of claim 46, wherein:
(a) the second antibody comprises a scFv and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19; and
(b) the transmembrane polypeptide of the second fusion protein comprises a transmembrane domain comprising an amino acid sequence at least about 80%, 85%, 90%, 95%, or 99% identical to one of SEQ ID NOs.: 44-52.

79. The antivirus of claim 46, wherein:
(a) the second antibody comprises a scFv and an amino acid sequence at least about 80%, 85%, 90%, 95%, or 99% identical to any one of SEQ ID NOs: 1-28; and
(b) the transmembrane polypeptide of the second fusion protein comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA).

80. The antivirus of claim 59, wherein:
(a) the second antibody comprises a scFv and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19; and
(b) the oligomerization domain of the second fusion protein comprises a leucine zipper dimerization domain, a D4 trimerization domain of VSV-G protein, a Dengue E protein post-fusion trimerization domain, a foldon trimerization domain, or an influenza neuraminidase stem tetramerization domain.

81. The antivirus of claim 59, wherein:
(a) the second antibody comprises a single chain variable fragment (scFv) and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19; and
(b) the oligomerization domain of the second fusion protein comprises an amino acid sequence at least about 80%, 85%, 90%, 95%, or 99% identical to any one of SEQ ID NOs: 30-43.

82. The antivirus of claim 59, wherein:
(a) the second antibody comprises a scFv and an amino acid sequence at least about 80%, 85%, 90%, 95%, or 99% identical to any one of SEQ ID NOs: 1-28; and
(b) the oligomerization domain of the second fusion protein comprises a leucine zipper dimerization domain, a D4 trimerization domain of VSV-G protein, a Dengue E protein post-fusion trimerization domain, a foldon trimerization domain, or an influenza neuraminidase stem tetramerization domain.

83. The antivirus of claim 59, wherein:
(a) the second antibody comprises a scFv and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19; and
(b) the transmembrane polypeptide of the second fusion protein comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA); and
(c) the oligomerization domain of the second fusion protein comprises a leucine zipper dimerization domain, a D4 trimerization domain of VSV-G protein, a Dengue E protein post-fusion trimerization domain, a foldon trimerization domain, or an influenza neuraminidase stem tetramerization domain.

84. The antivirus of claim 59, wherein:
(a) the second antibody comprises a scFv and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19;
(b) the transmembrane polypeptide of the second fusion protein comprises a transmembrane domain comprising an amino acid sequence at least about 80%, 85%, 90%, 95%, or 99% identical to one of SEQ ID NOs.: 44-52; and
(c) the oligomerization domain of the second fusion protein comprises a leucine zipper dimerization domain, a D4 trimerization domain of VSV-G protein, a Dengue E protein post-fusion trimerization domain, a foldon trimerization domain, an influenza neuraminidase stem tetramerization domain.

85. The antivirus of claim 59, wherein:
(a) the second antibody comprises a scFv and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19;
(b) the transmembrane polypeptide of the second fusion protein comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA); and
(c) the oligomerization domain of the second fusion protein comprises an amino acid sequence at least about 80%, 85%, 90%, 95%, or 99% identical to any one of SEQ ID NOs: 30-43.

86. The antivirus of claim 59, wherein:
(a) the second antibody comprises a scFv and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19;
(b) the transmembrane polypeptide of the second fusion protein comprises a transmembrane domain comprising an amino acid sequence at least about 80%, 85%, 90%, 95%, or 99% identical to one of SEQ ID NOs.: 44-52; and
(c) the oligomerization domain of the second fusion protein comprises an amino acid sequence at least about 80%, 85%, 90%, 95%, or 99% identical to any one of SEQ ID NOs: 30-43.

87. The antivirus of claim 59, wherein:
(a) the second antibody comprises a scFv and an amino acid sequence at least about 80%, 85%, 90%, 95%, or 99% identical to any one of SEQ ID NOs: 1-28 and 81-86;
(b) the transmembrane polypeptide of the second fusion protein comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA); and
(c) the oligomerization domain of the second fusion protein comprises a leucine zipper dimerization domain, a D4 trimerization domain of VSV-G protein, a Dengue E protein post-fusion trimerization domain, a foldon trimerization domain, an influenza neuraminidase stem tetramerization domain.

88. The antivirus of claim 59, wherein:
(a) the second antibody comprises a scFv and an amino acid sequence at least about 80%, 85%, 90%, 95%, or 99% identical to any one of SEQ ID NOs: 1-28 and 81-86;
(b) the transmembrane polypeptide of the second fusion protein comprises a transmembrane domain comprising an amino acid sequence at least about 80%, 85%, 90%, 95%, or 99% identical to one of SEQ ID NOs.: 44-52; and
(c) the oligomerization domain of the second fusion protein comprises a leucine zipper dimerization domain, a D4 trimerization domain of VSV-G protein, a Dengue E protein post-fusion trimerization domain, a foldon trimerization domain, an influenza neuraminidase stem tetramerization domain.

89. The antivirus of claim 59, wherein:
(a) the second antibody comprises a scFv and an amino acid sequence at least about 80%, 85%, 90%, 95%, or 99% identical to any one of SEQ ID NOs: 1-28 and 81-86;
(b) the transmembrane polypeptide of the second fusion protein comprises a transmembrane domain of a Vesicular Stomatitis virus glycoprotein (VSV-G), influenza Hemagglutinin (HA), HIV surface glycoprotein GP120 or GP41, measles virus surface glycoprotein hamagglutinin (H) protein, or influenza Neuraminidase (NA); and
(c) the oligomerization domain of the second fusion protein comprises an amino acid sequence at least about 80%, 85%, 90%, 95%, or 99% identical to any one of SEQ ID NOs: 30-43.

90. The antivirus of claim 19, wherein the antibody is multispecific antibody that comprises a tandem scFv format comprising an amino acid sequence from at least one complementarity determining region of 0304-4A10, 2M-14E5, 9A1, COV2-2021, COV2-2026, COV2-2146, 2M-10B11, C021, C018 and BG4-5 and an amino acid sequence from at least one complementarity determining region of 0304-4A10, 2M-14E5, 9A1, COV2-2021, COV2-2026, COV2-2146, 2M-10B11, C021, C018 and BG4-5 and the fusion protein oligomerization domain comprises the D4 trimerization domain of VSV-G protein.

91. The antivirus of claim 53, wherein the second antibody is multispecific antibody that comprises a tandem scFv format comprising an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19 and an amino acid sequence from at least one complementarity determining region of 80R, H4, 7D10, 1E01, FI6, 0304-4A10, 2M-14E5, 9A1, COV2-2021, COV2-2026, COV2-2146, 2M-10B11, C021, C018, BG4-5, BG7-15, and BG10-19 and the second fusion protein oligomerization domain comprises the D4 trimerization domain of VSV-G protein.

92. A composition comprising an antibody-based antivirus wherein the antibody-based antivirus comprises an enveloped particle that displays at least about 10 copies of an antibody on a surface of the antivirus, wherein the antibody binds to at least one surface protein of a virus, wherein one of the at least one surface protein of the virus comprises an oligomerized format, wherein the antibody neutralizes the virus when expressed on the surface of the antivirus, but does not neutralize the virus when expressed as an isolated antibody.

93. A composition comprising an antibody-based antivirus wherein the antibody-based antivirus comprises an enveloped particle that displays at least about 10 copies of an antibody on a surface of the antivirus, wherein the antibody comprises an oligomerized format and binds to at least one surface protein of a virus, wherein one of the at least one surface protein of the virus comprises an oligomerized format, wherein the antibody neutralizes the virus when expressed as an isolated antibody.


SEQUENCE LISTING (87 sequences)

SEQ ID NO: 1 - protein, 117 aa, Artificial Sequence
  Description of Artificial Sequence: Synthetic polypeptide
EVQLVQSGGGVVQPGKSLRLSCAASGFAFSSYAMHWVRQAPGKGLEWVAVISYDGSNKYYADSVKGRFTISRDNSKNTLYLQMNSLRAEDTAVYYCARDRSYYLDYWGQGTLVTVSS

SEQ ID NO: 2 - protein, 126 aa, Artificial Sequence
  Description of Artificial Sequence: Synthetic polypeptide
QVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARVPYCSSTSCHRDWYFDLWGRGTLVTVSS

SEQ ID NO: 3 - protein, 120 aa, Artificial Sequence
  Description of Artificial Sequence: Synthetic polypeptide
EVQLVESGAEVVKPGASVKMSCKASGYPFTSYNIHWIKQTPGQGLEWIGAIYPGNGDTSYTQKFKVKATLTSDKSSSTAYMQLSSLTSEDSAVYFCARYGNYPSYAMDYWGQGTSVTVSS

SEQ ID NO: 4 - protein, 130 aa, Artificial Sequence [sequence omitted]

SEQ ID NO: 5 - protein, 226 aa, Artificial Sequence
  Description of Artificial Sequence: Synthetic polypeptide
QVQLVESGGGVVQPGRSLRLSCAASGFTFSTYAMHWVRQAPGKGLEWVAVISYDANYKYYADSVKGRFTISRDNSKNTLYLQMNSLRAEDTAVYYCAKDSQLRSLLYFEWLSQGYFDYWGQGTLVTVSSASTKGPSVFPLAPSSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 6 - protein, 117 aa, Artificial Sequence
  Description of Artificial Sequence: Synthetic polypeptide
EVQLVESGGGLVQPGGSLRLSCAASGFTFSTYAMHWVRQAPGKGLEYVSGISSNGGSTYYANSVKGRFTISRDNSKNTLYIQMGSLRAEDMAVYYCARSSSRGFDYWGQGTLVTVSS

SEQ ID NO: 7 - protein, 121 aa, Artificial Sequence
  Description of Artificial Sequence: Synthetic polypeptide
EVQLVESGGGVVQPGRSLRLSCAASGFTFSTYSMHWVRQAPGKGLEWVAVISYDGSNKYYADSVKGRFTISRDNSKNTLYLQVNSLRAEDTAVYYCARSGGGSYRGPFDYWGQGTLVTVSS

SEQ ID NO: 8 - protein, 125 aa, Artificial Sequence
  Description of Artificial Sequence: Synthetic polypeptide
EVQLLESGGGVVQPGRSLRLSCVVSGFTFNNYGMHWVRQAPGKGLEWVAVISYEGSVKYYGDHVDGRFTISRDPFKNTLYLHMNNLRPDDTAVYYCAKVSAIFWLGQGLSPIDVWGQGTTVTVSS

SEQ ID NO: 9 - protein, 121 aa, Artificial Sequence
  Description of Artificial Sequence: Synthetic polypeptide
QVQLVQSGAEVKKPGASVKVSCKVSGYTLIELSIHWVRQAPGKGLEWMGGFDPEDVETIYAQKFQGRVTMTEDTSTDTAYMELSSLTSEDRAVYYCATQPAAIGGTPPYYWGQGTLVTVSS

SEQ ID NO: 10 - protein, 128 aa, Artificial Sequence
  Description of Artificial Sequence: Synthetic polypeptide
QVQLVQSGAEVKKPGASVKVSCKVSGYTLTELSIHWVRQAPGKGLEWMGGFDPEDGETVYAQKFQGRVTMTEDTSSDTAYMELSSLRSEDTAVYYCATSFPIRGDPSYYYYYYGMDVWGQGTTVTVSS

SEQ ID NO: 11 - protein, 124 aa, Artificial Sequence
  Description of Artificial Sequence: Synthetic polypeptide
EVQLVESGGGLVQPGGSLRLSCEASGFTFSSSEINWVRQAPGKGLEWVSHISSSGSIIYYADSVKGRFTISRDNAKNSLYLQMNSLRAEDTAVYYCARRSYRSSWYYYYGMDVWGQGTTVTVSS

SEQ ID NO: 12 - protein, 118 aa, Artificial Sequence
  Description of Artificial Sequence: Synthetic polypeptide
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCARATWLRGVMDVWGQGTTVTVSS

SEQ ID NO: 13 - protein, 123 aa, Artificial Sequence
  Description of Artificial Sequence: Synthetic polypeptide
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARVWQYYDSSGSFDYWGQGTLVTVSS

SEQ ID NO: 14 - protein, 121 aa, Artificial Sequence
  Description of Artificial Sequence: Synthetic polypeptide
EVQLVESGGGVVQPGRSLRLSCAASGFTFSNYAIHWVRQAPGKGLEWVAVISYDGSNKYYADSVKGRFTISRDNSKNTLYLQMNSLRAEDTAVYYCARDFDDSSFWAFDYWGQGTLVTVSS

SEQ ID NO: 15 - protein, 113 aa, Artificial Sequence
  Description of Artificial Sequence: Synthetic polypeptide
ETTLTQSPATLSLSPGERATLSCRASQSVRSNLAWYQQKPGQAPRPLIYDASTRATGIPDRFSGSGSGTDFTLTISRLEPEDFAVYYCQQRSNWPPTFGQGTKVEVKSGLVPR

SEQ ID NO: 16 - protein, 113 aa, Artificial Sequence
  Description of Artificial Sequence: Synthetic polypeptide
DIQMTQSPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPLTFGGGTKVEIK

SEQ ID NO: 17 - protein, 111 aa, Artificial Sequence
  Description of Artificial Sequence: Synthetic polypeptide
DIVMTQSPASLTVSLGQRATISCRASKSVSASGYNYLHWYQQRPGQPPKLLIYLAFNLESGVPARFNGSGSGTDFTLNIHPVEEEDAATYYCQHSRDLPFTFGSGTKLEIK

SEQ ID NO: 18 - protein, 108 aa, Artificial Sequence
  Description of Artificial Sequence: Synthetic polypeptide
DIQLTQSPSFLSASVGDRVTITCRASQDISSYLAWYQQKPGNAPKLLIYAASLLQSGVPSRFSAFGSGTEFTLTISSLQPEDFATYYCQHLKSYPLFTFGPGTKVDIK

SEQ ID NO: 19 - protein, 218 aa, Artificial Sequence
  Description of Artificial Sequence: Synthetic polypeptide
DIVMTQSPDSLAVSLGERATINCKSSQSVTFNYKNYLAWYQQKPGQPPKLLIYWASTRESGVPDRFSGSGSGTDFTLTISSLQAEDVAVYYCQQHYRTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 113 aa, Artificial Sequence
  Description of Artificial Sequence: Synthetic polypeptide
EIVLTQSPDSLAVSLGERATINCRSSQSVLYSSNNKNYLAWYQQKPGQPPKVLIYWASTRESGVPDRFSGSGSGTDFTLTISSLQAEDVAVYYCQQYYSSPYAFGPGTKVDIK

SEQ ID NO: 21 - protein, 106 aa, Artificial Sequence
  Description of Artificial Sequence: Synthetic polypeptide
AIRMTQSPSFLSVSVGDRVTITCRASQGISSYLAWYQQKPGKAPKLLIYAASTLQSGVPSRFSGSGSGTEFTLTISSLQPEDFATYYCQQLNSYVTFGGGTKVEIK

SEQ ID NO: 22 - protein, 107 aa, Artificial Sequence
  Description of Artificial Sequence: Synthetic polypeptide
DIVMTQSPATLSASPGERVTLSCRASQNIRNNLAWYQQKPGQAPRLLIHGASTRAAGAPARFSGSGSDTQFTLTVSSLQSEDFAVYYCHQYSKWPVTFGGGTKVDIK

SEQ ID NO: 23 - protein, 111 aa, Artificial Sequence
  Description of Artificial Sequence: Synthetic polypeptide
QSVLTQPPSASGTPGQRVTISCSGSSSNIGSNYVYWYQQLPGTAPKLLIYRNNQRPSGVPDRFSGSKSGTSASLAISGLRSEDEADYYCAAWDASLSGHVVFGGGTKLTVL

SEQ ID NO: 24 - protein, 107 aa, Artificial Sequence
  Description of Artificial Sequence: Synthetic polypeptide
DIQMTQSPSSLSASVGDRVTITCRASQGITNYLAWFQQKPGKAPKSLIYAVSSLQSGVPSKFSGSGSGTDFTLTISSLQPEDFATYYCQQYNSYPWTFGQGTKVEIK

SEQ ID NO: 25 - protein, 107 aa, Artificial Sequence
  Description of Artificial Sequence: Synthetic polypeptide
DIQLTQSPSFLSASVGDRVTITCRASQGISSYLAWYQQKPGKAPKLLIYAASTLQSGVPSRFSGSGSGTEFTLTISSLQPEDFATYYCQQLNSYPVTFGQGTKVEIK

SEQ ID NO: 26 - protein, 111 aa, Artificial Sequence
  Description of Artificial Sequence: Synthetic polypeptide
QSALTQPHSVSESPGKTVTISCTGSSGSIASNYVQWYQQRPGSAPTTVIYEDNQRPSGVPDRFSGSIDSSSNSASLTISGLKTEDEADYYCQSYDSSNHWVFGGGTKLTVL

SEQ ID NO: 27 - protein, 112 aa, Artificial Sequence
  Description of Artificial Sequence: Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGPGTKVDIK

SEQ ID NO: 28 - protein, 108 aa, Artificial Sequence
  Description of Artificial Sequence: Synthetic polypeptide
DIQLTQSPSSLSASVGDRVTITCRASQSIRSYLNWYQQKPGKAPKLLIYAASSLQSGVPSRFSGSGSGTDFTLTISSLQPDDFATYYCQQSYSTPPATFGQGTKLEIK

SEQ ID NO: 29 - protein, 495 aa, Vesiculovirus indiana
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 30 - protein, 150 aa, Artificial Sequence
  Description of Artificial Sequence: Synthetic polypeptide
IGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDI

SEQ ID NO: 31 - protein, 130 aa, Artificial Sequence
  Description of Artificial Sequence: Synthetic polypeptide
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNL

SEQ ID NO: 32 - protein, 115 aa, Artificial Sequence
  Description of Artificial Sequence: Synthetic polypeptide
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTT

SEQ ID NO: 33 - protein, 100 aa, Artificial Sequence
  Description of Artificial Sequence: Synthetic polypeptide
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFING

SEQ ID NO: 34 - protein, 84 aa, Artificial Sequence
  Description of Artificial Sequence: Synthetic polypeptide
IQADGWMCHASKWVTTCDFRWYGPKYITHSIRSFTPSVEQCKESIEQTKQGTWLNPGFPPQSCGYATVTDAEAVIVQVTPHHVL

SEQ ID NO: 35 - protein, 27 aa, Artificial Sequence
  Description of Artificial Sequence: Synthetic peptide
GYIPEAPRDGQAYVRKDGEWVLLSTFL

SEQ ID NO: 36 - protein, 33 aa, Artificial Sequence
  Description of Artificial Sequence: Synthetic polypeptide
RMKQLEDKVEELLSKQYHLENEVARLKKLVGER

SEQ ID NO: 37 - protein, 33 aa, Artificial Sequence
  Description of Artificial Sequence: Synthetic polypeptide
RMKQLEDKVEELLSKNYHLENEVARLKKLVGER

SEQ ID NO: 38 - protein, 40 aa, Artificial Sequence
  Description of Artificial Sequence: Synthetic polypeptide
MNPNQKIITIGSICLVVGLISLILQIGNIISIWISHSIQT

SEQ ID NO: 39 - protein, 75 aa, Artificial Sequence
  Description of Artificial Sequence: Synthetic polypeptide
MNPNQKIITIGSICMVTGIVSLMLQIGNMISIWVSHSIHTGNQHQSEPISNTNFLTEKAVASVKLAGNSSLCPIN

SEQ ID NO: 40 - protein, 68 aa, Artificial Sequence
  Description of Artificial Sequence: Synthetic polypeptide
KLCIEAKISNTTTDSRCPTQGEATLVEEQDTNFVCRRTFVDRGHGNGCGLFGKGSLITCAKFKCVTKL

SEQ ID NO: 41 - protein, 83 aa, Artificial Sequence
  Description of Artificial Sequence: Synthetic polypeptide
IELLKTEVTNPAVLRKLCIEAKISNTTTDSRCPTQGEATLVEEQDTNFVCRRTFVDRGHGNGCGLFGKGSLITCAKFKCVTKL

SEQ ID NO: 42 - protein, 83 aa, Artificial Sequence
  Description of Artificial Sequence: Synthetic polypeptide
KLCIEAKISNTTTDSRCPTQGEATLVEEQDTNFVCRRTFVDRGHGNGCGLFGKGSLITCAKFKCVTKLEGKIVQYENLKYSVI

SEQ ID NO: 43 - protein, 59 aa, Artificial Sequence
  Description of Artificial Sequence: Synthetic polypeptide
EAKISNTTTDSRCPTQGEATLVEEQDTNFVCRRTFVDRGHGNGCGLFGKGSLITCAKFK

SEQ ID NO: 44 - protein, 22 aa, Vesiculovirus indiana
IASFFFIIGLIIGLFLVLRVGI

SEQ ID NO: 45 - protein, 37 aa, Vesiculovirus indiana
PIELVEGWFSSWKSSIASFFFIIGLIIGLFLVLRVGI

SEQ ID NO: 46 - protein, 53 aa, Vesiculovirus indiana
DDESLFFGDTGLSKNPIELVEGWFSSWKSSIASFFFIIGLIIGLFLVLRVGIH

SEQ ID NO: 47 - protein, 81 aa, Vesiculovirus indiana
GMLDSDLHLSSKAQVFEHPHIQDAASQLPDDESLFFGDTGLSKNPIELVEGWFSSWKSSIASFFFIIGLIIGLFLVLRVGI

SEQ ID NO: 48 - protein, 25 aa, Vesiculovirus indiana
HLCIKLKHTKKRQIYTDIEMNRLGK

SEQ ID NO: 49 - protein, 23 aa, Influenza A virus
IITIGSVCMTIGMANLILQIGNI

SEQ ID NO: 50 - protein, 23 aa, Influenza A virus
LAIYSTVASSLVLVVSLGAISFW

SEQ ID NO: 51 - protein, 52 aa, Dengue virus
AYGVLFSGVSWTMKIGIGILLTWLGLNSRSTSLSMTCIAVGMVTLYLGVMVQ

SEQ ID NO: 52 - protein, 21 aa, Human immunodeficiency virus 1
FIMIVGGLVGLRIVFAVLSIV

SEQ ID NO: 53 - protein, 372 aa, Artificial Sequence
  Description of Artificial Sequence: Synthetic polypeptide
MEFGLSWVFLVALFRGVQSEVQLVQSGGGVVQPGKSLRLSCAASGFAFSSYAMHWVRQAPGKGLEWVAVISYDGSNKYYADSVKGRFTISRDNSKNTLYLQMNSLRAEDTAVYYCARDRSYYLDYWGQGTLVTVSSGGGGSGGGGSGGGGSETTLTQSPATLSLSPGERATLSCRASQSVRSNLAWYQQKPGQAPRPLIYDASTRATGIPDRFSGSGSGTDFTLTISRLEPEDFAVYYCQQRSNWPPTFGQGTKVEVKSGLVPRRSGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 54 - protein, 381 aa, Artificial Sequence
  Description of Artificial Sequence: Synthetic polypeptide
MEFGLSWVFLVALFRGVQSQVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARVPYCSSTSCHRDWYFDLWGRGTLVTVSSGGGGSGGGGSGGGGSDIQMTQSPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPLTFGGGTKVEIKSRGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 55 - protein, 511 aa, Artificial Sequence
  Description of Artificial Sequence: Synthetic polypeptide
MEFGLSWVFLVALFRGVQSQVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARVPYCSSTSCHRDWYFDLWGRGTLVTVSSGGGGSGGGGSGGGGSDIQMTQSPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPLTFGGGTKVEIKDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 56 - protein, 370 aa, Artificial Sequence
  Description of Artificial Sequence: Synthetic polypeptide
MEFGLSWVFLVALFRGVQSDIVMTQSPASLTVSLGQRATISCRASKSVSASGYNYLHWYQQRPGQPPKLLIYLAFNLESGVPARFNGSGSGTDFTLNIHPVEEEDAATYYCQHSRDLPFTFGSGTKLEIKGGGSGGGSGGGSEVQLVESGAEVVKPGASVKMSCKASGYPFTSYNIHWIKQTPGQGLEWIGAIYPGNGDTSYTQKFKVKATLTSDKSSSTAYMQLSSLTSEDSAVYFCARYGNYPSYAMDYWGQGTSVTVSSSRGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 57 - protein, 515 aa, Artificial Sequence
  Description of Artificial Sequence: Synthetic polypeptide
MEFGLSWVFLVALFRGVQSEVQLVESGGRVVRPGGSLRLSCAASGFTFDDYGMSWVRQPPGKGLEFVSGLNWNGDITAFTDSVKGRFTISRDNVKSSLYLQMNSLRADDTAFYYCARVRTWGDYTTGEEIINSWYFDLWGRGTLVTVSSGGGGSGGGGSGGGGSGGGGSDIQLTQSPSFLSASVGDRVTITCRASQDISSYLAWYQQKPGNAPKLLIYAASLLQSGVPSRFSAFGSGTEFTLTISSLQPEDFATYYCQHLKSYPLFTFGPGTKVDIKDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 58 - protein, 721 aa, Artificial Sequence
  Description of Artificial Sequence: Synthetic polypeptide
MEFGLSWVFLVALFRGVQSQVQLVESGGGVVQPGRSLRLSCAASGFTFSTYAMHWVRQAPGKGLEWVAVISYDANYKYYADSVKGRFTISRDNSKNTLYLQMNSLRAEDTAVYYCAKDSQLRSLLYFEWLSQGYFDYWGQGTLVTVSSASTKGPSVFPLAPSSGGTAALGCLVKDYFPEPVTVSWNSGALTSGVHTFPAVLQSSGLYSLSSVVTVPSSSLGTQTYICNVNHKPSNTKVDKRVEPKGGGGSGGGGSGGGGSGGGGSDIVMTQSPDSLAVSLGERATINCKSSQSVTFNYKNYLAWYQQKPGQPPKLLIYWASTRESGVPDRFSGSGSGTDFTLTISSLQAEDVAVYYCQQHYRTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 59 - protein, 507 aa, Artificial Sequence
  Description of Artificial Sequence: Synthetic polypeptide
MEFGLSWVFLVALFRGVQSEVQLVESGGGLVQPGGSLRLSCAASGFTFSTYAMHWVRQAPGKGLEYVSGISSNGGSTYYANSVKGRFTISRDNSKNTLYIQMGSLRAEDMAVYYCARSSSRGFDYWGQGTLVTVSSGGGGSGGGGSGGGGSGGGGSEIVLTQSPDSLAVSLGERATINCRSSQSVLYSSNNKNYLAWYQQKPGQPPKVLIYWASTRESGVPDRFSGSGSGTDFTLTISSLQAEDVAVYYCQQYYSSPYAFGPGTKVDIKDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 60 - protein, 504 aa, Artificial Sequence
  Description of Artificial Sequence: Synthetic polypeptide
MEFGLSWVFLVALFRGVQSEVQLVESGGGVVQPGRSLRLSCAASGFTFSTYSMHWVRQAPGKGLEWVAVISYDGSNKYYADSVKGRFTISRDNSKNTLYLQVNSLRAEDTAVYYCARSGGGSYRGPFDYWGQGTLVTVSSGGGGSGGGGSGGGGSGGGGSAIRMTQSPSFLSVSVGDRVTITCRASQGISSYLAWYQQKPGKAPKLLIYAASTLQSGVPSRFSGSGSGTEFTLTISSLQPEDFATYYCQQLNSYVTFGGGTKVEIKDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 61 - protein, 509 aa, Artificial Sequence
  Description of Artificial Sequence: Synthetic polypeptide
MEFGLSWVFLVALFRGVQSEVQLLESGGGVVQPGRSLRLSCVVSGFTFNNYGMHWVRQAPGKGLEWVAVISYEGSVKYYGDHVDGRFTISRDPFKNTLYLHMNNLRPDDTAVYYCAKVSAIFWLGQGLSPIDVWGQGTTVTVSSGGGGSGGGGSGGGGSGGGGSDIVMTQSPATLSASPGERVTLSCRASQNIRNNLAWYQQKPGQAPRLLIHGASTRAAGAPARFSGSGSDTQFTLTVSSLQSEDFAVYYCHQYSKWPVTFGGGTKVDIKDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 62 - protein, 378 aa, Artificial Sequence
  Description of Artificial Sequence: Synthetic polypeptide
MEFGLSWVFLVALFRGVQSQVQLVQSGAEVKKPGASVKVSCKVSGYTLIELSIHWVRQAPGKGLEWMGGFDPEDVETIYAQKFQGRVTMTEDTSTDTAYMELSSLTSEDRAVYYCATQPAAIGGTPPYYWGQGTLVTVSSGGGGSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAWDASLSGHVVFGGGTKLTVLRGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 63 - protein, 509 aa, Artificial Sequence
  Description of Artificial Sequence: Synthetic polypeptide
MEFGLSWVFLVALFRGVQSQVQLVQSGAEVKKPGASVKVSCKVSGYTLIELSIHWVRQAPGKGLEWMGGFDPEDVETIYAQKFQGRVTMTEDTSTDTAYMELSSLTSEDRAVYYCATQPAAIGGTPPYYWGQGTLVTVSSGGGGSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAWDASLSGHVVFGGGTKLTVLDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 64 - protein, 512 aa, Artificial Sequence
  Description of Artificial Sequence: Synthetic polypeptide
MEFGLSWVFLVALFRGVQSQVQLVQSGAEVKKPGASVKVSCKVSGYTLTELSIHWVRQAPGKGLEWMGGFDPEDGETVYAQKFQGRVTMTEDTSSDTAYMELSSLRSEDTAVYYCATSFPIRGDPSYYYYYYGMDVWGQGTTVTVSSGGGGSGGGGSGGGGSGGGGSDIQMTQSPSSLSASVGDRVTITCRASQGITNYLAWFQQKPGKAPKSLIYAVSSLQSGVPSKFSGSGSGTDFTLTISSLQPEDFATYYCQQYNSYPWTFGQGTKVEIKDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 65 - protein, 508 aa, Artificial Sequence
  Description of Artificial Sequence: Synthetic polypeptide
MEFGLSWVFLVALFRGVQSEVQLVESGGGLVQPGGSLRLSCEASGFTFSSSEINWVRQAPGKGLEWVSHISSSGSIIYYADSVKGRFTISRDNAKNSLYLQMNSLRAEDTAVYYCARRSYRSSWYYYYGMDVWGQGTTVTVSSGGGGSGGGGSGGGGSGGGGSDIQLTQSPSFLSASVGDRVTITCRASQGISSYLAWYQQKPGKAPKLLIYAASTLQSGVPSRFSGSGSGTEFTLTISSLQPEDFATYYCQQLNSYPVTFGQGTKVEIKDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 66 - protein, 506 aa, Artificial Sequence
  Description of Artificial Sequence: Synthetic polypeptide
MEFGLSWVFLVALFRGVQSEVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCARATWLRGVMDVWGQGTTVTVSSGGGGSGGGGSGGGGSGGGGSQSALTQPHSVSESPGKTVTISCTGSSGSIASNYVQWYQQRPGSAPTTVIYEDNQRPSGVPDRFSGSIDSSSNSASLTISGLKTEDEADYYCQSYDSSNHWVFGGGTKLTVLDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 67 - protein, 381 aa, Artificial Sequence
  Description of Artificial Sequence: Synthetic polypeptide
MEFGLSWVFLVALFRGVQSQVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARVWQYYDSSGSFDYWGQGTLVTVSSGGGGSGGGGSGGGGSGGGGSDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGPGTKVDIKRGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 68 - protein, 512 aa, Artificial Sequence
  Description of Artificial Sequence: Synthetic polypeptide
MEFGLSWVFLVALFRGVQSQVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARVWQYYDSSGSFDYWGQGTLVTVSSGGGGSGGGGSGGGGSGGGGSDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGPGTKVDIKDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 69 - protein, 376 aa, Artificial Sequence
  Description of Artificial Sequence: Synthetic polypeptide
MEFGLSWVFLVALFRGVQSEVQLVESGGGVVQPGRSLRLSCAASGFTFSNYAIHWVRQAPGKGLEWVAVISYDGSNKYYADSVKGRFTISRDNSKNTLYLQMNSLRAEDTAVYYCARDFDDSSFWAFDYWGQGTLVTVSSGGGGSGGGGSGGGGSGGGGSDIQLTQSPSSLSASVGDRVTITCRASQSIRSYLNWYQQKPGKAPKLLIYAASSLQSGVPSRFSGSGSGTDFTLTISSLQPDDFATYYCQQSYSTPPATFGQGTKLEIKSRGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 70 - protein, 506 aa, Artificial Sequence
  Description of Artificial Sequence: Synthetic polypeptide
MEFGLSWVFLVALFRGVQSEVQLVESGGGVVQPGRSLRLSCAASGFTFSNYAIHWVRQAPGKGLEWVAVISYDGSNKYYADSVKGRFTISRDNSKNTLYLQMNSLRAEDTAVYYCARDFDDSSFWAFDYWGQGTLVTVSSGGGGSGGGGSGGGGSGGGGSDIQLTQSPSSLSASVGDRVTITCRASQSIRSYLNWYQQKPGKAPKLLIYAASSLQSGVPSRFSGSGSGTDFTLTISSLQPDDFATYYCQQSYSTPPATFGQGTKLEIKDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 71 - protein, 998 aa, Artificial Sequence
  Description of Artificial Sequence: Synthetic polypeptide
MEFGLSWVFLVALFRGVQSEVQLVESGGRVVRPGGSLRLSCAASGFTFDDYGMSWVRQPPGKGLEFVSGLNWNGDITAFTDSVKGRFTISRDNVKSSLYLQMNSLRADDTAFYYCARVRTWGDYTTGEEIINSWYFDLWGRGTLVTVSSGGGGSGGGGSGGGGSGGGGSDIQLTQSPSFLSASVGDRVTITCRASQDISSYLAWYQQKPGNAPKLLIYAASLLQSGVPSRFSAFGSGTEFTLTISSLQPEDFATYYCQHLKSYPLFTFGPGTKVDIKGGGGSGGGGSGGGGSGGGGSQVQLVESGGGVVQPGRSLRLSCAASGFTFSTYAMHWVRQAPGKGLEWVAVISYDANYKYYADSVKGRFTISRDNSKNTLYLQMNSLRAEDTAVYYCAKDSQLRSLLYFEWLSQGYFDYWGQGTLVTVSSASTKGPSVFPLAPSSGGTAALGCLVKDYFPEPVTVSWNSGALTSGVHTFPAVLQSSGLYSLSSVVTVPSSSLGTQTYICNVNHKPSNTKVDKRVEPKGGGGSGGGGSGGGGSGGGGSDIVMTQSPDSLAVSLGERATINCKSSQSVTFNYKNYLAWYQQKPGQPPKLLIYWASTRESGVPDRFSGSGSGTDFTLTISSLQAEDVAVYYCQQHYRTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 72 - protein, 783 aa, Artificial Sequence
  Description of Artificial Sequence: Synthetic polypeptide
MEFGLSWVFLVALFRGVQSQVQLVQSGAEVKKPGASVKVSCKVSGYTLIELSIHWVRQAPGKGLEWMGGFDPEDVETIYAQKFQGRVTMTEDTSTDTAYMELSSLTSEDRAVYYCATQPAAIGGTPPYYWGQGTLVTVSSGGGGSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAWDASLSGHVVFGGGTKLTVLGGGGSGGGGSGGGGSGGGGSQVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARVWQYYDSSGSFDYWGQGTLVTVSSGGGGSGGGGSGGGGSGGGGSDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGPGTKVDIKIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 73 - skipped

SEQ ID NO: 74 - protein, 401 aa, Artificial Sequence
  Description of Artificial Sequence: Synthetic polypeptide
MEFGLSWVFLVALFRGVQSATGVHSQVQLMQSGAEVKKPGASVKVSCKASGDSFTDYYVHWVRQAPGQGLEWMGWISPYSGGTNYAPKFRGRVTMTRDTSITTAHMELGRLRSDDTAVYFCARSVGYDAFDVWGPGTMVTVSSASTGGGGSGGGGSGGGGSGGGGSTGSWAQSVLTQPPSASGTPGQRVTVSCSGSSSNILNNYVYWYQQLPGTAPKLLIYRNNQRPSGVPDRFSGSKSGTSASLAISGLRSEDEADYYCAAWDDSLFVVFGGGTKLTVLGQPKAAPSVTLFPPSRGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIEQKLISEEDL

SEQ ID NO: 75 - protein, 519 aa, Artificial Sequence
  Description of Artificial Sequence: Synthetic polypeptide
MEFGLSWVFLVALFRGVQSATGVHSQVQLVQSGAEVKKPGASVKVSCKASGYTFTSYAISWVRQAPGQGLEWMGWVSAYNGNTNYAQKLQGRVTMTTDTSTNTAYMELRSLRSDDTAIYYCAIPYSSVTFDCWGQGTLVTVSSASTGGGGSGGGGSGGGGSGGGGSATGVHSEIVLTQSPGTLSLSPGERATLSCRASQSVSGTFLAWYQQKPGQAPRLLISGASSRATGIPDRFSGSGSGTDFTLTISRLEPEDFAVYYCQQYGSSRPTFGQGTKLEIKRTIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 76 - protein, 541 aa, Artificial Sequence
  Description of Artificial Sequence: Synthetic polypeptide
MEFGLSWVFLVALFRGVQSATGVHSEVQLVQSGAEVKKPGESLKISCKGSGYSFTSYWIGWVRQMPGKGLEWMGVIYPGDSDTRYSPSFQGQVTISADKSISTAYLQWSSLKASDTAMYYCARTQWGYNYGSHFFYMDVWGKGTTVTVSSASTGGGGSGGGGSGGGGSGGGGSATGSWAQSVLTQPPSASGTPGQRVTISCSGSSSNIGSDHVYWYQQLPGTAPKLFIYRNNQRPSGVPDRFSGSKSGTSASLAISGLRSEDEADYYCAAWDASLSGYVFGTGTKVTVLGQPKANPTVTLFPPSIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 77 - skipped

SEQ ID NO: 78 - protein, 382 aa, Artificial Sequence
  Description of Artificial Sequence: Synthetic polypeptide
MEFGLSWVFLVALFRGVQSQVQLVQSGAEVKKPGASVKVSCKVSGYTLTELSIHWVRQAPGKGLEWMGGFDPEDGETVYAQKFQGRVTMTEDTSSDTAYMELSSLRSEDTAVYYCATSFPIRGDPSYYYYYYGMDVWGQGTTVTVSSGGGGSGGGGSGGGGSGGGGSDIQMTQSPSSLSASVGDRVTITCRASQGITNYLAWFQQKPGKAPKSLIYAVSSLQSGVPSKFSGSGSGTDFTLTISSLQPEDFATYYCQQYNSYPWTFGQGTKVEIKSRGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 79-80 - skipped (2 entries)

SEQ ID NO: 81 - protein, 127 aa, Artificial Sequence
  Description of Artificial Sequence: Synthetic polypeptide
ATGVHSQVQLMQSGAEVKKPGASVKVSCKASGDSFTDYYVHWVRQAPGQGLEWMGWISPYSGGTNYAPKFRGRVTMTRDTSITTAHMELGRLRSDDTAVYFCARSVGYDAFDVWGPGTMVTVSSAST

SEQ ID NO: 82 - protein, 129 aa, Artificial Sequence
  Description of Artificial Sequence: Synthetic polypeptide
TGSWAQSVLTQPPSASGTPGQRVTVSCSGSSSNILNNYVYWYQQLPGTAPKLLIYRNNQRPSGVPDRFSGSKSGTSASLAISGLRSEDEADYYCAAWDDSLFVVFGGGTKLTVLGQPKAAPSVTLFPPS

SEQ ID NO: 83 - protein, 127 aa, Artificial Sequence
  Description of Artificial Sequence: Synthetic polypeptide
ATGVHSQVQLVQSGAEVKKPGASVKVSCKASGYTFTSYAISWVRQAPGQGLEWMGWVSAYNGNTNYAQKLQGRVTMTTDTSTNTAYMELRSLRSDDTAIYYCAIPYSSVTFDCWGQGTLVTVSSAST

SEQ ID NO: 84 - protein, 116 aa, Artificial Sequence
  Description of Artificial Sequence: Synthetic polypeptide
ATGVHSEIVLTQSPGTLSLSPGERATLSCRASQSVSGTFLAWYQQKPGQAPRLLISGASSRATGIPDRFSGSGSGTDFTLTISRLEPEDFAVYYCQQYGSSRPTFGQGTKLEIKRT

SEQ ID NO: 85 - protein, 134 aa, Artificial Sequence
  Description of Artificial Sequence: Synthetic polypeptide
ATGVHSEVQLVQSGAEVKKPGESLKISCKGSGYSFTSYWIGWVRQMPGKGLEWMGVIYPGDSDTRYSPSFQGQVTISADKSISTAYLQWSSLKASDTAMYYCARTQWGYNYGSHFFYMDVWGKGTTVTVSSAST

SEQ ID NO: 86 - protein, 131 aa, Artificial Sequence
  Description of Artificial Sequence: Synthetic polypeptide
ATGSWAQSVLTQPPSASGTPGQRVTISCSGSSSNIGSDHVYWYQQLPGTAPKLFIYRNNQRPSGVPDRFSGSKSGTSASLAISGLRSEDEADYYCAAWDASLSGYVFGTGTKVTVLGQPKANPTVTLFPPS

SEQ ID NO: 87 - protein, 20 aa, Artificial Sequence
  Description of Artificial Sequence: Synthetic peptide
GGGGSGGGGSGGGGSGGGGS